SEQUENCE LISTING (101 sequences)

SEQ ID NO: 1 - DNA, 2322 bp, Homo sapiens
ctcttccggtttcatatgaactctcccgccacccgggaacagtggctgccaccgtttgtgttttcccgagtttgaattcttgcaggtgaccaagatggagttttctggaagaaagtggaggaagctgaggttggcaggtgaccagaggaatgcttcctaccctcattgccttcagttttacttgcagccaccttctgaaaacatatctttaatagaatttgaaaacttggctattgatagagttaaattgttaaaatcagttgaaaatcttggagtgagctatgtgaaaggaactgaacaataccagagtaagttggagagtgagcttcggaagctcaagttttcctacagagaaaacttagaagatgaatatgaaccacgaagaagagatcatatttctcattttattttgcggcttgcttattgccagtctgaagaacttagacgctggttcattcaacaagaaatggatctccttcgatttagatttagtattttacccaaggataaaattcaggatttcttaaaggatagccaattgcagtttgaggctataagtgatgaagagaagactcttcgagaacaggagattgttgcctcatcaccaagtttaagtggacttaagttggggttcgagtccatttataagatcccttttgctgatgctctggatttgtttcgaggaaggaaagtctatttggaagatggctttgcttacgtaccacttaaggacattgtggcaatcatcctgaatgaatttagagccaaactgtccaaggctttggcattaacagccaggtccttgcctgctgtgcagtctgatgaaagacttcagcctctgctcaatcacctcagtcattcctacactggccaagattacagtacccagggaaatgttgggaagatttctttagatcagattgatttgctttctaccaaatccttcccaccttgcatgcgtcagttacataaagccttgcgggaaaatcaccatcttcgtcatggaggccgaatgcagtatggcctatttctgaagggcattggtttaactttggaacaggcattgcagttctggaagcaagaatttatcaaaggaaagatggatccagacaagtttgataaaggttactcttacaacatccgtcacagctttggaaaggaaggcaagaggacagactatacacctttcagttgcctgaagattattctgtccaatccaccaagccaaggggattatcatgggtgcccattccgtcacagtgatccagagctgctgaagcaaaagttgcagtcatacaagatctctcctggagggataagccagattttggatttagtaaaggggacacattaccaggtagcctgtcaaaaatactttgagatgatacacaatgtggatgattgtggcttttctttgaatcatcctaatcagttcttttgtgagagccaacgtattctaaatggtggtaaagacataaagaaggaacctatccaaccagaaactcctcaacccaaaccaagtgtccagaaaaccaaggatgcatcatctgctctggcctctttaaattcctctctggaaatggatatggaaggactagaagattactttagtgaagattcttaggcagttttataaccctttttcctcaatagcctgtttcctgtttttaagattttgcctttgttgttgaaaaagggtttcactctgtcaccaaggcttagtgcagtgacacaattacagctgattgcagccttgaccttcccagctcaagtgatcctcctacctcagcctcccaagtagttaggaccacaggtgtgcacctcatatccagataatttttttcaatttttttttgtagaggtggggggtctccctatgttgcccaggcagatctcagactcctgggctcaagcgatcctcacacctcagcgtcccagagtgctgggattacggttgtgagccactgtgcctggccttttttttttttttaacctttttgtttaacttctctcttcactgcatcccaatccatctacaggcatgcacacttattaggaaaggaggtttgaggtaacaacagagactttcactatattttgctttgacagaaggaaagaggaggagtttctattaaaatctgtcacttgagtgatgtcatttaagtcctattttaggagataaaaacagctttggggactggttaaagtcccccagaaactacaataaagaacaacttttgttttaactcttaatcactttgtaattttgactcaatccttttctggaccatttttgttaataaatatcaaagtgtaaaaaaa

SEQ ID NO: 2 - protein, 509 aa, Homo sapiens
MEFSGRKWRKLRLAGDQRNASYPHCLQFYLQPPSENISLIEFENLAIDRVKLLKSVENLGVSYVKGTEQYQSKLESELRKLKFSYRENLEDEYEPRRRDHISHFILRLAYCQSEELRRWFIQQEMDLLRFRFSILPKDKIQDFLKDSQLQFEAISDEEKTLREQEIVASSPSLSGLKLGFESIYKIPFADALDLFRGRKVYLEDGFAYVPLKDIVAIILNEFRAKLSKALALTARSLPAVQSDERLQPLLNHLSHSYTGQDYSTQGNVGKISLDQIDLLSTKSFPPCMRQLHKALRENHHLRHGGRMQYGLFLKGIGLTLEQALQFWKQEFIKGKMDPDKFDKGYSYNIRHSFGKEGKRTDYTPFSCLKIILSNPPSQGDYHGCPFRHSDPELLKQKLQSYKISPGGISQILDLVKGTHYQVACQKYFEMIHNVDDCGFSLNHPNQFFCESQRILNGGKDIKKEPIQPETPQPKPSVQKTKDASSALASLNSSLEMDMEGLEDYFSEDS

SEQ ID NO: 3 - DNA, 871 bp, Homo sapiens
ctcttccggtttcatatgaactctcccgccacccgggaacagtggctgccaccgtttgtgttttcccgagtttgaattcttgcaggtgaccaagatggagttttctggaagaaagtggaggaagctgaggttggcaggtgaccagaggaatgcttcctaccctcattgccttcagttttacttgcagccaccttctgaaaacatatctttaatagaatttgaaaacttggctattgatagagttaaattgttaaaatcagttgaaaatcttggagtgagctatgtgaaaggaactgaacaataccagagtaagttggagagtgagcttcggaagctcaagttttcctacagagaaaacttagaagatgaatatgaaccacgaagaagagatcatatttctcattttattttgcggcttgcttattgccagtctgaagaacttagacgctggttcattcaacaagaaatggatctccttcgatttagatttagtattttacccaaggataaaattcaggatttcttaaaggatagccaattgcagtttgaggctgtaagtatatttttgtagttatttctaattgttctcaccattcatttttcccttctgtcttaagtgctggtactaatgtgtagtgtgttctctttacattctccaagtacctgcctgaaacagtagctaatagctttggataacaatatatctttccttctctatgactagaagaaaaggactccttaataacaaagtctcacacttacttgctctgttaaatgtgtgctttattaaagcacaagaaggtttagtttataaaggttcatctttaaccaaaattttgtcggccaaaataaagctaataatgtgttaaac

SEQ ID NO: 4 - protein, 158 aa, Homo sapiens
MEFSGRKWRKLRLAGDQRNASYPHCLQFYLQPPSENISLIEFENLAIDRVKLLKSVENLGVSYVKGTEQYQSKLESELRKLKFSYRENLEDEYEPRRRDHISHFILRLAYCQSEELRRWFIQQEMDLLRFRFSILPKDKIQDFLKDSQLQFEAVSIFL

SEQ ID NO: 5 - DNA, 902 bp, Homo sapiens
gatttactgaacatggctgctgaaatgtataacattattgtgcattattgctaccgtgatatgcggatttatattgcaattgcctctggattcagaaaaacacccttcacagtgaggtgaccaagatggagttttctggaagaaagtggaggaagctgaggttggcaggtgaccagaggaatgcttcctaccctcattgccttcagttttacttgcagccaccttctgaaaacatatctttaatagaatttgaaaacttggctattgatagagttaaattgttaaaatcagttgaaaatcttggagtgagctatgtgaaaggaactgaacaataccagagtaagttggagagtgagcttcggaagctcaagttttcctacagagaaaacttagaagatgaatatgaaccacgaagaagagatcatatttctcattttattttgcggcttgcttattgccagtctgaagaacttagacgctggttcattcaacaagaaatggatctccttcgatttagatttagtattttacccaaggataaaattcaggatttcttaaaggatagccaattgcagtttgaggctgtaagtatatttttgtagttatttctaattgttctcaccattcatttttcccttctgtcttaagtgctggtactaatgtgtagtgtgttctctttacattctccaagtacctgcctgaaacagtagctaatagctttggataacaatatatctttccttctctatgactagaagaaaaggactccttaataacaaagtctcacacttacttgctctgttaaatgtgtgctttattaaagcacaagaaggtttagtttataaaggttcatctttaaccaaaattttgtcggccaaaataaagctaataatgtgttaaac

SEQ ID NO: 6 - protein, 158 aa, Homo sapiens; identical to SEQ ID NO: 4

SEQ ID NO: 7 - DNA, 5765 bp, Homo sapiens
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 8 - protein, 188 aa, Homo sapiens [sequence omitted]

SEQ ID NO: 9 - DNA, 5889 bp, Homo sapiens
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 10 - protein, 189 aa, Homo sapiens [sequence omitted]

SEQ ID NO: 11 - DNA, 4317 bp, Homo sapiens
agagtgaaaaaggcgagccaccaaaacccatctccagtctcctcccgggggcccccagcccgcctctgtgccactttgcatcccacgccggaggaggcattaacgagaccgggtaaggctttttaaacggtccaaggtgtagagccatacttcaggaggatcctcagaagttttggacaagcctccccaaatgtggcaggtgctgtgctggccattggtgacccaaagatgatgaaaaatatgttcctgcccacaaggagttagcgacctactgggctttcctcttgctgatgacatgattcctgtttgaatctgttgacaagattctgaaagctgaacagagaattctggcactgcactgggtaggaaaaagcatttcaagaaatagataatatcaaggacatcaggacaccgggagtgggagagattggactgggagactcagcaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaactgataagggtcaaaagattgttgtgaccttaggacttaaagaagccctaactggttcatccttaccagtggccaagcacattaactttctcatacactgactgttactttaactgttagtcttaaatagttgggacatcagctgactaatagacctcagcctcaaaaggcttggaaagaaaaaacaaatacaacaagcaaacaacaatatcaacaacaagagattgaaataagctatgggtaaaataatgccagtaattcagctgctacatccaagcactgaagtcttacccgtcaacttttttttttttttaaataaactttatggctgtttgttctacaatgttctagaaattctcactcaggtacacagtgccaacaagtggcttgtgaatgtgttttgttgttttgtgctacaatttttaaaaagaaaaaagttttgttttgttttttggggtttctgggttttttccttttctttttctttcctttcattttttttctttgtaatgcacctgacagaaaaaaaagaaaaatgaatttctctttacttctctccaccttctccatctctctactttaaagatggaagtctgtgcatgaggggaaagagggaaaaagagcctgtttttaacttccttgctatccaccacaaaataagcaattattttctttagaggactttatctattgcacaccacactacatctttgagcaagtgccaaatttgtactgaagtgttgaccaagttcattttttctctttactttttccttttccttcttaagttaggacagtgttaaatcttagacaatcccttgaaaaacctgaaataccagcagctggtgagatttgacttttttttttaatggaaactgtaggtgctgttctcaggtgaaaagagagagagagagagagacataagaaatttagagaaaaatattttctgatcttggatttttgtgtgtatgtatgtatgtgattatggtactaataataggaataacgttggaccattgtgagttaaacccacatctggggatgaaatcccacatcctcccaagtgactggtctagaaataatcttgaccttgactttgcacttcaaatgacaacttaaccaagtatagggctcagaaattatatttttaaatgtctgattattattggatggatcaggtggccctgtgtaatagaggtgtgcatgtataacatggaagctactagcaaactgctcccagatgtcctttctccctggtcagttggttccattaacgtttgctacttagtgatttttgtttttcctgttgatattttgagcaaaacaatcattgttttcattgaatatatttggccattttttcagacaaatagaattagcttatttcttcaacattccatcctttcccgatcaggaaatgaaactgatgattttataaggtatttttcacccctccatgaagtgaggtggaggcctttagcatttcagaagtgtgggccatatgtagttcatgccataaaaagtaggatttaattaaaagtcattgcagcccaataaaatggagcctggctgcacccagggatccttgccactgctcttcccttgctgtcagattaatccactgaagtccaactttggttcaagcagagtatttgcaaagagcaacaactgaatgtgatgggactgcttatgtagattttgccagccaaatgccaaggcagttgtagggcctgtacaaataaatgcaaaatcatttcaagtcaattgccattatttgtattgaagtatcagatagatagtaaatactgcaactagtagcttgatgtgctatagttttcactccagtcatcattttcctatctcaccccccgaaacaccaccctaaagttggatttttacatataaataaaaaaagaatcccttttaaaaaaaaaaaaaaaaaa

SEQ ID NO: 12 - protein, 642 aa, Homo sapiens
MSSKRPASPYGEADGEVAMVTSRQKVEEEESDGLPAFHLPLHVSFPNKPHSEEFQPVSLLTQETCGHRTPTSQHNTMEVDGNKVMSSFAPHNSSTSPQKAEEGGRQSGESLSSTALGTPERRKGSLADVVDTLKQRKMEELIKNEPEETPSIEKLLSKDWKDKLLAMGSGNFGEIKGTPESLAEKERQLMGMINQLTSLREQLLAAHDEQKKLAASQIEKQRQQMELAKQQQEQIARQQQQLLQQQHKINLLQQQIQVQGQLPPLMIPVFPPDQRTLAAAAQQGFLLPPGFSYKAGCSDPYPVQLIPTTMAAAAAATPGLGPLQLQQLYAAQLAAMQVSPGGKLPGIPQGNLGAAVSPTSIHTDKSTNSPPPKSKEKTTLESLTQQLAVKQNEEGKFSHAMMDFNLSGDSDGSAGVSESRIYRESRGRGSNEPHIKRPMNAFMVWAKDERRKILQAFPDMHNSNISKILGSRWKAMTNLEKQPYYEEQARLSKQHLEKYPDYKYKPRPKRTCLVDGKKLRIGEYKAIMRNRRQEMRQYFNVGQQAQIPIATAGVVYPGAIAMAGMPSPHLPSEHSSVSSSPEPGMPVIQSTYGVKGEEPHIKEEIQAEDINGEIYDEYDEEEDDPDVDYGSDSENHIAGQAN

SEQ ID NO: 13 - DNA, 4357 bp, Homo sapiens
agtaggagtggagagcgtgcgtgagtgagtgtgtgtgtgtgtgtgtgtgtgtgtgcatgcgtgtgtgaagaatgcacactatctcctgttggtaagtgtgtgcttactccctgaccagatgctgcggtgcacggggcagccaccatctctgcatgcatgtctgtgatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaactgataagggtcaaaagattgttgtgaccttaggacttaaagaagccctaactggttcatccttaccagtggccaagcacattaactttctcatacactgactgttactttaactgttagtcttaaatagttgggacatcagctgactaatagacctcagcctcaaaaggcttggaaagaaaaaacaaatacaacaagcaaacaacaatatcaacaacaagagattgaaataagctatgggtaaaataatgccagtaattcagctgctacatccaagcactgaagtcttacccgtcaacttttttttttttttaaataaactttatggctgtttgttctacaatgttctagaaattctcactcaggtacacagtgccaacaagtggcttgtgaatgtgttttgttgttttgtgctacaatttttaaaaagaaaaaagttttgttttgttttttggggtttctgggttttttccttttctttttctttcctttcattttttttctttgtaatgcacctgacagaaaaaaaagaaaaatgaatttctctttacttctctccaccttctccatctctctactttaaagatggaagtctgtgcatgaggggaaagagggaaaaagagcctgtttttaacttccttgctatccaccacaaaataagcaattattttctttagaggactttatctattgcacaccacactacatctttgagcaagtgccaaatttgtactgaagtgttgaccaagttcattttttctctttactttttccttttccttcttaagttaggacagtgttaaatcttagacaatcccttgaaaaacctgaaataccagcagctggtgagatttgacttttttttttaatggaaactgtaggtgctgttctcaggtgaaaagagagagagagagagagacataagaaatttagagaaaaatattttctgatcttggatttttgtgtgtatgtatgtatgtgattatggtactaataataggaataacgttggaccattgtgagttaaacccacatctggggatgaaatcccacatcctcccaagtgactggtctagaaataatcttgaccttgactttgcacttcaaatgacaacttaaccaagtatagggctcagaaattatatttttaaatgtctgattattattggatggatcaggtggccctgtgtaatagaggtgtgcatgtataacatggaagctactagcaaactgctcccagatgtcctttctccctggtcagttggttccattaacgtttgctacttagtgatttttgtttttcctgttgatattttgagcaaaacaatcattgttttcattgaatatatttggccattttttcagacaaatagaattagcttatttcttcaacattccatcctttcccgatcaggaaatgaaactgatgattttataaggtatttttcacccctccatgaagtgaggtggaggcctttagcatttcagaagtgtgggccatatgtagttcatgccataaaaagtaggatttaattaaaagtcattgcagcccaataaaatggagcctggctgcacccagggatccttgccactgctcttcccttgctgtcagattaatccactgaagtccaactttggttcaagcagagtatttgcaaagagcaacaactgaatgtgatgggactgcttatgtagattttgccagccaaatgccaaggcagttgtagggcctgtacaaataaatgcaaaatcatttcaagtcaattgccattatttgtattgaagtatcagatagatagtaaatactgcaactagtagcttgatgtgctatagttttcactccagtcatcattttcctatctcaccccccgaaacaccaccctaaagttggatttttacatataaataaaaaaagaatcccttttaaaaaaaaaaaaaaaaaa

SEQ ID NO: 14 - protein, 753 aa, Homo sapiens
MSVMSSKRPASPYGEADGEVAMVTSRQKVEEEESDGLPAFHLPLHVSFPNKPHSEEFQPVSLLTQETCGHRTPTSQHNTMEVDGNKVMSSFAPHNSSTSPQKAEEGGRQSGESLSSTALGTPERRKGSLADVVDTLKQRKMEELIKNEPEETPSIEKLLSKDWKDKLLAMGSGNFGEIKGTPESLAEKERQLMGMINQLTSLREQLLAAHDEQKKLAASQIEKQRQQMELAKQQQEQIARQQQQLLQQQHKINLLQQQIQVQGQLPPLMIPVFPPDQRTLAAAAQQGFLLPPGFSYKAGCSDPYPVQLIPTTMAAAAAATPGLGPLQLQQLYAAQLAAMQVSPGGKLPGIPQGNLGAAVSPTSIHTDKSTNSPPPKSKDEVAQPLNLSAKPKTSDGKSPTSPTSPHMPALRINSGAGPLKASVPAALASPSARVSTIGYLNDHDAVTKAIQEARQMKEQLRREQQVLDGKVAVVNSLGLNNCRTEKEKTTLESLTQQLAVKQNEEGKFSHAMMDFNLSGDSDGSAGVSESRIYRESRGRGSNEPHIKRPMNAFMVWAKDERRKILQAFPDMHNSNISKILGSRWKAMTNLEKQPYYEEQARLSKQHLEKYPDYKYKPRPKRTCLVDGKKLRIGEYKAIMRNRRQEMRQYFNVGQQAQIPIATAGVVYPGAIAMAGMPSPHLPSEHSSVSSSPEPGMPVIQSTYGVKGEEPHIKEEIQAEDINGEIYDEYDEEEDDPDVDYGSDSENHIAGQAN

SEQ ID NO: 15 - DNA, 4333 bp, Homo sapiens
gagaaaatcaattggtttagaaggtttggactcacttgacaggttcagttggagacgatcataggtggctgctgtgacaaagggaaattgtgcttttccagcatgcttactgaccctgatttacctcaggagtttgaaaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaactgataagggtcaaaagattgttgtgaccttaggacttaaagaagccctaactggttcatccttaccagtggccaagcacattaactttctcatacactgactgttactttaactgttagtcttaaatagttgggacatcagctgactaatagacctcagcctcaaaaggcttggaaagaaaaaacaaatacaacaagcaaacaacaatatcaacaacaagagattgaaataagctatgggtaaaataatgccagtaattcagctgctacatccaagcactgaagtcttacccgtcaacttttttttttttttaaataaactttatggctgtttgttctacaatgttctagaaattctcactcaggtacacagtgccaacaagtggcttgtgaatgtgttttgttgttttgtgctacaatttttaaaaagaaaaaagttttgttttgttttttggggtttctgggttttttccttttctttttctttcctttcattttttttctttgtaatgcacctgacagaaaaaaaagaaaaatgaatttctctttacttctctccaccttctccatctctctactttaaagatggaagtctgtgcatgaggggaaagagggaaaaagagcctgtttttaacttccttgctatccaccacaaaataagcaattattttctttagaggactttatctattgcacaccacactacatctttgagcaagtgccaaatttgtactgaagtgttgaccaagttcattttttctctttactttttccttttccttcttaagttaggacagtgttaaatcttagacaatcccttgaaaaacctgaaataccagcagctggtgagatttgacttttttttttaatggaaactgtaggtgctgttctcaggtgaaaagagagagagagagagagacataagaaatttagagaaaaatattttctgatcttggatttttgtgtgtatgtatgtatgtgattatggtactaataataggaataacgttggaccattgtgagttaaacccacatctggggatgaaatcccacatcctcccaagtgactggtctagaaataatcttgaccttgactttgcacttcaaatgacaacttaaccaagtatagggctcagaaattatatttttaaatgtctgattattattggatggatcaggtggccctgtgtaatagaggtgtgcatgtataacatggaagctactagcaaactgctcccagatgtcctttctccctggtcagttggttccattaacgtttgctacttagtgatttttgtttttcctgttgatattttgagcaaaacaatcattgttttcattgaatatatttggccattttttcagacaaatagaattagcttatttcttcaacattccatcctttcccgatcaggaaatgaaactgatgattttataaggtatttttcacccctccatgaagtgaggtggaggcctttagcatttcagaagtgtgggccatatgtagttcatgccataaaaagtaggatttaattaaaagtcattgcagcccaataaaatggagcctggctgcacccagggatccttgccactgctcttcccttgctgtcagattaatccactgaagtccaactttggttcaagcagagtatttgcaaagagcaacaactgaatgtgatgggactgcttatgtagattttgccagccaaatgccaaggcagttgtagggcctgtacaaataaatgcaaaatcatttcaagtcaattgccattatttgtattgaagtatcagatagatagtaaatactgcaactagtagcttgatgtgctatagttttcactccagtcatcattttcctatctcaccccccgaaacaccaccctaaagttggatttttacatataaataaaaaaagaatcccttttaaaaaaaaaaaaaaaaaa

SEQ ID NO: 16 - protein, 763 aa, Homo sapiens [sequence omitted]

SEQ ID NO: 17 - DNA, 4566 bp, Homo sapiens
agagtgaaaaaggcgagccaccaaaacccatctccagtctcctcccgggggcccccagcccgcctctgtgccactttgcatcccacgccggaggaggcattaacgagaccgggtaaggctttttaaacggtccaaggtgtagagccatacttcaggaggatcctcagaagttttggacaagcctccccaaatgtggcaggtgctgtgctggccattggtgacccaaagatgatgaaaaatatgttcctgcccacaaggagttagcgacctactgggctttcctcttgctgatgacatgattcctgtttgaatctgttgacaagattctgaaagctgaacagagaattctggcactgcactgggtaggaaaaaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaactgataagggtcaaaagattgttgtgaccttaggacttaaagaagccctaactggttcatccttaccagtggccaagcacattaactttctcatacactgactgttactttaactgttagtcttaaatagttgggacatcagctgactaatagacctcagcctcaaaaggcttggaaagaaaaaacaaatacaacaagcaaacaacaatatcaacaacaagagattgaaataagctatgggtaaaataatgccagtaattcagctgctacatccaagcactgaagtcttacccgtcaacttttttttttttttaaataaactttatggctgtttgttctacaatgttctagaaattctcactcaggtacacagtgccaacaagtggcttgtgaatgtgttttgttgttttgtgctacaatttttaaaaagaaaaaagttttgttttgttttttggggtttctgggttttttccttttctttttctttcctttcattttttttctttgtaatgcacctgacagaaaaaaaagaaaaatgaatttctctttacttctctccaccttctccatctctctactttaaagatggaagtctgtgcatgaggggaaagagggaaaaagagcctgtttttaacttccttgctatccaccacaaaataagcaattattttctttagaggactttatctattgcacaccacactacatctttgagcaagtgccaaatttgtactgaagtgttgaccaagttcattttttctctttactttttccttttccttcttaagttaggacagtgttaaatcttagacaatcccttgaaaaacctgaaataccagcagctggtgagatttgacttttttttttaatggaaactgtaggtgctgttctcaggtgaaaagagagagagagagagagacataagaaatttagagaaaaatattttctgatcttggatttttgtgtgtatgtatgtatgtgattatggtactaataataggaataacgttggaccattgtgagttaaacccacatctggggatgaaatcccacatcctcccaagtgactggtctagaaataatcttgaccttgactttgcacttcaaatgacaacttaaccaagtatagggctcagaaattatatttttaaatgtctgattattattggatggatcaggtggccctgtgtaatagaggtgtgcatgtataacatggaagctactagcaaactgctcccagatgtcctttctccctggtcagttggttccattaacgtttgctacttagtgatttttgtttttcctgttgatattttgagcaaaacaatcattgttttcattgaatatatttggccattttttcagacaaatagaattagcttatttcttcaacattccatcctttcccgatcaggaaatgaaactgatgattttataaggtatttttcacccctccatgaagtgaggtggaggcctttagcatttcagaagtgtgggccatatgtagttcatgccataaaaagtaggatttaattaaaagtcattgcagcccaataaaatggagcctggctgcacccagggatccttgccactgctcttcccttgctgtcagattaatccactgaagtccaactttggttcaagcagagtatttgcaaagagcaacaactgaatgtgatgggactgcttatgtagattttgccagccaaatgccaaggcagttgtagggcctgtacaaataaatgcaaaatcatttcaagtcaattgccattatttgtattgaagtatcagatagatagtaaatactgcaactagtagcttgatgtgctatagttttcactccagtcatcattttcctatctcaccccccgaaacaccaccctaaagttggatttttacatataaataaaaaaagaatcccttttaaaaaaaaaaaaaaaaaa

SEQ ID NO: 18 - protein, 750 aa, Homo sapiens
MSSKRPASPYGEADGEVAMVTSRQKVEEEESDGLPAFHLPLHVSFPNKPHSEEFQPVSLLTQETCGHRTPTSQHNTMEVDGNKVMSSFAPHNSSTSPQKAEEGGRQSGESLSSTALGTPERRKGSLADVVDTLKQRKMEELIKNEPEETPSIEKLLSKDWKDKLLAMGSGNFGEIKGTPESLAEKERQLMGMINQLTSLREQLLAAHDEQKKLAASQIEKQRQQMELAKQQQEQIARQQQQLLQQQHKINLLQQQIQVQGQLPPLMIPVFPPDQRTLAAAAQQGFLLPPGFSYKAGCSDPYPVQLIPTTMAAAAAATPGLGPLQLQQLYAAQLAAMQVSPGGKLPGIPQGNLGAAVSPTSIHTDKSTNSPPPKSKDEVAQPLNLSAKPKTSDGKSPTSPTSPHMPALRINSGAGPLKASVPAALASPSARVSTIGYLNDHDAVTKAIQEARQMKEQLRREQQVLDGKVAVVNSLGLNNCRTEKEKTTLESLTQQLAVKQNEEGKFSHAMMDFNLSGDSDGSAGVSESRIYRESRGRGSNEPHIKRPMNAFMVWAKDERRKILQAFPDMHNSNISKILGSRWKAMTNLEKQPYYEEQARLSKQHLEKYPDYKYKPRPKRTCLVDGKKLRIGEYKAIMRNRRQEMRQYFNVGQQAQIPIATAGVVYPGAIAMAGMPSPHLPSEHSSVSSSPEPGMPVIQSTYGVKGEEPHIKEEIQAEDINGEIYDEYDEEEDDPDVDYGSDSENHIAGQAN

SEQ ID NO: 19 - DNA, 3133 bp, Homo sapiens
aaacagtcagttatagtgggttcaccagactgttggagatttgtgccataggagctgtgcatgcatgatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaactgataagggtcaaaagattgttgtgaccttaggacttaaagaagccctaactggttcatccttaccagtggccaagcacattaactttctcatacactgactgttactttaactgttagtcttaaatagttgggacatcagctgactaatagacctcagcctcaaaaggcttggaaagaaaaaacaaatacaacaagcaaacaacaatatcaacaacaagagattgaaataagctatgggtaaaataatgccagtaattcagctgctacatccaagcactgaagtcttacccgtcaacttttttttttttttaaataaactttatggctgtttgttctacaatgttctagaaattctcactcaggtacacagtgccaacaagtggcttgtgaatgtgttttgttgttttgtgctacaatttttaaaaagaaaaaagttttgttttgttttttggggtttctgggttttttccttttctttttctttcctttcattttttttctttgtaatgcacctgacagaaaaaaaagaaaaatgaatttctctttacttctctccaccttctccatctctctactttaaagatggaagtctgtgcatgaggggaaagagggaaaaagagcctgtttttaacttccttgctatccaccacaaaataagcaattattttctttagaggactttatctattgcacaccacactacatctttgagcaagtgccaaatttgtactgaagtgttgaccaagttcattttttctctttactttttccttttccttcttaagttaggacagtgttaaatcttagacaatcccttgaaaaacctgaaataccagcagctggtgagatttgacttttttttttaatggaaactgtaggtgctgttctcaggtgaaaagagagagagagagagagacataagaaatttagagaaaaatattttctgatcttggatttttgtgtgtatgtatgtatgtgattatggtactaataataggaataacgttggaccattgtgagttaaacccacatctggggatgaaatcccacatcctcccaagtgactggtctagaaataatcttgaccttgactttgcacttcaaatgacaacttaaccaagtatagggctcagaaattatatttttaaatgtctgattattattggatggatcaggtggccctgtgtaatagaggtgtgcatgtataacatggaagctactagcaaactgctcccagatgtcctttctccctggtcagttggttccattaacgtttgctacttagtgatttttgtttttcctgttgatattttgagcaaaacaatcattgttttcattgaatatatttggccattttttcagacaaatagaattagcttatttcttcaacattccatcctttcccgatcaggaaatgaaactgatgattttataaggtatttttcacccctccatgaagtgaggtggaggcctttagcatttcagaagtgtgggccatatgtagttcatgccataaaaagtaggatttaattaaaagtcattgcagcccaataaaatggagcctggctgcacccagggatccttgccactgctcttcccttgctgtcagattaatccactgaagtccaactttggttcaagcagagtatttgcaaagagcaacaactgaatgtgatgggactgcttatgtagattttgccagccaaatgccaaggcagttgtagggcctgtacaaataaatgcaaaatcatttcaagtcaattgccattatttgtattgaagtatcagatagatagtaaatactgcaactagtagcttgatgtgctatagttttcactccagtcatcattttcctatctcaccccccgaaacaccaccctaaagttggatttttacatataaataaaaaaagaatcccttttaaaaaaaaaaaaaaaaaa

SEQ ID NO: 20 - protein, 377 aa, Homo sapiens
MHDEVAQPLNLSAKPKTSDGKSPTSPTSPHMPALRINSGAGPLKASVPAALASPSARVSTIGYLNDHDAVTKAIQEARQMKEQLRREQQVLDGKVAVVNSLGLNNCRTEKEKTTLESLTQQLAVKQNEEGKFSHAMMDFNLSGDSDGSAGVSESRIYRESRGRGSNEPHIKRPMNAFMVWAKDERRKILQAFPDMHNSNISKILGSRWKAMTNLEKQPYYEEQARLSKQHLEKYPDYKYKPRPKRTCLVDGKKLRIGEYKAIMRNRRQEMRQYFNVGQQAQIPIATAGVVYPGAIAMAGMPSPHLPSEHSSVSSSPEPGMPVIQSTYGVKGEEPHIKEEIQAEDINGEIYDEYDEEEDDPDVDYGSDSENHIAGQAN

SEQ ID NO: 21 - DNA, 12599 bp, Homo sapiens
cttccagctcccgccgggcggctgtgacggccgcagcgggtcgcagagcagggagtggacacggagtggggagcggagagggaaggaggaggaggaggagcaaaggtgttggagagaaaacttcagaaaggaacaggaaacctgccggggaggcggcggcggctgcggcttctctgggcgcctgggctgcgctcttcgcggggtgtccgcagctgcggcttggccccggtctggcttccccggcgcgcacgcacggctgagccgcgacgctcagtggctcgggccgtgccctccgccgcggctcttggccgctgtagtcccgcgatccgatccgcttctgccgcggcggctccctggagagagggcggcgagggcgcagggaagaagagggacgtccactgtggaacatgaagcagcatgactgagaaaatgtccagcttcctctacataggggacatcgtgtccctgtacgcggagggctcggtcaacggcttcatcagcaccttggggttagtggatgacagatgtgtggtgcacccagaggccggggaccttgccaaccctcccaagaagttcagagactgccttttcaaggtgtgccctatgaacagatattctgcccagaagcaatattggaaagcaaagcaagccaaacaagggaaccacaccgaggcagccttgctgaagaaactacagcacgctgcagaactggaacaaaaacaaaatgaatcggagaataagaaactgttgggagaaattgtaaaatacagtaatgttatacaactactgcatataaaaagcaacaaatatcttactgtcaacaagagattacctgctttactggagaagaatgccatgcgtgtgtccttggatgctgcaggaaatgaagggtcttggttttatattcatccgttctggaaactgagaagcgagggtgacaatattgttgtaggagataaagttgttttgatgcctgtgaatgcagggcagccactacatgccagcaacatagagcttcttgataacccagggtgtaaagaggtgaatgctgtcaattgcaacaccagctggaaaatcactttattcatgaaatatagttcctatcgagaggatgtattaaaaggaggggacgttgttagattatttcatgcggaacaagagaagtttttgacttgtgatgaatatgagaaaaaacagcacattttccttcgtacgaccttgcgccaatcagctacttctgctactagttctaaagcactctgggaaatagaggtggttcatcatgacccatgccgtgggggtgcaggacagtggaacagcttgttcagatttaagcatcttgcaactggaaactatttagctgcagagcttaatcctgattatcgagatgcccaaaatgaaggaaaaaatgtgagagatggagtccctccaacttcaaagaaaaaacgccaggcaggggagaagatcatgtatactttggtttcagtcccgcatggcaatgacattgcatccctttttgaactagatgccacaactcttcagagagctgactgcctggttccaaggaactcatatgttcggttaaggcatttatgcaccaacacatgggtaaccagtactagtatccccatagacacagatgaagagaggcctgttatgttaaagattggaacctgccaaaccaaagaagataaagaagcgttcgcaatcgtgtctgttccactgtctgaagttcgagacttagactttgccaatgatgccaataaagtactagcgaccacagttaaaaagctagaaaacggcacaataactcagaatgaaaggaggtttgtaaccaaattattggaagatctcatattctttgttgctgatgtgcctaataatggacaagaagttctggatgtggttatcactaagccaaaccgagagcgtcaaaaattgatgagggaacaaaacatactggcacaggtatttggaattcttaaagcaccctttaaagagaaagcaggagaaggctcgatgctgagacttgaagatctgggggatcaaagatatgcaccctacaagtacatgctgcggctctgttaccgcgtcctgagacactcgcagcaggattaccggaaaaatcaggaatatattgctaagaatttctgtgtcatgcagtcccagattggctatgatattttggcagaagatactatcacagctttgttgcacaacaacagaaaactactagagaaacatatcacagcaaaagaaatagaaacatttgtcagtttactcaggagaaatcgggagccaaggtttttggattatttgtcagatctgtgtgtgtctaataccactgctatccctgtaactcaagaactcatctgtaaatttatgttgagtccaggcaatgcagacattctcattcaaactaaggtggtctcaatgcaagcagacaaccccatggagagctccatcctttcagatgacattgatgatgaagaagtttggctctattggattgacagcaacaaggaacctcatggcaaagctatcaggcaccttgctcaagaggcaaaagaaggcaccaaagctgacttagaagttcttacctattacaggtaccagctaaacctctttgcaaggatgtgcttggatcgccagtatctggccataaaccagatttctacacagctgtctgtagacctgatcctgcggtgtgtgtcggatgagagcctgccgttcgacctccgagcgtccttctgtcgcctcatgctccacatgcacgttgaccgggatccccaggagtccgtggtgcctgttcgctatgccaggctctggacagaaatccccacaaagatcacaattcatgaatatgattctataacagactcttccagaaatgatatgaagaggaaatttgccctgacaatggaatttgttgaagaatatttgaaagaagttgtaaaccagccctttccttttggggataaagaaaaaaataaactgacatttgaggtggtccacttggctcggaatcttatatactttggattttatagtttcagtgagttattaaggctaacaagaacacttctggctattttagacattgtacaggcccccatgtcatcatactttgaaagattaagcaaatttcaagatggaggaaacaatgtgatgagaaccattcatggggtgggagagatgatgacccagatggtactcagtagaggctccatcttccccatgagcgtgccggatgtgccacccagcatccacccgagcaagcaagggagccccaccgagcacgaggatgtgactgtgatggacaccaagctgaagatcattgagattttgcagtttatcctgagtgtcagactggattataggatctcatatatgctgtcaatatataagaaggagtttggagaggacaatgacaatgcggagacatctgccagtggatctccagacactttactaccatcagctattgttcctgatatagatgaaattgcagctcaggcagaaactatgtttgcgggaagaaaagaaaaaaatccagttcaacttgacgatgaaggaggcaggacgtttttacgggtcctcattcatctgatcatgcacgactacccgcctttgctgtctggagccctgcagctgttgtttaagcacttcagccagagggcagaggttttacaggcatttaagcaggtgcaattactggtgtctaatcaagacgtagataactacaagcaaatcaaggcagatctagaccagcttcgactgacagtagaaaagtctgagctatgggtggagaagagcagcaactatgagaatggagaaataggggaaagtcaagtgaaaggtggtgaagagccaattgaggaatcaaacattttaagtccagtgcaggatggaacaaagaaacctcagattgacagcaacaagagcaataactaccggattgtaaaggagattttgatcaggctaagtaaactctgtgtgcagaataaaaagtgtcggaatcaacatcaacgattactgaaaaatatgggggcgcattcggtggtgttggatcttctgcagataccctatgaaaagaatgatgaaaagatgaatgaagtaatgaatctagcccatacatttctgcagaatttctgtcgaggaaatccacagaatcaagttcttcttcataaacatctgaatttgtttttaactccaggtctccttgaagcagaaaccatgcggcacatcttcatgaacaattaccatctgtgcaacgaaattagcgagagagttgtacaacactttgtgcactgcattgagacacatggccgccacgtggagtacctgaggtttttgcaaacaattgtaaaagcagatggtaaatatgtgaagaaatgccaggatatggtaatgacagagttgataaatgggggtgaagacgtgctgatattttacaatgatagagcatcatttccaatccttctccatatgatgtgttcagagagagaccgaggggatgagagtggccccttagcctaccacatcaccctggtggagttgctggcagcatgcacagaggggaaaaatgtctacactgaaatcaagtgtaattcccttctcccgctggacgacatagtgagggtggtgacccatgacgactgcatccctgaggttaaaattgcttatgtgaactttgttaatcactgttatgttgacactgaagtggaaatgaaagaaatctatacaagtaaccacatttggaaattatttgagaacttcttggtggatatggcaagggtttgcaacacaactacagacaggaaacatgcagacatctttttggaaaagtgtgttactgagtcaataatgaatattgtgagcggcttctttaattctcccttttcagacaatagtaccagcctccagacacatcagccagtttttattcagctactgcaatctgccttcagaatttacaattgcacctggccaaacccagcgcagaaagcctcagtggaatcctgtatcagaactttggctgaagtggcaaaaaatcgtggaattgccattccagtggatttggacagccaagttaatactcttttcatgaagagccattcaaatatggtgcagagagcagcaatgggttggagactatcagctcgctctgggccacgctttaaggaagctcttggagggcctgcttgggattacagaaatattattgaaaagttacaggatgtagtggcctccttggagcaccagttcagcccaatgatgcaggctgaattctcagtgttggttgatgtattgtacagtccagaactgctgttccctgagggaagcgatgcaagaataagatgtggcgctttcatgtcgaagttgattaatcatacaaagaaactaatggagaaagaagaaaaactgtgcattaaaattcttcagacattacgagaaatgttagagaagaaagacagctttgtggaagagggtaacacattaagaaagatacttctgaatcgatactttaaaggtgattatagtattggtgtgaatggacacctatcaggagcctactccaaaactgcacaggtgggaggaagcttttctggacaagattcagataagatggggatatcaatgtcagacattcagtgtctgctggataaagaaggtgcatcagaacttgtcatcgatgttatagtgaacaccaaaaatgacagaattttttcagaaggcattttcctcggcattgccttgcttgaaggaggaaatacacaaacacagtattctttctaccagcagttgcatgaacaaaaaaagtcagaaaaattctttaaagttctctatgatcgaatgaaggctgctcagaaagaaataagatcaacagtgacagttaataccatagatttaggtaacaaaaaaagggacgatgacaatgaattgatgacatctggtccacgaatgagagtaagagattcaacactacatttaaaagagggaatgaaagggcaattaacagaagcttcttcagcaacatccaaagcatattgtgtatacagaagagaaatggatccagaaatagacattatgtgcacaggaccagaagcgggaaacactgaggaaaaatccgcagaggaagtaacaatgagtcccgcaattgccatcatgcagccaatactgagatttcttcagttactgtgtgagaatcacaaccgggaattgcagaacttcttgaggaatcaaaacaacaaaacaaattacaacctagtctgtgagacccttcagtttctggactgcatttgtggaagtacaaccggtggcctgggcctgttgggtctctacatcaatgagaagaatgtagcgctggtcaaccagaacctggagagcttgactgagtattgccagggcccttgccatgaaaatcagacctgtatcgctacacatgagtctaatgggattgatatcatcattgctttgattctgaatgacataaaccctcttggtaaataccgaatggacctggtgctccagctaaagaacaatgcatctaaacttttgctggccattatggaaagcagacatgacagtgagaatgcagaaagaattctttttaacatgagacccagagaactggtggatgtgatgaagaatgcctataaccaaggattggaatgtgaccatggggatgatgagggtggagatgatggtgtttctccaaaagatgttggacacaatatctatattctggcccatcagttggcccgccacaataaactgttgcagcagatgctcaaaccaggatcggatccagatgaaggagatgaagccttaaagtattatgccaaccacactgcacagattgagattgtccggcatgataggaccatggaacaaatagtttttcctgtccccaatatatgtgaatacctcactcgagaatccaagtgccgtgtgttcaatacaactgaaagggatgaacaaggaagtaaagtgaatgactttttccagcaaacagaagatctctacaatgaaatgaagtggcagaagaaaatcaggaataaccctgcactgttctggttctcgaggcacatctctctctgggggagcatttccttcaacctggctgtgttcatcaatttagctgttgctctcttctacccatttggggatgatggagatgaaggtacactttctccattgttctcggttcttctttggatagcagttgcgatctgcacatctatgctgtttttcttctccaagcctgtgggtattcggccgtttcttgtatcaataatgctcagatcaatatatacaataggtcttgggcctacattaatacttcttggtgcagctaatctttgtaataaaattgtttttctggtgagttttgttggaaatcgtggcacgttcacccgtgggtaccgagcagtcatcctggatatggcctttctctatcacgtggcgtatgtcctggtttgcatgctgggcctttttgtccatgaattcttctatagcttcctgctttttgatttggtgtacagggaagagactttgctgaatgtcataaaaagtgtcacacgaaatggccgctctattattctaactgcagtcctggctctcatcctcgtctacctgttttccattattgggttcctttttttgaaggatgacttcactatggaagttgataggctgaaaaaccgaactcctgttacaggcagtcatcaagtgcctactatgactttaactaccatgatggaagcatgtgccaaggagaactgttcacccacaattccagcttcaaatacagctgatgaagagtatgaagatggaattgaaaggacgtgtgacactctccttatgtgcattgtcaccgtgctgaaccagggcctcaggaatggcggtggtgtgggggatgtgctaagaaggccatcgaaagatgagcccttgtttgctgcccgagtggtttatgaccttcttttttatttcattgttatcattattgttctgaacttgatttttggtgttatcatcgatacttttgctgatctcagaagcgaaaaacagaaaaaagaagaaattctaaagacaacttgtttcatctgtggacttgagagagacaagtttgataataaaacggtttcatttgaggagcacattaagtcagaacacaatatgtggcattatttgtacttcatagtcctggtgaaagttaaagacccaacagaatacactggacctgaaagttatgtggctcaaatgattgtggagaagaatttggattggtttcctcggatgcgagccatgtccctcgttagcaatgaaggcgacagtgagcaaaatgaaattcggagccttcaggagaagttggaatcgaccatgagtctggtcaaacagctgtcgggtcagctggcggagctcaaggagcagatgacagaacaaaggaagaataagcagagactgggcttcctcggatcaaacacaccccatgtgaatcatcacatgccaccacactgataccatggggggaagccgtgactagcctttcatcagtgtcctgcctgatcactgaataaagaactgagatggaggggagtgaacagtgcctattgttgaaaagttaaaaacaaccaagtgccaagatgttgagtgggttagctccgagaacaatttataactgtgttttcatggttgcgaagacctaacctcaaatgcatctgctagaaagcgtacatcacacattcgcaatgcatcaggaagaaaaggcttgcccaaaaggctggagagggcagggagcggcaggatggaaggagacacggggcagggagaactctcttctgctaaatcgataggagtcagttttgtcttaaatgctgactacagccactgacatggttggctggaatttctttcttttaattgtggcatataggtttgtgacacaagaagtcatactttggtggctaagttttactaaggaaaataactgaaaagattaaaagtgagagctgaaaagagaaatgataatgcttccaaactgtagctgtcacagggcaatttctttatttataacatgaagcacaatggatttacagctctaggaacttagtactttggagcttttgcctctcacactgacaacataacaggatgtgattgccttctctgggattcagacaggctctgtcaatgtggagcacaaaaggagattttcatataacttgttaaaaacatgttctaagtcatgtataggctaagattttaagaagatctgggggaataaaaagccaacagtaagccccaggaaagggttttttgagaccatatgtatgctattaaaatatattgagattaatacatgaaaatgcttaaaagtgataggaattattgagaaacttatgatggtggcattgccttttataaacatggagtgaaagccatttgactcaacgtttgctgtgcttaaagaattgcttcaggacccgagcgtttttatgtatgctgttcctgcagttaggaaaaaaaaatccaagaaatgtattgaatactcaagaaaatgccacatttcaattacatatttaaaactgtatctgtaagggcttttcaaaatgtagcaagataaaaatctcttattcaaattgtttttgtattaaatcatgcactcagaatttgtcgagggagagaataacctggtgtgttcaggttattcttagagactacacatttggaatagcagagcaaaatatggataatgaaagtgttgggaaaaaagtgatgtgacagggagtgaagacacttgataccagactctgggagatactctgcaagttgacctggcctctcccccacaggaacaaaacactgcctccagagtctttaaattctcagttatcaacgccaaggtttaaggtctagatagggtttgctatagggtttgctctgacaatttttaaagcttttggattgttctaacatagcttaagctattggttcctaaaaatcccaaatcaagatctatgtagaatataaaggaagcctgaaccaatccttccacatactgttaagatgtagacttggaacaaagctgttgggacccagagcaatgaatttttgaactgaagctactggtactccccagcaccacctcatactaagaattcctcactctgacatgacagtattttttctgaccaggagctgaaagaccctgacattcatgatccaaagatataagaaattttgatatgtctgcatgcagcacagaactttgaacctaggaggcagtcaataaatacatgagaaattcagctgtcattcagttactcttaattctttataaaactttaaatgataccacatatttgtttgtttaaaatggctttcccaaaatcaaagtagactaaagcagccatctttaaaatccaggatcatcaatgctattaacagtatcaggtaaaaagtacactttaaaatattttaatcaggcagagttttatggatagagaatagaagagaaaggtagtaaatattgaacatattccaatataggaacctatctctgttttagtacaaaatatttctgacatctgaactagaggtcaagagaataaattcatttgtatacatctgagcaacctgtctttcagatgataaagtatctagccttttctgacaccataatagttcattttgtagggaataagccattaggtgtatataattgctttctagaaatgacctaatgtccccaaccactttgtagtggcagatcactgtttcacagcatattttctcccaaggaaagtattcaaaagagactgcaactaacaagactcttatttcatcaaaatttaaatatttctgagttgtatttttaatgcctctttcttttctgcctaaatgcttagaaattataaagcaaaaaaaaaaaaaacagcaacaaaaaatcgaagcagacaaaaaaggcacttttcagaacatcaaattcctaatgaagaagaggagaagataatggggaaatttgacatttgatataaatttatatttgttatgtgtatgtttgttaatgcaactggaatatttgacttaggtgagtatcagttaacatccttgtatttatatagtgacatcaaaataaatgcaatcatcttgaactttgatgttaagggagattttgaaaaaatatttagttatttcaaattcatattggttcaaaagtatcagtttctgctgaataatatttttaatttcaaaagggttttgttccctctgtgctccattctgaggctcaagagcttaatgccagtatgttttctgagttaaaataacacttttagatgagaaaatgcttgtatcatacagggctataatataaataaataaattgtatgtatgcaaaatttatcatattttctaccccttaaaaaattaagttagaaataagcttattttcttgcacatcaatatttttctgttggtaaagaggacacaatttctagtagattgttaataacatcaggaagatatttctttcgtcagaactaattgtgtgcttattcccatattctcagctcataactccctgttttggctgctctctttattttacaattgtttcattgcaaacagcaaggcagtatagcccacactcagccactagcccccagtccccaggactgagaatgagtggggaggctggggagttggtgagagaaggtggagatagggatttctgcttcccagtactctatcattagagaaaaggaggggtaaatattagctaagaagaagaaaaaagcttattcttcagggacccagtagattggacgtaagcaagtaaatattgcacaggcatcatgatgtaatagttgatggaccccagagatacccctcacccatgcagagaacaaagctgtgtggaagagcaactaccataagatggtgtgttccttccagcggctgttaaagccatcctgaataacaaatcagctattcctacctacagacccattccaatatgctgtgatacacgacacggtgcaccgcagaggagacagacatcttcagcacaaacacagatgcctgcaggagcttggcaagtcactaaattgcatgaaattgtgaggtgcacaccaaatgcagggatgggggaggccgtgggaagctctgtattctcaaaatttgacagctaattccggtttttagaaaatgcctgaggccagtagaggccctctagtcactcactgctgctgtttctgatataattattgagaaagctatctcacttaatagaagaaaacacgcactatcaaaaccagatagcctaacgtgcatgtgaaaatcgagaaagctgaaaacaaatccaggtacccttctctgaactggagtgtttccacagacttgaataatttatgaaattatcacaccagtatttctcatatcaccaagaagactttctctcctgcagtagaggattgttatatttgcctaaaaaacacgattccaatatatgacaagggcagataatttataagtgaatgttaataaaattggatgtgtataacttttttgtttgcaaaaaaaaaaaaaaaaaa

SEQ ID NO: 22 - protein, 2701 aa, Homo sapiens
MTEKMSSFLYIGDIVSLYAEGSVNGFISTLGLVDDRCVVHPEAGDLANPPKKFRDCLFKVCPMNRYSAQKQYWKAKQAKQGNHTEAALLKKLQHAAELEQKQNESENKKLLGEIVKYSNVIQLLHIKSNKYLTVNKRLPALLEKNAMRVSLDAAGNEGSWFYIHPFWKLRSEGDNIVVGDKVVLMPVNAGQPLHASNIELLDNPGCKEVNAVNCNTSWKITLFMKYSSYREDVLKGGDVVRLFHAEQEKFLTCDEYEKKQHIFLRTTLRQSATSATSSKALWEIEVVHHDPCRGGAGQWNSLFRFKHLATGNYLAAELNPDYRDAQNEGKNVRDGVPPTSKKKRQAGEKIMYTLVSVPHGNDIASLFELDATTLQRADCLVPRNSYVRLRHLCTNTWVTSTSIPIDTDEERPVMLKIGTCQTKEDKEAFAIVSVPLSEVRDLDFANDANKVLATTVKKLENGTITQNERRFVTKLLEDLIFFVADVPNNGQEVLDVVITKPNRERQKLMREQNILAQVFGILKAPFKEKAGEGSMLRLEDLGDQRYAPYKYMLRLCYRVLRHSQQDYRKNQEYIAKNFCVMQSQIGYDILAEDTITALLHNNRKLLEKHITAKEIETFVSLLRRNREPRFLDYLSDLCVSNTTAIPVTQELICKFMLSPGNADILIQTKVVSMQADNPMESSILSDDIDDEEVWLYWIDSNKEPHGKAIRHLAQEAKEGTKADLEVLTYYRYQLNLFARMCLDRQYLAINQISTQLSVDLILRCVSDESLPFDLRASFCRLMLHMHVDRDPQESVVPVRYARLWTEIPTKITIHEYDSITDSSRNDMKRKFALTMEFVEEYLKEVVNQPFPFGDKEKNKLTFEVVHLARNLIYFGFYSFSELLRLTRTLLAILDIVQAPMSSYFERLSKFQDGGNNVMRTIHGVGEMMTQMVLSRGSIFPMSVPDVPPSIHPSKQGSPTEHEDVTVMDTKLKIIEILQFILSVRLDYRISYMLSIYKKEFGEDNDNAETSASGSPDTLLPSAIVPDIDEIAAQAETMFAGRKEKNPVQLDDEGGRTFLRVLIHLIMHDYPPLLSGALQLLFKHFSQRAEVLQAFKQVQLLVSNQDVDNYKQIKADLDQLRLTVEKSELWVEKSSNYENGEIGESQVKGGEEPIEESNILSPVQDGTKKPQIDSNKSNNYRIVKEILIRLSKLCVQNKKCRNQHQRLLKNMGAHSVVLDLLQIPYEKNDEKMNEVMNLAHTFLQNFCRGNPQNQVLLHKHLNLFLTPGLLEAETMRHIFMNNYHLCNEISERVVQHFVHCIETHGRHVEYLRFLQTIVKADGKYVKKCQDMVMTELINGGEDVLIFYNDRASFPILLHMMCSERDRGDESGPLAYHITLVELLAACTEGKNVYTEIKCNSLLPLDDIVRVVTHDDCIPEVKIAYVNFVNHCYVDTEVEMKEIYTSNHIWKLFENFLVDMARVCNTTTDRKHADIFLEKCVTESIMNIVSGFFNSPFSDNSTSLQTHQPVFIQLLQSAFRIYNCTWPNPAQKASVESCIRTLAEVAKNRGIAIPVDLDSQVNTLFMKSHSNMVQRAAMGWRLSARSGPRFKEALGGPAWDYRNIIEKLQDVVASLEHQFSPMMQAEFSVLVDVLYSPELLFPEGSDARIRCGAFMSKLINHTKKLMEKEEKLCIKILQTLREMLEKKDSFVEEGNTLRKILLNRYFKGDYSIGVNGHLSGAYSKTAQVGGSFSGQDSDKMGISMSDIQCLLDKEGASELVIDVIVNTKNDRIFSEGIFLGIALLEGGNTQTQYSFYQQLHEQKKSEKFFKVLYDRMKAAQKEIRSTVTVNTIDLGNKKRDDDNELMTSGPRMRVRDSTLHLKEGMKGQLTEASSATSKAYCVYRREMDPEIDIMCTGPEAGNTEEKSAEEVTMSPAIAIMQPILRFLQLLCENHNRELQNFLRNQNNKTNYNLVCETLQFLDCICGSTTGGLGLLGLYINEKNVALVNQNLESLTEYCQGPCHENQTCIATHESNGIDIIIALILNDINPLGKYRMDLVLQLKNNASKLLLAIMESRHDSENAERILFNMRPRELVDVMKNAYNQGLECDHGDDEGGDDGVSPKDVGHNIYILAHQLARHNKLLQQMLKPGSDPDEGDEALKYYANHTAQIEIVRHDRTMEQIVFPVPNICEYLTRESKCRVFNTTERDEQGSKVNDFFQQTEDLYNEMKWQKKIRNNPALFWFSRHISLWGSISFNLAVFINLAVALFYPFGDDGDEGTLSPLFSVLLWIAVAICTSMLFFFSKPVGIRPFLVSIMLRSIYTIGLGPTLILLGAANLCNKIVFLVSFVGNRGTFTRGYRAVILDMAFLYHVAYVLVCMLGLFVHEFFYSFLLFDLVYREETLLNVIKSVTRNGRSIILTAVLALILVYLFSIIGFLFLKDDFTMEVDRLKNRTPVTGSHQVPTMTLTTMMEACAKENCSPTIPASNTADEEYEDGIERTCDTLLMCIVTVLNQGLRNGGGVGDVLRRPSKDEPLFAARVVYDLLFYFIVIIIVLNLIFGVIIDTFADLRSEKQKKEEILKTTCFICGLERDKFDNKTVSFEEHIKSEHNMWHYLYFIVLVKVKDPTEYTGPESYVAQMIVEKNLDWFPRMRAMSLVSNEGDSEQNEIRSLQEKLESTMSLVKQLSGQLAELKEQMTEQRKNKQRLGFLGSNTPHVNHHMPPH

SEQ ID NO: 23 - DNA, 2988 bp, Homo sapiens
gagtttttccaggggaaaccggcctgggtggagagacagagaaggggagaccgggtgggtgggtcgtacgcttagggggccgtggttggtacttcgctgttggggaggctttcaggtcgcccagatcctgcttcccaatgggtgactgggaaaattagcgggtggaaaatctcgaggggtggaggctttttttttcctccttggccccgccctcttcctgttcggcgactgggacgcctggccctacgagggggaagggaggcttggccgggctatcggagagcgctgtgcgcacgcgcgagcccgagtgcgtgtgtgtgtgtgcacgcgcacgcgcgctcctgctcttaggaagcctgggaaggaccggtgtgctaggagatgatcggggaaagcatagtcccctgtctgtggcaccagacactcccgactgtgcgctgactctccccgcccagccagcagccttttccagagaggctgtggtccatagcctctgttcgttttcactgcaggaccaggcacgaaagttaaaacaaaatgaagattttttctgaatctcataaaacagtgtttgttgtggatcactgcccttatatggcagaatcttgcaggcagcatgtcgagtttgatatgctggtgaagaatagaacccaaggaatcattcctttggcccccatatctaaatcattgtggacttgctcagtagaatcttccatggaatattgtagaataatgtatgatatatttcctttcaaaaagctggtgaattttattgtgagtgactctggagcacatgttttaaattcttggactcaagaagaccaaaatttacaggagctaatggcagcattagccgctgttgggcctcctaatcctcgggcagatccagagtgctgcagtattctgcatggccttgttgcagcagtggaaactctctgcaaaattactgaataccaacatgaggctcgtactctactcatggagaatgcagaacgtgttggaaatagaggacgaataatctgtattactaatgcaaaaagtgatagtcatgtgcgaatgcttgaagactgtgtccaggaaacgattcatgaacataacaagcttgctgcaaattcagatcatctcatgcagattcaaaaatgtgagttggtcttgatccacacctacccagttggtgaagacagccttgtatctgatcgttctaaaaaagagttgtccccggttttaaccagtgaagttcatagtgttcgtgcaggacggcatcttgctaccaaattgaatattttagtacagcaacattttgacttggcttcaactactattacaaatattccaatgaaggaagaacagcatgctaacacatctgccaattatgatgtggagctacttcatcacaaagatgcacatgtagatttcctgaaaagtggtgattcgcatctaggtggcggcagtcgagaaggctcgtttaaagaaacaataacattaaagtggtgtacaccaaggacaaataacattgaattacactattgtactggagcttatcggatttcacctgtagatgtaaatagtagaccttcctcctgccttactaattttcttctaaatggtcgttctgttttattggaacaaccacgaaagtcaggttctaaagtcattagtcatatgcttagtagccatggaggagagatttttttgcacgtccttagcagttctcgatccattctagaagatccaccttcaattagtgaaggatgtggaggaagagttacagactaccggattacagattttggtgaatttatgagggaaaacagattaactccttttctagaccccagatataaaatcgatggaagtcttgaggtccctttggaacgagcaaaagatcagttagaaaaacatacccgttactggcctatgatcatttcacaaaccaccatttttaacatgcaagcggtagttccattagccagtgttattgtgaaagaatctctgacagaagaagatgtgttaaactgtcaaaaaacaatatacaacttagttgatatggaaagaaaaaatgatcctctacctatttccacagttggtacaagaggaaagggccctaaaagagatgaacaataccgtatcatgtggaatgaattagaaacccttgtcagagcccatatcaacaactcagagaaacatcaaagagtcttggaatgtctgatggcatgcaggagcaaacccccagaagaggaagaacgaaagaaacgaggaagaaagagggaagacaaagaggacaagtcagagaaagcagtgaaagattatgaacaggaaaagtcttggcaagactcagagagattaaaaggaatcttagagcgtggaaaagaagaattggctgaagctgagattataaaagattcgcctgattccccagaacctccaaacaaaaaaccccttgttgaaatggatgaaactccacaagtggaaaaatcaaaagggccagtgtcgttattatccttgtggagtaatagaatcaatactgccaattccagaaaacatcaggaatttgctggacgtttgaactctgttaataacagagctgaactatatcaacatcttaaagaggaaaatgggatggagacaacagaaaatggaaaagccagccggcagtgaagagtgacttgaagaactaaatttagcatattgcaaaaatattttgtgcggaattcgatataagtacttttacagcaagatggtatagttatgttgcctggactggtttttacatttttaaaatatttcagctgtcatttttgtactaattataaaattggcacataattcaaaaatatacatttgagatgatttgtcctcccaaattatacaagtttattttatggtataaagtgttctctctggaaatgtttttaaaaaaattcttaggcttctctttgcgaaataaaactattaaaatatttgaaatgcaaaacaaaaaaaaaaaaa

SEQ ID NO: 24 - protein, 706 aa, Homo sapiens
MKIFSESHKTVFVVDHCPYMAESCRQHVEFDMLVKNRTQGIIPLAPISKSLWTCSVESSMEYCRIMYDIFPFKKLVNFIVSDSGAHVLNSWTQEDQNLQELMAALAAVGPPNPRADPECCSILHGLVAAVETLCKITEYQHEARTLLMENAERVGNRGRIICITNAKSDSHVRMLEDCVQETIHEHNKLAANSDHLMQIQKCELVLIHTYPVGEDSLVSDRSKKELSPVLTSEVHSVRAGRHLATKLNILVQQHFDLASTTITNIPMKEEQHANTSANYDVELLHHKDAHVDFLKSGDSHLGGGSREGSFKETITLKWCTPRTNNIELHYCTGAYRISPVDVNSRPSSCLTNFLLNGRSVLLEQPRKSGSKVISHMLSSHGGEIFLHVLSSSRSILEDPPSISEGCGGRVTDYRITDFGEFMRENRLTPFLDPRYKIDGSLEVPLERAKDQLEKHTRYWPMIISQTTIFNMQAVVPLASVIVKESLTEEDVLNCQKTIYNLVDMERKNDPLPISTVGTRGKGPKRDEQYRIMWNELETLVRAHINNSEKHQRVLECLMACRSKPPEEEERKKRGRKREDKEDKSEKAVKDYEQEKSWQDSERLKGILERGKEELAEAEIIKDSPDSPEPPNKKPLVEMDETPQVEKSKGPVSLLSLWSNRINTANSRKHQEFAGRLNSVNNRAELYQHLKEENGMETTENGKASRQ

SEQ ID NO: 25 - DNA, 1975 bp, Homo sapiens
aagctactcagataagaggctccaagaggacatttttggatgtgaaaaacaatgagaaggaggacaacacacatttacaatcgtcttaattttgtactcagaaaaaggatgtgaagacaatgcacagggaatacaatagtttcagatctgtgtacagtttccttttgcttcatctcctgcaacaatgtaatgaagacaccatgatatcattaacatttcacacaaaaggaaaatgaggctgaaatggtgtgggcaaggcccaggaatctggagcatccctaaccaagcaggagagcacctgggatagagaaagtgctcaagaatgttcacttactgattactacaatcaaaaaaagatacgacactaatttaccacattcttcttacttattttatgagatactattcttccaaggtggagaaagtggagaaagtagagtgacgcagctaagggagtaaatcgaccctcagccaacaagtggcaaaagcctgaagaaagtgatcaagatcactgatgaccccgctgcccatctccaagggggcgggtatcacaaccccgacgccacaccacgtatcattccgcaaaactcccgcgcctcccacgcagaactggcaagagggaaggcgagacagcagtgaacagctggtacgcagcacccacagcaccgcggcagcagctagtgccgactcccgcctagctcttttgactctgttcgcgggaagaatggggaaacagtaaggttgcggcgcctcccgcgagacgaggtacctgaggctggccccgcagtcccccgccgcaccagcaccggagcttcacaccccacttccggggtcaagtcaccgccgggaatcctgtgatcgcagaaaggtagtctcaggttccgcccctatccaagtcccgcctccactgcctctcgccctgtatctgtcaacttccgggacgccgcgcgtcactaagcagccaatctccacttccggactcatccagccccttctccacccctttcagagacagcgcgattgcgatttaggtttccgcgcatttaattggcgaagctggagcgctagtcttcgctgattggtgccgagaaatctgccccatagacacccgcggggcgcacagtttcagtcgtccgtgggtttcccgccagccgcagtcttggaccataatcatggtggacatgatggacttgcccaggtcgcgcatcaacgccggcatgctagctcaattcatcgacaagcctgtctgcttcgtagggaggctggaaaagattcatcccaccggaaaaatgtttattctttcagatggagaaggaaaaaatggaaccatcgagttgatggaaccccttgatgaagaaatctctggaattgtggaagtggttggaagagtaaccgccaaggccaccatcttgtgtacatcttatgtccagtttaaagaagatagccatccttttgatcttggactttacaatgaagctgtgaaaattatccatgacttccctcagttttatcctttagggattgtgcaacatgattgatcttgatggattttcatacgattgtaaatgagctatattaaagtctattaaaggaagcccttcttgtttgagggagagatttctgtgctttctcatatttaatttgctgtttttaagatattccaacctagagtttttgatggaactgatatattgacagttctcaccgaagtccttttataaagaattgctactccaatatatggtcagattagatgcaagaataaagcagttgtccgagtctaagtttctattttattaataaaaactaaaatggtacgtactatcggtcatttcattttcattcttttaatcatgtattcaagcacaaacttgaaatttcatagccataaggtcaagatttagacctaccaaataaaaccttgggccagctgtgttaaggatttgctcaccttttcccaaactataccttgata

SEQ ID NO: 26 - protein, 121 aa, Homo sapiens
MVDMMDLPRSRINAGMLAQFIDKPVCFVGRLEKIHPTGKMFILSDGEGKNGTIELMEPLDEEISGIVEVVGRVTAKATILCTSYVQFKEDSHPFDLGLYNEAVKIIHDFPQFYPLGIVQHD

SEQ ID NO: 27 - DNA, 3447 bp, Homo sapiens
aggaagcgggaagttacttagcacggttccgggtttctcgcgccccgcctgtcccccctccctatcactgctactggctcttggtccctccgttggactgtcctgcggagagaaaccccagcccatcggtctgcgctgggaccgcccgccgcgcatctgcccttcttcgctgactccgccccgcatctggccagacccgcctcgcgtcagagctgacccactcactgcgcgtttgccagtcagtctctccggacctgcctcgagcctcaggctgctgaaatcaccgcgcctcactcgcctcgacagtgattctgagtctgcttttagcttccttttgcctgccttggctttttctgttcgtgaacagctgtttggcccatagcttagagaaagcagccttttttctcttcaaagagaacctcctcccagtgctcagagagatggggagcggggagcctaatcctgctggcaagaaaaagaagtatctcaaggccgctctgtacgtgggtgacttggacccagatgtcaccgaggacatgctctataagaagttcaggcctgctggccctctgcgattcacccgaatctgccgtgatccggtgacccgcagccccctgggctatgggtatgttaacttccgctttcccgcggatgcagagtgggccttgaacaccatgaattttgatttgattaatggaaaaccattccgccttatgtggtctcagccagatgaccgcttaagaaagtctggagtgggaaatatattcatcaaaaacctggacaaatccatagacaatagggccctgttttacttattttctgcttttgggaacattctgtcctgcaaagtcgtatgcgatgacaacggctctaagggttatgcctatgttcactttgacagcctggccgctgccaatagagccatctggcacatgaatggagtgcggctcaacaaccgccaggtgtatgttggcagattcaaattcccagaagagcgggcggctgaggtcagaaccagggatagagcaactttcaccaatgttttcgttaaaaacattggagacgacatagatgacgaaaaactgaaggaacttttctgtgaatatgggccaactgagagtgttaaagtaataagagatgccagtgggaaatctaaaggctttggatttgtgagatatgagacacacgaggctgcccaaaaggctgtgctagacttgcatggaaagtccatcgatggaaaagtcctctatgtagggcgagcacagaagaaaattgaacgcctggctgagttgaggcggagatttgaacggctgaggttaaaagaaaaaagtcggcccccaggggtgcctatctatattaagaacttggatgagacaatcaatgatgaaaaactgaaggaggaattttcttcctttgggtcaattagtcgggccaaagtgatgatggaagtggggcaaggcaaaggatttggtgtggtctgcttttcctcttttgaagaggctaccaaagcagtggatgagatgaatggccgcatagtgggctccaagcccctgcatgtcaccctgggccaggccaggcgcaggtgctgagaataagaatgctcagtttgtttcagccttagttggtgcctccttagtttgggctcctttgtgataaggggttattttatgctaattcacaagtttttttttgaagtgaattcttttgaaaaaaaaatgcaaaactagaaaactttattcattttagaatagaacataatttctaactgtaaaattgtcattttgtactttttttgatgtaatatccttagaaatctgtagaataaagtgtattcctccacttttttttcctgaacagtcaaggtgaggcaattgattgagtatatttcccttcttatttcagtaatactctattttttttcatgaaaatgtcaacatggttcttctgaatctatcacagtgaaaagttctaacttgtttttgagaagtcagtacagcaggggaaaacatatgtgatgcaattaacatctgcataatttcacttaaaattattatgcaaaaatgaatgttttttcaaaaaatgtgaaatgtattttattttctttatttgtattcttgtttcattttttaatatgttgtgaacatgctacagatttgatagtacttttgactaaatgttgggagtggtcgtattaacttcttgcccaaagaagtaagcatattggtgttttctcaattagtcactgagaaaattaacactttaggcagtggctatttaaagtaggaattgcatcttaaaaacctttcctaagagatttggtatgtgaggatactttcagtaccactcctaccattcatttttctaaattccttagtacatatacttggatcatgttaaattaacaagaaagatgaataactgcgctgaattgcctttacctataaataatttaatattttaccttcgggttttatcaactgtcaatataaaaggcagtactccacagaatgatgttgaaaaacttcttcgaagaacaccttctattaaacttgttatctcttgtaaattattgtgtgtgtccttttgataatattcacaggtgtttcaaaggtaaggaataggttgtctcttggattaagtcatatgcctccagccattatatgagaactgtgaaaccaatatggttttctttatgtcttggctgcttgaaaattaaaaaaaaaaaaggtttgttcaatattgccgttacatttattagcctgtaatttctaaattggagattctctacatttcacttgcagtttcctgttctcctcattgcctgccttccattcatattacacttatttttctattttttgtattacctttttaaaaatatataccagttcaagtccttttaggaagaagaaaatacctaatttatgtaaaatttaaataattacttttttataatatgattcacttatgccacagattcaacattagaatatgttttatctctactgtcagttttattaccttatatacaaatcttcattttcatacatagtacaatgtaaatatataactttgttaacacttttgttagctctttgaccataaaataatgacaataagctgtttctatgtatttgtttatctacaaattacaggtttatccatttgcaaatattttcaaaatggaaatcactgtttatattgattataaacataagacatgctcattgtaaaaaatgtacacaaggcagaaggaagtaaaatttccacagttcagaaataccacaattaatattttcaatgtgtaaatatcttttcataatttttcctacgtatacacaaacattttgaccaaaaatccacactatatgtactgttctgtatttttaattttaaactgaacaataatcatcttttcctgacaataaatatcaatctctatcatca

SEQ ID NO: 28 - protein, 382 aa, Homo sapiens
MGSGEPNPAGKKKKYLKAALYVGDLDPDVTEDMLYKKFRPAGPLRFTRICRDPVTRSPLGYGYVNFRFPADAEWALNTMNFDLINGKPFRLMWSQPDDRLRKSGVGNIFIKNLDKSIDNRALFYLFSAFGNILSCKVVCDDNGSKGYAYVHFDSLAAANRAIWHMNGVRLNNRQVYVGRFKFPEERAAEVRTRDRATFTNVFVKNIGDDIDDEKLKELFCEYGPTESVKVIRDASGKSKGFGFVRYETHEAAQKAVLDLHGKSIDGKVLYVGRAQKKIERLAELRRRFERLRLKEKSRPPGVPIYIKNLDETINDEKLKEEFSSFGSISRAKVMMEVGQGKGFGVVCFSSFEEATKAVDEMNGRIVGSKPLHVTLGQARRRC

SEQ ID NO: 29 - DNA, 28 bp, Artificial Sequence
  Description of Artificial Sequence Synthetic probe
gagtagatttcaatgaaattgaaacaag

SEQ ID NO: 30 - DNA, 25 bp, Artificial Sequence
  Description of Artificial Sequence Synthetic probe
cctctattttttcttgttagcctag

SEQ ID NO: 31 - DNA, 2175 bp, Homo sapiens
gttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 32 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 33 - DNA, 2119 bp, Homo sapiens
aacatgttgagctctggcatagaagaggctggtggctattttgtccttgggctgcctgttttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaa

SEQ ID NO: 34 - protein, 164 aa, Homo sapiens; identical to SEQ ID NO: 32

SEQ ID NO: 35 - DNA, 2284 bp, Homo sapiens
agctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaa

SEQ ID NO: 36 - protein, 164 aa, Homo sapiens; identical to SEQ ID NO: 32

SEQ ID NO: 37 - DNA, 2325 bp, Homo sapiens
atggtaggagacaggagacctctaaagaccccagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaa

SEQ ID NO: 38 - protein, 198 aa, Homo sapiens
MWGVFRRQTTHSSNPPLPGQQSCCNHRDFFCSGAMSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 39 - DNA, 2122 bp, Homo sapiens
ggtggctattttgtccttgggctgcctgttttcagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 40 - protein, 164 aa, Homo sapiens; identical to SEQ ID NO: 32

SEQ ID NO: 41 - DNA, 4353 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 42 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 43 - DNA, 4353 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 44 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 45 - DNA, 1431 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctgggtaagttgaccatatatcctcacctcatggatattgaattggttatgatataaattggggatttgaagaagagtttctccttttgaccaaataaagtaccattagttga

SEQ ID NO: 46 - protein, 297 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLGKLTIYPHLMDIELVMI

SEQ ID NO: 47 - DNA, 4274 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 48 - protein, 307 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESLSTCWRRCLYWTQIRELQRPKPLHMPTLLSTTILMMNQWPILMISPLKAGTSL

SEQ ID NO: 49 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 50 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 51 - DNA, 86 bp, Homo sapiens
gtagaggagatggcgcaggggacacgggcaaagacttgggggttcctgggaccctcagacgtgtgtcctcttctccctcctcccag

SEQ ID NO: 52 - DNA, 3474 bp, Homo sapiens
gcgccagcttggagagccagccccatcggggttccccgccgccggaagcggaaatagcaccgggcgccgccacagtagctgtaactgccaccgcgatgccgaaggcgcccaagcagcagccgccggagcccgagtggatcggggacggagagagcacgagcccatcagacaaagtggtgaagaaagggaagaaggacaagaagatcaaaaaaacgttctttgaagagctggcagtagaagataaacaggctggggaagaagagaaagtgctcaaggagaaggagcagcagcagcagcaacagcaacagcagcaaaaaaaaaagcgagatacccgaaaaggcaggcggaagaaggatgtggatgatgatggagaagagaaagagctcatggagcgtcttaagaagctctcagtgccaaccagtgatgaggaggatgaagtacccgccccaaaaccccgcggagggaagaaaaccaagggtggtaatgtttttgcagccctgattcaggatcagagtgaggaagaggaggaggaagaaaaacatcctcctaagcctgccaagccggagaagaatcggatcaataaggccgtatctgaggaacagcagcctgcactcaagggcaaaaagggaaaggaagagaagtcaaaagggaaggctaagcctcaaaataaattcgctgctctggacaatgaagaggaggataaagaagaagaaattataaaggaaaaggagcctcccaaacaagggaaggagaaggccaagaaggcagagcagggttcagaggaagaaggagaaggggaagaagaggaggaggaaggaggagagtctaaggcagatgatccctatgctcatcttagcaaaaaggagaagaaaaagctgaaaaaacagatggagtatgagcgccaagtggcttcattaaaagcagccaatgcagctgaaaatgacttctccgtgtcccaggcggagatgtcctcccgccaagccatgttagaaaatgcatctgacatcaagctggagaagttcagcatctccgctcatggcaaggagctgttcgtcaatgcagacctgtacattgtagccggccgccgctacgggctggtaggacccaatggcaagggcaagaccacactcctcaagcacattgccaaccgagccctgagcatccctcccaacattgatgtgttgctgtgtgagcaggaggtggtagcagatgagacaccagcagtccaggctgttcttcgagctgacaccaagcgattgaagctgctggaagaggagcggcggcttcagggacagctggaacaaggggatgacacagctgctgagaggctagagaaggtgtatgaggaattgcgggccactggggcggcagctgcagaggccaaagcacggcggatcctggctggcctgggctttgaccctgaaatgcagaatcgacccacacagaagttctcagggggctggcgcatgcgtgtctccctggccagggcactgttcatggagcccacactgctgatgctggatgagcccaccaaccacctggacctcaacgctgtcatctggcttaataactacctccagggctggcggaagaccttgctgatcgtctcccatgaccagggcttcttggatgatgtctgcactgatatcatccacctcgatgcccagcggctccactactataggggcaattacatgaccttcaaaaagatgtaccagcagaagcagaaagaactgctgaaacagtatgagaagcaagagaaaaagctgaaggagctgaaggcaggcgggaagtccaccaagcaggcggaaaaacaaacgaaggaagccctgactcggaagcagcagaaatgccgacggaaaaaccaagatgaggaatcccaggaggcccctgagctcctgaagcgccctaaggagtacactgtgcgcttcacttttccagaccccccaccactcagccctccagtgctgggtctgcatggtgtgacattcggctaccagggacagaaaccactctttaagaacttggattttggcatcgacatggattcaaggatttgcattgtgggccctaatggtgtggggaagagtacgctactcctgctgctgactggcaagctgacaccgacccatggggaaatgagaaagaaccaccggctgaaaattggcttcttcaaccagcagtatgcagagcagctgcgcatggaggagacgcccactgagtacctgcagcggggcttcaacctgccctaccaggatgcccgcaagtgcctgggccgcttcggcctggagagtcacgcccacaccatccagatctgcaaactctctggtggtcagaaggcgcgagttgtgtttgctgagctggcctgtcgggaacctgatgtcctcatcttggacgagccaaccaataacctggacatagagtctattgatgctctaggggaggccatcaatgaatacaagggtgctgtgatcgttgtcagccatgatgcccgactcatcacagaaaccaattgccagctgtgggtggtggaggagcagagtgttagccaaatcgatggtgactttgaagactacaagcgggaggtgttggaggccctgggtgaagtcatggtcagccggccccgagagtgagctttccttcccagaagtctcccgagagacatatttgtgtggcctagaagtcctctgtggtctcccctcctctgaagactgcctctggcctgcagctgacctggcaaccattcaggcacatgaaggtggagtgtgaccttgatgtgaccgggatcccactctgattgcatccatttctctgaaagacttgtttgttctgcttctcttcatataactgagctggccttatccttggcatccccctaaacaaacaagaggtgaccaccttattgtgaggttccatccagccaagtttatgtggcctattgtctcaggactctcatcactcagaagcctgcctctgatttaccctacagcttcaggcccagctgccccccagtctttgggtggtgctgttcttttctggtggatttaatgctgactcactggtacaaacagctgttgaagctcagagctggaggtgagcttctgaggcctttgccattatccagcccaagatttggtgcctgcagcctcttgtctggttgaggacttggggcaggaaaggaatgctgctgaacttgaatttccctttacaaggggaagaaataaaggaaaggagttgctgccgacctgtcactgtttggagattgatgggagttggaactgttctcagtcttgatttgctttattcagttttctagcagcttttaatagtcccctcttccccactaaatggatcttgtttgcagtcttgctgacagtgtttgctgtttaaggatcataggattcctttcccccaacccttcacgcaaggaaaaagcaaagtgattcataccttctatcttggaaaaaaaaaaaa

SEQ ID NO: 53 - protein, 845 aa, Homo sapiens
MPKAPKQQPPEPEWIGDGESTSPSDKVVKKGKKDKKIKKTFFEELAVEDKQAGEEEKVLKEKEQQQQQQQQQQKKKRDTRKGRRKKDVDDDGEEKELMERLKKLSVPTSDEEDEVPAPKPRGGKKTKGGNVFAALIQDQSEEEEEEEKHPPKPAKPEKNRINKAVSEEQQPALKGKKGKEEKSKGKAKPQNKFAALDNEEEDKEEEIIKEKEPPKQGKEKAKKAEQGSEEEGEGEEEEEEGGESKADDPYAHLSKKEKKKLKKQMEYERQVASLKAANAAENDFSVSQAEMSSRQAMLENASDIKLEKFSISAHGKELFVNADLYIVAGRRYGLVGPNGKGKTTLLKHIANRALSIPPNIDVLLCEQEVVADETPAVQAVLRADTKRLKLLEEERRLQGQLEQGDDTAAERLEKVYEELRATGAAAAEAKARRILAGLGFDPEMQNRPTQKFSGGWRMRVSLARALFMEPTLLMLDEPTNHLDLNAVIWLNNYLQGWRKTLLIVSHDQGFLDDVCTDIIHLDAQRLHYYRGNYMTFKKMYQQKQKELLKQYEKQEKKLKELKAGGKSTKQAEKQTKEALTRKQQKCRRKNQDEESQEAPELLKRPKEYTVRFTFPDPPPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 54 - DNA, 3360 bp, Homo sapiens
gcgccagcttggagagccagccccatcggggttccccgccgccggaagcggaaatagcaccgggcgccgccacagtagctgtaactgccaccgcgatgccgaaggcgcccaagcagcagccgccggagcccgagtggatcggggacggagagagcacgagcccatcagacaaagtggtgaagaaagggaagaaggacaagaagatcaaaaaaacgttctttgaagagctggcagtagaagataaacaggctggggaagaagagaaagtgctcaaggagaaggagcagcagcagcagcaacagcaacagcagcaaaaaaaaaagcgagatacccgaaaaggcaggcggaagaaggatgtggatgatgatggagaagagaaagagctcatggagcgtcttaagaagctctcagtgccaaccagtgatgaggaggatgaagtacccgccccaaaaccccgcggagggaagaaaaccaagggtggtaatgtttttgcagccctgattcaggatcagagtgaggaagaggaggaggaagaaaaacatcctcctaagcctgccaagccggagaagaatcggatcaataaggccgtatctgaggaacagcagcctgcactcaagggcaaaaagggaaaggaagagaagtcaaaagggaaggctaagcctcaaaataaattcgctgctctggacaatgaagaggaggataaagaagaagaaattataaaggaaaaggagcctcccaaacaagggaaggagaaggccaagaaggcagagcagatggagtatgagcgccaagtggcttcattaaaagcagccaatgcagctgaaaatgacttctccgtgtcccaggcggagatgtcctcccgccaagccatgttagaaaatgcatctgacatcaagctggagaagttcagcatctccgctcatggcaaggagctgttcgtcaatgcagacctgtacattgtagccggccgccgctacgggctggtaggacccaatggcaagggcaagaccacactcctcaagcacattgccaaccgagccctgagcatccctcccaacattgatgtgttgctgtgtgagcaggaggtggtagcagatgagacaccagcagtccaggctgttcttcgagctgacaccaagcgattgaagctgctggaagaggagcggcggcttcagggacagctggaacaaggggatgacacagctgctgagaggctagagaaggtgtatgaggaattgcgggccactggggcggcagctgcagaggccaaagcacggcggatcctggctggcctgggctttgaccctgaaatgcagaatcgacccacacagaagttctcagggggctggcgcatgcgtgtctccctggccagggcactgttcatggagcccacactgctgatgctggatgagcccaccaaccacctggacctcaacgctgtcatctggcttaataactacctccagggctggcggaagaccttgctgatcgtctcccatgaccagggcttcttggatgatgtctgcactgatatcatccacctcgatgcccagcggctccactactataggggcaattacatgaccttcaaaaagatgtaccagcagaagcagaaagaactgctgaaacagtatgagaagcaagagaaaaagctgaaggagctgaaggcaggcgggaagtccaccaagcaggcggaaaaacaaacgaaggaagccctgactcggaagcagcagaaatgccgacggaaaaaccaagatgaggaatcccaggaggcccctgagctcctgaagcgccctaaggagtacactgtgcgcttcacttttccagaccccccaccactcagccctccagtgctgggtctgcatggtgtgacattcggctaccagggacagaaaccactctttaagaacttggattttggcatcgacatggattcaaggatttgcattgtgggccctaatggtgtggggaagagtacgctactcctgctgctgactggcaagctgacaccgacccatggggaaatgagaaagaaccaccggctgaaaattggcttcttcaaccagcagtatgcagagcagctgcgcatggaggagacgcccactgagtacctgcagcggggcttcaacctgccctaccaggatgcccgcaagtgcctgggccgcttcggcctggagagtcacgcccacaccatccagatctgcaaactctctggtggtcagaaggcgcgagttgtgtttgctgagctggcctgtcgggaacctgatgtcctcatcttggacgagccaaccaataacctggacatagagtctattgatgctctaggggaggccatcaatgaatacaagggtgctgtgatcgttgtcagccatgatgcccgactcatcacagaaaccaattgccagctgtgggtggtggaggagcagagtgttagccaaatcgatggtgactttgaagactacaagcgggaggtgttggaggccctgggtgaagtcatggtcagccggccccgagagtgagctttccttcccagaagtctcccgagagacatatttgtgtggcctagaagtcctctgtggtctcccctcctctgaagactgcctctggcctgcagctgacctggcaaccattcaggcacatgaaggtggagtgtgaccttgatgtgaccgggatcccactctgattgcatccatttctctgaaagacttgtttgttctgcttctcttcatataactgagctggccttatccttggcatccccctaaacaaacaagaggtgaccaccttattgtgaggttccatccagccaagtttatgtggcctattgtctcaggactctcatcactcagaagcctgcctctgatttaccctacagcttcaggcccagctgccccccagtctttgggtggtgctgttcttttctggtggatttaatgctgactcactggtacaaacagctgttgaagctcagagctggaggtgagcttctgaggcctttgccattatccagcccaagatttggtgcctgcagcctcttgtctggttgaggacttggggcaggaaaggaatgctgctgaacttgaatttccctttacaaggggaagaaataaaggaaaggagttgctgccgacctgtcactgtttggagattgatgggagttggaactgttctcagtcttgatttgctttattcagttttctagcagcttttaatagtcccctcttccccactaaatggatcttgtttgcagtcttgctgacagtgtttgctgtttaaggatcataggattcctttcccccaacccttcacgcaaggaaaaagcaaagtgattcataccttctatcttggaaaaaaaaaaaa

SEQ ID NO: 55 - protein, 807 aa, Homo sapiens
MPKAPKQQPPEPEWIGDGESTSPSDKVVKKGKKDKKIKKTFFEELAVEDKQAGEEEKVLKEKEQQQQQQQQQQKKKRDTRKGRRKKDVDDDGEEKELMERLKKLSVPTSDEEDEVPAPKPRGGKKTKGGNVFAALIQDQSEEEEEEEKHPPKPAKPEKNRINKAVSEEQQPALKGKKGKEEKSKGKAKPQNKFAALDNEEEDKEEEIIKEKEPPKQGKEKAKKAEQMEYERQVASLKAANAAENDFSVSQAEMSSRQAMLENASDIKLEKFSISAHGKELFVNADLYIVAGRRYGLVGPNGKGKTTLLKHIANRALSIPPNIDVLLCEQEVVADETPAVQAVLRADTKRLKLLEEERRLQGQLEQGDDTAAERLEKVYEELRATGAAAAEAKARRILAGLGFDPEMQNRPTQKFSGGWRMRVSLARALFMEPTLLMLDEPTNHLDLNAVIWLNNYLQGWRKTLLIVSHDQGFLDDVCTDIIHLDAQRLHYYRGNYMTFKKMYQQKQKELLKQYEKQEKKLKELKAGGKSTKQAEKQTKEALTRKQQKCRRKNQDEESQEAPELLKRPKEYTVRFTFPDPPPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 56 - DNA, 2269 bp, Homo sapiens
acagcgcgtgcgccgccgcaagcatggctggtgatgattggacgactggtaacagggggcggagggctccgaagtctggttttgggcgggaattgaaaccgccgctgaagccaacaagaatttgagaactgtaaataccaagccttgaaagggaccatggtgcggcctgtgagacataagaaaccagtcaattactcacagtttgaccactctgacagtgatgatgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaagactccctgaaggtacttttagtattccagctagtgcagtgccttgtacaaagatggctttagatgacaagctctaccagagagacttagaagttgcactagctttatcagtgaaggaacttccaacagtcaccactaatgtgcagaactctcaagataaaagcattgaaaaacatggcagtagtaaaatagaaacaatgaataagtctcctcatatctctaattgcagtgtagccagtgattatttagatttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttggtgacttcggtggactctgctccagctgccgtcaaatcagaatctcagtccttgccaaaaaaggtttctctgtcttcagataccactaggaaaccattagaaatacgcagtccttcagctgaaagcaagaaacctaaatgggtcccaccagcggcatctggaggtagcagaagtagcagcagcccactggtggtagtgtctgtgaagtctcccaatcagagtctccgccttggcttgtccagattagcacgagttaaacctttgcatccaaatgccactagcacctgagtgtggtacaggaggaatgtttggttgggagaatcacagctttacaagggtgtttatatttgatttgtgtttatatttgaggcaggtattgtaatataaaggaatccattaccatgtcctataaatgacctctagccattttatgattatgttctctgtaaaactcttcaagacttcaatgagaagtttgtttataagaattatcttctcatacctttccttgtgaagagcgtattctgtttttctatcagttcgacatgaagtccacatcacatgctgttcttttctagttacatgatgtgcctttctagctttgtctagtttatagcaccttaactttaactgttcagttttatctggcagaggaaaacattcttatttctttcagaagacatttctgaaatcttataagctacttaagctacgttgtcagttttatcgcaaagatgttttgtattttagccaaatctttttatagtacaaacttagaattattttacacactaaaatggttgcagttttatggcatatgtctccgatttagatggttattctctagaaaatagtatttaaagacattttatgaaatcttcattgtcaaaacctttaataaaagtggaaatattttgaaatgccctttttcttgataccactcatccacgtgttcctgattgtccacatttcatgataaaatgagagctccgcagagaatgttagcctttctgttgtaaatgtaatcttcaagtagtcactttttgttaagttctttagaaagtagttgtcaagtacttagtcatccctattatgatatgagatagtacagcttttcaggaagcttagatctgaatttactttgaaaaacaattgtaatgaatattttatatttacattgagaatttcaactagcttctgatcaatttttaataaaaaattttcaaatcatgttagctgttaaaaaatgtataataactcagtttttcttggtttatggaaatatctatattaatgtgaaaataattaatttagaattgtgattaaagtgagcatttgtcta

SEQ ID NO: 57 - protein, 352 aa, Homo sapiens
MVRPVRHKKPVNYSQFDHSDSDDDFVSATVPLNKKSRTAPKELKQDKPKPNLNNLRKEEIPVQEKTPKKRLPEGTFSIPASAVPCTKMALDDKLYQRDLEVALALSVKELPTVTTNVQNSQDKSIEKHGSSKIETMNKSPHISNCSVASDYLDLDKITVEDDVGGVQGKRKAASKAAAQQRKILLEGSDGDSANDTEPDFAPGEDSEDDSDFCESEDNDEDFSMRKSKVKEIKKKEVKVKSPVEKKEKKSKSKCNALVTSVDSAPAAVKSESQSLPKKVSLSSDTTRKPLEIRSPSAESKKPKWVPPAASGGSRSSSSPLVVVSVKSPNQSLRLGLSRLARVKPLHPNATST

SEQ ID NO: 58 - DNA, 2218 bp, Homo sapiens
acagcgcgtgcgccgccgcaagcatggctggtgatgattggacgactggtaacagggggcggagggctccgaagtctggttttgggcgggaattgaaaccgccgctgaagccaacaagaatttgagaactgtaaataccaagccttgaaagggaccatggtgcggcctgtgagacataagaaaccagtcaattactcacagtttgaccactctgacagtgatgatgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaaggatggctttagatgacaagctctaccagagagacttagaagttgcactagctttatcagtgaaggaacttccaacagtcaccactaatgtgcagaactctcaagataaaagcattgaaaaacatggcagtagtaaaatagaaacaatgaataagtctcctcatatctctaattgcagtgtagccagtgattatttagatttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttggtgacttcggtggactctgctccagctgccgtcaaatcagaatctcagtccttgccaaaaaaggtttctctgtcttcagataccactaggaaaccattagaaatacgcagtccttcagctgaaagcaagaaacctaaatgggtcccaccagcggcatctggaggtagcagaagtagcagcagcccactggtggtagtgtctgtgaagtctcccaatcagagtctccgccttggcttgtccagattagcacgagttaaacctttgcatccaaatgccactagcacctgagtgtggtacaggaggaatgtttggttgggagaatcacagctttacaagggtgtttatatttgatttgtgtttatatttgaggcaggtattgtaatataaaggaatccattaccatgtcctataaatgacctctagccattttatgattatgttctctgtaaaactcttcaagacttcaatgagaagtttgtttataagaattatcttctcatacctttccttgtgaagagcgtattctgtttttctatcagttcgacatgaagtccacatcacatgctgttcttttctagttacatgatgtgcctttctagctttgtctagtttatagcaccttaactttaactgttcagttttatctggcagaggaaaacattcttatttctttcagaagacatttctgaaatcttataagctacttaagctacgttgtcagttttatcgcaaagatgttttgtattttagccaaatctttttatagtacaaacttagaattattttacacactaaaatggttgcagttttatggcatatgtctccgatttagatggttattctctagaaaatagtatttaaagacattttatgaaatcttcattgtcaaaacctttaataaaagtggaaatattttgaaatgccctttttcttgataccactcatccacgtgttcctgattgtccacatttcatgataaaatgagagctccgcagagaatgttagcctttctgttgtaaatgtaatcttcaagtagtcactttttgttaagttctttagaaagtagttgtcaagtacttagtcatccctattatgatatgagatagtacagcttttcaggaagcttagatctgaatttactttgaaaaacaattgtaatgaatattttatatttacattgagaatttcaactagcttctgatcaatttttaataaaaaattttcaaatcatgttagctgttaaaaaatgtataataactcagtttttcttggtttatggaaatatctatattaatgtgaaaataattaatttagaattgtgattaaagtgagcatttgtcta

SEQ ID NO: 59 - protein, 335 aa, Homo sapiens
MVRPVRHKKPVNYSQFDHSDSDDDFVSATVPLNKKSRTAPKELKQDKPKPNLNNLRKEEIPVQEKTPKKRMALDDKLYQRDLEVALALSVKELPTVTTNVQNSQDKSIEKHGSSKIETMNKSPHISNCSVASDYLDLDKITVEDDVGGVQGKRKAASKAAAQQRKILLEGSDGDSANDTEPDFAPGEDSEDDSDFCESEDNDEDFSMRKSKVKEIKKKEVKVKSPVEKKEKKSKSKCNALVTSVDSAPAAVKSESQSLPKKVSLSSDTTRKPLEIRSPSAESKKPKWVPPAASGGSRSSSSPLVVVSVKSPNQSLRLGLSRLARVKPLHPNATST

SEQ ID NO: 60 - DNA, 68 bp, Homo sapiens
ccctcgtcttacccagcagtgtttgggtgcggttgggagtctctaatactgccgggtaatgatggagg

SEQ ID NO: 61 - DNA, 95 bp, Homo sapiens
cggccggccctgggtccatcttccagtacagtgttggatggtctaattgtgaagctcctaacactgtctggtaaagatggctcccgggtgggttc

SEQ ID NO: 62 - DNA, 2535 bp, Homo sapiens
ttaaggccgcgctcgccagcctcggcggggcggctcccgccgccgcaaccaatggatctcctcctctgtttaaatagactcgccgtgtcaatcattttcttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggctgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaaacagctcgaattaagaatatgtttccttgtttatcagatacatcactgcttgatgaagcaaggaagatatacatgaaaattttaaaaatacatatcgctgacttcatggaatggacatcctgtataagcactgaaaaacaacaacacaataacactaaaattttaggcactcttaaatgatctgcctctaaaagcgttggatgtagcattatgcaattaggtttttccttatttgcttcattgtactacctgtgtatatagtttttaccttttatgtagcacataaactttggggaagggagggcagggtggggctgaggaactgacgtggagcggggtatgaagagcttgctttgatttacagcaagtagataaatatttgacttgcatgaagagaagcaattttggggaagggtttgaattgttttctttaaagatgtaatgtccctttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaataattctaaatccctcgatatttttaaagatctgtaagtaacttcacattaaaaaatgaaatattttttaatttaaagcttactctgtccatttatccacaggaaagtgttatttttcaaggaaggttcatgtagagaaaagcacacttgtaggataagtgaaatggatactacatctttaaacagtatttcattgcctgtgtatggaaaaaccatttgaagtgtacctgtgtacataactctgtaaaaacactgaaaaattatactaacttatttatgttaaaagattttttttaatctagacaatatacaagccaaagtggcatgttttgtgcatttgtaaatgctgtgttgggtagaataggttttcccctcttttgttaaataatatggctatgcttaaaaggttgcatactgagccaagtataattttttgtaatgtgtgaaaaagatgccaattattgttacacattaagtaatcaataaagaaaacttccatagctattcattgagtcaaaaaaaaaaaaaaa

SEQ ID NO: 63 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 64 - DNA, 1821 bp, Homo sapiens
gagttgcggcgatgggcggggcaggcgcgcggggattggcgggatgcggcgcgccgcgcgggtgagacatcggtatccaggcacgataaatttccaagtggacacaatgtctggtgtcaactacagctgttctccttcttttcccagtatcctttgggtgcagtgagacaccaggagagctgctgctttgggggatggacaggggcagcaggaatgcctttgtgttttcgcagtgaacctccttggcctgggcgaagctgtgtggaccaagcaagtcaggagtgtggccatgttttctgagcaggctgcccagagggcccacactctactgtccccaccatcagccaacaatgccacctttgcccgggtgccagtggcaacctacaccaactcctcacaacccttccggctaggagagcgcagctttagccggcagtatgcccacatttatgccacccgcctcatccaaatgagacccttcctggagaaccgggcccagcagcactggggcagtggagtgggagtgaagaagctgtgtgaactgcagcctgaggagaagtgctgtgtggtgggcactctgttcaaggccatgccgctgcagccctccatcctgcgggaggtcagcgaggagcacaacctgctcccccagcctcctcggagtaaatacatacacccagatgacgagctggtcttggaagatgaactgcagcgtatcaaactaaaaggcaccattgacgtgtcaaagctggttacggggactgtcctggctgtgtttggctccgtgagagacgacgggaagtttctggtggaggactattgctttgctgaccttgctccccagaagcccgcacccccacttgacacagataggtttgtgctactggtgtccggcctgggcctgggtggcggtggaggcgagagcctgctgggcacccagctgctggtggatgtggtgacggggcagcttggggacgaaggggagcagtgcagcgccgcccacgtctcccgggttatcctcgctggcaacctcctcagccacagcacccagagcagggattctatcaataaggccaaatacctcaccaagaaaacccaggcagccagcgtggaggctgttaagatgctggatgagatcctcctgcagctgagcgcctcagtgcccgtggacgtgatgccaggcgagtttgatcccaccaattacacgctcccccagcagcccctccacccctgcatgttcccgctggccactgcctactccacgctccagctggtcaccaacccctaccaggccaccattgatggagtcagatttttggggacatcaggacagaacgtgagtgacattttccgatacagcagcatggaggatcacttggagatcctggagtggaccctgcgggtccgtcacatcagccccacagcccctgacactctaggttgttaccccttctacaaaactgacccgttcatcttcccagagtgcccgcatgtctacttttgtggcaacacccccagctttggctccaaaatcatccgaggtcctgaggaccagacagtgctgttggtgactgtccctgacttcagtgccacgcagaccgcctgccttgtgaacctgcgcagcctggcctgccagcccatcagcttctcgggcttcggggcagaggacgatgacctgggaggcctggggctgggcccctgactcaaaaaagtggttttgaccagagaggcccagatggaggctgttcattccctgcagtgtcggcattgtaaataaagcctgagcacttgctgatgcgagccttgaaaaaaaaaaaaaaaaaa

SEQ ID NO: 65 - protein, 469 aa, Homo sapiens
MFSEQAAQRAHTLLSPPSANNATFARVPVATYTNSSQPFRLGERSFSRQYAHIYATRLIQMRPFLENRAQQHWGSGVGVKKLCELQPEEKCCVVGTLFKAMPLQPSILREVSEEHNLLPQPPRSKYIHPDDELVLEDELQRIKLKGTIDVSKLVTGTVLAVFGSVRDDGKFLVEDYCFADLAPQKPAPPLDTDRFVLLVSGLGLGGGGGESLLGTQLLVDVVTGQLGDEGEQCSAAHVSRVILAGNLLSHSTQSRDSINKAKYLTKKTQAASVEAVKMLDEILLQLSASVPVDVMPGEFDPTNYTLPQQPLHPCMFPLATAYSTLQLVTNPYQATIDGVRFLGTSGQNVSDIFRYSSMEDHLEILEWTLRVRHISPTAPDTLGCYPFYKTDPFIFPECPHVYFCGNTPSFGSKIIRGPEDQTVLLVTVPDFSATQTACLVNLRSLACQPISFSGFGAEDDDLGGLGLGP

SEQ ID NO: 66 - DNA, 2182 bp, Homo sapiens
gagtggggtccagggaaacggggtcagctgggggtggcagttccaggccgcgaggccgggctcctgggtcggtgggctggtgtcttggcggacgtcccgcagctgccgcgtggatccgagccggggcacccgccgtgactgggacagcccccagggcgctctcggccccatcccgagtagcgcggcctggctgctgccgccatcaagcacgttcgagccaaaagctcctaacgagtcactcgttagacacgtgtgcggagcctgtgtcccaggccagtgctgtcccgtggagatagattgcaagccgctagggaattttttaactttctagtgccggagagctggatggaggcagatcgggaattccatttggggcaaactgaacttgattgagaccctggtagttgtccagatggaacaggacacctgagtctagggttcgggaagaactccagatgggacaaacactcctagctttccttttctctttttggatgaccgctacaggtatcctttgggtgcagtgagacaccaggagagctgctgctttgggggatggacaggggcagcaggaatgcctttgtgttttcgcagtgaacctccttggcctgggcgaagctgtgtggaccaagcaagtcaggagtgtggccatgttttctgagcaggctgcccagagggcccacactctactgtccccaccatcagccaacaatgccacctttgcccgggtgccagtggcaacctacaccaactcctcacaacccttccggctaggagagcgcagctttagccggcagtatgcccacatttatgccacccgcctcatccaaatgagacccttcctggagaaccgggcccagcagcactggggcagtggagtgggagtgaagaagctgtgtgaactgcagcctgaggagaagtgctgtgtggtgggcactctgttcaaggccatgccgctgcagccctccatcctgcgggaggtcagcgaggagcacaacctgctcccccagcctcctcggagtaaatacatacacccagatgacgagctggtcttggaagatgaactgcagcgtatcaaactaaaaggcaccattgacgtgtcaaagctggttacggggactgtcctggctgtgtttggctccgtgagagacgacgggaagtttctggtggaggactattgctttgctgaccttgctccccagaagcccgcacccccacttgacacagataggtttgtgctactggtgtccggcctgggcctgggtggcggtggaggcgagagcctgctgggcacccagctgctggtggatgtggtgacggggcagcttggggacgaaggggagcagtgcagcgccgcccacgtctcccgggttatcctcgctggcaacctcctcagccacagcacccagagcagggattctatcaataaggccaaatacctcaccaagaaaacccaggcagccagcgtggaggctgttaagatgctggatgagatcctcctgcagctgagcgcctcagtgcccgtggacgtgatgccaggcgagtttgatcccaccaattacacgctcccccagcagcccctccacccctgcatgttcccgctggccactgcctactccacgctccagctggtcaccaacccctaccaggccaccattgatggagtcagatttttggggacatcaggacagaacgtgagtgacattttccgatacagcagcatggaggatcacttggagatcctggagtggaccctgcgggtccgtcacatcagccccacagcccctgacactctaggttgttaccccttctacaaaactgacccgttcatcttcccagagtgcccgcatgtctacttttgtggcaacacccccagctttggctccaaaatcatccgaggtcctgaggaccagacagtgctgttggtgactgtccctgacttcagtgccacgcagaccgcctgccttgtgaacctgcgcagcctggcctgccagcccatcagcttctcgggcttcggggcagaggacgatgacctgggaggcctggggctgggcccctgactcaaaaaagtggttttgaccagagaggcccagatggaggctgttcattccctgcagtgtcggcattgtaaataaagcctgagcacttgctgatgcgagccttgaaaaaaaaaaaaaaaaaa

SEQ ID NO: 67 - protein, 469 aa, Homo sapiens; identical to SEQ ID NO: 65

SEQ ID NO: 68 - DNA, 1648 bp, Homo sapiens
gagttgcggcgatgggcggggcaggcgcgcggggattggcgggatgcggcgcgccgcgcgtgaacctccttggcctgggcgaagctgtgtggaccaagcaagtcaggagtgtggccatgttttctgagcaggctgcccagagggcccacactctactgtccccaccatcagccaacaatgccacctttgcccgggtgccagtggcaacctacaccaactcctcacaacccttccggctaggagagcgcagctttagccggcagtatgcccacatttatgccacccgcctcatccaaatgagacccttcctggagaaccgggcccagcagcactggggcagtggagtgggagtgaagaagctgtgtgaactgcagcctgaggagaagtgctgtgtggtgggcactctgttcaaggccatgccgctgcagccctccatcctgcgggaggtcagcgaggagcacaacctgctcccccagcctcctcggagtaaatacatacacccagatgacgagctggtcttggaagatgaactgcagcgtatcaaactaaaaggcaccattgacgtgtcaaagctggttacggggactgtcctggctgtgtttggctccgtgagagacgacgggaagtttctggtggaggactattgctttgctgaccttgctccccagaagcccgcacccccacttgacacagataggtttgtgctactggtgtccggcctgggcctgggtggcggtggaggcgagagcctgctgggcacccagctgctggtggatgtggtgacggggcagcttggggacgaaggggagcagtgcagcgccgcccacgtctcccgggttatcctcgctggcaacctcctcagccacagcacccagagcagggattctatcaataaggccaaatacctcaccaagaaaacccaggcagccagcgtggaggctgttaagatgctggatgagatcctcctgcagctgagcgcctcagtgcccgtggacgtgatgccaggcgagtttgatcccaccaattacacgctcccccagcagcccctccacccctgcatgttcccgctggccactgcctactccacgctccagctggtcaccaacccctaccaggccaccattgatggagtcagatttttggggacatcaggacagaacgtgagtgacattttccgatacagcagcatggaggatcacttggagatcctggagtggaccctgcgggtccgtcacatcagccccacagcccctgacactctaggttgttaccccttctacaaaactgacccgttcatcttcccagagtgcccgcatgtctacttttgtggcaacacccccagctttggctccaaaatcatccgaggtcctgaggaccagacagtgctgttggtgactgtccctgacttcagtgccacgcagaccgcctgccttgtgaacctgcgcagcctggcctgccagcccatcagcttctcgggcttcggggcagaggacgatgacctgggaggcctggggctgggcccctgactcaaaaaagtggttttgaccagagaggcccagatggaggctgttcattccctgcagtgtcggcattgtaaataaagcctgagcacttgctgatgcgagccttgaaaaaaaaaaaaaaaaaa

SEQ ID NO: 69 - protein, 504 aa, Homo sapiens
MGGAGARGLAGCGAPRVNLLGLGEAVWTKQVRSVAMFSEQAAQRAHTLLSPPSANNATFARVPVATYTNSSQPFRLGERSFSRQYAHIYATRLIQMRPFLENRAQQHWGSGVGVKKLCELQPEEKCCVVGTLFKAMPLQPSILREVSEEHNLLPQPPRSKYIHPDDELVLEDELQRIKLKGTIDVSKLVTGTVLAVFGSVRDDGKFLVEDYCFADLAPQKPAPPLDTDRFVLLVSGLGLGGGGGESLLGTQLLVDVVTGQLGDEGEQCSAAHVSRVILAGNLLSHSTQSRDSINKAKYLTKKTQAASVEAVKMLDEILLQLSASVPVDVMPGEFDPTNYTLPQQPLHPCMFPLATAYSTLQLVTNPYQATIDGVRFLGTSGQNVSDIFRYSSMEDHLEILEWTLRVRHISPTAPDTLGCYPFYKTDPFIFPECPHVYFCGNTPSFGSKIIRGPEDQTVLLVTVPDFSATQTACLVNLRSLACQPISFSGFGAEDDDLGGLGLGP

SEQ ID NO: 70 - DNA, 1346 bp, Homo sapiens
gaagccccacctggaggagcggccgggatgggcctccgggacggtgtgccaggccggggccaagtcggaggcccctcgctctgggtgggcgctggggcccgcgagggctactgaaacaagctcacatcttcctgtgggaaaccttctagcaacaggatgagtctgcagtggactgcagttgccaccttcctctatgcggaggtctttgttgtgttgcttctctgcattcccttcatttctcctaaaagatggcagaagattttcaagtcccggctggtggagttgttagtgtcctatggcaacaccttctttgtggttctcattgtcatccttgtgctgttggtcatcgatgccgtgcgcgaaattcggaagtatgatgatgtgacggaaaaggtgaacctccagaacaatcccggggccatggagcacttccacatgaagcttttccgtgcccagaggaatctctacattgctggcttttccttgctgctgtccttcctgcttagacgcctggtgactctcatttcgcagcaggccacgctgctggcctccaatgaagcctttaaaaagcaggcggagagtgctagtgaggcggccaagaagtacatggaggagaatgaccagctcaagaagggagctgctgttgacggaggcaagttggatgtcgggaatgctgaggtgaagttggaggaagagaacaggagcctgaaggctgacctgcagaagctaaaggacgagctggccagcactaagcaaaaactagagaaagctgaaaaccaggttctggccatgcggaagcagtctgagggcctcaccaaggagtacgaccgcttgctggaggagcacgcaaagctgcaggctgcagtagatggtcccatggacaagaaggaagagtaagggcctccttcctcccctgcctgcagctggcttccacctggcacgtgcctgctgcttcctgagagcccggcctctccctccagtacttctgtttgtgcccttctgcttcccccattcccttccacagctcatagctcgtcatctcggcccttgtccacactctccaagcacattacaggggacctgattgctacacgttcagaatgcgtttgctgtcatcctgcttggcctggccaggcctggcacagccttggcttccacgcctgagcgtggagagcacgagttagttgtagtccggcttgcggtggggctgacttcctgttggtttgagcccctttttgttttgccctctgggtgttttctttggtcccgcaggagggtgggtggagcaggtggactggagtttctcttgagggcaataaaagttgtcatggtgtgtacgtggaaaa

SEQ ID NO: 71 - protein, 246 aa, Homo sapiens
MSLQWTAVATFLYAEVFVVLLLCIPFISPKRWQKIFKSRLVELLVSYGNTFFVVLIVILVLLVIDAVREIRKYDDVTEKVNLQNNPGAMEHFHMKLFRAQRNLYIAGFSLLLSFLLRRLVTLISQQATLLASNEAFKKQAESASEAAKKYMEENDQLKKGAAVDGGKLDVGNAEVKLEEENRSLKADLQKLKDELASTKQKLEKAENQVLAMRKQSEGLTKEYDRLLEEHAKLQAAVDGPMDKKEE

SEQ ID NO: 72 - DNA, 1828 bp, Homo sapiens
gatgggcctccgggacggtgtgccaggccggggccaagtcggaggcccctcgctctgggtgggcgctggggcccgcgagggctactgtaaggacccctggcttctgaggatactgcgtctagaactttctccgtatggggccttgaggtgcttggtcgagacctgcctttgcgcttggtcccgaatcctgccctctaggagtcgctcttgcgggcctccagcccaccggaggcgaagcggccccgggcggaaggccgctggatcctcgagggaggtgccggtttctctccgcgggcgccgtggggacggtgggaggcgggggcgtcggcagcgcttggactaggtgcggccttgggcctgcctggtagcggggatttgggcccgcagagcgcccgcctctgcggctgagttctgcctggcggggaagggagcgcccgatgggtgccgaggcgtcctcctcttggtgccctggcactgctcttcccgaagaacgcctttcagttaaacgggcgtcggaaatctcgggcttcctggggcagggatcgtcgggagaggccgctctggacgtgttgacacacgtgctggagggggcaggaaacaagctcacatcttcctgtgggaaaccttctagcaacaggatgagtctgcagtggactgcagttgccaccttcctctatgcggaggtctttgttgtgttgcttctctgcattcccttcatttctcctaaaagatggcagaagattttcaagtcccggctggtggagttgttagtgtcctatggcaacaccttctttgtggttctcattgtcatccttgtgctgttggtcatcgatgccgtgcgcgaaattcggaagtatgatgatgtgacggaaaaggtgaacctccagaacaatcccggggccatggagcacttccacatgaagcttttccgtgcccagaggaatctctacattgctggcttttccttgctgctgtccttcctgcttagacgcctggtgactctcatttcgcagcaggccacgctgctggcctccaatgaagcctttaaaaagcaggcggagagtgctagtgaggcggccaagaagtacatggaggagaatgaccagctcaagaagggagctgctgttgacggaggcaagttggatgtcgggaatgctgaggtgaagttggaggaagagaacaggagcctgaaggctgacctgcagaagctaaaggacgagctggccagcactaagcaaaaactagagaaagctgaaaaccaggttctggccatgcggaagcagtctgagggcctcaccaaggagtacgaccgcttgctggaggagcacgcaaagctgcaggctgcagtagatggtcccatggacaagaaggaagagtaagggcctccttcctcccctgcctgcagctggcttccacctggcacgtgcctgctgcttcctgagagcccggcctctccctccagtacttctgtttgtgcccttctgcttcccccattcccttccacagctcatagctcgtcatctcggcccttgtccacactctccaagcacattacaggggacctgattgctacacgttcagaatgcgtttgctgtcatcctgcttggcctggccaggcctggcacagccttggcttccacgcctgagcgtggagagcacgagttagttgtagtccggcttgcggtggggctgacttcctgttggtttgagcccctttttgttttgccctctgggtgttttctttggtcccgcaggagggtgggtggagcaggtggactggagtttctcttgagggcaataaaagttgtcatggtgtgtacgtggaaaa

SEQ ID NO: 73 - protein, 313 aa, Homo sapiens
MGAEASSSWCPGTALPEERLSVKRASEISGFLGQGSSGEAALDVLTHVLEGAGNKLTSSCGKPSSNRMSLQWTAVATFLYAEVFVVLLLCIPFISPKRWQKIFKSRLVELLVSYGNTFFVVLIVILVLLVIDAVREIRKYDDVTEKVNLQNNPGAMEHFHMKLFRAQRNLYIAGFSLLLSFLLRRLVTLISQQATLLASNEAFKKQAESASEAAKKYMEENDQLKKGAAVDGGKLDVGNAEVKLEEENRSLKADLQKLKDELASTKQKLEKAENQVLAMRKQSEGLTKEYDRLLEEHAKLQAAVDGPMDKKEE

SEQ ID NO: 74 - DNA, 1647 bp, Homo sapiens
accctgttctcgcccctcggcggggcccgcccacacccccacctcccgttctcgcccctcggcggggcccctcccgcaccacagagacgtgggacggccgcgcggactagagaagcggccctcgccggcccgacaaagccccgccccgccccgcccgcgtgctcgctccaccccgccctgctcgcccggagcccgaggcgcctcttcctctcccgctcggagcgccggccctccgctccggggcggggtgggaagcggaggtgggcggagcctccggggcctcgcgagggctggtgggcggtgcccggcgggcgcttccggtttccggccgcggtatgaggggcggggccggggctgctgtgggagagttctgttgctgcggcggggcctgcacgttgactgtgggaaactcggaaacaagctcacatcttcctgtgggaaaccttctagcaacaggatgagtctgcagtggactgcagttgccaccttcctctatgcggaggtctttgttgtgttgcttctctgcattcccttcatttctcctaaaagatggcagaagattttcaagtcccggctggtggagttgttagtgtcctatggcaacaccttctttgtggttctcattgtcatccttgtgctgttggtcatcgatgccgtgcgcgaaattcggaagtatgatgatgtgacggaaaaggtgaacctccagaacaatcccggggccatggagcacttccacatgaagcttttccgtgcccagaggaatctctacattgctggcttttccttgctgctgtccttcctgcttagacgcctggtgactctcatttcgcagcaggccacgctgctggcctccaatgaagcctttaaaaagcaggcggagagtgctagtgaggcggccaagaagtacatggaggagaatgaccagctcaagaagggagctgctgttgacggaggcaagttggatgtcgggaatgctgaggtgaagttggaggaagagaacaggagcctgaaggctgacctgcagaagctaaaggacgagctggccagcactaagcaaaaactagagaaagctgaaaaccaggttctggccatgcggaagcagtctgagggcctcaccaaggagtacgaccgcttgctggaggagcacgcaaagctgcaggctgcagtagatggtcccatggacaagaaggaagagtaagggcctccttcctcccctgcctgcagctggcttccacctggcacgtgcctgctgcttcctgagagcccggcctctccctccagtacttctgtttgtgcccttctgcttcccccattcccttccacagctcatagctcgtcatctcggcccttgtccacactctccaagcacattacaggggacctgattgctacacgttcagaatgcgtttgctgtcatcctgcttggcctggccaggcctggcacagccttggcttccacgcctgagcgtggagagcacgagttagttgtagtccggcttgcggtggggctgacttcctgttggtttgagcccctttttgttttgccctctgggtgttttctttggtcccgcaggagggtgggtggagcaggtggactggagtttctcttgagggcaataaaagttgtcatggtgtgtacgtggaaaa

SEQ ID NO: 75 - protein, 246 aa, Homo sapiens; identical to SEQ ID NO: 71

SEQ ID NO: 76 - DNA, 1417 bp, Homo sapiens
gagagttctgttgctgcggcggggcctgcacgttgactgtgggaaactcggtgagcgggctccgcgcgccgggctgggctccgggaccgcggaggctccccggcccatcgacgagggagagaggcgagcggcgcggggaggcccgggggccggggaatctcggggcccgcagcctacctgcgtgaaacaagctcacatcttcctgtgggaaaccttctagcaacaggatgagtctgcagtggactgcagttgccaccttcctctatgcggaggtctttgttgtgttgcttctctgcattcccttcatttctcctaaaagatggcagaagattttcaagtcccggctggtggagttgttagtgtcctatggcaacaccttctttgtggttctcattgtcatccttgtgctgttggtcatcgatgccgtgcgcgaaattcggaagtatgatgatgtgacggaaaaggtgaacctccagaacaatcccggggccatggagcacttccacatgaagcttttccgtgcccagaggaatctctacattgctggcttttccttgctgctgtccttcctgcttagacgcctggtgactctcatttcgcagcaggccacgctgctggcctccaatgaagcctttaaaaagcaggcggagagtgctagtgaggcggccaagaagtacatggaggagaatgaccagctcaagaagggagctgctgttgacggaggcaagttggatgtcgggaatgctgaggtgaagttggaggaagagaacaggagcctgaaggctgacctgcagaagctaaaggacgagctggccagcactaagcaaaaactagagaaagctgaaaaccaggttctggccatgcggaagcagtctgagggcctcaccaaggagtacgaccgcttgctggaggagcacgcaaagctgcaggctgcagtagatggtcccatggacaagaaggaagagtaagggcctccttcctcccctgcctgcagctggcttccacctggcacgtgcctgctgcttcctgagagcccggcctctccctccagtacttctgtttgtgcccttctgcttcccccattcccttccacagctcatagctcgtcatctcggcccttgtccacactctccaagcacattacaggggacctgattgctacacgttcagaatgcgtttgctgtcatcctgcttggcctggccaggcctggcacagccttggcttccacgcctgagcgtggagagcacgagttagttgtagtccggcttgcggtggggctgacttcctgttggtttgagcccctttttgttttgccctctgggtgttttctttggtcccgcaggagggtgggtggagcaggtggactggagtttctcttgagggcaataaaagttgtcatggtgtgtacgtggaaaa

SEQ ID NO: 77 - protein, 246 aa, Homo sapiens; identical to SEQ ID NO: 71

SEQ ID NO: 78 - DNA, 77 bp, Homo sapiens
ggcagtgctctactcaaaaagctgtcagtcacttagattacatgtgactgacacctctttgggtgaaggaaggctca

SEQ ID NO: 79 - DNA, 81 bp, Homo sapiens
gggctttcaagtcactagtggttccgtttagtagatgattgtgcattgtttcaaaatggtgccctagtgactacaaagccc

SEQ ID NO: 80 - DNA, 85 bp, Homo sapiens
gctaagcacttacaactgtttgcagaggaaactgagactttgtaactatgtctcagtctcatctgcaaagaagtaagtgctttgc

SEQ ID NO: 81 - DNA, 3167 bp, Homo sapiens
gccagagcgtgagccgcgacctccgcgcaggtggtcgcgccggtctccgcggaaatgttgtccaaagttcttccagtcctcctaggcatcttattgatcctccagtcgagggtcgagggacctcagactgaatcaaagaatgaagcctcttcccgtgatgttgtctatggcccccagccccagcctctggaaaatcagctcctctctgaggaaacaaagtcaactgagactgagactgggagcagagttggcaaactgccagaagcctctcgcatcctgaacactatcctgagtaattatgaccacaaactgcgccctggcattggagagaagcccactgtggtcactgttgagatctccgtcaacagccttggtcctctctctatcctagacatggaatacaccattgacatcatcttctcccagacctggtacgacgaacgcctctgttacaacgacacctttgagtctcttgttctgaatggcaatgtggtgagccagctatggatcccggacaccttttttaggaattctaagaggacccacgagcatgagatcaccatgcccaaccagatggtccgcatctacaaggatggcaaggtgttgtacacaattaggatgaccattgatgccggatgctcactccacatgctcagatttccaatggattctcactcttgccctctatctttctctagcttttcctatcctgagaatgagatgatctacaagtgggaaaatttcaagcttgaaatcaatgagaagaactcctggaagctcttccagtttgattttacaggagtgagcaacaaaactgaaataatcacaaccccagttggtgacttcatggtcatgacgattttcttcaatgtgagcaggcggtttggctatgttgcctttcaaaactatgtcccttcttccgtgaccacgatgctctcctgggtttccttttggatcaagacagagtctgctccagcccggacctctctagggatcacctctgttctgaccatgaccacgttgggcaccttttctcgtaagaatttcccgcgtgtctcctatatcacagccttggatttctatatcgccatctgcttcgtcttctgcttctgcgctctgttggagtttgctgtgctcaacttcctgatctacaaccagacaaaagcccatgcttctcctaaactccgccatcctcgtatcaatagccgtgcccatgcccgtacccgtgcacgttcccgagcctgtgcccgccaacatcaggaagcttttgtgtgccagattgtcaccactgagggaagtgatggagaggagcgcccgtcttgctcagcccagcagccccctagcccaggtagccctgagggtccccgcagcctctgctccaagctggcctgctgtgagtggtgcaagcgttttaagaagtacttctgcatggtccccgattgtgagggcagtacctggcagcagggccgcctctgcatccatgtctaccgcctggataactactcgagagttgttttcccagtgactttcttcttcttcaatgtgctctactggcttgtttgccttaacttgtaggtaccagctggtaccctgtggggcaacctctccagttccccaggaggtccaagccccttgccaagggagttgggggaaagcagcagcagcagcaggagcgactagagtttttcctgccccattccccaaacagaagcttgcagagggtttgtctttgctgcccctctcccctacctggcccattcactgagtcttctcagcagaccatttcaaattattaataaatgggccacctccctcttcttcaaggagcatccgtgatgctcagtgttcaaaaccacagccacttagtgatcagctccctaaaaccatgcctaagtacaggcggattagctatcttccaacaatgctgaccaccagacaattactgcatttttccagaagcccactattgcctttgtagtgctttcggcccagttctggcctcagcctcaaagtgcaccgactagttgcttgcctatacctggcacctcattaagatgctgggcagcagtataacaggaggaagagatccctctcctttggtcagattattatgttctcagttctctctccctgctacccctttctctgcagatagatagacactggcattatccctttaggaagaggggggggcagcaagagagcctatttgggacagcattcctctctctctgctgctgtgacatctccctctccttgctggctccatctttcgtctgcactaccaattcaatgcccttcatccaatgggtatctatttttgtgtgtgattatagtaactactccctgctttatatgccaccctcttccttctctttgacccctgtgactctttctgtaactttcccagtgacttcccctagccctgacccaggcactaggccttggtgacttcctggggccaagaaactaaggaaactcggctttgcaacaggcattactcgccattgattggtgcccacccagggcacactgtcggagttctatcacttgcttgacccctggacccataaaccagtccactgttatacccggggcactctaaccatcacaatcaatcaatcaaattcccttaaatttgtatggcactggaactttggcaaagcacttttgacaagttgtgtctgattggagcttcatgatagccttgtgacatctttagggcaggattcttatccccattttgcagatgaaaaccctgagtcacagatttctgtgggactgtggatctcactggaagctatccaagagcccactgtcaccttctagaccacatgatagggctagacagctcagttcaccatgattctcttctgtcacctctgctggcacaccagtggcaaggcccagaatggcgacctctctttagctcaatttctgggcctgaggtgctcagactgcccccaagatcaaatctctcctggctgtagtaacccagtggaatgaatttggacatgccccaatgcttctatatgctaagtgaaatctgtgtctgtaatttgttggggggtggatagggtggggtctccatctactttttgtcaccatcatctgaaatggggaaatatgtaaataaatatatcagcaaagcaaaaaaaaaaaaaaaa

SEQ ID NO: 82 - protein, 506 aa, Homo sapiens
MLSKVLPVLLGILLILQSRVEGPQTESKNEASSRDVVYGPQPQPLENQLLSEETKSTETETGSRVGKLPEASRILNTILSNYDHKLRPGIGEKPTVVTVEISVNSLGPLSILDMEYTIDIIFSQTWYDERLCYNDTFESLVLNGNVVSQLWIPDTFFRNSKRTHEHEITMPNQMVRIYKDGKVLYTIRMTIDAGCSLHMLRFPMDSHSCPLSFSSFSYPENEMIYKWENFKLEINEKNSWKLFQFDFTGVSNKTEIITTPVGDFMVMTIFFNVSRRFGYVAFQNYVPSSVTTMLSWVSFWIKTESAPARTSLGITSVLTMTTLGTFSRKNFPRVSYITALDFYIAICFVFCFCALLEFAVLNFLIYNQTKAHASPKLRHPRINSRAHARTRARSRACARQHQEAFVCQIVTTEGSDGEERPSCSAQQPPSPGSPEGPRSLCSKLACCEWCKRFKKYFCMVPDCEGSTWQQGRLCIHVYRLDNYSRVVFPVTFFFFNVLYWLVCLNL

SEQ ID NO: 83 - DNA, 3717 bp, Homo sapiens
gagcgcatgcccgcatctgctgtccgacaggcggaagacgagcccagaggcggagcagggccgtcgcgccttggtgacgtctgccgccggcgcgggcgggtgacgcgactgggcccgttgtctgtgtgtgggactgaggggccccgggggcggtgggggctcccggtgggggcagcggtggggagggagggcctggacatggcgctgaggggccgccccgcgggaagatgaataagggctggctggagctggagagcgacccaggcctcttcaccctgctcgtggaagatttcggtgtcaagggggtgcaagtggaggagatctacgaccttcagagcaaatgtcagggccctgtatatggatttatcttcctgttcaaatggatcgaagagcgccggtcccggcgaaaggtctctaccttggtggatgatacgtccgtgattgatgatgatattgtgaataacatgttctttgcccaccagctgatacccaactcttgtgcaactcatgccttgctgagcgtgctcctgaactgcagcagcgtggacctgggacccaccctgagtcgcatgaaggacttcaccaagggtttcagccctgagagcaaaggatatgcgattggcaatgccccggagttggccaaggcccataatagccatgccaggcccgagccacgccacctccctgagaagcagaatggccttagtgcagtgcggaccatggaggcgttccactttgtcagctatgtgcctatcacaggccggctctttgagctggatgggctgaaggtctaccccattgaccatgggccctggggggaggacgaggagtggacagacaaggcccggcgggtcatcatggagcgtatcggcctcgccactgcaggggagccctaccacgacatccgcttcaacctgatggcagtggtgcccgaccgcaggatcaagtatgaggccaggctgcatgtgctgaaggtgaaccgtcagacagtactagaggctctgcagcagctgataagagtaacacagccagagctgattcagacccacaagtctcaagagtcacagctgcctgaggagtccaagtcagccagcaacaagtccccgctggtgctggaagcaaacagggcccctgcagcctctgagggcaaccacacagatggtgcagaggaggcggctggttcatgcgcacaagccccatcccacagccctcccaacaaacccaagctagtggtgaagcctccaggcagcagcctcaatggggttcaccccaaccccactcccattgtccagcggctgccggcctttctagacaatcacaattatgccaagtcccccatgcaggaggaagaagacctggcggcaggtgtgggccgcagccgagttccagtccgcccaccccagcagtactcagatgatgaggatgactatgaggatgacgaggaggatgacgtgcagaacaccaactctgcccttaggtataaggggaagggaacagggaagccaggggcattgagcggttctgctgatgggcaactgtcagtgctgcagcccaacaccatcaacgtcttggctgagaagctcaaagagtcccagaaggacctctcaattcctctgtccatcaagactagcagcggggctgggagtccggctgtggcagtgcccacacactcgcagccctcacccacccccagcaatgagagtacagacacggcctctgagatcggcagtgctttcaactcgccactgcgctcgcctatccgctcagccaacccgacgcggccctccagccctgtcacctcccacatctccaaggtgctttttggagaggatgacagcctgctgcgtgttgactgcatacgctacaaccgtgctgtccgtgatctgggtcctgtcatcagcacaggcctgctgcacctggctgaggatggggtgctgagtcccctggcgctgacagagggtgggaagggttcctcgccctccatcagaccaatccaaggcagccaggggtccagcagcccagtggagaaggaggtcgtggaagccacggacagcagagagaagacggggatggtgaggcctggcgagcccttgagtggggagaaatactcacccaaggagctgctggcactgctgaagtgtgtggaggctgagattgcaaactatgaggcgtgcctcaaggaggaggtagagaagaggaagaagttcaagattgatgaccagagaaggacccacaactacgatgagttcatctgcacctttatctccatgctggctcaggaaggcatgctggccaacctagtggagcagaacatctccgtgcggcggcgccaaggggtcagcatcggccggctccacaagcagcggaagcctgaccggcggaaacgctctcgcccctacaaggccaagcgccagtgaggactgctggccctgactctgcagcccactcttgccgtgtggccctcaccagggtccttccctgccccacttccccttttcccagtattactgaatagtcccagctggagagtccaggccctgggaatgggaggaaccaggccacattccttccatcgtgccctgaggcctgacacggcagatcagccccatagtgctcaggaggcagcatctggagttggggcacagcgaggtactgcagcttcctccacagccggctgtggagcagcaggacctggcccttctgcctgggcagcagaatatatattttacctatcagagacatctatttttctgggctccaacccaacatgccaccatgttgacataagttcctacctgactatgctttctctcctaggagctgtcctggtgggcccaggtccttgtatcatgccacggtcccaactacagggtcctagctgggggcctgggtgggccctgggctctgggccctgctgctctagccccagccaccagcctgtccctgttgtaaggaagccaggtcttctctcttcattcctcttaggagagtgccaaactcagggacccagcactgggctgggttgggagtagggtgtcccagtggggttggggtgagcaggctgctgggatcccatggcctgagcagagcatgtgggaactgttcagtggcctgtgaactgtcttccttgttctagccaggctgttcaagactgctctccatagcaaggttctagggctcttcgccttcagtgttgtggccctagctatgggcctaaattgggctctaggtctctgtccctggcgcttgaggctcagaagagcctctgtccagcccctcagtattaccatgtctccctctcaggggtagcagagacagggttgcttataggaagctggcaccactcagctcttcctgctactccagtttcctcagcctctgcaaggcactcagggtgggggacagcaggatcaagacaacccgttggagcccctgtgttccagaggacctgatgccaaggggtaatgggcccagcagtgcctctggagcccaggccccaacacagccccatggcctctgccagatggctttgaaaaaggtgatccaagcaggcccctttatctgtacatagtgactgagtggggggtgctggcaagtgtggcagctgcctctgggctgagcacagcttgacccctctagcccctgtaaatactggatcaatgaatgaataaaactctcctaagaatctcctgagaaatgaaaaaaaaaaaaaaaaaa

SEQ ID NO: 84 - protein, 729 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 85 - DNA, 7224 bp, Homo sapiens
gtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 86 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 87 - DNA, 7132 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 88 - protein, 1816 aa, Homo sapiens
MLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 89 - DNA, 3699 bp, Homo sapiens
ttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 90 - protein, 759 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 91 - DNA, 3800 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 92 - protein, 699 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTGCPPNCGCAARCLDRGQWLPCNWADV

SEQ ID NO: 93 - DNA, 7287 bp, Homo sapiens
gtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagattcgcatatacatggccaaaggaacaactccatgttttctaaaaggcctagagaacatatatcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 94 - protein, 1884 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKDSHIHGQRNNSMFSKRPREHISVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 95 - DNA, 7128 bp, Homo sapiens
agataactgggcccctgcgctcaggaggccttcaccctctgctctgggtaaaggtagtagagtcccgggaaagggacagggggcccaagtgatgctctggggtactggcgtgggagagtggatttccgaagctgacagatggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 96 - DNA, 4077 bp, Homo sapiens
gccagtgagcccccgcgacggtggcccggacggaaaagatacctcggcggcgtgggcccggctccctgctccaggacctagggatcttggccttccaccctcctccgagcaccaggactccctccagttccgtacccgaggcctccgtggtgaagaggtgccggacccgatgagctcgggagtccaccatcgctctgcaagccgcagttaaacgagaagattcatcaccgctttgatggctgcctcacaaacttcacaaactgttgcatctcacgttccttttgcagatttgtgttcaactttagaacgaatacagaaaagtaaaggacgtgcagaaaaaatcagacacttcagggaatttttagattcttggagaaaatttcatgatgctcttcataagaaccacaaagatgtcacagactctttttatccagcaatgagactaattcttcctcagctagaaagagagagaatggcctatggaattaaagaaactatgcttgctaagctttatattgagttgcttaatttacctagagatggaaaagatgccctcaaacttttaaactacagaacacccactggaactcatggagatgctggagactttgcaatgattgcatattttgtgttgaagccaagatgtttacagaaaggaagtttaaccatacagcaagtaaacgaccttttagactcaattgccagcaataattctgctaaaagaaaagacctaataaaaaagagccttcttcaacttataactcagagttcagcacttgagcaaaagtggcttatacggatgatcataaaggatttaaagcttggtgttagtcagcaaactatcttttctgtttttcataatgatgctgctgagttgcataatgtcactacagatctggaaaaagtctgtaggcaactgcatgatccttctgtaggactcagtgatatttctatcactttattttctgcatttaaaccaatgctagctgctattgcagatattgagcacattgagaaggatatgaaacatcagagtttctacatagaaaccaagctagatggtgaacgtatgcaaatgcacaaagatggagatgtatataaatacttctctcgaaatggatataactacactgatcagtttggtgcttctcctactgaaggttctcttaccccattcattcataatgcattcaaagcagatatacaaatctgtattcttgatggtgagatgatggcctataatcctaatacacaaactttcatgcaaaagggaactaagtttgatattaaaagaatggtagaggattctgatctgcaaacttgttattgtgtttttgatgtattgatggttaataataaaaagctagggcatgagactctgagaaagaggtatgagattcttagtagtatttttacaccaattccaggtagaatagaaatagtgcagaaaacacaagctcatactaagaatgaagtaattgatgcattgaatgaagcaatagataaaagagaagagggaattatggtaaaacaacctctatccatctacaagccagacaaaagaggtgaagggtggttaaaaattaaaccagagtatgtcagtggactaatggatgaattggacattttaattgttggaggatattggggtaaaggatcacggggtggaatgatgtctcattttctgtgtgcagtagcagagaagccccctcctggtgagaagccatctgtgtttcatactctctctcgtgttgggtctggctgcaccatgaaagaactgtatgatctgggtttgaaattggccaagtattggaagccttttcatagaaaagctccaccaagcagcattttatgtggaacagagaagccagaagtatacattgaaccttgtaattctgtcattgttcagattaaagcagcagagatcgtacccagtgatatgtataaaactggctgcaccttgcgttttccacgaattgaaaagataagagatgacaaggagtggcatgagtgcatgaccctggacgacctagaacaacttagggggaaggcatctggtaagctcgcatctaaacacctttatataggtggtgatgatgaaccacaagaaaaaaagcggaaagctgccccaaagatgaagaaagttattggaattattgagcacttaaaagcacctaaccttactaacgttaacaaaatttctaatatatttgaagatgtagagttttgtgttatgagtggaacagatagccagccaaagcctgacctggagaacagaattgcagaatttggtggttatatagtacaaaatccaggcccagacacgtactgtgtaattgcagggtctgagaacatcagagtgaaaaacataattttgtcaaataaacatgatgttgtcaagcctgcatggcttttagaatgttttaagaccaaaagctttgtaccatggcagcctcgctttatgattcatatgtgcccatcaaccaaagaacattttgcccgtgaatatgattgctatggtgatagttatttcattgatacagacttgaaccaactgaaggaagtattctcaggaattaaaaattctaacgagcagactcctgaagaaatggcttctctgattgctgatttagaatatcggtattcctgggattgctctcctctcagtatgtttcgacgccacaccgtttatttggactcgtatgctgttattaatgacctgagtaccaaaaatgaggggacaaggttagctattaaagccttggagcttcggtttcatggagcaaaagtagtttcttgtttagctgagggagtgtctcatgtaataattggggaagatcatagtcgtgttgcagattttaaagcttttagaagaacttttaagagaaagtttaaaatcctaaaagaaagttgggtaactgattcaatagacaagtgtgaattacaagaagaaaaccagtatttgatttaaagctaggtttcctagtgaggaaagcctctgatctggcagactcattgcagcaggtggtaatgataaaatactaaactacattttatttttgtatcttaaaaatctatgcctaaaaagtatcattacatataggaaaacaataattttaacttttaaggttgaaaagacaatagcccaaagccaagaaagaaaaattatcttgaatgtagtattcaatgattttttatgatcaaggtgaaataaacagtctaaagaagaggtgtttttataatatccatatagaaatctagaatttttacttagatactaataaaatacatttagaaacttttaaagtcatgaaaaagcattaaccttctaaacagtatattctaaaaagtcaaaacgttaacaatagtttttatctaataaaagcactgcaagaaaatagggtagaattgttacagctggacttgtaaaaatatgtctttttactcagggtttaaaatgtcccatttaaatatgaaatgtaaacaaatttgttttttaaggttaaggccaaatgtaacaataaaaccctgtcgatggttttagctaaattagaggaagttgtatgagacttaatgatctaaaaacttaaaattgaattggtttgattaaaaataaagcttgcaattttaaaagtagctcacatttaatttcttgtgtgaaatagaacatgctttaaaggaagtatttttatgtgaatttgcattccagtataaatagtattcacaaaaaagattttcctagattttatctattgaataggtgtcaatatggcatgcatattgtaactttcattagaaataagttgctttgacttttaaaaatgacatagttagattatttaaagtcaatgtatatagtatatattatgtatggatttatataccaaattttggaatacagcctatctcatgaccatattgaaatgtacggaatttgatccatgcgatactatgtgtgcattatttgaaagttattggaaattttattcaaaccgtggaacaaatgtatgtgattttgttatacttcttaatttaaataaaatatttaatgcactattaaaaaaaaaaaaaaaaaaa

SEQ ID NO: 97 - protein, 911 aa, Homo sapiens
MAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDEPQEKKRKAAPKMKKVIGIIEHLKAPNLTNVNKISNIFEDVEFCVMSGTDSQPKPDLENRIAEFGGYIVQNPGPDTYCVIAGSENIRVKNIILSNKHDVVKPAWLLECFKTKSFVPWQPRFMIHMCPSTKEHFAREYDCYGDSYFIDTDLNQLKEVFSGIKNSNEQTPEEMASLIADLEYRYSWDCSPLSMFRRHTVYLDSYAVINDLSTKNEGTRLAIKALELRFHGAKVVSCLAEGVSHVIIGEDHSRVADFKAFRRTFKRKFKILKESWVTDSIDKCELQEENQYLI

SEQ ID NO: 98 - DNA, 4115 bp, Homo sapiens
ccacagcgctgtagactgcgccgcattagaagcctggcctcctgatgctgtgctcttcatctagacccaagccccaggtcgtgggacgatttctcccgtttttgactccctggaactgtattgcctgctttacctgcgtacatgttgattctttctcatggcaaccccgcaggaaaccatcaagatctcattttacagctgggattctctggttcacagaggtaacggagcttgcccgaggccagttaaacgagaagattcatcaccgctttgatggctgcctcacaaacttcacaaactgttgcatctcacgttccttttgcagatttgtgttcaactttagaacgaatacagaaaagtaaaggacgtgcagaaaaaatcagacacttcagggaatttttagattcttggagaaaatttcatgatgctcttcataagaaccacaaagatgtcacagactctttttatccagcaatgagactaattcttcctcagctagaaagagagagaatggcctatggaattaaagaaactatgcttgctaagctttatattgagttgcttaatttacctagagatggaaaagatgccctcaaacttttaaactacagaacacccactggaactcatggagatgctggagactttgcaatgattgcatattttgtgttgaagccaagatgtttacagaaaggaagtttaaccatacagcaagtaaacgaccttttagactcaattgccagcaataattctgctaaaagaaaagacctaataaaaaagagccttcttcaacttataactcagagttcagcacttgagcaaaagtggcttatacggatgatcataaaggatttaaagcttggtgttagtcagcaaactatcttttctgtttttcataatgatgctgctgagttgcataatgtcactacagatctggaaaaagtctgtaggcaactgcatgatccttctgtaggactcagtgatatttctatcactttattttctgcatttaaaccaatgctagctgctattgcagatattgagcacattgagaaggatatgaaacatcagagtttctacatagaaaccaagctagatggtgaacgtatgcaaatgcacaaagatggagatgtatataaatacttctctcgaaatggatataactacactgatcagtttggtgcttctcctactgaaggttctcttaccccattcattcataatgcattcaaagcagatatacaaatctgtattcttgatggtgagatgatggcctataatcctaatacacaaactttcatgcaaaagggaactaagtttgatattaaaagaatggtagaggattctgatctgcaaacttgttattgtgtttttgatgtattgatggttaataataaaaagctagggcatgagactctgagaaagaggtatgagattcttagtagtatttttacaccaattccaggtagaatagaaatagtgcagaaaacacaagctcatactaagaatgaagtaattgatgcattgaatgaagcaatagataaaagagaagagggaattatggtaaaacaacctctatccatctacaagccagacaaaagaggtgaagggtggttaaaaattaaaccagagtatgtcagtggactaatggatgaattggacattttaattgttggaggatattggggtaaaggatcacggggtggaatgatgtctcattttctgtgtgcagtagcagagaagccccctcctggtgagaagccatctgtgtttcatactctctctcgtgttgggtctggctgcaccatgaaagaactgtatgatctgggtttgaaattggccaagtattggaagccttttcatagaaaagctccaccaagcagcattttatgtggaacagagaagccagaagtatacattgaaccttgtaattctgtcattgttcagattaaagcagcagagatcgtacccagtgatatgtataaaactggctgcaccttgcgttttccacgaattgaaaagataagagatgacaaggagtggcatgagtgcatgaccctggacgacctagaacaacttagggggaaggcatctggtaagctcgcatctaaacacctttatataggtggtgatgatgaaccacaagaaaaaaagcggaaagctgccccaaagatgaagaaagttattggaattattgagcacttaaaagcacctaaccttactaacgttaacaaaatttctaatatatttgaagatgtagagttttgtgttatgagtggaacagatagccagccaaagcctgacctggagaacagaattgcagaatttggtggttatatagtacaaaatccaggcccagacacgtactgtgtaattgcagggtctgagaacatcagagtgaaaaacataattttgtcaaataaacatgatgttgtcaagcctgcatggcttttagaatgttttaagaccaaaagctttgtaccatggcagcctcgctttatgattcatatgtgcccatcaaccaaagaacattttgcccgtgaatatgattgctatggtgatagttatttcattgatacagacttgaaccaactgaaggaagtattctcaggaattaaaaattctaacgagcagactcctgaagaaatggcttctctgattgctgatttagaatatcggtattcctgggattgctctcctctcagtatgtttcgacgccacaccgtttatttggactcgtatgctgttattaatgacctgagtaccaaaaatgaggggacaaggttagctattaaagccttggagcttcggtttcatggagcaaaagtagtttcttgtttagctgagggagtgtctcatgtaataattggggaagatcatagtcgtgttgcagattttaaagcttttagaagaacttttaagagaaagtttaaaatcctaaaagaaagttgggtaactgattcaatagacaagtgtgaattacaagaagaaaaccagtatttgatttaaagctaggtttcctagtgaggaaagcctctgatctggcagactcattgcagcaggtggtaatgataaaatactaaactacattttatttttgtatcttaaaaatctatgcctaaaaagtatcattacatataggaaaacaataattttaacttttaaggttgaaaagacaatagcccaaagccaagaaagaaaaattatcttgaatgtagtattcaatgattttttatgatcaaggtgaaataaacagtctaaagaagaggtgtttttataatatccatatagaaatctagaatttttacttagatactaataaaatacatttagaaacttttaaagtcatgaaaaagcattaaccttctaaacagtatattctaaaaagtcaaaacgttaacaatagtttttatctaataaaagcactgcaagaaaatagggtagaattgttacagctggacttgtaaaaatatgtctttttactcagggtttaaaatgtcccatttaaatatgaaatgtaaacaaatttgttttttaaggttaaggccaaatgtaacaataaaaccctgtcgatggttttagctaaattagaggaagttgtatgagacttaatgatctaaaaacttaaaattgaattggtttgattaaaaataaagcttgcaattttaaaagtagctcacatttaatttcttgtgtgaaatagaacatgctttaaaggaagtatttttatgtgaatttgcattccagtataaatagtattcacaaaaaagattttcctagattttatctattgaataggtgtcaatatggcatgcatattgtaactttcattagaaataagttgctttgacttttaaaaatgacatagttagattatttaaagtcaatgtatatagtatatattatgtatggatttatataccaaattttggaatacagcctatctcatgaccatattgaaatgtacggaatttgatccatgcgatactatgtgtgcattatttgaaagttattggaaattttattcaaaccgtggaacaaatgtatgtgattttgttatacttcttaatttaaataaaatatttaatgcactattaaaaaaaaaaaaaaaaaaa

SEQ ID NO: 99 - protein, 911 aa, Homo sapiens; identical to SEQ ID NO: 97

SEQ ID NO: 100 - DNA, 3994 bp, Homo sapiens
cttctggcgccagcttccggcttagcggctgagcttcaggcttgacgtcaggaaaccatcaagatctcattttacagctgggattctctggttcacagaggtaacggagcttgcccgaggccagttaaacgagaagattcatcaccgctttgatggctgcctcacaaacttcacaaactgttgcatctcacgttccttttgcagatttgtgttcaactttagaacgaatacagaaaagtaaaggacgtgcagaaaaaatcagacacttcagggaatttttagattcttggagaaaatttcatgatgctcttcataagaaccacaaagatgtcacagactctttttatccagcaatgagactaattcttcctcagctagaaagagagagaatggcctatggaattaaagaaactatgcttgctaagctttatattgagttgcttaatttacctagagatggaaaagatgccctcaaacttttaaactacagaacacccactggaactcatggagatgctggagactttgcaatgattgcatattttgtgttgaagccaagatgtttacagaaaggaagtttaaccatacagcaagtaaacgaccttttagactcaattgccagcaataattctgctaaaagaaaagacctaataaaaaagagccttcttcaacttataactcagagttcagcacttgagcaaaagtggcttatacggatgatcataaaggatttaaagcttggtgttagtcagcaaactatcttttctgtttttcataatgatgctgctgagttgcataatgtcactacagatctggaaaaagtctgtaggcaactgcatgatccttctgtaggactcagtgatatttctatcactttattttctgcatttaaaccaatgctagctgctattgcagatattgagcacattgagaaggatatgaaacatcagagtttctacatagaaaccaagctagatggtgaacgtatgcaaatgcacaaagatggagatgtatataaatacttctctcgaaatggatataactacactgatcagtttggtgcttctcctactgaaggttctcttaccccattcattcataatgcattcaaagcagatatacaaatctgtattcttgatggtgagatgatggcctataatcctaatacacaaactttcatgcaaaagggaactaagtttgatattaaaagaatggtagaggattctgatctgcaaacttgttattgtgtttttgatgtattgatggttaataataaaaagctagggcatgagactctgagaaagaggtatgagattcttagtagtatttttacaccaattccaggtagaatagaaatagtgcagaaaacacaagctcatactaagaatgaagtaattgatgcattgaatgaagcaatagataaaagagaagagggaattatggtaaaacaacctctatccatctacaagccagacaaaagaggtgaagggtggttaaaaattaaaccagagtatgtcagtggactaatggatgaattggacattttaattgttggaggatattggggtaaaggatcacggggtggaatgatgtctcattttctgtgtgcagtagcagagaagccccctcctggtgagaagccatctgtgtttcatactctctctcgtgttgggtctggctgcaccatgaaagaactgtatgatctgggtttgaaattggccaagtattggaagccttttcatagaaaagctccaccaagcagcattttatgtggaacagagaagccagaagtatacattgaaccttgtaattctgtcattgttcagattaaagcagcagagatcgtacccagtgatatgtataaaactggctgcaccttgcgttttccacgaattgaaaagataagagatgacaaggagtggcatgagtgcatgaccctggacgacctagaacaacttagggggaaggcatctggtaagctcgcatctaaacacctttatataggtggtgatgatgaaccacaagaaaaaaagcggaaagctgccccaaagatgaagaaagttattggaattattgagcacttaaaagcacctaaccttactaacgttaacaaaatttctaatatatttgaagatgtagagttttgtgttatgagtggaacagatagccagccaaagcctgacctggagaacagaattgcagaatttggtggttatatagtacaaaatccaggcccagacacgtactgtgtaattgcagggtctgagaacatcagagtgaaaaacataattttgtcaaataaacatgatgttgtcaagcctgcatggcttttagaatgttttaagaccaaaagctttgtaccatggcagcctcgctttatgattcatatgtgcccatcaaccaaagaacattttgcccgtgaatatgattgctatggtgatagttatttcattgatacagacttgaaccaactgaaggaagtattctcaggaattaaaaattctaacgagcagactcctgaagaaatggcttctctgattgctgatttagaatatcggtattcctgggattgctctcctctcagtatgtttcgacgccacaccgtttatttggactcgtatgctgttattaatgacctgagtaccaaaaatgaggggacaaggttagctattaaagccttggagcttcggtttcatggagcaaaagtagtttcttgtttagctgagggagtgtctcatgtaataattggggaagatcatagtcgtgttgcagattttaaagcttttagaagaacttttaagagaaagtttaaaatcctaaaagaaagttgggtaactgattcaatagacaagtgtgaattacaagaagaaaaccagtatttgatttaaagctaggtttcctagtgaggaaagcctctgatctggcagactcattgcagcaggtggtaatgataaaatactaaactacattttatttttgtatcttaaaaatctatgcctaaaaagtatcattacatataggaaaacaataattttaacttttaaggttgaaaagacaatagcccaaagccaagaaagaaaaattatcttgaatgtagtattcaatgattttttatgatcaaggtgaaataaacagtctaaagaagaggtgtttttataatatccatatagaaatctagaatttttacttagatactaataaaatacatttagaaacttttaaagtcatgaaaaagcattaaccttctaaacagtatattctaaaaagtcaaaacgttaacaatagtttttatctaataaaagcactgcaagaaaatagggtagaattgttacagctggacttgtaaaaatatgtctttttactcagggtttaaaatgtcccatttaaatatgaaatgtaaacaaatttgttttttaaggttaaggccaaatgtaacaataaaaccctgtcgatggttttagctaaattagaggaagttgtatgagacttaatgatctaaaaacttaaaattgaattggtttgattaaaaataaagcttgcaattttaaaagtagctcacatttaatttcttgtgtgaaatagaacatgctttaaaggaagtatttttatgtgaatttgcattccagtataaatagtattcacaaaaaagattttcctagattttatctattgaataggtgtcaatatggcatgcatattgtaactttcattagaaataagttgctttgacttttaaaaatgacatagttagattatttaaagtcaatgtatatagtatatattatgtatggatttatataccaaattttggaatacagcctatctcatgaccatattgaaatgtacggaatttgatccatgcgatactatgtgtgcattatttgaaagttattggaaattttattcaaaccgtggaacaaatgtatgtgattttgttatacttcttaatttaaataaaatatttaatgcactattaaaaaaaaaaaaaaaaaaa

SEQ ID NO: 101 - protein, 911 aa, Homo sapiens; identical to SEQ ID NO: 97

(19) United States
(12) Patent Application Publication
(10) Pub. No.: US 2018/0122507 A1
(43) Pub. Date: May 3, 2018

(54) GENETIC ALTERATIONS IN OVARIAN CANCER

(71) Applicant: University of Utah Research Foundation, Salt Lake City, UT (US)

(72) Inventor: Orly ALTER, Salt Lake City, UT (US)

(21) Appl. No.: 15/566,294

(22) PCT Filed: Apr. 14, 2016
(86) PCT No.: PCT/US16/27641

§ 371 Date: Oct. 13, 2017

Related U.S. Application Data
(60) Provisional application No. 62/147,545, filed on Apr. 14, 2015.
(60) Provisional application No. 62/147,555, filed on Apr. 14, 2015.

(51) Int. Cl. G16H 50/20; G06F 19/18; G01N 33/574

(52) U.S. Cl. CPC G16H 50/20; G01N 33/57449; G06F 19/18

(57) ABSTRACT

According to various embodiments herein, methods for performing diagnosis and prognosis of OV have been provided. In embodiments, a method of determining an estimated outcome or predicting a clinical response to chemotherapy for a patient having ovarian serous cystadenocarcinoma (OV), comprises obtaining a biological sample from a patient diagnosed with OV, said sample comprising at least one of nucleic acids and proteins from the patient; detecting in said sample a value of an indicator of a differential expression; and calculating, by a processor, a weighted sum pattern based on the value of one or more of the indicators of differential expression; and estimating, by the processor and based on the weighted sum pattern, a predicted length of survival of the patient or a predicted clinical response to chemotherapy for the patient.

### CROSS REFERENCE TO RELATED APPLICATIONS

This application claims priority to U.S. Provisional Application No. 62/147,555, entitled “Advanced Tensor Decompositions for Computational Assessment in Ovarian Cancer,” and U.S. Provisional Application No. 62/147,545, entitled “Genetic Alterations in Ovarian Cancer,” each filed Apr. 14, 2015, the disclosures of which are hereby incorporated by reference in their entireties.

### GOVERNMENT LICENSE RIGHTS

This invention was made with government support under the Utah Science Technology and Research (USTAR) Initiative and the National Human Genome Research Institute (NHGRI) RO1 Grant HG-004302; and the National Science Foundation (NSF) CAREER Award DMS-0847173. The government has certain rights in this invention.

### FIELD

The subject technology relates generally to computational biology and its use to identify genetic patterns related to cancer.

### BACKGROUND

In many areas of science, especially in biotechnology, the number of high-dimensional datasets recording multiple aspects of a single phenomenon is increasing. This increase is accompanied by a fundamental need for mathematical frameworks that can compare multiple large-scale matrices with different row dimensions. In the field of biotechnology, these matrices may represent biological reality through large-scale molecular biological data such as, for example, mRNA expression measured by DNA microarray.

Recent efforts have focused on developing ways of modeling and analyzing large-scale molecular biological data through the use of the matrices and their generalizations in different types of genomic data. One of the goals of these efforts is to computationally predict mechanisms that govern the activity of DNA and RNA. For example, matrices have been used to predict global causal coordination between DNA replication origin activity and mRNA expression from mathematical modeling of DNA microarray data. The mathematical variables, that is patterns, uncovered in the data correlate with activities of cellular elements such as regulators or transcription factors. The operations, such as classification, rotation, or reconstruction in subspaces of these patterns, simulate experimental observation of the correlations and possibly even the causal coordination of these activities.

Recently, a generalized singular value decomposition was demonstrated in comparative modeling of patient-matched but probe-independent glioblastoma (GBM) brain tumor and normal DNA copy-number profiles in the TCGA. Analysis showed and validated a pattern correlated with a GBM patient's prognosis and response to chemotherapy.

These types of analyses also have the potential to be extended to the study of pathological diseases to identify patterns that correlate and possibly coordinate with the diseases.

### SUMMARY

Ovarian serous cystadenocarcinoma (OV) accounts for about 90% of all ovarian cancers. Most of the OV tumors, i.e. greater than 95%, are high-grade tumors. OV exhibits a range of copy-number alterations (CNA), some of which are believed to play a role in the cancer's pathogenesis. OV copy number alteration data are available from The Cancer Genome Atlas (TCGA).

Despite recent large-scale profiling efforts, the best predictor of OV survival to date has remained the tumor's stage at diagnosis, a pathological assessment of the spread of the cancer numbering I to IV. Other indicators of prognosis are dense adherence and the presence of large-volume ascites. Traditional treatments of OV include, but are not limited to, platinum-based chemotherapy, radiation, radiosurgery, surgery, etc. About 25% of primary OV tumors are resistant to platinum-based chemotherapy. Further, most recurrent OV tumors develop resistance to platinum-based chemotherapy. Even though drugs exist for platinum-based chemotherapy resistant OV, no pathology laboratory diagnostic currently exists that distinguishes between resistant and sensitive tumors before treatment. OV tumors exhibit significant CNA variation, much more so than, e.g., GBM tumors. Further, very few frequent CNAs typical of OV have been identified so far.

Therefore, there is a need to model and analyze the large scale molecular biological data of OV patients in order to identify genomic features or factors (e.g., genes) and mechanisms that allow one to make predictions on the course of the disease and/or possible treatments. The subject technology identifies and utilizes such genomic features that are useful in the diagnosis and prognosis of OV.

According to various embodiments of the subject technology, methods for performing diagnosis and prognosis of OV have been provided. In embodiments, a method of determining an estimated outcome or predicting a clinical response to chemotherapy for a patient having ovarian serous cystadenocarcinoma (OV), comprises obtaining a biological sample from a patient diagnosed with OV, said sample comprising at least one of nucleic acids and proteins from the patient; detecting in said sample a value of an indicator of a differential expression of at least one of (a) a nucleotide sequence having at least 90% sequence identity to at least one of the genes selected from Ckdn1A, Mapk14, Kras, Rad51AP1, Tnf, Itpr2, Rpa3, Pold2, Lig4, Pabpc5, Bcap31, and Gabre; (b) a protein encoded by the genes of (a); (c) a nucleotide sequence having at least 90% sequence identity to at least one of cytogenic bands 1-7 and 11-17; (d) a microRNA sequence selected from miR-877, miR-877*, miR-200c, miR-141, miR-888, miR-452, and miR-224; (e) a segment overlapping with the Prim2 gene; and (f) a nucleotide sequence having at least 90% sequence identity to DSX214; calculating, by a processor, a weighted sum pattern based on the value of one or more of the indicators of differential expression; and estimating, by the processor and based on the weighted sum pattern, a predicted length of survival of the patient or a predicted clinical response to chemotherapy for the patient. In embodiments, the method further comprises recommending administering a treatment regimen based on the predicted length of survival of the patient or clinical response to chemotherapy. In embodiments, the method comprises administering a treatment regimen based on the predicted length of survival or clinical response to chemotherapy of the patient. In embodiments, the method further comprises recommending a treatment regimen based on the predicted length of survival or clinical response to chemotherapy of the patient.

In embodiments, at least one nucleotide sequence has at least 90% sequence identity to at least one of the genes selected from Ckdn1A, Mapk14, Kras, Rad51AP1, Tnf, Itpr2, Rpa3, Pold2, Lig4, Pabpc5, Bcap31, and Gabre; and wherein the indicator of differential expression is differential copy number relative to a copy number of the at least one nucleotide sequence in normal cells. In embodiments, at least one nucleotide sequence has at least 90% sequence identity to at least one of cytogenic band 1-7 and cytogenic band 11-17; and wherein the indicator of differential expression is differential copy number relative to a copy number of the at least one nucleotide sequence in normal cells. In embodiments, the at least one protein encoded by the genes of (a) is selected from CKDN1A, MAPK14, KRAS, RAD51AP1, TNF, ITPR2, RPA3, POLD2, LIG4, PABPC5, BCAP31, and GABRE; and wherein the indicator of differential expression is differential protein expression relative to protein expression of the at least one protein in normal cells. In embodiments, the microRNA sequence is at least one of miR-877, miR-877*, miR-200c, miR-141, miR-888, miR-452, and miR-224; and wherein the indicator of differential expression is differential copy number relative to a copy number of the at least one nucleotide sequence in normal cells.

In embodiments, the differential copy number is an increase in copy number relative to a copy number of the at least one nucleotide sequence in normal cells. In embodiments, the differential copy number is a decrease in copy number relative to a copy number of the at least one nucleotide sequence in normal cells. In embodiments, the differential protein expression is an increase in protein expression relative to protein expression in normal cells. In embodiments, the differential protein expression is a decrease in protein expression relative to protein expression in normal cells. In embodiments, the differential copy number is an increase in copy number relative to a copy number of the at least one nucleotide sequence in normal cells. In embodiments, the differential copy number is a decrease in copy number relative to a copy number of the at least one nucleotide sequence in normal cells. In embodiments, the differential expression is microRNA expression. In embodiments, the differential microRNA expression is an increase in microRNA expression relative to microRNA expression of the at least one nucleotide sequence in normal cells. In embodiments, the differential microRNA expression is a decrease in microRNA expression relative to microRNA expression of the at least one nucleotide in normal cells.

In embodiments, the method comprises correlating at least one of the indicators of differential expression selected from (a)-(f) below:
a) co-occurring copy-number loss of Pabpc5, and gain, or mRNA overexpression of Bcap31; or
b) co-occurring copy number loss of Pabpc5, and gain, or mRNA overexpression of Bcap31, and gain, or microRNA overexpression of miR-888, and miR-452; or
c) co-occurring copy number loss of Pabpc5, and gain, or mRNA overexpression of Bcap31, and gain, or microRNA overexpression of miR-888, miR-452, and miR-224; or
d) co-occurring copy-number copy number loss of Pabpc5 and sequence tag site (STS) DXS214, and gain, or mRNA overexpression of Bcap31; or
e) co-occurring copy number loss of Pabpc5, and gain, or mRNA overexpression of Bcap31 and Gabre; or
f) co-occurring copy-number loss from cytogenetic bands 1-14, and gain in cytogenetic bands 16-24;
with at least one of longer survival time and sensitivity to platinum-based chemotherapy.

In embodiments the differential expression of (c) further includes correlating copy-number loss of sequence tag site DXS214 and gain or mRNA overexpression of Bcap31 and Gabre with at least one of longer survival time and sensitivity of platinum-based chemotherapy.

In embodiments, the method comprises correlating at least one of the indicators of differential expression selected from (a1)-(d1) below:
a1) co-occurring copy-number loss, or mRNA underexpression of Rpa3, and copy-number gain, or mRNA overexpression of Pold2; or
b1) co-occurring copy-number loss, or mRNA underexpression of Rpa3 on 7p and Lig4 on 13q, and copy-number gain, or mRNA overexpression of Pold2; or
c1) co-occurring copy-number loss, or mRNA underexpression of Lig4 on chromosome 13q, and copy-number gain, or mRNA overexpression of Pold2; or
d1) co-occurring copy-number loss from cytogenetic bands 1-7, and gain in cytogenetic bands 11-17;
with at least one of a longer survival time and sensitivity to platinum-based chemotherapy.

In embodiments, the method comprises correlating at least one of the indicators of differential expression selected from (a2)-(g2) below:
a2) co-occurring copy-number loss on chromosome 6p and gain on chromosome 12p; or
b2) co-occurring copy-number loss, or mRNA or protein under-expression of Cdkn1A and Mapk14 on chromosome 6p, and copy-number gain, or mRNA or protein overexpression of Kras on chromosome 12p; or
c2) co-occurring copy-number loss, or mRNA or protein under-expression of Cdkn1A and Mapk14 on 6p, and copy-number gain, or mRNA or protein overexpression of Kras and Rad51AP1 on 12p; or
d2) co-occurring copy-number loss, or mRNA or protein under-expression of Cdkn1A, Mapk14, and Tnf on chromosome 6p, and copy-number gain, or mRNA or protein overexpression of Kras, Rad51AP1, and Itpr2 on chromosome 12p; or
e2) co-occurring copy-number loss, or microRNA under-expression of miR-877* on chromosome 6p, and copy-number gain, or microRNA overexpression, of miR-200c, miR-200c*, miR-141, or miR-141* on chromosome 12p;
(f2) co-occurring copy-number loss, or mRNA or protein under-expression of Cdkn1A and Mapk14 on chromosome 6p, and copy-number gain, or mRNA or protein overexpression of Rad51AP1 on chromosome 12p;
(g2) co-occurring copy-number loss, or mRNA or protein under-expression of Tnf on chromosome 6p, and copy-number gain, or mRNA or protein overexpression of Itpr2 on chromosome 12p;
with at least one of shorter survival time and resistance to platinum-based chemotherapy.

In embodiments, the method comprises the differential expression of at least one of (a2)-(g2) and further comprises correlating at least one of:
(h2) a gain in copy numbers or mRNA or protein overexpression of Sox5; or
(i2) a gain in copy numbers or mRNA or protein overexpression of Asun; or
(j2) a gain in copy numbers or mRNA or protein overexpression of Abcf1; or
(k2) a gain in copy numbers or mRNA or protein overexpression of Cdkn1B; or
(l2) an mRNA or protein under-expression or loss in copy numbers of Bap1; or
(m2) a reduced abundance of Brca1-associated genome surveillance protein complex (BASC);
with at least one of a patient's shorter survival time and resistance to platinum-based chemotherapy.

In embodiments, the method comprises correlating at least one of the indicators of differential expression selected from (a)-(f), (a1)-(d1), and (a2)-(e2).

In embodiments, the method further comprises correlating at least one of:

(1) an increase in copy number of the segment overlapping with SEQ ID NO: 1 with at least one of reduced length of patient survival and resistance to platinum-based chemotherapy;

(2) an increase in copy number of SEQ ID NO: 7 with at least one of reduced length of patient survival and resistance to platinum-based chemotherapy;

(3) an increase in copy number of SEQ ID NO: 10 with at least one of reduced length of patient survival and resistance to platinum-based chemotherapy;

(4) an increase in copy number of SEQ ID NO: 21 with at least one of reduced length of patient survival and resistance to platinum-based chemotherapy;

(5) an increase in copy number of SEQ ID NO: 23 with at least one of reduced length of patient survival and resistance to platinum-based chemotherapy;

(6) a decrease in copy number of SEQ ID NO: 25 with at least one of increased length of patient survival and sensitivity to platinum-based chemotherapy;

(7) a decrease in copy number of SEQ ID NO: 27 with at least one of increased length of patient survival and sensitivity to platinum-based chemotherapy;

(8) a decrease in copy number of SEQ ID NO: 29 with at least one of increased length of patient survival and sensitivity to platinum-based chemotherapy;

(9) a decrease in copy number of SEQ ID NO: 31 with at least one of reduced length of patient survival and resistance to platinum-based chemotherapy;

(10) a decrease in copy number of SEQ ID NO: 41 with at least one of reduced length of patient survival and resistance to platinum-based chemotherapy;

(11) a decrease in copy number of SEQ ID NO: 39 with at least one of reduced length of patient survival and resistance to platinum-based chemotherapy;

(12) a decrease in copy number of SEQ ID NO: 51 with at least one of reduced length of patient survival and resistance to platinum-based chemotherapy;

(13) a decrease in copy number of SEQ ID NO: 52 with at least one of reduced length of patient survival and resistance to platinum-based chemotherapy;

(14) an increase in copy number of SEQ ID NO: 56 with at least one of reduced length of patient survival and resistance to platinum-based chemotherapy;

(15) an increase in copy number of SEQ ID NO: 60 with at least one of reduced length of patient survival and resistance to platinum-based chemotherapy;

(16) an increase in copy number of SEQ ID NO: 61 with at least one of reduced length of patient survival and resistance to platinum-based chemotherapy;

(17) an increase in copy number of SEQ ID NO: 62 with at least one of reduced length of patient survival and resistance to platinum-based chemotherapy;

(18) an increase in copy number of SEQ ID NO: 64 with at least one of increased length of patient survival and sensitivity to platinum-based chemotherapy;

(19) an increase in copy number of SEQ ID NO: 70 with at least one of increased length of patient survival and sensitivity to platinum-based chemotherapy;

(20) an increase in copy number of SEQ ID NO: 78 with at least one of increased length of patient survival and sensitivity to platinum-based chemotherapy;

(21) an increase in copy number of SEQ ID NO: 79 with at least one of increased length of patient survival and sensitivity to platinum-based chemotherapy;

(22) an increase in copy number of SEQ ID NO: 80 with at least one of increased length of patient survival and sensitivity to platinum-based chemotherapy;

(23) an increase in copy number of SEQ ID NO: 81 gene with at least one of increased length of patient survival and sensitivity to platinum-based chemotherapy;

(24) a decrease in copy number of SEQ ID NO: 96 gene with at least one of increased length of patient survival and sensitivity to platinum-based chemotherapy;

or any combination of (1)-(24). In embodiments, the method comprises: (i) correlating at least two of (2), (4), (6), (9)-(12), (14)-(16), (18), and (24);

(ii) correlating at least two of (2), (4), (7), (9)-(12), (14)-(16), (19)-(23); or

(iii) correlating at least two of (6)-(7), and (18)-(24).

In some embodiments, methods of estimating an outcome for a patient having an OV tumor, comprises: obtaining a biological sample from a patient diagnosed with OV, said sample comprising at least one of nucleic acids and proteins from the patient; detecting in said sample a value of an indicator of a differential copy number of each of at least one of (a) a nucleotide sequence, each sequence having at least 90% sequence identity to at least one gene selected from Ckdn1A, Mapk14, Kras, Rad51AP1, Tnf, Itpr2, Rpa3, Pold2, Lig4, Pabpc5, Bcap31, and Gabre; (b) a protein encoded by the genes of (a); (c) a nucleotide sequence having at least 90% sequence identity to at least one of cytogenic bands 1-7 and 11-17; (d) a microRNA sequence selected from miR-877, miR-877*, miR-200c, miR-141, miR-888, miR-452, and miR-224; (e) a segment overlapping with the Prim2 gene; and (f) a nucleotide sequence having at least 90% sequence identity to DSX214; calculating, by a processor, a weighted sum pattern based on the value of one or more of the differential copy number; and estimating, by the processor and based on the weighted sum pattern, a predicted length of survival of the patient or a predicted clinical response to chemotherapy for the patient. In embodiments, the at least one nucleotide sequence has at least 90% sequence identity to at least one of the genes selected from Rad51AP1, Cdkn1B, Kras, Itpr2, Rpa3, and Pabpc5, wherein the copy number of one or more of the genes is increased relative to a copy number of the at least one nucleotide sequence in normal cells and reflects an enhanced probability of length of survival of the patient relative to a probability of length of survival of patients without the increased copy number. In embodiments, the at least one nucleotide has at least 90% sequence identity to at least one of the genes selected from Rad51AP1, Cdkn1B, Kras, Itpr2, Rpa3, and Pabpc5; and wherein the copy number of one or more of the genes is decreased relative to a copy number of the at least one nucleotide sequence in normal cells and reflects an enhanced probability of length of survival of the patient relative to a probability of length of survival of patients without the decreased copy number. In embodiments, the copy number of the nucleotide sequence having at least 90% sequence identity to at least one of the genes selected from Cdkn1A, Mapk14, Tnf, Pold2, Bcap31 is increased relative to a copy number of the gene in normal cells which reflects an enhanced probability of length of survival of the patient relative to a probability of length of survival of patients without the increased copy number.

Alternatively, the nucleotide sequences may have at least about 85 percent sequence identity, at least about 95% sequence identity, at least about 96% sequence identity, at least about 97% sequence identity, at least about 98% sequence identity, at least about 99% sequence identity, or 100% sequence identity to at least one of the genes selected from Cdkn1A, Mapk14, Tnf, Pold2, Bcap31. Sequence similarity or identity can be identified using a suitable sequence alignment algorithm, such as ClustalW2 (http://www.ebi.ac.uk/Tools/clustalw2/index.html) or “BLAST 2 Sequences” using default parameters (Tatusova, T. et al., FEMS Microbiol. Lett., 174:187-188 (1999)).

In some embodiments, the copy number of one or more of the genes is increased relative to a copy number of the at least one nucleotide sequence in normal cells and reflects an enhanced probability of length of survival of the patient relative to a probability of length of survival of patients without the increased copy number. In other embodiments, the copy number of one or more of the genes is decreased relative to a copy number of the at least one nucleotide sequence in normal cells and reflects an enhanced probability of length of survival of the patient relative to a probability of length of survival of patients without the decreased copy number. In one particular embodiment, where the copy number of the nucleotide sequence having at least 90% sequence identity to at least one of the genes selected from Rad51AP1, Cdkn1B, Kras, Itpr2, Rpa3, Pabpc5 is decreased relative to a copy number of the gene in normal cells reflects an enhanced probability of length survival of the patient relative to a probability of length survival of patients without the decreased copy number. In another embodiment, where the copy number of the nucleotide sequence having at least 90% sequence identity to at least one of the genes selected from Cdkn1A, Mapk14, Tnf, Pold2, Bcap31 is increased relative to a copy number of the gene in normal cells reflects an enhanced probability of length of survival of the patient relative to a probability of length of survival of patients without the increased copy number. In a further embodiment, where the copy number of the nucleotide sequence having at least 90% sequence identity to Cdkn1A, Mapk14, Tnf is decreased relative to a copy number of the gene in normal cells and wherein the copy number of the nucleotide sequence having at least 90% sequence identity to Kras, Rad51AP1 and ITPR2 is increased relative to a copy number of the gene in normal cells reflects a decreased probability of length of survival relative to a probability of length of survival of patients without this pattern of increased and decreased copy number. In yet another embodiment, where the copy number of the nucleotide sequence having at least 90% sequence identity to Cdkn1A and Mapk14 is decreased relative to a copy number of the gene in normal cells, and the copy number of the nucleotide sequence having at least 90% sequence identity to Kras and Rad51AP1 is increased relative to a copy number of the gene in normal cells reflects a decreased probability of length of survival relative to a probability of length of survival of patients without this pattern of increased and decreased copy number. In another embodiment, where wherein the copy number of the nucleotide sequence having at least 90% sequence identity to Rpa3 is decreased relative to a copy number of the gene in normal cells, and the copy number of the nucleotide sequence having at least 90% sequence identity to Pold2 is increased relative to a copy number of the gene in normal cells reflects an increased probability of length of survival relative to a probability of length of survival of patients without this pattern of increased and decreased copy number. In a further embodiment, where the copy number of the nucleotide sequence having at least 90% sequence identity to Pabpc5 is decreased relative to a copy number of the gene in normal cells, and the copy number of the nucleotide sequence having at least 90% sequence identity to Bcap31 is increased relative to a copy number of the gene in normal cells reflects an increased probability of length of survival relative to a probability of length of survival of patients without this pattern of increased and decreased copy number.

In some embodiments, the nucleotide sequence comprises DNA. In some embodiments, the nucleotide sequence comprises mRNA.

In some embodiments, the indicator comprises at least one of a mRNA level, a gene product quantity (such as the expression level of a protein encoded by the gene), a gene product activity level (such as the activity level of a protein encoded by the gene), or a copy number of: at least one of (i) the at least one gene or (ii) the one or more chromosome segments.

In some embodiments, the indicator of increased expression reflects an enhanced probability of survival of the patient relative to a probability of survival of patients without the increased expression. In other embodiments, the indicator of increased expression reflects a decreased probability of survival of the patient relative to a probability of survival of patients without the increased expression.

In some embodiments, the estimating comprises comparing the copy number to a copy number of the at least one nucleotide sequence found in cells of at least one person who does not have an OV tumor. In some embodiments, the copy number is determined by a technique selected from the group consisting of: fluorescent in-situ hybridization, complementary genomic hybridization, array complementary genomic hybridization, fluorescence microscopy, and any combination thereof. In further embodiments, a further indicator, including but not limited to, an evaluation at least one of tumor stage at diagnosis, residual disease after surgery, therapy outcome, and neoplasm status is used in conjunction with the indicator of copy number in evaluating a patient's probability of survival. In one embodiment, a tumor stage at diagnosis of III or IV reflects a decreased probability of length of survival relative to a probability of length of survival of patients with the tumor stage at diagnosis of I or II; or no macroscopic residual disease after surgery reflects an increased probability of length of survival relative to a probability of length of survival of patients with macroscopic residual disease after surgery; or the therapy outcome of complete remission after therapy reflects an increased probability of length of survival relative to a probability of length of survival of patients not in complete remission after therapy; or the neoplasm status of no tumor after therapy reflects an increased probability of length of survival relative to a probability of length of survival of patients with tumor after therapy. In embodiments, the therapy comprises chemotherapy including, but not limited to, platinum-based chemotherapy.

In some embodiments, A method of estimating an outcome for a patient having a high-grade ovarian serous cystadenocarcinoma (OV) tumor, comprises obtaining a biological sample from a patient diagnosed with OV, said sample comprising nucleic acids from the patient; detecting in said nucleic acids a value of an indicator of a differential expression of at least one nucleotide sequence, each sequence having at least 90% sequence identity to at least one gene selected from Ckdn1A, Mapk14, Tnf, Rad51AP1, Cdkn1B, Kras, Itpr2, Rpa3, Pold2, Pabpc5, and Bcap31; and estimating, by a processor and based on the value of the indicators of differential expression, a predicted length of survival of the patient.

In some embodiments, the nucleotide sequence comprises DNA. In some embodiments, the nucleotide sequence comprises mRNA.

In some embodiments, the indicator comprises at least one of an mRNA level, a gene product quantity, a gene product activity level, or a copy number of at least one of the at least one gene.

In some embodiments, the indicator of differential expression is an indicator of increased expression. In these embodiments, the indicator of increased expression may indicate increased expression of one or more gene selected from Rad51AP1, Kras, Rpa3, and Pabpc5 which reflects a decreased probability of survival of the patient relative to a probability of survival of patients without the increased expression. In other embodiments, the indicator of increased expression indicates increased expression of one or more gene selected from Cdkn1A, Mapk14, Pold2, and Bcap31 which reflects an increased probability of survival of the patient relative to a probability of survival of patients without the increased expression.

In other embodiments, the indicator of differential expression is an indicator of decreased expression. In some of these embodiments, the indicator of decreased expression indicates decreased expression of one or more gene selected from Rad51AP1, Kras, Rpa3, and Pabpc5, which reflects an increased probability of length of survival of the patient relative to a probability of length of survival of patients without the decreased expression. In other embodiments, the indicator of decreased expression indicates increased expression of one or more gene selected from Cdkn1A, Mapk14, Pold2, and Bcap31, which reflects an increased probability of length of survival of the patient relative to a probability of length of survival of patients without the decreased expression.

In some particular embodiments, the indicator of differential expression comprises increased expression of the Cdkn1B gene, which reflects a decreased probability of length of survival of the patient relative to a probability of length of survival of patients without the increased expression. In other embodiments, the indicator of differential expression comprises increased expression of the Kras and Rad51AP1 genes and decreased expression of the Cdkn1A, and Mapk14 genes, which reflects a decreased probability of length of survival of the patient relative to a probability of length of survival of patients without the differential expression. In further embodiments, the indicator of differential expression comprises increased expression of the Pold2 gene and decreased expression of the Rpa3 gene, which reflects an increased probability of length of survival of the patient relative to a probability of length of survival of patients without the differential expression.

In some embodiments, the therapy comprises at least one of chemotherapy or radiotherapy.

In some embodiments, the mRNA level is measured by a technique selected from the group consisting of: northern blotting, gene expression profiling, serial analysis of gene expression, and any combination thereof. In some embodiments, the gene product level is measured by a technique selected from the group consisting of enzyme-linked immunosorbent assay, fluorescence microscopy, and any combination thereof.

In other embodiments, a method of predicting a clinical response to platinum-based chemotherapy for a patient diagnosed with a cancer, comprises obtaining a biological sample from a patient diagnosed with the cancer, said sample comprising nucleic acids from the patient; detecting in said nucleic acids a value of an indicator of a differential expression of at least one nucleotide sequence, each sequence having at least 90% sequence identity to at least one gene selected from Ckdn1A, Mapk14, Tnf, Rad51AP1, Cdkn1B, Kras, Itpr2, Rpa3, Pold2, Pabpc5, and Bcap31; and estimating, by a processor and based on the value of the indicators of differential expression, the likelihood for the patient to have a beneficial clinical response to the platinum-based chemotherapy.

In some embodiments, the nucleotide sequence comprises DNA. In some embodiments, the nucleotide sequence comprises mRNA.

In some embodiments, the mRNA level is measured by a technique selected from the group consisting of: northern blotting, gene expression profiling, serial analysis of gene expression, and any combination thereof. In some embodiments, the gene product level is measured by a technique selected from the group consisting of enzyme-linked immunosorbent assay, fluorescence microscopy, and any combination thereof.

In some embodiments, wherein the indicator of differential expression is an indicator of increased expression. In some particular embodiments, the indicator of increased expression indicates increased expression of one or more gene selected from Rad51AP1, Kras, Rpa3, and Pabpc5 which reflects a likelihood for the patient to have a beneficial clinical response to the platinum-based chemotherapy of the patient relative to a likelihood for patients without the increased expression. In other embodiments, the indicator of increased expression indicates increased expression of one or more gene selected from Cdkn1A, Mapk14, Pold2, and Bcap31 which reflects an increased likelihood for the patient to have a beneficial clinical response to the platinum-based chemotherapy relative to a likelihood for patients without the increased expression.

In some embodiments, the indicator of differential expression is an indicator of decreased expression. In some particular embodiments, the indicator of decreased expression indicates decreased expression of one or more gene selected from Rad51AP1, Kras, Rpa3, and Pabpc5, which reflects an increased likelihood for the patient to have a beneficial clinical response to the platinum-based chemotherapy relative to a likelihood for patients without the decreased expression. In other embodiments, the indicator of decreased expression indicates increased expression of one or more gene selected from Cdkn1A, Mapk14, Pold2, and Bcap31, which reflects an increased likelihood for the patient to have a beneficial clinical response to the platinum-based chemotherapy relative to a likelihood for patients without the decreased expression. In further embodiments, the indicator of differential expression comprises increased expression of the Kras and Rad51AP1 genes and decreased expression of the Cdkn1A, and Mapk14 genes, which reflects a decreased likelihood for the patient to have a beneficial clinical response to the platinum-based chemotherapy relative to a likelihood for patients without the decreased expression. In additional embodiments, the indicator of differential expression comprises increased expression of the Pold2 gene and decreased expression of the Rpa3 gene, which reflects an increased likelihood for the patient to have a beneficial clinical response to the platinum-based chemotherapy relative to a likelihood for patients without the decreased expression. Use of an inhibitor in treating an ovarian serous cystadenocarcinoma (OV) tumor cell, wherein said inhibitor (i) down-regulates the expression level of a nucleic acid sequence selected from the group consisting SEQ ID NO: 56, SEQ ID NO: 7, SEQ ID NO: 25, and SEQ ID NO: 27, or a combination thereof; or (ii) down-regulates the activity of an amino acid sequence selected from SEQ ID NO: 57, SEQ ID NO: 8, SEQ ID NO: 26, and SEQ ID NO: 28, or a combination thereof; and/or Use of an activator in treating an ovarian serous cystadenocarcinoma (OV) tumor cell, wherein said activator (i) up-regulates the expression level of a nucleic acid sequence selected from the group consisting of SEQ ID NO: 31, SEQ ID NO: 41, SEQ ID NO: 64, and SEQ ID NO: 70, or a combination thereof; or (ii) up-regulates the activity of an amino acid sequence selected from SEQ ID NO: 32, SEQ ID NO: 42, SEQ ID NO: 65, and SEQ ID NO: 71, and a combination thereof.

In embodiments, Use of an inhibitor in the manufacture of a medicament for reducing the proliferation or viability of an ovarian serous cystadenocarcinoma (OV) tumor cell, wherein said inhibitor (i) down-regulates the expression level of nucleic acid sequence selected from the group consisting of SEQ ID NO: 56, SEQ ID NO: 7, SEQ ID NO: 25, or a combination thereof; or (ii) down-regulates the activity of an amino acid sequence selected from SEQ ID NO: 57, SEQ ID NO: 8, SEQ ID NO: 26, and SEQ ID NO: 28, or a combination thereof.

In embodiments, Use of an activator in the manufacture of a medicament for reducing the proliferation or viability of an ovarian serous cystadenocarcinoma (OV) tumor cell, wherein said activator (i) up-regulates the expression level of a nucleic acid sequence selected from the group consisting of SEQ ID NO: 31, SEQ ID NO: 41, SEQ ID NO: 64, and SEQ ID NO: 70, or a combination thereof; or (ii) up-regulates the activity of an amino acid sequence selected from SEQ ID NO: 32, SEQ ID NO: 42, SEQ ID NO: 65, and SEQ ID NO: 71, or a combination thereof.

In some embodiments, the cancer is an ovarian serous cystadenocarcinoma (OV) tumor. In other embodiments, the cancer is selected from small cell lung cancer, non-small cell lung cancer, testicular cancer, stomach cancer, bladder cancer, colon cancer, breast cancer, adrenocortical cancer, anal cancer, endometrial cancer, non-Hodgkin lymphoma, melanoma, and head and neck cancers.

In other embodiments, A method for reducing the proliferation or viability of an ovarian serous cystadenocarcinoma (OV) tumor cell, comprises contacting the cancer cell with (i) an inhibitor that down-regulates the expression level of a gene selected from the group consisting of Rad51AP1, Kras, Rpa3, and Pabpc5, and a combination thereof; and/or (ii) an activator that up-regulates the expression level of a gene selected from the group consisting of Cdkn1A, Mapk14, Pold2, and Bcap31, or a combination thereof.

In some embodiments, the inhibitor is an RNA effector molecule that down-regulates expression of a gene selected from the group consisting of Rad51AP1, Kras, Rpa3, and Pabpc5, or a combination thereof. In further embodiments, the RNA effector molecule is an siRNA or snRNA that targets Rad51AP1, Kras, Rpa3, and Pabpc5, or a combination thereof.

In some embodiments, non-transitory machine-readable mediums encoded with instructions executable by a processing system to perform a method of estimating an outcome for a patient having a high-grade ovarian serous cystadenocarcinoma (OV) tumor, are provided. The instructions comprise code for: receiving a value of an indicator of a copy number of each of at least one nucleotide sequence, each sequence having at least 90 percent sequence identity to at least one of (i) a respective chromosome segment in cells of the OV, and (ii) at least one gene on the segment; and estimating, by a processor and based on the value, at least one of a predicted length of survival of the patient, a probability of survival of the patient, or a predicted response of the patient to a therapy for the OV.

In some embodiments, a method for treating a patient having ovarian serous cystadenocarcinoma (OV) comprises administering, in a patient diagnosed with OV, a treatment regimen based on predicted length of survival or clinical response to chemotherapy, wherein predicting estimated outcome or clinical response comprises: (1) detecting, in a biological sample from a patient having OV, differential expression of at least one of (a) a nucleic acid sequence having sequence identity to at least two of the genes selected from Ckdn1A, Mapk14, Kras, Rad51AP1, Tnf, Itpr2, Rpa3, Pold2, Lig4, Pabpc5, Bcap31, and Gabre; (b) a protein encoded by one or more of the genes of (a); (c) a cytogenic band of one or more of the genes of (a) selected from the group consisting of bands 1-7 and 11-17; (d) one or more micro RNAs selected from miR-877, miR-877*, miR-200c, miR-141, miR-888, miR-452, and miR-224; (e) a segment overlapping with the Prim2 gene; or (f) the nucleic acid sequence tag site DSX214; (2) calculating, by a processor, a weighted sum pattern based on the value of one or more of the indicators of differential expression; and (3) estimating, by the processor and based on the weighted sum pattern, a predicted length of survival of the patient or a predicated clinical response to chemotherapy for the patient. In embodiments, wherein the at least one nucleic acid has sequence identity to one of the genes selected from Ckdn1A, Mapk14, Kras, Rad51AP1, Tnf, Itpr2, Rpa3, Pold2, Lig4, Pabpc5, Bcap31, and Gabre; and wherein the indicator of differential expression is differential copy number relative to a copy number of the at least one nucleic acid sequence in normal cells.

In embodiments, the differential copy number is an increase or decrease in copy number relative to a copy number of the at least one nucleic acid sequence in normal cells. In embodiments, the at least one protein encoded by the genes of (a) is selected from CKDN1A, MAPK14, KRAS, RAD51AP1, TNF, ITPR2, RPA3, POLD2, LIG4, PABPC5, BCAP31, and GABRE; and the indicator of differential expression is differential protein expression relative to protein expression of the at least one protein in normal cells.

In embodiments, the microRNA sequence is at least one of miR-877, miR-877*, miR-200c, miR-141, miR-888, miR-452, and miR-224; and wherein the indicator of differential expression is differential copy number relative to a copy number of the at least one nucleotide sequence in normal cells. In embodiments, the differential copy number is an increase in copy number relative to a copy number of the at least one nucleotide sequence in normal cells. In embodiments, the differential copy number is a decrease in copy number relative to a copy number of the at least one nucleotide sequence in normal cells. In embodiments, the differential protein expression is an increase in protein expression relative to protein expression in normal cells. In embodiments, the differential protein expression is a decrease in protein expression relative to protein expression in normal cells. In embodiments, the differential copy number is an increase in copy number relative to a copy number of the at least one nucleotide sequence in normal cells. In embodiments, the differential copy number is a decrease in copy number relative to a copy number of the at least one nucleotide sequence in normal cells. In embodiments, the differential expression is microRNA expression. In embodiments, the differential microRNA expression is an increase in microRNA expression relative to microRNA expression of the at least one nucleotide sequence in normal cells. In embodiments, the differential microRNA expression is a decrease in microRNA expression relative to microRNA expression of the at least one nucleotide in normal cells.

In embodiments, the method comprises correlating at least one of the indicators of differential expression selected from (a)-(f) below:
a) co-occurring copy-number loss of Pabpc5, and gain, or mRNA overexpression of Bcap31; or
b) co-occurring copy number loss of Pabpc5, and gain, or mRNA overexpression of Bcap31, and gain, or microRNA overexpression of miR-888, and miR-452; or
c) co-occurring copy number loss of Pabpc5, and gain, or mRNA overexpression of Bcap31, and gain, or microRNA overexpression of miR-888, miR-452, and miR-224; or
d) co-occurring copy-number loss of Pabpc5 and sequence tag site (STS) DXS214, and gain, or mRNA overexpression of Bcap31; or
e) co-occurring copy number loss of Pabpc5, and gain, or mRNA overexpression of Bcap31 and Gabre; or
f) co-occurring copy-number loss from cytogenetic bands 1-14, and gain in cytogenetic bands 16-24;
with at least one of longer survival time and sensitivity to platinum-based chemotherapy.

In embodiments the differential expression of (c) further includes correlating copy-number loss of sequence tag site DXS214 and gain or mRNA overexpression of Bcap31 and Gabre with at least one of longer survival time and sensitivity of platinum-based chemotherapy.

In embodiments, the method comprises correlating at least one of the indicators of differential expression selected from (a1)-(d1) below:
a1) co-occurring copy-number loss, or mRNA underexpression of Rpa3, and copy-number gain, or mRNA overexpression of Pold2; or
b1) co-occurring copy-number loss, or mRNA underexpression of Rpa3 on 7p and Lig4 on 13q, and copy-number gain, or mRNA overexpression of Pold2; or
c1) co-occurring copy-number loss, or mRNA underexpression of Lig4 on chromosome 13q, and copy-number gain, or mRNA overexpression of Pold2; or
d1) co-occurring copy-number loss from cytogenetic bands 1-7, and gain in cytogenetic bands 11-17;
with at least one of a longer survival time and sensitivity to platinum-based chemotherapy.

In embodiments, the method comprises correlating at least one of the indicators of differential expression selected from (a2)-(g2) below:
a2) co-occurring copy-number loss on chromosome 6p and gain on chromosome 12p; or
b2) co-occurring copy-number loss, or mRNA or protein under-expression of Cdkn1A and Mapk14 on chromosome 6p, and copy-number gain, or mRNA or protein overexpression of Kras on chromosome 12p; or
c2) co-occurring copy-number loss, or mRNA or protein under-expression of Cdkn1A and Mapk14 on 6p, and copy-number gain, or mRNA or protein overexpression of Kras and Rad51AP1 on 12p; or
d2) co-occurring copy-number loss, or mRNA or protein under-expression of Cdkn1A, Mapk14, and Tnf on chromosome 6p, and copy-number gain, or mRNA or protein overexpression of Kras, Rad51AP1, and Itpr2 on chromosome 12p; or
e2) co-occurring copy-number loss, or microRNA under-expression of miR-877* on chromosome 6p, and copy-number gain, or microRNA overexpression, of miR-200c, miR-200c*, miR-141, or miR-141* on chromosome 12p;
(f2) co-occurring copy-number loss, or mRNA or protein under-expression of Cdkn1A and Mapk14 on chromosome 6p, and copy-number gain, or mRNA or protein overexpression of Rad51AP1 on chromosome 12p;
(g2) co-occurring copy-number loss, or mRNA or protein under-expression of Tnf on chromosome 6p, and copy-number gain, or mRNA or protein overexpression of Itpr2 on chromosome 12p;
with at least one of shorter survival time and resistance to platinum-based chemotherapy.

In embodiments, the method comprises the differential expression of at least one of (a2)-(g2) and further comprises correlating at least one of:
(h2) a gain in copy numbers or mRNA or protein overexpression of Sox5; or
(i2) a gain in copy numbers or mRNA or protein overexpression of Asun; or
(j2) a gain in copy numbers or mRNA or protein overexpression of Abcf1; or
(k2) a gain in copy numbers or mRNA or protein overexpression of Cdkn1B; or
(l2) an mRNA or protein under-expression or loss in copy numbers of Bap1; or
(m2) a reduced abundance of Brca1-associated genome surveillance protein complex (BASC);
with at least one of a patient's shorter survival time and resistance to platinum-based chemotherapy.

In embodiments, the method comprises correlating at least one of the indicators of differential expression selected from (a)-(f), (a1)-(d1), and (a2)-(e2).

In embodiments, the method further comprises correlating at least one of:

(1) an increase in copy number of the segment overlapping with SEQ ID NO: 1 with at least one of reduced length of patient survival and resistance to platinum-based chemotherapy;

(2) an increase in copy number of SEQ ID NO: 7 with at least one of reduced length of patient survival and resistance to platinum-based chemotherapy;

(3) an increase in copy number of SEQ ID NO: 10 with at least one of reduced length of patient survival and resistance to platinum-based chemotherapy;

(4) an increase in copy number of SEQ ID NO: 21 with at least one of reduced length of patient survival and resistance to platinum-based chemotherapy;

(5) an increase in copy number of SEQ ID NO: 23 with at least one of reduced length of patient survival and resistance to platinum-based chemotherapy;

(6) a decrease in copy number of SEQ ID NO: 25 with at least one of increased length of patient survival and sensitivity to platinum-based chemotherapy;

(7) a decrease in copy number of SEQ ID NO: 27 with at least one of increased length of patient survival and sensitivity to platinum-based chemotherapy;

(8) a decrease in copy number of SEQ ID NO: 29 with at least one of increased length of patient survival and sensitivity to platinum-based chemotherapy;

(9) a decrease in copy number of SEQ ID NO: 31 with at least one of reduced length of patient survival and resistance to platinum-based chemotherapy;

(10) a decrease in copy number of SEQ ID NO: 41 with at least one of reduced length of patient survival and resistance to platinum-based chemotherapy;

(11) a decrease in copy number of SEQ ID NO: 39 with at least one of reduced length of patient survival and resistance to platinum-based chemotherapy;

(12) a decrease in copy number of SEQ ID NO: 51 with at least one of reduced length of patient survival and resistance to platinum-based chemotherapy;

(13) a decrease in copy number of SEQ ID NO: 52 with at least one of reduced length of patient survival and resistance to platinum-based chemotherapy;

(14) an increase in copy number of SEQ ID NO: 56 with at least one of reduced length of patient survival and resistance to platinum-based chemotherapy;

(15) an increase in copy number of SEQ ID NO: 60 with at least one of reduced length of patient survival and resistance to platinum-based chemotherapy;

(16) an increase in copy number of SEQ ID NO: 61 with at least one of reduced length of patient survival and resistance to platinum-based chemotherapy;

(17) an increase in copy number of SEQ ID NO: 62 with at least one of reduced length of patient survival and resistance to platinum-based chemotherapy;

(18) an increase in copy number of SEQ ID NO: 64 with at least one of increased length of patient survival and sensitivity to platinum-based chemotherapy;

(19) an increase in copy number of SEQ ID NO: 70 with at least one of increased length of patient survival and sensitivity to platinum-based chemotherapy;

(20) an increase in copy number of SEQ ID NO: 78 with at least one of increased length of patient survival and sensitivity to platinum-based chemotherapy;

(21) an increase in copy number of SEQ ID NO: 79 with at least one of increased length of patient survival and sensitivity to platinum-based chemotherapy;

(22) an increase in copy number of SEQ ID NO: 80 with at least one of increased length of patient survival and sensitivity to platinum-based chemotherapy;

(23) an increase in copy number of SEQ ID NO: 81 gene with at least one of increased length of patient survival and sensitivity to platinum-based chemotherapy;

(24) a decrease in copy number of SEQ ID NO: 96 gene with at least one of increased length of patient survival and sensitivity to platinum-based chemotherapy;

or any combination of (1)-(24). In embodiments, the method comprises: (i) correlating at least two of (2), (4), (6), (9)-(12), (14)-(16), (18), and (24);

(ii) correlating at least two of (2), (4), (7), (9)-(12), (14)-(16), (19)-(23); or

(iii) correlating at least two of (6)-(7), and (18)-(24).

In some embodiments, a method for treating a patient having ovarian serous cystadenocarcinoma (OV), comprises administering, in a patient having OV, a treatment regimen based on predicted length of survival or clinical response to chemotherapy, wherein the predicted length of survival or predicted clinical response to chemotherapy was derived from: detecting, in a biological sample from a patient having OV, a differential expression of at least one of (a) at least two nucleic acid sequences selected from the group consisting of SEQ ID NO: 1, SEQ ID NO: 7, SEQ ID NO: 21, SEQ ID NO: 25, SEQ ID NO: 27, SEQ ID NO: 29, SEQ ID NO: 31, SEQ ID NO: 41, SEQ ID NO: 47, SEQ ID NO: 56, SEQ ID NO: 64, SEQ ID NO: 70, SEQ ID NO: 81, SEQ ID NO: 96; (b) at least one amino acid sequence encoded by one or more of (a); or (c) at least one micro RNA selected from SEQ ID NO: 51, SEQ ID NO: 60, SEQ ID NO: 61, SEQ ID NO: 78, SEQ ID NO: 79, and SEQ ID NO: 80; calculating, by a processor, a weighted sum based on the value of one or more of the indicators of differential expression; and estimating, by a processor and based on the weighted sum, the predicted length of survival of the patient or the predicted clinical response to chemotherapy. In embodiments, the indicator of differential expression for the nucleic acid sequences is differential copy number relative to copy number of the nucleic acid sequences in normal cells. In embodiments, the differential copy number is an increase in copy number relative to a copy number of the nucleic acid sequences in normal cells. In embodiments, the differential copy number is a decrease in copy number relative to a copy number of the nucleic acid sequences in normal cells.

In some embodiments, the amino acid sequences is proteins selected from SEQ ID NO: 8, SEQ ID NO: 22, SEQ ID NO: 26, SEQ ID NO: 28, SEQ ID NO: 32, SEQ ID NO: 42, SEQ ID NO: 50, SEQ ID NO: 57, SEQ ID NO: 65, SEQ ID NO: 71, and SEQ ID NO: 82, SEQ ID NO: 97; and wherein the indicator of differential expression is differential protein expression relative to protein expression of the at least one protein in normal cells. In embodiments, the differential protein expression is an increase in protein expression relative to protein expression in normal cells. In embodiments, the differential protein expression is a decrease in protein expression relative to protein expression in normal cells. In some embodiments, the microRNA sequence is at least one SEQ ID NO: 51, SEQ ID NO: 60, SEQ ID NO: 61, SEQ ID NO: 78, SEQ ID NO: 79, and SEQ ID NO: 80; and wherein the indicator of differential expression is differential copy number relative to a copy number of the at least one nucleic acid sequence in normal cells. In embodiments, the differential copy number is an increase in copy number relative to a copy number of the at least one nucleic acid sequence in normal cells. In embodiments, the differential copy number is a decrease in copy number relative to a copy number of the at least one nucleic acid sequence in normal cells. In embodiments, the microRNA sequence is at least one SEQ ID NO: 51, SEQ ID NO: 60, SEQ ID NO: 61, SEQ ID NO: 78, SEQ ID NO: 79, and SEQ ID NO: 80; and wherein the indicator of differential expression is differential microRNA expression relative to microRNA expression of the sequence in normal cells. In further embodiments, the differential microRNA expression is an increase in microRNA expression relative to microRNA expression of the at least one nucleic acid sequence in normal cells.

In embodiments, the differential microRNA expression is a decrease in microRNA expression relative to microRNA expression of the at least one nucleic acid in normal cells. In embodiments, the method further comprises correlating at least one of the indicators of differential expression selected from (a)-(f) below:
a) co-occurring copy-number loss of Pabpc5, and gain, or mRNA overexpression of Bcap31; or
b) co-occurring copy number loss of Pabpc5, and gain, or mRNA overexpression of Bcap31, and gain, or microRNA overexpression of miR-888, and miR-452; or
c) co-occurring copy number loss of Pabpc5, and gain, or mRNA overexpression of Bcap31, and gain, or microRNA overexpression of miR-888, miR-452, and miR-224; or
d) co-occurring copy-number loss of Pabpc5 and sequence tag site (STS) DXS214, and gain, or mRNA overexpression of Bcap31; or
e) co-occurring copy number loss of Pabpc5, and gain, or mRNA overexpression of Bcap31 and Gabre; or
f) co-occurring copy-number copy number loss from cytogenetic bands 1-14, and gain in cytogenetic bands 16-24;
with at least one of longer survival time and sensitivity to platinum-based chemotherapy.

In embodiments the differential expression of (c) further includes correlating copy-number loss of sequence tag site DXS214 and gain or mRNA overexpression of Bcap31 and Gabre with at least one of longer survival time and sensitivity of platinum-based chemotherapy.

In embodiments, the method comprises correlating at least one of the indicators of differential expression selected from (a1)-(d1) below:
a1) co-occurring copy-number loss, or mRNA underexpression of Rpa3, and copy-number gain, or mRNA overexpression of Pold2; or
b1) co-occurring copy-number loss, or mRNA underexpression of Rpa3 on 7p and Lig4 on 13q, and copy-number gain, or mRNA overexpression of Pold2; or
c1) co-occurring copy-number loss, or mRNA underexpression of Lig4 on chromosome 13q, and copy-number gain, or mRNA overexpression of Pold2; or
d1) co-occurring copy-number loss from cytogenetic bands 1-7, and gain in cytogenetic bands 11-17;
with at least one of a longer survival time and sensitivity to platinum-based chemotherapy.

In embodiments, the method comprises correlating at least one of the indicators of differential expression selected from (a2)-(g2) below:
a2) co-occurring copy-number loss on chromosome 6p and gain on chromosome 12p; or
b2) co-occurring copy-number loss, or mRNA or protein under-expression of Cdkn1A and Mapk14 on chromosome 6p, and copy-number gain, or mRNA or protein overexpression of Kras on chromosome 12p; or
c2) co-occurring copy-number loss, or mRNA or protein under-expression of Cdkn1A and Mapk14 on 6p, and copy-number gain, or mRNA or protein overexpression of Kras and Rad51AP1 on 12p; or
d2) co-occurring copy-number loss, or mRNA or protein under-expression of Cdkn1A, Mapk14, and Tnf on chromosome 6p, and copy-number gain, or mRNA or protein overexpression of Kras, Rad51AP1, and Itpr2 on chromosome 12p; or
e2) co-occurring copy-number loss, or microRNA under-expression of miR-877* on chromosome 6p, and copy-number gain, or microRNA overexpression, of miR-200c, miR-200c*, miR-141, or miR-141* on chromosome 12p;
(f2) co-occurring copy-number loss, or mRNA or protein under-expression of Cdkn1A and Mapk14 on chromosome 6p, and copy-number gain, or mRNA or protein overexpression of Rad51AP1 on chromosome 12p;
(g2) co-occurring copy-number loss, or mRNA or protein under-expression of Tnf on chromosome 6p, and copy-number gain, or mRNA or protein overexpression of Itpr2 on chromosome 12p;
with at least one of shorter survival time and resistance to platinum-based chemotherapy.

In embodiments, the method comprises the differential expression of at least one of (a2)-(g2) and further comprises correlating at least one of:
(h2) a gain in copy numbers or mRNA or protein overexpression of Sox5; or
(i2) a gain in copy numbers or mRNA or protein overexpression of Asun; or
(j2) a gain in copy numbers or mRNA or protein overexpression of Abcf1; or
(k2) a gain in copy numbers or mRNA or protein overexpression of Cdkn1B; or
(l2) an mRNA or protein under-expression or loss in copy numbers of Bap1; or
(m2) a reduced abundance of Brca1-associated genome surveillance protein complex (BASC);
with at least one of a patient's shorter survival time and resistance to platinum-based chemotherapy.

In embodiments, the method comprises correlating at least one of the indicators of differential expression selected from (a)-(f), (a1)-(d1), and (a2)-(e2).

In some embodiments, a method of treating a patient having a high-grade ovarian serous cystadenocarcinoma (OV) tumor, comprises administering, in a patient having high-grade OV, a treatment regimen based on the predicted length of survival of the patient, wherein the predicting length of survival comprises: (1) detecting, in a biological sample from a patient having OV, an indicator of differential expression comprising at least two nucleic acid sequences selected from the group consisting of SEQ ID NO: 7, SEQ ID NO: 21, SEQ ID NO: 25, SEQ ID NO: 27, SEQ ID NO: 31, SEQ ID NO: 41, SEQ ID NO: 47, SEQ ID NO: 56, SEQ ID NO: 64, SEQ ID NO: 70, SEQ ID NO: 81, SEQ ID NO: 96; (b) level of expression of the nucleic acid sequences in (a); or (c) copy number of at least one of (a); and (2) calculating, by a processor, a weighted sum pattern based on the value of one or more indicators of differential expression; and (3) estimating, by the processor and based on the weighted sum pattern, a predicted length of survival of the patient. In embodiments, the nucleic acid sequence comprises DNA or mRNA.

In embodiments, the indicator of differential expression is an indicator of increased expression. In embodiments, the indicator of increase in expression indicates increased expression of at least two nucleic acid sequences selected from SEQ ID NO 56. SEQ ID NO: 7, SEQ ID NO: 25. SEQ ID NO: 27, SEQ ID NO: 31, SEQ ID NO: 41, SEQ ID NO: 64, and SEQ ID NO: 70, which reflects a decreased probability of survival of the patient relative to a probability of survival of patients without the increased expression. In further embodiments, the indicator of differential expression is an indicator of decreased expression. In embodiments, the indicator of decreased expression indicates decreased expression of the nucleic acid sequences selected from SEQ ID NO: 31, SEQ ID NO: 41, SEQ ID NO: 64, SEQ ID NO: 56, SEQ ID NO: 7, SEQ ID NO: 25, SEQ ID NO 27, and SEQ ID NO: 70, which reflects an increased probability of length of survival of the patient relative to a probability of length of survival of patients without the decreased expression. In embodiments, the indicator of differential expression comprises increased expression of SEQ ID NO: 62, which reflects a decreased probability of length of survival of the patient relative to a probability of length of survival of patients without the increased expression. In some embodiments, the indicator of differential expression comprises increased expression of SEQ ID NO: 7 and SEQ ID NO: 56 and decreased expression of SEQ ID NO: 31 and SEQ ID NO: 41, which reflects a decreased probability of length of survival of the patient relative to a probability of length of survival of patients without the differential expression. In embodiments, the indicator of differential expression comprises increased expression of SEQ ID NO: 64 and decreased expression of SEQ ID NO: 25, which reflects an increased probability of length of survival of the patient relative to a probability of length of survival of patients without the differential expression.

In some embodiments, the treatment regimen comprises at least one of chemotherapy or radiotherapy. In embodiments, expression level of the nucleic acid sequences is measured by a technique selected from the group consisting of: northern blotting, gene expression profiling, serial analysis of gene expression, enzyme-linked immunosorbent assay, fluorescence microscopy, and any combination thereof.

In some embodiments, a method of treating a patient with a cancer comprises administering, in a patient diagnosed with a cancer, a treatment regimen based on clinical response to platinum-based chemotherapy, wherein predicting clinical response comprises: (1) detecting, in a biological sample from a patient having with OV, an indicator of differential expression consisting of at least two nucleotide sequences selected from of SEQ ID NO: 7, SEQ ID NO: 21, SEQ ID NO: 25, SEQ ID NO: 27, SEQ ID NO: 31, SEQ ID NO: 41, SEQ ID NO: 47, SEQ ID NO: 56, SEQ ID NO: 64, SEQ ID NO: 70, SEQ ID NO: 96; (b) level of expression of the nucleic acid sequences in (a); or (c) copy number of at least one of (a); and (2) calculating, by a processor, a weighted sum pattern based on the value of one or more indicators of differential expression; and (3) estimating, by the processor and based on the value of the indicators of differential expression, the likelihood for the patient to have a beneficial response to the platinum-based chemotherapy. In embodiments, the method comprises recommending one of (i) a platinum-based chemotherapy or (ii) an alternative treatment regimen based on the predicted clinical response to platinum-based chemotherapy. In embodiments, the method further comprises administering one of (i) a platinum-based chemotherapy or (ii) an alternative treatment regimen based on the predicted clinical response to platinum-based chemotherapy.

In embodiments, the nucleotide sequence comprises DNA. In embodiments, the nucleotide sequence comprises mRNA. In embodiments, the indicator of differential expression is an indicator of increased expression. In embodiments, the indicator of increase in expression indicates increased expression of the nucleic acid sequences selected from SEQ ID NO: 31, SEQ ID NO: 41, SEQ ID NO: 64, SEQ ID NO: 56, SEQ ID NO: 7, SEQ ID NO: 25, SEQ ID NO: 27, and SEQ ID NO: 70 which reflects a likelihood for the patient to have a beneficial clinical response to the platinum-based chemotherapy of the patient relative to a likelihood for patients without the increased expression. In some embodiments, the indicator of differential expression is an indicator of decreased expression. In embodiments, the indicator of decreased expression indicates decreased expression of the nucleic acid sequences selected from SEQ ID NO: 56, SEQ ID NO: 7; SEQ ID NO: 25, SEQ ID NO: 27, which reflects an increased likelihood for the patient to have a beneficial clinical response to the platinum-based chemotherapy relative to a likelihood for patients without the decreased expression. In embodiments, the indicator of decreased expression indicates increased expression of the nucleic acid sequences selected from SEQ ID NO: 31, SEQ ID NO: 41, SEQ ID NO: 64, and SEQ ID NO: 70, which reflects an increased likelihood for the patient to have a beneficial clinical response to the platinum-based chemotherapy relative to a likelihood for patients without the decreased expression. In embodiments, the indicator of differential expression comprises increased expression of SEQ ID NO: 7 and SEQ ID NO: 56 and decreased expression of SEQ ID NO: 31 and SEQ ID NO: 41, which reflects a decreased likelihood for the patient to have a beneficial clinical response to the platinum-based chemotherapy relative to a likelihood for patients without the decreased expression. In further embodiments, the indicator of differential expression comprises increased expression of SEQ ID NO: 64 and decreased expression of SEQ ID NO: 25, which reflects an increased likelihood for the patient to have a beneficial clinical response to the platinum-based chemotherapy relative to a likelihood for patients without the decreased expression.

In some embodiments, the cancer is an ovarian serous cystadenocarcinoma (OV) tumor. In other embodiments, the cancer is selected from small cell lung cancer, non-small cell lung cancer, testicular cancer, stomach cancer, bladder cancer, colon cancer, breast cancer, adrenocortical cancer, anal cancer, endometrial cancer, non-Hodgkin lymphoma, melanoma, and head and neck cancers.

In embodiments, a method for reducing the proliferation or viability of an ovarian serous cystadenocarcinoma (OV) tumor cell, comprises contacting the cancer cell with (i) an inhibitor that down-regulates the expression level of a gene selected from the group consisting of SEQ ID NO: 56, SEQ ID NO: 7, SEQ ID NO: 25, SEQ ID NO: 27, and a combination thereof; and/or (ii) an activator that up-regulates the expression level of a gene selected from the group consisting of SEQ ID NO: 31, SEQ ID NO: 41, SEQ ID NO: 64, SEQ ID NO: 70, or a combination thereof. In embodiments, said inhibitor is an RNA effector molecule that down-regulates expression of a gene selected from the group consisting of SEQ ID NO: 56, SEQ ID NO: 7, SEQ ID NO: 25, SEQ ID NO: 27, or a combination thereof. In embodiments, said RNA effector molecule is an siRNA or shRNA that targets SEQ ID NO: 56, SEQ ID NO: 7, SEQ ID NO: 25, SEQ ID NO: 27, or a combination thereof.

In some embodiments, use of an inhibitor in treating an ovarian serous cystadenocarcinoma (OV) tumor cell, wherein said inhibitor (i) down-regulates the expression level of a gene selected from the group consisting of SEQ ID NO: 56, SEQ ID NO: 7, SEQ ID NO: 25, SEQ ID NO: 27, or a combination thereof; or (ii) down-regulates the activity of a protein selected from SEQ ID NO: 57, SEQ ID NO: 8, SEQ ID NO: 26, SEQ ID NO: 28, or a combination thereof. I embodiments, use of an activator in treating an ovarian serous cystadenocarcinoma (OV) tumor cell, wherein said activator (i) up-regulates the expression level of a gene selected from the group consisting of SEQ ID NO: 31, SEQ ID NO: 41, SEQ ID NO: 64, and SEQ ID NO: 70, or a combination thereof; or (ii) up-regulates the activity of a protein selected from SEQ ID NO: 32, SEQ ID NO: 42, SEQ ID NO: 65, and SEQ ID NO: 71, and a combination thereof. In embodiments, use of an inhibitor in the manufacture of a medicament for reducing the proliferation or viability of an ovarian serous cystadenocarcinoma (OV) tumor cell, wherein said inhibitor (i) down-regulates the expression level of a gene selected from the group consisting of SEQ ID NO: 56, SEQ ID NO: 7, SEQ ID NO: 25, and SEQ ID NO: 27, or a combination thereof; or (ii) down-regulates the activity of a protein selected from SEQ ID NO: 57, SEQ ID NO: 8, SEQ ID NO: 26, and SEQ ID NO: 28, or a combination thereof. In embodiments, use of an activator in the manufacture of a medicament for reducing the proliferation or viability of an ovarian serous cystadenocarcinoma (OV) tumor cell, wherein said activator (i) up-regulates the expression level of a gene selected from the group consisting of SEQ ID NO: 31, SEQ ID NO: 41, SEQ ID NO: 64, and SEQ ID NO: 70, or a combination thereof; or (ii) up-regulates the activity of a protein selected from SEQ ID NO: 32, SEQ ID NO: 42, SEQ ID NO: 65, and SEQ ID NO: 71, and a combination thereof.

In some embodiments, the indicator of differential expression is an indicator of decreased expression. In some particular embodiments, the indicator of decreased expression indicates decreased expression of one or more gene selected from Rad51AP1, Kras, Rpa3, and Pabpc5, which reflects an increased likelihood for the patient to have a beneficial clinical response to the platinum-based chemotherapy relative to a likelihood for patients without the decreased expression. In other embodiments, the indicator of decreased expression indicates increased expression of one or more gene selected from Cdkn1A, Mapk14, Pold2, and Bcap31, which reflects an increased likelihood for the patient to have a beneficial clinical response to the platinum-based chemotherapy relative to a likelihood for patients without the decreased expression. In further embodiments, the indicator of differential expression comprises increased expression of the Kras and Rad51AP1 genes and decreased expression of the Cdkn1A, and Mapk14 genes, which reflects a decreased likelihood for the patient to have a beneficial clinical response to the platinum-based chemotherapy relative to a likelihood for patients without the decreased expression. In additional embodiments, the indicator of differential expression comprises increased expression of the Pold2 gene and decreased expression of the Rpa3 gene, which reflects an increased likelihood for the patient to have a beneficial clinical response to the platinum-based chemotherapy relative to a likelihood for patients without the decreased expression. Use of an inhibitor in treating an ovarian serous cystadenocarcinoma (OV) tumor cell, wherein said inhibitor (i) down-regulates the expression level of a nucleic acid sequence selected from the group consisting SEQ ID NO: 56, SEQ ID NO: 7, SEQ ID NO: 25, and SEQ ID NO: 27, or a combination thereof; or (ii) down-regulates the activity of an amino acid sequence selected from SEQ ID NO: 57, SEQ ID NO: 8, SEQ ID NO: 26, and SEQ ID NO: 28, or a combination thereof; and/or Use of an activator in treating an ovarian serous cystadenocarcinoma (OV) tumor cell, wherein said activator (i) up-regulates the expression level of a nucleic acid sequence selected from the group consisting of SEQ ID NO: 31, SEQ ID NO: 41, SEQ ID NO: 64, and SEQ ID NO: 70, or a combination thereof; or (ii) up-regulates the activity of an amino acid sequence selected from SEQ ID NO: 32, SEQ ID NO: 42, SEQ ID NO: 65, and SEQ ID NO: 71, and a combination thereof.

In embodiments, Use of an inhibitor in the manufacture of a medicament for reducing the proliferation or viability of an ovarian serous cystadenocarcinoma (OV) tumor cell, wherein said inhibitor (i) down-regulates the expression level of nucleic acid sequence selected from the group consisting of SEQ ID NO: 56, SEQ ID NO: 7, SEQ ID NO: 25, or a combination thereof; or (ii) down-regulates the activity of an amino acid sequence selected from SEQ ID NO: 57, SEQ ID NO: 8, SEQ ID NO: 26, and SEQ ID NO: 28, or a combination thereof.

In embodiments, Use of an activator in the manufacture of a medicament for reducing the proliferation or viability of an ovarian serous cystadenocarcinoma (OV) tumor cell, wherein said activator (i) up-regulates the expression level of a nucleic acid sequence selected from the group consisting of SEQ ID NO: 31, SEQ ID NO: 41, SEQ ID NO: 64, and SEQ ID NO: 70, or a combination thereof; or (ii) up-regulates the activity of an amino acid sequence selected from SEQ ID NO: 32, SEQ ID NO: 42, SEQ ID NO: 65, and SEQ ID NO: 71, or a combination thereof.

The term “normal cell” (or “healthy cell”) as used herein, refers to a cell that does not exhibit a disease phenotype. For example, in a diagnosis of OV, a normal cell (or a non-cancerous cell) refers to a cell that is not a tumor cell (non-malignant, non-cancerous, or without DNA damage characteristic of a tumor or cancerous cell). The term a “tumor cell” (or “cancer cell”) refers to a cell displaying one or more phenotype of a tumor, such as OV. The terms “tumor” or “cancer” refer to the presence of cells possessing characteristics typical of cancer-causing cells, such as uncontrolled proliferation, immortality, metastatic potential, rapid growth or proliferation rate, and certain characteristic morphological features.

Normal cells can be cells from a healthy subject. Alternatively, normal cells can be non-malignant, non-cancerous cells from a subject having OV.

The comparison of the mRNA level, the gene product level, or the copy number of a particular nucleotide sequence between a normal cell and a tumor cell can be determined in parallel experiments, in which one sample is based on a normal cell, and the other sample is based on a tumor cell. Alternatively, the mRNA level, the gene product level, or the copy number of a particular nucleotide sequence in a normal cell can be a pre-determined “control,” such as a value from other experiments, a known value, or a value that is present in a database (e.g., a table, electronic database, spreadsheet, etc.).

In general, standard gene and protein nomenclature is followed herein. Unless the description indicates otherwise, gene symbols are generally italicized, with first letter in upper case all the rest in lower case; and a protein encoded by a gene generally uses the same symbol as the gene, but without italics and all in upper case.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

FIGS. 1A-1C are illustrations of high-level diagrams illustrating examples of tensors including biological datasets, according to some embodiments.

FIG. 2 is an illustration of a high-level diagram illustrating a linear transformation of a three-dimensional array, according to some embodiments.

FIG. 3 depicts diagrams illustrating tensor GSVD of patient-matched and platform-matched DNA copy-number profiles for the 6p+12p chromosome, according to some embodiments.

FIG. 4 depicts diagrams illustrating the tensor GSVD of TCGA patient-matched and platform-matched tumor and normal DNA copy-number profiles for the 7p chromosome, according to some embodiments.

FIG. 5 depicts diagrams illustrating the tensor GSVD of TCGA patient-matched and platform-matched tumor and normal DNA copy-number profiles for the Xq chromosome, according to some embodiments.

FIG. 6 depicts diagrams illustrating tumor-exclusive and platform-consistent DNA CNA correlated with OV patients' survival for the 6p+12p chromosome, according to some embodiments.

FIG. 7 depicts diagrams illustrating tumor-exclusive and platform-consistent DNA CNA correlated with OV patients' survival for the 7p chromosome, according to some embodiments.

FIG. 8 depicts diagrams illustrating tumor-exclusive and platform-consistent DNA CNA correlated with OV patients' survival for the Xq chromosome, according to some embodiments.

FIG. 9 is an illustration of bar charts illustrating the most significant probelets in tumor and normal data sets for the 6p+12p, 7p, and Xq chromosomes, according to some embodiments. The X-axis (a, c, e) is the tumor generalized fraction. The X-axis (b, d, f) is the normal generalized fraction. The Y-axis (all charts) are the subtensors.

FIG. 10 shows illustrations of graphs illustrating survival analyses of 249 patients classified by the standard OV indicators: tumor stage (a), residual disease (b), outcome of subsequent therapy (c) and neoplasm status (d), according to some embodiments. X-axis (all graphs): survival time (months); Y-axis, graphs (all graphs): Fraction of surviving patients from the discovery set.

FIG. 11 shows illustrations of graphs illustrating survival analyses of the validation set of patients classified by the standard OV indicators: tumor stage (a), residual disease (b), outcome of subsequent therapy (c) and neoplasm status (d), according to some embodiments. X-axis (all graphs): survival time (months); Y-axis, graphs (all graphs): Fraction of surviving patients from the validation set.

FIG. 12 is a diagram illustrating survival analyses of discovery and validation sets of patients classified by GSVD or tensor GSVD and tumor stage at diagnosis, according to some embodiments.

FIGS. 13A-13I are diagrams illustrating survival analyses of platinum-based chemotherapy patients in a discovery set (FIGS. 13A-13F) and a validation set (FIGS. 13G-13I) of a number of patients classified by tensor GSVD (FIGS. 13A-13C) or tensor GSVD and tumor stage at diagnosis (FIGS. 13D-13I), according to some embodiments. X-axis (all graphs): survival time (months); Y-axis (all graphs): Fraction of surviving patients.

FIGS. 14A-14C are diagrams illustrating survival analyses of a validation set of a number of patients classified by tensor GSVD and tumor stage at diagnosis, according to some embodiments. X-axis (all graphs): survival time (months); Y-axis (all graphs): Fraction of surviving patients.

FIGS. 15A-15I are diagrams illustrating survival analyses of the fraction of surviving platinum-based chemotherapy patients in the discovery set classified by tensor GSVD and residual disease (FIGS. 15A-15C), tensor GSVD and therapy outcome (FIGS. 15D-15F), or tensor GSVD and neoplasm status (FIGS. 15G-15I), according to some embodiments. X-axis (all graphs): survival time (months); Y-axis (all graphs): Fraction of surviving patients.

FIGS. 16A-16I are diagrams illustrating survival analyses of the fraction of surviving platinum-based chemotherapy patients in the discovery set of a number of patients classified by tensor GSVD and residual disease (FIGS. 16A-16C), tensor GSVD and therapy outcome (FIGS. 16D-16F), or tensor GSVD and neoplasm status (FIGS. 16G-16I), according to some embodiments. X-axis (all graphs): survival time (months); Y-axis (all graphs): Fraction of surviving patients.

FIGS. 17A-17F are diagrams illustrating the Kaplan-Meier (KM) curves for survival analyses of discovery and validations sets of patients classified by copy number changes in selected segments, according to some embodiments. X-axis (all graphs): survival time (months); Y-axis (all graphs): Fraction of surviving patients from the discovery and validation sets.

FIG. 18 is a diagram illustrating survival analyses of discovery and validation sets of patients classified by 6p+12p, 7p, and Xq tensor GSVD combined, according to some embodiments.

FIGS. 19A-19I are diagrams illustrating differences in relative mRNA expression between the tensor GSVD classes for selected segments, according to some embodiments. X-axis (all graphs): high or low x-probelet coefficient or arraylet correlation; Y-axis (all graphs): relative mRNA expression.

FIGS. 20A-20H are diagrams illustrating differences in relative microRNA expression between the tensor GSVD classes for selected segments, according to some embodiments. X-axis (all graphs): high or low x-probelet coefficient or arraylet correlation; Y-axis (all graphs): relative mRNA expression.

FIGS. 21A-21B are diagrams illustrating differences in relative protein expression between the tensor GSVD classes for selected segments, according to some embodiments. X-axis (all graphs): high or low x-probelet coefficient or arraylet correlation; Y-axis (all graphs): relative protein expression.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

U.S. Provisional Application No. 61/553,840, entitled “Genomic Tensor Analysis for Medical Assessment and Prediction,” was filed on Oct. 31, 2011 and published on Mar. 14, 2013 as WO 2013/036874. U.S. Provisional Application No. 61/553,870, entitled “Genetic Alterations in Glioblastoma,” was filed on Oct. 31, 2011 and published on May 10, 2013 as WO 2013/067050. The technical subject matter of U.S. Provisional Application Nos. 61/553,840 and 61/553,870, and the corresponding publications, WO 2013/036874 and WO 2013/067050, are hereby incorporated by reference in their entirety.

### I. Overview of Ovarian Serous Cystadenocarcinoma

Ovarian serous cystadenocarcinoma (OV) is a tumor arising from epithelial cells and originating in the ovaries. OV tumors are typically categorized according to their stage. The most common adopted staging system for ovarian cancer including OV tumors is the FIGO staging system: stage I tumors are limited to the ovaries, stage II tumors involve one or both ovaries with pelvic extension; stage III tumors involve one or both ovaries with peritoneal implants outside the pelvis or with retroperitoneal lymph node metastasis; stage IV tumors present with distant metastases, including liver parenchyma (Radiopaedia.org).

OV tumors are further categorized according to their grade, as determined by pathologic evaluation of the tumor; residual macroscopic disease after surgery, outcome of subsequent therapy, i.e. complete remission or not, and neoplasm status, i.e., with or without tumor. Low-grade tumors (WHO grade II) are well-differentiated (not anaplastic), portending a better prognosis. High-grade (WHO grade III-IV) tumors are undifferentiated or anaplastic; these are malignant and carry a worse prognosis.

For about 30 years, the best predictor of an OV patient's survival has been tumor stage, i.e. the spread of disease at diagnosis. Additional indicators, such as the residual disease after surgery, the outcome of subsequent therapy, and the neoplasm status, which is the last known status of the disease, are determined during treatment. Other factors considered for more favorable prognosis include younger age, cell type other than mucinous and clear cell, smaller disease volume, and absence of ascites.

### II. Genomic Tensor Analysis for Medical Assessment and Prediction

The subject technology provides tensor mathematical models that can compare and integrate different types of large-scale molecular biological datasets, such as, but not limited to, mRNA expression levels, DNA microarray data, DNA copy number alterations, protein expression, etc.

Additional possible applications of the tensor GSVD in personalized medicine include comparative modeling of two patient- and tissue-matched datasets, each corresponding to (i) a set of large-scale molecular biological profiles, e.g., DNA copy numbers, acquired by a high-throughput technology, e.g., DNA microarrays; (ii) a set of biomedical images or signals; or (iii) a set of cellular pathological observations, e.g., a tumor's stage. Such tensor GSVD comparative models can uncover variations across the patients and tissues that are common to, possibly causally coordinated between the two aspects of the disease. In clinical settings, such tensor GSVD comparative models can determine an individual patient's medical status in relation to all the other patients in a set, and inform the patient's diagnosis, prognosis and treatment.

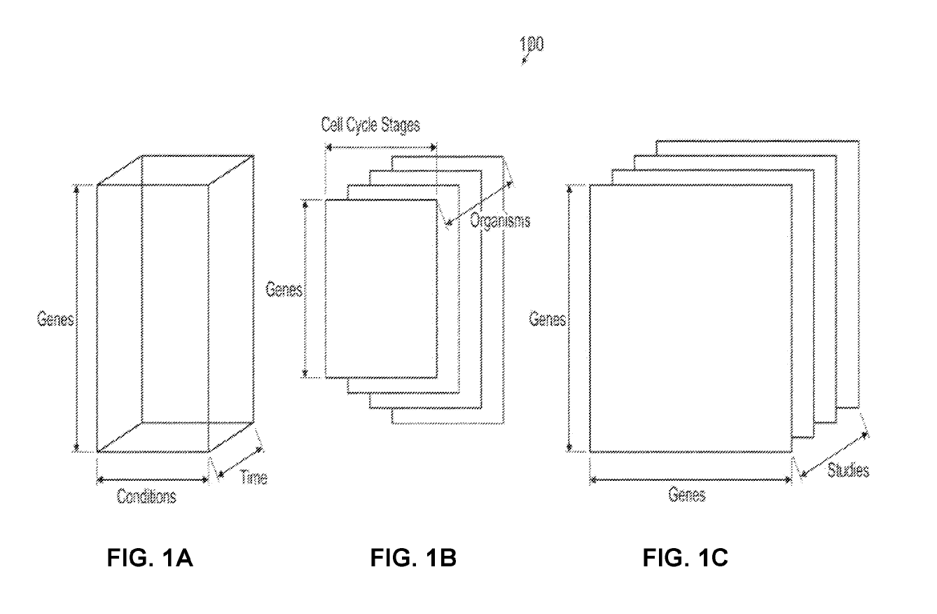

FIGS. 1A-1C are high-level diagrams illustrating suitable examples of tensors 100, according to some embodiments of the subject technology. In general, a tensor representing a number of biological datasets may comprise an N^{th}-order tensor including a number of multi-dimensional (e.g., two or three dimensional) matrices. Datasets may relate to biological information as shown in FIG. 1. An N^{th}-order tensor may include a number of biological datasets. Some of the biological datasets may correspond to one or more biological samples. Some of the biological dataset may include a number of biological data arrays, some of which may be associated with one or more subjects.

Referring to the specific embodiments illustrated in FIG. 1A, tensor represents a third order tensor (i.e., a cuboid), in which each dimension (e.g., gene, conditions, and time) represents a degree of freedom in the cuboid. If the cuboid is unfolded into a matrix, these degrees of freedom and along with it, most of the data included in the tensor may be lost. However, decomposing the cuboid using a tensor decomposition technique, such as a higher-order eigen-value decomposition (HOEVD) or a higher-order single value decomposition (HOSVD) may uncover patterns of variations (e.g., of mRNA expression) across genes, time points and conditions.

As shown in FIG. 1B, the tensor is a biological dataset that may be associated with genes across one or more organisms. Each data array also includes cell cycle stages. In this case, the tensor decomposition may allow, for example, the integration of global mRNA expressions measured for one or more organisms, the removal of experimental artifacts, and the identification of significant combinations of patterns of expression variation across the genes, for various organisms and for different cell cycle stages.

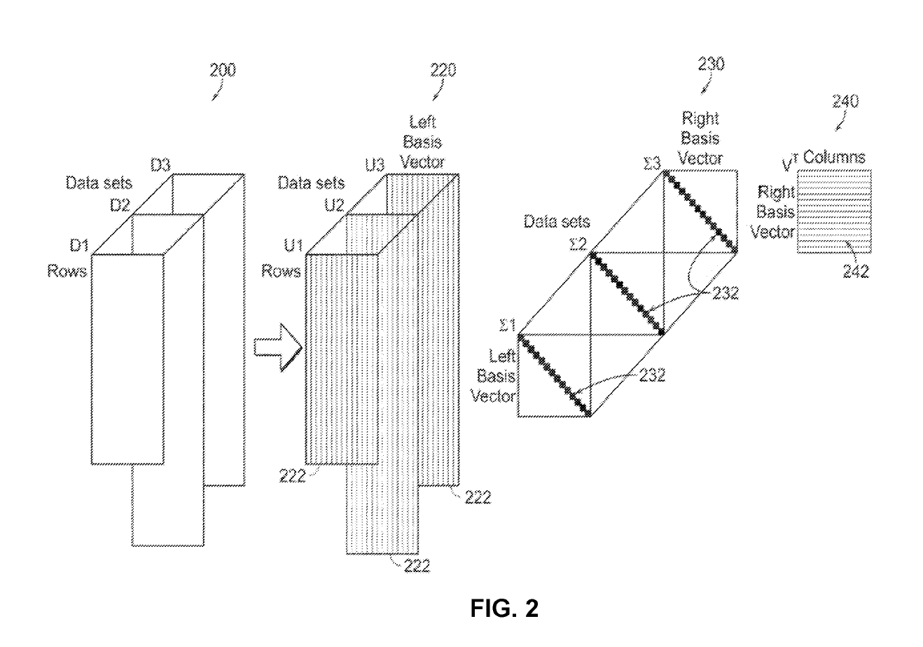

Similarly, as seen in FIG. 1C, the tensor contains biological datasets associated with a network K of N-genes by N-genes. The network K represents the number of studies on the genes. The tensor decomposition (e.g., HOEVD) in this case may allow, for example, uncovering important relationships among the genes (e.g., pheromone-response-dependent relation or orthogonal cell-cycle-dependent relation). An example of a tensor comprising a three-dimensional array is discussed below in reference to FIG. 2.

FIG. 2 is a high-level diagram illustrating a linear transformation of a number of two dimensional (2-D) arrays forming a three-dimensional (3-D) array 200, according to some embodiments. The 3-D array 200 may be stored in a memory. The 3-D array 200 may include an N number of biological datasets (e.g., D1, D2, and D3) that correspond to, for example, genetic sequences. In some cases, the 3-D array 200 may comprise an N number of 2-D data arrays (D1, D2, D3, . . . D_{N}) (for clarity only D1-D3 are shown in FIG. 2). In this case, N is equal to 3. However, this is not intended to be limiting as N may be any number (1 or greater). In some embodiments, N is greater than 2.

In some cases, each biological dataset may correspond to a tissue type and include an M number of biological data arrays. Each biological data array may be associated with a patient or, more generally, an organism. Each biological data array may include a plurality of data units (e.g., genes, chromosome segments, chromosomes). Each 2-D data array can store one set of the biological datasets and includes M columns. Each column can store one of the M biological data arrays corresponding to a subject such as a patient.

A linear transformation such as a tensor decomposition algorithm may be applied to the 3-D array 200 to generate a plurality of eigen 2-D arrays 220, 230, and 240. The eigen 2-D arrays 220, 230, and 240 can then be analyzed to determine one or more characteristics related to a disease.

Each data array generally comprises measurable data. In some embodiments, each data array may comprise biological data that represent a physical reality such as the specific stage of a cell cycle. In some embodiments the biological data may be measured by, for example, DNA microarray technology, sequencing technology, protein microarray, mass spectrometry in which protein abundance levels are measured on a large proteomic scale as well as traditional measurement technologies (e.g., immunohistochemical staining). Suitable examples of biological data include, but are not limited to, mRNA expression level, gene product level, DNA copy number, micro-RNA expression, presence of DNA methylation, binding of proteins to DNA or RNA, protein expression, and the like. In some embodiments, the biological data may be derived from a patient-specific sample including a normal tissue, a disease-related tissue or a culture of a patient's cell (normal and/or disease-related).

In some embodiments, the biological datasets may comprise genes from one or more subjects along with time points and/or other conditions. A tensor decomposition of the N^{th}-order tensor may allow for the identification of abnormal patterns (e.g., abnormal copy number variations) in a subject. In some cases, these patterns may identify genes that may correlate or possibly coordinate with a particular disease. Once these genes are identified, they may be useful in the diagnosis, prognosis, and potentially treatment of the disease.

For example, a tensor decomposition may identify genes that enables classification of patients into subgroups based on patient-specific genomic data. In some cases, the tensor decomposition may allow for the identification of a particular disease subtype. In some cases, the subtype may be a patient's increased response to a therapeutic method such as chemotherapy, lack of increased response to chemotherapy, increased life expectancy, lack of increased life expectancy and the like. Thus, the tensor decomposition may be advantageous in the treatment of patient's disease by allowing subgroup- or subtype-specific therapies (e.g., chemotherapy, surgery, radiotherapy, etc.) to be designed. Moreover, these therapies may be tailored based on certain criteria, such as, the correlation between an outcome of a therapeutic method and a global genomic predictor.

In facilitating or enabling prognosis of a disease, the tensor decomposition may also predict a patient's survival. An N^{th}-order tensor may include a patient's routine examinations data, in which case decomposition of the tensor may allow for the designing of a personalized preventive regimen for the patient based on analyses of the patient's routine examinations data. In some embodiments, the biological datasets may be associated with imaging data including magnetic resonance imaging (MRI) data, electro cardiogram (ECG) data, electromyography (EMG) data or electroencephalogram (EEG) data. A biological datasets may also be associated with vital statistics, phenotypical data, as well as molecular biological data (e.g., DNA copy number, mRNA expression level, gene product level, etc.). In some cases, prognosis may be estimated based on an analysis of the biological data in conjunction with traditional risk factors such as, age, sex, race, etc.

Tensor decomposition may also identify genes useful for performing diagnosis, prognosis, treatment, and tracking of a particular disease. Once these genes are identified, the genes may be analyzed by any known techniques in the relevant art. For example, in order to perform a diagnosis, prognosis, treatment, or tracking of a disease, the DNA copy number may be measured by a technique such as, but not limited to, fluorescent in-situ hybridization, complementary genomic hybridization, array complementary genomic hybridization, and fluorescence microscopy. Other commonly used techniques to determine copy number variations include, e.g. oligonucleotide genotyping, sequencing, southern blotting, dynamic allele-specific hybridization (DASH), paralogue ratio test (PRT), multiple amplicon quantification (MAQ), quantitative polymerase chain reaction (QPCR), multiplex ligation dependent probe amplification (MLPA), multiplex amplification and probe hybridization (MAPH), quantitative multiplex PCR of short fluorescent fragment (QMPSF), dynamic allele-specific hybridization, fluorescence in situ hybridization (FISH), semiquantitative fluorescence in situ hybridization (SQ-FISH) and the like. For more detail description of some of the methods described herein, see, e.g. Sambrook, Molecular Cloning—A Laboratory Manual, Cold Spring Harbor Laboratory, Cold Spring Harbor, N.Y., (1989), Kallioniemi et al., Proc. Natl. Acad Sci USA, 89:5321-5325 (1992), and PCR Protocols, A Guide to Methods and Applications, Innis et al., Academic Press, Inc. N.Y., (1990).

The mRNA level may be measured by a technique such as, northern blotting, gene expression profiling, and serial analysis of gene expression. Other commonly used techniques include RT-PCR and microarray technology. In a typical microarray experiment, a microarray is hybridized with differentially labeled RNA or DNA populations derived from two different samples. Ratios of fluorescence intensity (red/green, R/G) represent the relative expression levels of the mRNA corresponding to each cDNA/gene represented on the microarray. Real-time polymerase chain reaction, also called quantitative real time PCR (QRT-PCR) or kinetic polymerase chain reaction, may be highly useful to determine the expression level of a mRNA because the technique can simultaneously quantify and amplify a specific part of a given polynucleotide.

The gene product level may be measured by a technique such as, enzyme-linked immunosorbent assay (ELISA) and fluorescence microscopy. When the gene product is a protein, traditional methodologies for protein quantification include 2-D gel electrophoresis, mass spectrometry and antibody binding. Commonly used antibody-based techniques include immunoblotting (western blotting), immunohistological assay, enzyme linked immunosorbent assay (ELISA), radioimmunoassay (RIA), or protein chips. Gel electrophoresis, immunoprecipitation and mass spectrometry may be carried out using standard techniques, for example, such as those described in Molecular Cloning A Laboratory Manual, 2nd Ed., ed. by Sambrook, Fritsch and Maniatis (Cold Spring Harbor Laboratory Press: 1989), Harlow and Lane, Antibodies: A Laboratory Manual (1988 Cold Spring Harbor Laboratory), G. Suizdak, Mass Spectrometry for Biotechnology (Academic Press 1996), as well as other references cited herein.

In some embodiments, the tensor decomposition of the N^{th}-order tensor may allow for the removal of normal pattern copy number alterations and/or an experimental variation from a genomic sequence. Thus, a tensor decomposition of the N^{th}-order tensor may permit an improved prognostic prediction of the disease by revealing real disease-associated changes in chromosome copy numbers, focal copy number alterations (CNAs), non-focal CNAs and the like. A tensor decomposition of the N^{th}-order tensor may also allow integrating global mRNA expressions measured in multiple time courses, removal of experimental artifacts, and identification of significant combinations of patterns of expression variation across genes, time points and conditions.

In some embodiments, applying the tensor decomposition algorithm may comprise applying at least one of a higher-order singular value decomposition (HOSVD), a higher-order generalized singular value decomposition (HO GSVD), a higher-order eigen-value decomposition (HOEVD), or parallel factor analysis (PARAFAC) to the N^{th}-order tensor. The PARAFAC method is known in the art and will not be described with respect to the present embodiments. In some embodiments, HOSVD may be utilized to decompose a 3-D array 200, as described in more detail herein.

Referring again to FIG. 2, eigen 2-D arrays generated by HOSVD may comprise a set of N left-basis 2-D arrays 220. Each of the left-basis arrays 220 (e.g., U1, U2, U3, . . . U_{N}) (for clarity, only U1-U3 are shown in FIG. 2) may correspond, for example, to a tissue type and can include an M number of columns, each of which stores a left-basis vector 222 associated with a patient. The eigen 2-D arrays 230 comprise a set of N diagonal arrays (Σ1, Σ2, Σ3 . . . ΣN) (for clarity only Σ1-Σ3 are shown in FIG. 2). Each diagonal array (e.g., Σ1, Σ2, Σ3 . . . or ΣN) may correspond to a tissue type and can include an N number of diagonal elements 232. The 2-D array 240 comprises a right-basis array, which can include a number of right-basis vectors 242.

In some embodiments, decomposition of the N^{th}-order tensor may be employed for disease related characterization such as identifying genes or chromosomal segments useful for diagnosing, tracking a clinical course, estimating a prognosis or treating the disease.

In some embodiments, the biological data characterization system may be a computer system as known in the art. The system will typically include a processor, memory, an analysis module, and a display module. The processor may include one or more processors and may be coupled to the memory. Information related to the N^{th}-order tensors 100 of FIG. 1 or the 3-D array 200 of FIG. 2 may be retrieved from a database coupled to the system and store tensors 100 or the 3-D array 200 along with 2-D eigen-arrays 220, 230, and 240 of FIG. 2. A database may be coupled to the system via a network (e.g., Internet, wide area network (WAN), local area network (LAN), etc.). In some embodiments, the system may encompass the database.

Such systems are known in the art and include computer systems as described, for example, in U.S. Publication No. 2014/0249762 and 2014/0303029, both of which are incorporated herein by reference.

The processor can apply a tensor decomposition algorithm, such as HOSVD, HO GSVD, or HOEVD, to tensor 100 or 3-D array 200 in order to generate eigen 2-D arrays 220, 230 and 240. In some embodiments, the processor may apply the HOSVD or HO GSVD algorithms to data obtained from array comparative genomic hybridization (aCGH) of patient-matched normal and ovarian serous cystadenocarcinoma (OV) blood samples (see Example 2). Application of HOSVD algorithm may remove one or more normal pattern copy number alterations (PCAs) or experimental variations from the aCGH data. A HOSVD algorithm can also reveal OV-associated changes in at least one of chromosome copy numbers, focal CNAs, and unreported CNAs existing in the aCGH data. Analysis may be performed for disease related characterizations as discussed above. For example, various analyses of eigen 2-D arrays 230 of FIG. 2 may be facilitated by assigning each diagonal element 232 of FIG. 2 to an indicator of a significance of a respective element of a right-basis vector 222 of FIG. 2, as described herein in more detail. A display module 240 can display 2-D arrays 220, 230, 240 and any other graphical or tabulated data resulting from analyses performed by an analysis module. A display module may comprise software and/or firmware and may use one or more display units such as cathode ray tubes (CRTs) or flat panel displays.

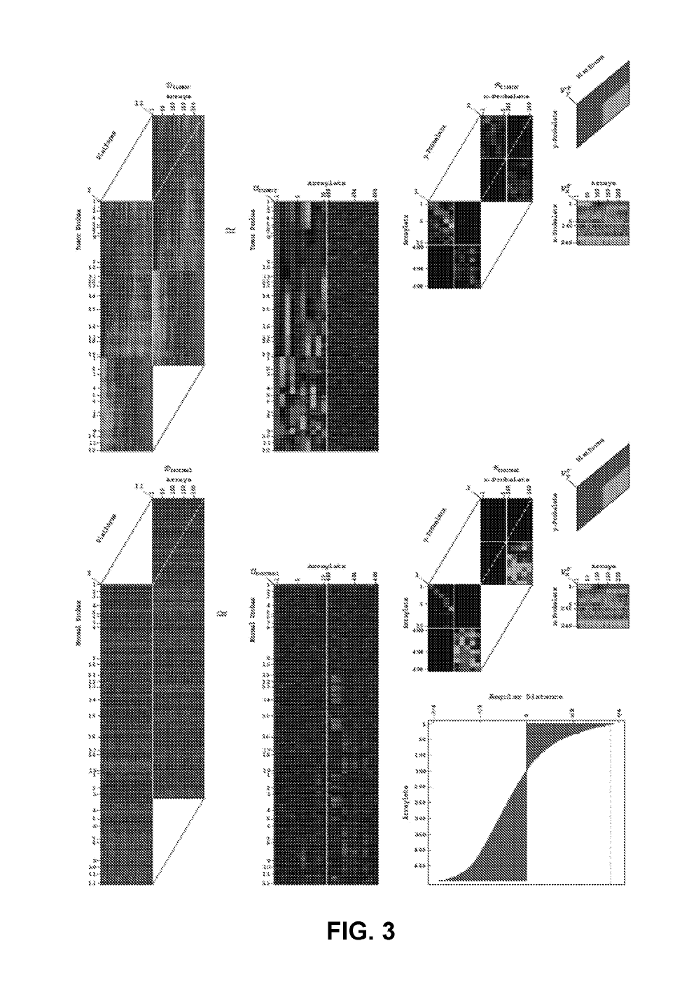

In some embodiments a method for genomic prognostic prediction is provided. The method includes storing the N^{th}-tensors 100 of FIG. 1 or 3-D array 200 of FIG. 2 in a memory. A tensor decomposition algorithm such as HOSVD, HO GSVD or HOEVD may be applied by a processor to the datasets stored in tensors 100 or 3-D array 200 to generate eigen 2-D arrays 220, 230, and 240 of FIG. 2. A generated eigen 2-D arrays 220, 230, and 240 may be analyzed, e.g. by an analysis module, to determine one or more disease-related characteristics.

A HOSVD algorithm is mathematically described herein with respect to N>2 matrices (i.e., arrays D₁-D_{N}) of 3-D array 200. Each matrix can be a real mᵢ×n matrix. Each matrix is exactly factored as Dᵢ=Uᵢ ΣᵢV^{T}, where V, identical in all factorizations, is obtained from the balanced eigensystem SV=VΛ of the arithmetic mean S of all pairwise quotients AᵢAⱼ⁻¹ of the matrices Aᵢ=Dᵢ^{T}Di, where i is not equal to j, independent of the order of the matrices D₁. It can be proved that this decomposition extends to higher orders, all of the mathematical properties of the GSVD except for column-wise orthogonality of the matrices Uᵢ (e.g., 2-D arrays 120 of FIG. 1). It can be proved that matrix S is nondefective. In other words, S has n independent eigenvectors and that V is real and the eigenvalues of S (i.e., λ₁, λ₂, . . . λ_{N}) satisfy λₖ≥1.

In the described HO GSVD comparison of two matrices, the kth diagonal element of Σᵢ=diag(σ_{1,k}) (e.g., the kₜₕ element 132 of FIG. 1) is interpreted in the factorization of the iₜₕ matrix Dᵢ as indicating the significance of the kₜₕ right basis vector vₖ in Dᵢ in terms of the overall information that vₖ captures in Dᵢ. The ratio σ_{i,k}/σ_{j,k} indicates the significance of vₖ in Dᵢ relative to its significance in Dⱼ. It can also be proved that an eigenvalue λₖ=1 corresponds to a right basis vector vₖ of equal significance in all matrices Dᵢ and Dⱼ for all i and j when the corresponding left basis vector u_{i,k} is orthonormal to all other left basis vectors in Uᵢ for all i. Detailed description of various analysis results corresponding to application of the HOSVD to a number of datasets obtained from patients and other subjects will be discussed below. For clarity, a more detailed treatment of the mathematical aspects of HOSVD is skipped here but provided in the attached Appendices A, B, and C. Disclosures in Appendix A have also been published as Lee et al., (2012) GSVD Comparison of Patient-Matched Normal and Tumor aCGH Profiles Reveals Global Copy-Number Alterations Predicting Glioblastoma Multiforme Survival, in PLoS ONE 7(1): e30098. doi:10.1371/journal.pone.0030098. Disclosures in Appendices B and C have been published as Ponnapalli et al., (2011) A Higher-Order Generalized Singular Value Decomposition for Comparison of Global mRNA Expression from Multiple Organisms in PLoS ONE 6(12): e28072. doi: 10.1371/journal.pone.0028072.

A HOEVD tensor decomposition method can be used for decomposition of higher order tensors. Herein, as an example, the HOEVD tensor decomposition method is described in relation with a the third-order tensor of size K-networks× N-genes× N-genes as follows:

### Higher-Order EVD (HOEVD).

Let the third-order tensor {\hat{α}ₖ} of size K-networks×N-genes×N-genes tabulate a series of K genome-scale networks computed from a series of K genome-scale signals {êₖ}, of size N-genes×Mₖ-arrays each, such that \hat{α}ₖ=êₖêₖ^{T}, for all k=1, 2, . . . , K. We define and compute a HOEVD of the tensor of networks {\hat{α}ₖ},

$$\hat{a} ≡ {\sum }_{k = 1}^{K} {\hat{a}}_{k} = \hat{u} ( {\sum }_{k = 1}^{K} {\hat{ɛ}}_{k}^{2} ) {\hat{u}}^{T} = \hat{u} {\hat{ɛ}}^{2} {\hat{u}}^{T} , \qquad [ 5 ]$$

using the SVD of the appended signals ê≡(ê₁, ê₂, . . . , ê_{K})=û\hat{ϵ}\hat{v}^{T}, where the mth column of û, |αₘ [symbol] ≡û|m [symbol] , lists the genome-scale expression of the mth eigenarray of ê. Whereas the matrix EVD is equivalent to the matrix SVD for a symmetric nonnegative matrix, this tensor HOEVD is different from the tensor higher-order SVD (14-16) for the series of symmetric nonnegative matrices {\hat{α}ₖ}, where the higher-order SVD is computed from the SVD of the appended networks (\hat{α}₁, \hat{α}₂, . . . \hat{α}_{K}) rather than the appended signals. This HOEVD formulates the overall network computed from the appended signals \hat{α}=êê^{T} as a linear superposition of a series of M≡Σₖ₌₁^{K} Mₖ rank-1 symmetric “subnetworks” that are decorrelated of each other, \hat{α}=Σₘ₌₁^{M} ϵₘ²|αₘ [symbol] [symbol] αₘ|. Each subnetwork is also decoupled of all other subnetworks in the overall network \hat{α}, since \hat{ϵ} is diagonal.

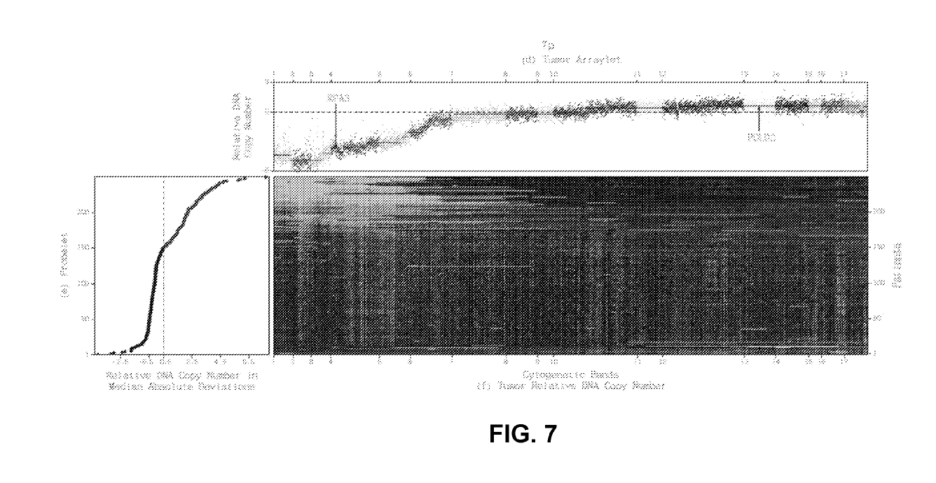

This HOEVD formulates each individual network in the tensor {\hat{α}ₖ} as a linear superposition of this series of M rank-1 symmetric decorrelated subnetworks and the series of M(M-1)/2 rank-2 symmetric couplings among these subnetworks (FIG. 7 in Supporting Appendix), such that

$${\hat{a}}_{k} = {\sum }_{m = 1}^{M} {ɛ}_{k , m}^{2} \vert {α}_{m} 〉 〈 {α}_{m} \vert$$

$$+ {\sum }_{m = 1}^{M} {\sum }_{l = m + 1}^{M} {ɛ}_{k , lm}^{2} ( \vert {α}_{l} 〉 〈 {α}_{m} \vert + \vert {α}_{m} 〉 〈 {α}_{l} \vert ) , \qquad [ 6 ]$$

for all k=1, 2, . . . , K. The subnetworks are not decoupled in any one of the networks {\hat{α}ₖ}, since, in general, {\hat{ϵ}ₖ²} are symmetric but not diagonal, such that ϵ_{k,lm}²≡ [symbol] l|\hat{ϵ}ₖ²|m [symbol] = [symbol] m|\hat{ϵ}ₖ²|l [symbol] ≠0. The significance of the mth subnetwork in the kth network is indicated by the mth fraction of eigen expression of the kth network ρ_{k,m}=ϵ_{k,m}²/(Σₖ₌₁^{K} Σₘ₌₁^{M} ϵ_{k,m}²)≥0, i.e., the expression correlation captured by the mth subnetwork in the kth network relative to that captured by all subnetworks (and all couplings among them, where Σₖ₌₁^{K} ϵ_{k,m}²=0 for all 1≠m) in all networks. Similarly, the amplitude of the fraction ρ_{k,lm}=ϵ_{k,lm}²/(Σₖ₌₁^{K} Σₘ₌₁^{M} ϵ_{k,m}²) indicates the significance of the coupling between the lth and mth subnetworks in the kth network. The sign of this fraction indicates the direction of the coupling, such that ρ_{k,lm}>0 corresponds to a transition from the lth to the mth subnetwork and ρ_{k,lm}<0 corresponds to the transition from the mth to the metric distribution of the annotations among the N-genes and the subsets of n⊆N genes with largest and smallest levels of expression in this eigenarray. The corresponding eigengene might be inferred to represent the corresponding biological process from its pattern of expression.

For visualization, we set the x correlations among the X pairs of genes largest in amplitude in each subnetwork and coupling equal to ±1, i.e., correlated or anticorrelated, respectively, according to their signs. The remaining correlations are set equal to 0, i.e., decorrelated. We compare the discretized subnetworks and couplings using Boolean functions (6).

Interpretation of the Subnetworks and their Couplings.

We parallel- and antiparallel-associate each subnetwork or coupling with most likely expression correlations, or none thereof, according to the annotations of the two groups of x pairs of genes each, with largest and smallest levels of correlations in this subnetwork or coupling among all X=N(N−1)/2 pairs of genes, respectively. The P value of a given association by annotation is calculated by using combinatorics and assuming hypergeometric probability distribution of the Y pairs of annotations among the X pairs of genes, and of the subset of y⊆Y pairs of annotations among the subset of x⊆X pairs of genes,

$$P ( x ; y , Y , X ) = {( \begin{matrix}X \\ x\end{matrix} )}^{- 1}$$

$${\sum }_{z = y}^{x} ( \begin{matrix}Y \\ z\end{matrix} ) ( \begin{matrix}X - Y \\ x - z\end{matrix} ) ,$$

where

$$( \begin{matrix}X \\ x\end{matrix} ) = X ! {x !}^{- 1} {( X - x )}^{- 1}$$

is the binomial coefficient (17). The most likely association of a subnetwork with a pathway or of a coupling between two subnetworks with a transition between two pathways is that which corresponds to the smallest P value. Independently, we also parallel- and antiparallel-associate each eigenarray with most likely cellular states, or none thereof, assuming hypergeometric distribution of the annotations among the N-genes and the subsets of n⊆N genes with largest and smallest levels of expression in this eigenarray. The corresponding eigengene might be inferred to represent the corresponding biological process from its pattern of expression.

For visualization, we set the x correlations among the X pairs of genes largest in amplitude in each subnetwork and coupling equal to ±1, i.e., correlated or anticorrelated, respectively, according to their signs. The remaining correlations are set equal to 0, i.e., decorrelated. We compare the discretized subnetworks and couplings using Boolean functions (6).

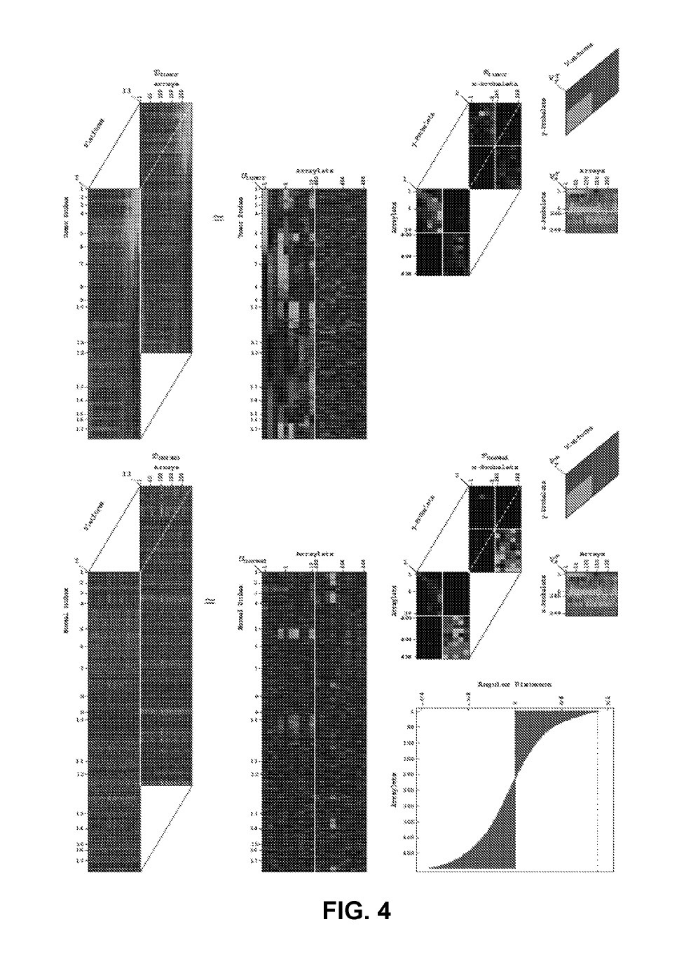

With reference to FIG. 39 as shown in U.S. Published Application No. 2014/0303029, incorporated herein by reference, a higher-order EVD (HOEVD) of the third-order series of the three networks {\hat{α}₁, \hat{α}₂, \hat{α}₃}. The network \hat{α}₃ is the pseudoinverse projection of the network \hat{α}₁ onto a genome-scale proteins' DNA-binding basis signal of 2,476-genes×12-samples of development transcription factors [3] (Mathematica Notebook 3 and Data Set 4), computed for the 1,827 genes at the intersection of \hat{α}₁ and the basis signal. The HOEVD is computed for the 868 genes at the intersection of \hat{α}₁, \hat{α}₂ and \hat{α}₃. Raster display of \hat{α}ₖ≈Σₘ₌₁³ϵ_{k,m}²|αₘ [symbol] [symbol] αₘ|+Σₘ₌₁³ Σₗ₌ₘ₊₁³ϵ_{k,lm}² (|αₗ [symbol] [symbol] αₘ|+αₘ [symbol] [symbol] αₗ|), for all k=1, 2, 3, visualizing each of the three networks as an approximate superposition of only the three most significant HOEVD subnetworks and the three couplings among them, in the subset of 26 genes which constitute the 100 correlations in each subnetwork and coupling that are largest in amplitude among the 435 correlations of 30 traditionally-classified cell cycle-regulated genes. This tensor HOEVD is different from the tensor higher-order SVD [14-16] for the series of symmetric nonnegative matrices {\hat{α}₁, \hat{α}₂, \hat{α}₃}. The subnetworks correlate with the genomic pathways that are manifest in the series of networks. The most significant subnetwork correlates with the response to the pheromone. This subnetwork does not contribute to the expression correlations of the cell cycle-projected network \hat{α}₂, where ϵ_{2,1}²≈0. The second and third subnetworks correlate with the two pathways of antipodal cell cycle expression oscillations, at the cell cycle stage G₁ vs. those at G₂, and at S vs. M, respectively. These subnetworks do not contribute to the expression correlations of the development-projected network \hat{α}₃, where ϵ_{3,2}²≈ϵ_{3,3}²≈0. The couplings correlate with the transitions among these independent pathways that are manifest in the individual networks only. The coupling between the first and second subnetworks is associated with the transition between the two pathways of response to pheromone and cell cycle expression oscillations at G₁ vs. those G₂, i.e., the exit from pheromone-induced arrest and entry into cell cycle progression. The coupling between the first and third subnetworks is associated with the transition between the response to pheromone and cell cycle expression oscillations at S vs. those at M, i.e., cell cycle expression oscillations at G₁/S vs. those at M. The coupling between the second and third subnetworks is associated with the transition between the orthogonal cell cycle expression oscillations at G₁ vs. those at G₂ and at S vs. M, i.e., cell cycle expression oscillations at the two antipodal cell cycle checkpoints of G₁/S vs. G₂/M. All these couplings add to the expression correlation of the cell cycle-projected \hat{α}₂, where ϵ_{2,12}², ϵ_{2,13}², ϵ_{2,23}²>0; their contributions to the expression correlations of \hat{α}₁ and the development-projected \hat{α}₃ are negligible (see also FIG. 4 of US 2014/0303029).

In embodiments, a tensor GSVD arranged in two higher-than-second-order tensors of matched column dimensions but independent row dimensions is used in the methods herein. For clarity, a more detailed treatment of the mathematical aspects of this tensor GSVD provided in the attached Appendix A.

Primary OV tumor and normal DNA copy-number profiles of a set of 249 TCGA patients were selected. Each profile was measured in two replicates by the same set of two DNA microarray platforms. For each chromosome arm or combination of two chromosome arms, the structure of these tumor and normal discovery datasets D₁ and D₂, of K₁-tumor and K₂-normal probes×L-patients, i.e., arrays×M-platforms, is that of two third-order tensors with one-to-one mappings between the column dimensions L and M, but different row dimensions K₁ and K₂, where K₁, K₂≥LM.

This tensor GSVD simultaneously separates the paired datasets into weighted sums of LM paired “subtensors,” i.e., combinations or outer products of three patterns each: Either one tumor-specific pattern of copy-number variation across the tumor probes, i.e., a “tumor arraylet” u_{1,a}, or the corresponding normal-specific pattern across the normal probes, i.e., the “normal arraylet” u_{2,a}, combined with one pattern of copy-number variation across the patients, i.e., an “x-probelet” v_{x,b}^{T} and one pattern across the platforms, i.e., a “y-probelet” v_{y,c}^{T}, which are identical for both the tumor and normal datasets (see FIGS. 3-5),

$${𝒟}_{i} = {R}_{i} \times {}_{a}^{}{U}{}_{i}^{} \times {}_{b}^{}{V}{}_{x}^{} \times {}_{c}^{}{V}{}_{y}^{} = {\sum }_{a = 1}^{LM} {\sum }_{b = 1}^{L} {\sum }_{c = 1}^{M} {R}_{i , abc} {S}_{i} ( a , b , c ) \\ {S}_{i} ( a , b , c ) = {u}_{i , a} ⊗ {v}_{x , b}^{T} ⊗ {v}_{y , c}^{T} , i = 1 , 2 , \qquad ( 1 )$$

where xₐUᵢ, x_{b}Vₓ and x_{c}V_{y} denote tensor-matrix multiplications, which contract the LM-arraylet, L-x-probelet, and M-y-probelet dimensions of the “core tensor” [symbol] ᵢ with those of Uᵢ, Vₓ, and V_{y}, respectively, and where ⊗ denotes an outer product.

It was found that unfolding (or matricizing) both tensors Dᵢ into matrices, each preserving the Kᵢ-row dimension, e.g., by appending the LM columns D_{i:lm} of the corresponding tensor, gives two full column-rank matrices Dᵢ ϵ [symbol] ^{k}^{ᵢ}^{×LM}. The column bases vectors Uᵢ were obtained from the GSVD of Dᵢ, i.e., the “row mode GSVD”

Dᵢ=( . . . ,D_{i:lm}, . . . )=UᵢΣᵢV^{T},i=1,2. (2)

Similarly, that unfolding both tensors Dᵢ into matrices, each preserving the L-x- (or M-y-) column dimension, e.g., by appending the KᵢM rows D_{i,k}_{ᵢ}_{:m}^{T}(or the KᵢL rows D_{i,k}_{ᵢ}_{l:}^{T}) of the corresponding tensor, gives two full column-rank matrices Dᵢₓ ϵ [symbol] ^{k}^{ᵢ}^{M×L} (or D_{iy} ϵ [symbol] ^{k}^{ᵢ}^{L×M}). We obtain the x- (or y-) row basis vectors Vₓ^{T} (or V_{y}^{T}), from the GSVD of Dᵢₓ (or D_{iy}), i.e., the x- (or y-) column mode GSVD,

Dᵢₓ=( . . . ,Dᵢ^{T}_{k;m}, . . . )=UᵢₓΣᵢₓVₓ^{T},

D_{iy}=( . . . ,Dᵢ^{T}_{k;l}, . . . )=U_{iy}Σ_{iy}V_{y}^{T},i=1,2. (3)

Note that the x- and y-row bases vectors are, in general, non-orthogonal but normalized, and Vₓ and V_{y} are invertible. The column bases vectors are normalized and orthogonal, i.e., uncorrelated, such that Uᵢ^{T}Uᵢ=I.

The generalized singular values are positive, and are arranged in Σᵢ, Σᵢₓ, and Σ_{iy} in decreasing orders of the corresponding “GSVD angular distances,” i.e., decreasing orders of the ratios σ_{1,a}/σ_{2,a}, σ_{1x,b}/σ_{2x,b}, and σ_{1y,c}/σ_{2y,c}, respectively. We then compute the core tensors [symbol] ᵢ by contracting the row-, x-, and y-column dimensions of the tensors Dᵢ with those of the matrices Uᵢ, Vₓ⁻¹, and V_{y}⁻¹, respectively. For real tensors, the “tensor generalized singular values” [symbol] _{i,abc} tabulated in the core tensors are real but not necessarily positive. Our tensor GSVD construction generalizes the GSVD to higher orders in analogy with the generalization of the singular value decomposition (SVD) by the HOSVD, and is different from other approaches to the decomposition of two tensors.

It is proven herein that the tensor GSVD exists for two tensors of any order because it is constructed from the GSVDs of the tensors unfolded into full column-rank matrices (Lemma A Example 5). The tensor GSVD has the same uniqueness properties as the GSVD, where the column bases vectors u_{i,a} and the row bases vectors u_{x,b}^{T} and u_{y,c}^{T} are unique, except in degenerate subspaces, defined by subsets of equal generalized singular values σᵢ, σᵢₓ, and σ_{iy}, respectively, and up to phase factors of ±1, such that each vector captures both parallel and antiparallel patterns (Lemma B in S1 Appendix). The tensor GSVD of two second-order tensors reduces to the GSVD of the corresponding matrices (see Example 5). The tensor GSVD of the tensor D₁ Σ [symbol] ^{LM×L×M}, which row mode unfolding gives the identity matrix D₁=I ϵ [symbol] ^{LM×LM}, and a tensor D₂ of the same column dimensions reduces to the HOSVD of D₂ (Theorem A in Example 5).

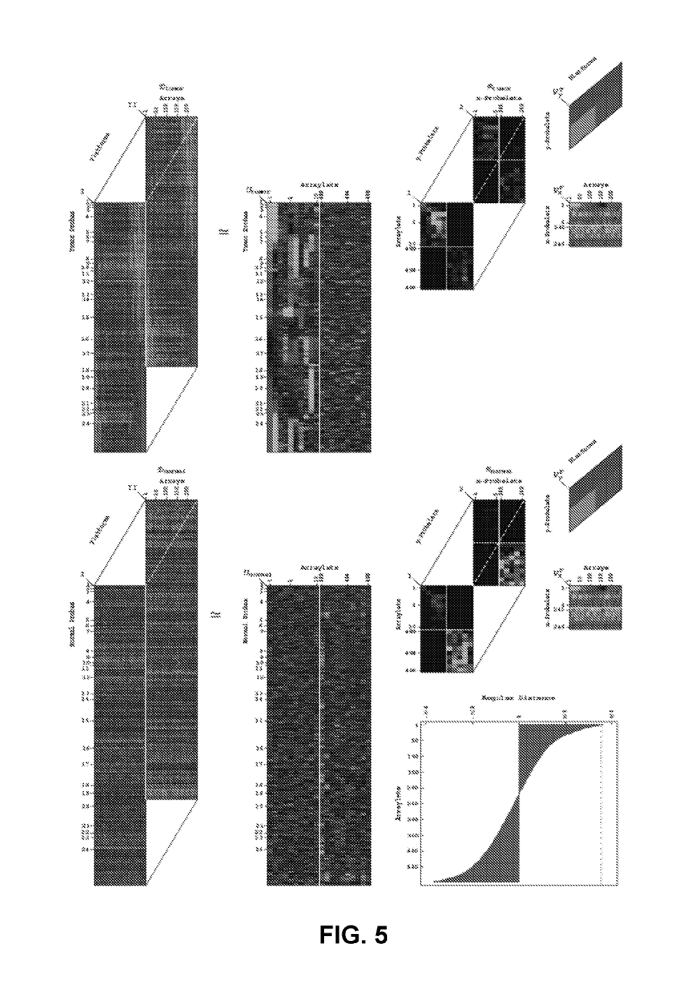

The significance of the subtensor Sᵢ(a, b, c) in the tensor Dᵢ is defined proportional to the magnitude of the corresponding tensor generalized singular values R_{i,abc} (FIG. 5), in analogy with the HOSVD,

P_{i,abc}=R_{i,abc}²/Σₐ₌₁^{LM}Σ_{b=1}^{L}Σ_{c=1}^{M}R_{i,abc}²,i=1,2. (4)

The significance of S₁(a, b, c) in D₁ relative to that of S₂(a, b, c) in D₂ is defined by the “tensor GSVD angular distance” Θ_{abc} as a function of the ratio R_{1,abc}/R_{2,abc}. This is in analogy with, e.g., the row mode GSVD angular distance θₐ, which defines the significance of the column basis vector u_{1,a} in the matrix D₁ of Eq. (2) relative to that of u_{2,a} in D₂ as a function of the ratio σ_{1,a}/σ_{2,a},

Θ_{abc}=arctan(R_{1,abc}/R_{2,abc})−π/4,

θₐ=arctan(σ_{1,a}/σ_{2,a})−π/4. (5)

Because the ratios of the positive generalized singular values satisfy σ_{1,a}/σ_{2,a}ϵ [0, ∞), the row mode GSVD angular distances satisfy θₐ ϵ [−π/4, π/4]. The maximum (or minimum) angular distance, i.e., θₐ=π/4, which corresponds to σ_{1,a}/σ_{2,a}>>1 (or −π/4, which corresponds to σ_{1,a}/σ_{2,a}<<1), indicates that the row basis vector uₐ^{T} of Eq. (2), which corresponds to the column basis vectors u_{1,a} in D₁ and u_{2,a} in D₂, is exclusive to D₁ (or D₂). An angular distance of θₐ=0, which corresponds to σ_{1,a}/σ_{2,a}=1, indicates a row basis vector uₐ^{T} which is of equal significance in, i.e., common to both D₁ and D₂.

Thus, while the ratio σ_{1,a}/σ_{2,a} indicates the significance of u_{1,a} in D₁ relative to the significance of u_{2,a} in D₂, this relative significance is defined, as previously described, by the angular distance θₐ, a function of the ratio σ_{1,a}/σ_{2,a}, which is antisymmetric in D₁ and D₂. Note also that while other functions of the ratio σ_{1,a}/σ_{2,a} exist that are antisymmetric in D₁ and D₂, the angular distance θₐ, which is a function of the arctangent of the ratio, i.e., arctan(σ_{1,a}/σ_{2,a}), is the natural function to use, because the GSVD is related to the cosine-sine (CS) decomposition, as previously described, and, thus, σ_{1,a} and σ_{2,a} are related to the sine and the cosine functions of the angle θₐ, respectively.

Theorem 1. The tensor GSVD angular distance equals the row mode GSVD angular distance, i.e., Θ_{abc}=θₐ.

Proof. The unfolding of Dᵢ of Eq. (1) into Dᵢ of Eq. (2) unfolds the core tensors [symbol] ᵢ of Eq. (1) into matrices [symbol] ᵢ, which preserve the row dimensions, i.e., the LM-column bases dimensions of [symbol] ᵢ, and gives

Dᵢ=UᵢRᵢ(Vₓ^{T}⊗V_{y}^{T}

Rᵢ=(ΣᵢV^{T}(Vₓ^{-T}⊗V_{y}^{-T}), i=1,2, (6)

where ⊗ denotes a Kronecker product. Because Σᵢ are positive diagonal matrices, it follows that [symbol] _{1,abc}/ [symbol] _{2,abc}= [symbol] _{1,a}/ [symbol] _{2,a}=σ_{1,a}/σ_{2,a}. Substituting this in Eq. (5) gives Θ_{abc}=θₐ. Note that the proof holds for tensors of higher-than-third order.

From this it follows that the tensor GSVD angular distance |Θ_{abc}|≤π/4, and that, therefore, the ratio of the tensor generalized singular values [symbol] _{1,abc}/ [symbol] _{2,abc}>0, even though [symbol] _{1,abc} and [symbol] _{2,abc} are not necessarily positive. It also follows that Θ_{abc}=±π/4 indicate a subtensor exclusive to either D₁ or D₂, respectively, and that Θ_{abc}=0 indicates a subtensor common to both.

Note that in this embodiment since the generalized singular values are arranged in Σᵢ of Eq. (2) in a decreasing order of the row mode GSVD angular distances θₐ, the most tumor-exclusive tumor subtensors, i.e., S₁(a, b, c) where a maximizes θₐ of Eq. (5), correspond to a=1, whereas the most normal-exclusive normal sub-tensors, i.e., S₂(a, b, c) where a minimizes θₐ, correspond to a=LM.

III. Prediction of OV Survival and/or Response to Therapy Such as Platinum-Based Chemotherapy

In some embodiments, a tensor GSVD, i.e., an exact simultaneous decomposition of datasets, arranged in two higher-than-second-order tensors of matched column dimensions but independent row dimensions is used to create a model for OV.

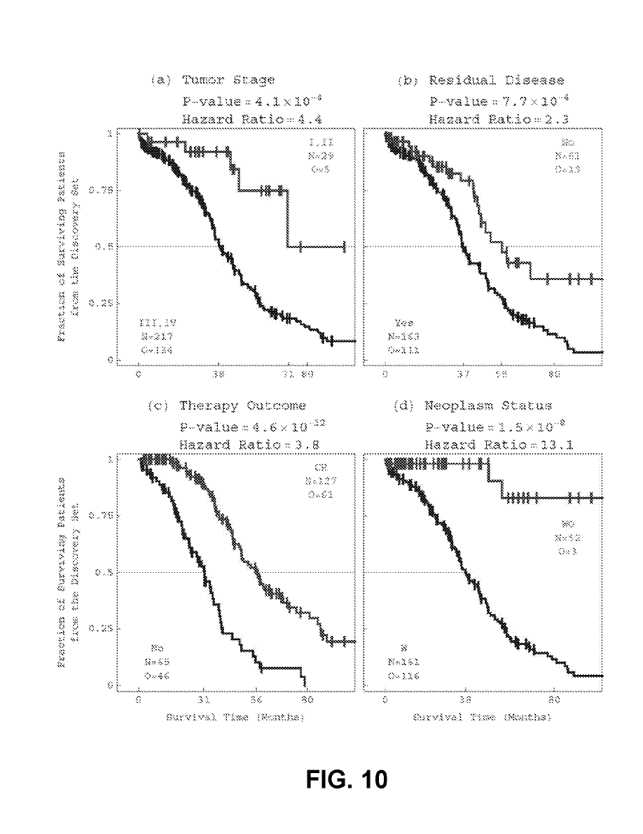

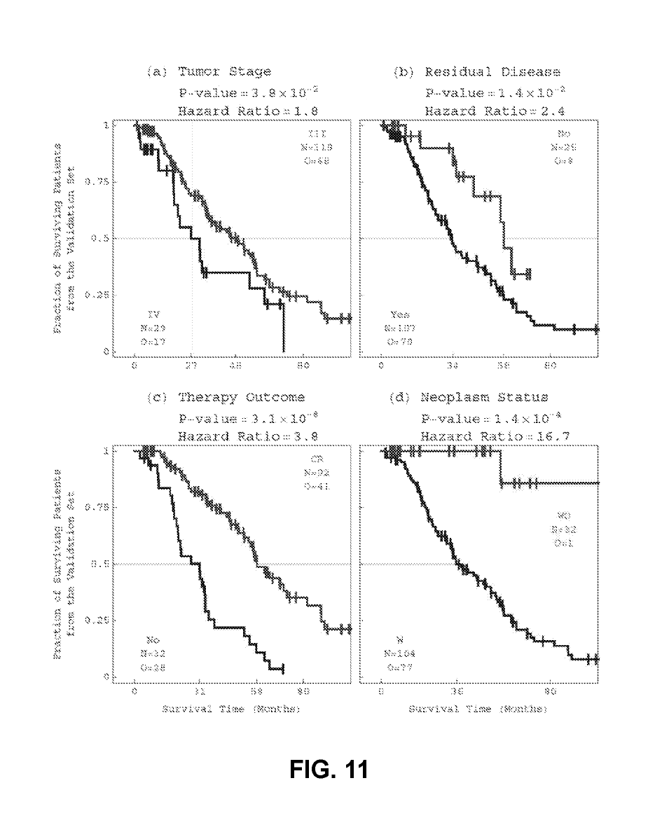

To date, the best predictor of OV survival has remained the tumor's stage at diagnosis (FIGS. 10 and 11). Additional indicators, such as the residual disease after surgery, the outcome of subsequent therapy, and the neoplasm status, which is the last known status of the disease, are determined during treatment. No diagnostic exists that distinguishes between platinum-based chemotherapy-resistant and -sensitive tumors before the treatment.

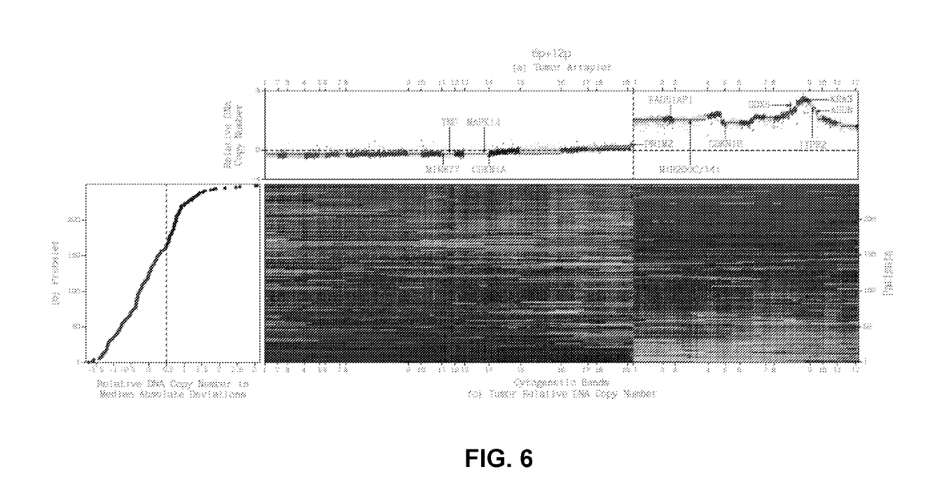

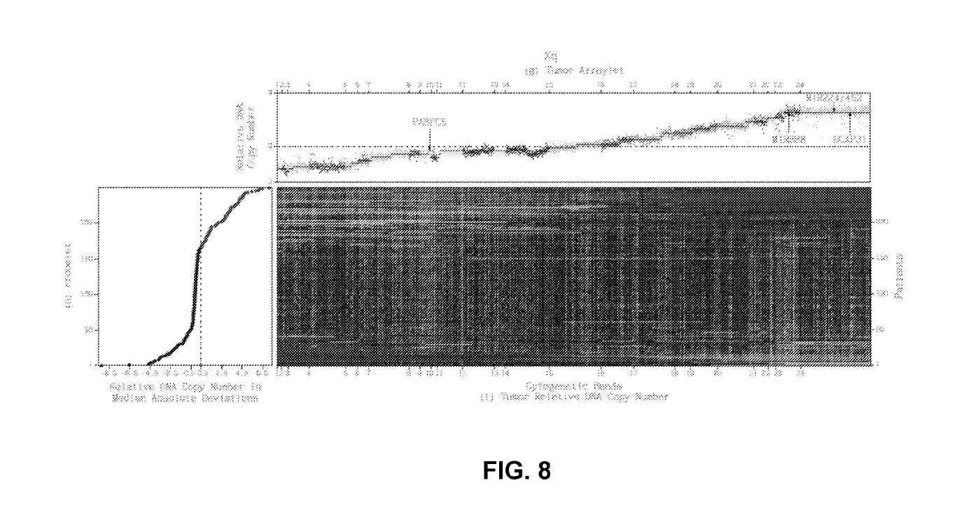

In one aspect, a method for predicting the survival of OV patients and/or predicting an OV patient's response to a therapy such as platinum-based chemotherapy is provided. In embodiments, analysis of changes in genomic features (e.g. copy number alterations, changes in protein expression, and changes in mRNA expression) provides patterns that are correlated with or indicate a prediction for survival and/or prediction to a clinical response to a particular therapy. In embodiments, the therapy is a platinum-based chemotherapy and the methods are used to predict a clinical response to the chemotherapy. As seen in FIGS. 6-8, indicators of differential expression (here CNA) were found for several genes and miRNA on the 6p, 12p, 7p, and Xq chromosomes. It will be appreciated that the patterns shown in FIGS. 6-8 show mathematical patterns extracted from measured, biological data. FIGS. 6-8 show across a region of DNA probes, a weighted sum of the pattern of CNAs for the relevant chromosome. FIG. 6 shows the increase or decrease in CNA for Tnf Mapk14, CdkN1A, Rad51AP1, Prim2, Cdkn1B, Sox5, Kras, Asun, Itpr2, miR-877, miR-200c, and miR-141 having at least one segment on the 6p or 12p chromosome. FIG. 7 shows the increase or decrease in CNA for Rpa3 and Pold2 having at least one segment on the 7p chromosome. FIG. 8 shows the increase or decrease for Pabpc5, Bcap31, miR-888, miR224, and miR-452 having at least one segment on the Xq chromosome. It will be appreciated that deletion of a chromosome that comprises at least a portion of a gene will result in differential expression of that gene. Further, only certain segments of a particular gene may be differentially expressed, e.g. Sox5. However, this may still result in differential expression of the gene. In embodiments, at least some segments comprising at least one of Tnf Mapk14, CdkN1A, Rad51AP1, Prim2, Cdkn1B, Sox5, Kras, Asun, Itpr2, Rpa3, Pold2, Pabpc5, Bcap31, miR-877, miR-200c, miR-141, miR-888, miR-224, and miR-452 are differentially expressed. In embodiments, the antisense of the microRNA sequence (designated by *) is differentially expressed.

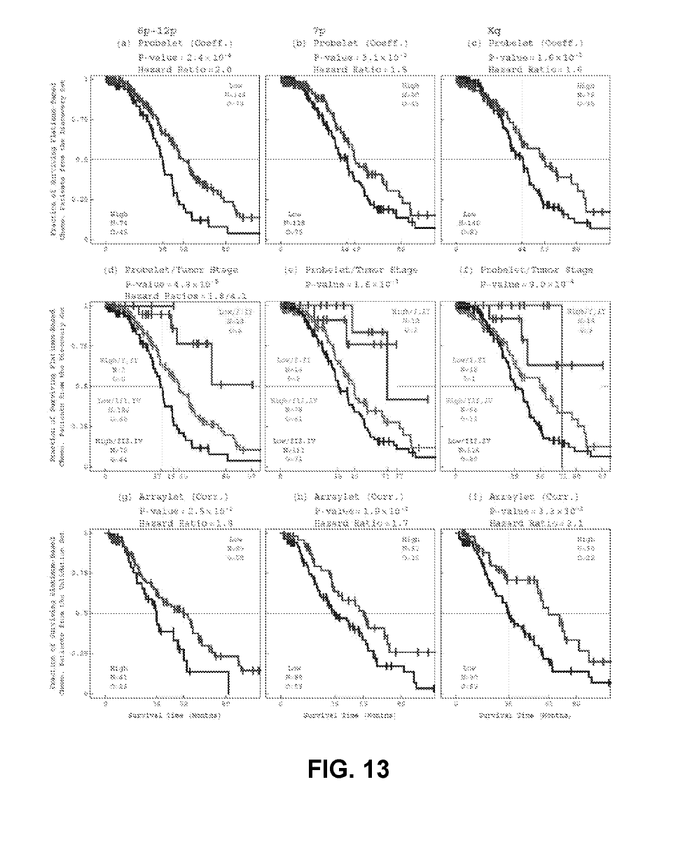

Using survival analyses of a discovery and, separately, validation set of patients, as well as only the 88% and 95% platinum-based chemotherapy patients in the discovery and validation sets, respectively (FIG. 13), it was found and validated that each of the patterns, across chromosomes 6p+12, 7p, and Xq, is correlated with an OV patient's prognosis and response to platinum-based chemotherapy, is independent of stage, and together with stage makes a better predictor than stage alone.

It was further found and validated that each of these three tensor GSVDs is independent of each of the additional standard indicators (see Tables 1 and 2, below).

TABLE 1
Cox univariate proportional hazard models of the discovery
and validation sets of patients classified by any one
of the tensor GSVDs or the standard OV indicators.

Discovery and Validation Sets

| | | |
|---|---|---|
| Predictor | Hazard Ratio | P-value |

| | | | |
|---|---|---|---|
| Tensor GSVD | 6p + 12p | 1.8 | 1.0 × 10⁻⁴ |
| | 7p | 1.7 | 1.7 × 10⁻⁴ |
| | Xq | 1.7 | 4.8 × 10⁻⁴ |

| | | |
|---|---|---|
| Tumor Stage | 4.1 | 1.8 × 10⁻³ |
| Residual Disease | 2.3 | 8.4 × 10⁻⁵ |
| Therapy Outcome | 3.8 | 8.3 × 10⁻¹⁷ |
| Neoplasm Status | 14.0 | 1.8 × 10⁻⁷ |

TABLE 2
Cox bivariate proportional hazard models of the patients
in the discovery and validation sets classified by both
tensor GSVD and the standard OV indicators.

| | | Discovery and Validation Sets | |
|---|---|---|---|
| Chromosome Arm | Predictor | Hazard Ratio | P-value |
| 6p + 12p | Tensor Stage | 1.7 | 4.4 × 10⁻⁴ |
| | Tumor Stage | 3.7 | 3.9 × 10⁻³ |
| | Tensor GSVD | 1.6 | 2.5 × 10⁻³ |
| | Residual Disease | 2.2 | 1.2 × 10⁻⁴ |
| | Tensor GSVD | 1.7 | 1.2 × 10⁻³ |
| | Therapy Outcome | 3.7 | 1.9 × 10⁻¹⁵ |
| | Tensor GSVD | 1.6 | 1.2 × 10⁻³ |
| | Neoplasm Status | 13.0 | 3.9 × 10⁻⁷ |
| 7p | Tensor Stage | 1.7 | 4.2 × 10⁻⁴ |
| | Tumor Stage | 3.9 | 2.4 × 10⁻³ |
| | Tensor GSVD | 1.6 | 1.3 × 10⁻³ |
| | Residual Disease | 2.2 | 1.1 × 10⁻⁴ |
| | Tensor GSVD | 1.5 | 1.6 × 10⁻² |
| | Therapy Outcome | 3.5 | 2.4 × 10⁻¹⁴ |
| | Tensor GSVD | 1.7 | 6.0 × 10⁻⁴ |
| | Neoplasm Status | 13.3 | 3.0 × 10⁻⁷ |
| Xq | Tensor Stage | 1.6 | 1.7 × 10⁻³ |
| | Tumor Stage | 3.8 | 3.2 × 10⁻³ |
| | Tensor GSVD | 1.9 | 1.1 × 10⁻⁴ |
| | Residual Disease | 2.2 | 9.3 × 10⁻⁵ |
| | Tensor GSVD | 1.8 | 8.5 × 10⁻⁴ |
| | Therapy Outcome | 3.8 | 1.1 × 10⁻¹⁶ |
| | Tensor GSVD | 1.7 | 6.7 × 10⁻⁴ |
| | Neoplasm Status | 14.5 | 1.3 × 10⁻⁷ |

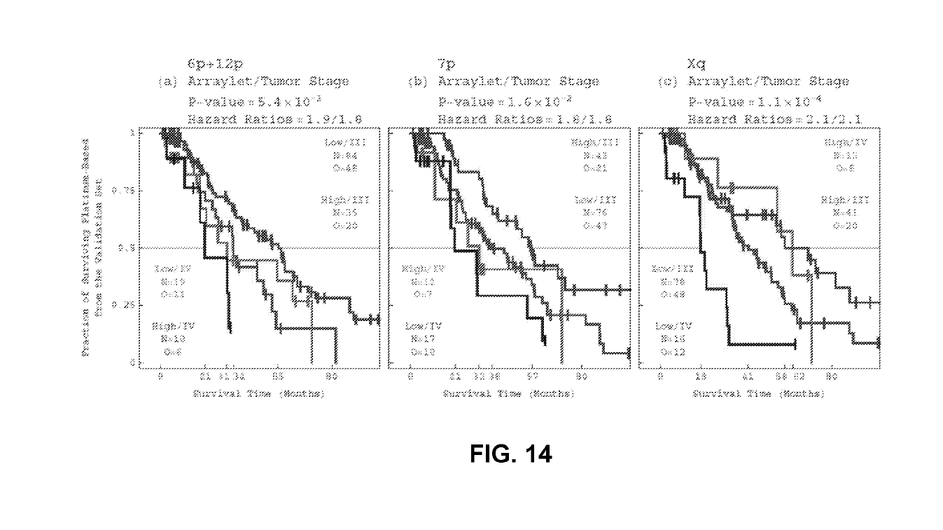

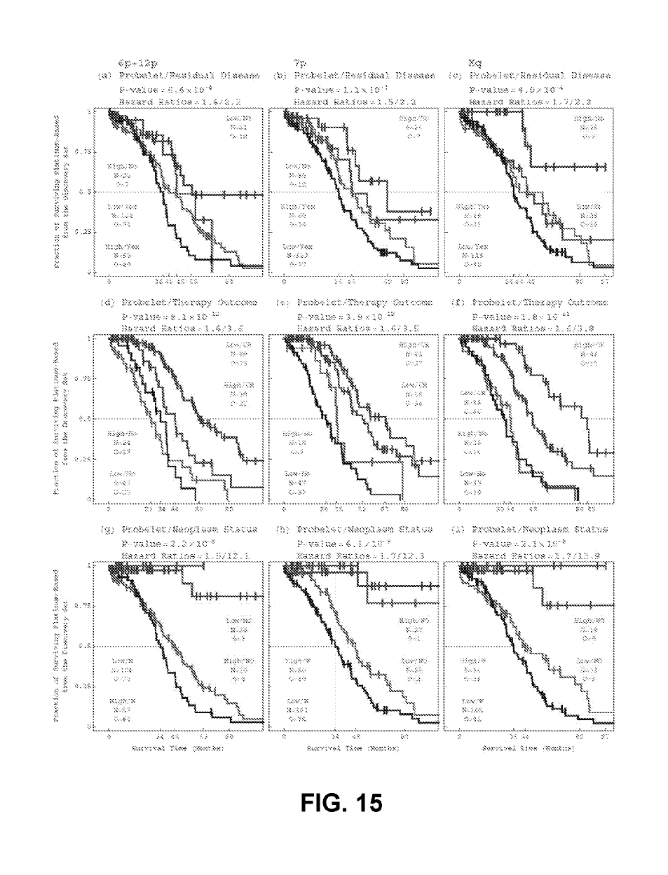

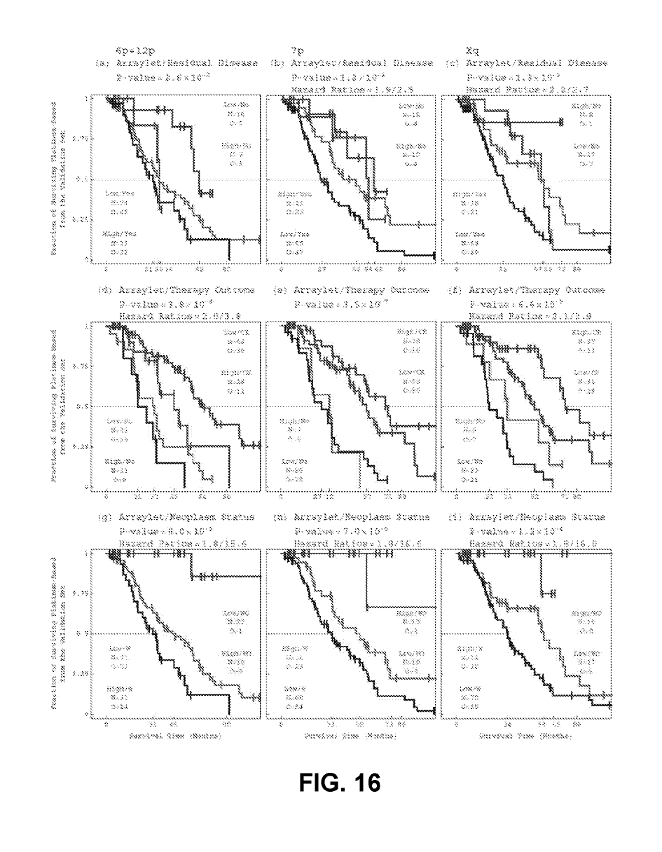

For example, survival analyses of the discovery set classified by the 6p+12p tensor GSVD into high and low x-probelet coefficients, and by pathology at diagnosis into tumor stages I-II and III-IV, give the bivariate Cox hazard ratios of 1.5 and 4.0, which are similar to the corresponding univariate ratios of 1.7 and 4.4, respectively. Similarly, survival analyses of the validation set classified by the 6p+12p tensor GSVD into high and low arraylet correlation coefficients, and by pathology at diagnosis into tumor stages III and IV, give the bivariate Cox hazard ratios of 1.9 and 1.8, which are the same as the corresponding univariate ratios (FIG. 14). This means that the 6p+12p tensor GSVD and stage are independent predictors of survival. Therefore, combined with any one of the standard indicators, each of the three tensor GSVDs makes a better predictor than the standard indicator alone (FIGS. 15 and 16). The Kaplan-Meier (KM) median survival time difference of 61 months among the discovery set of patients classified by both the 6p+12p tensor GSVD and stage, is about 85% and more than two years greater than the 33 month difference between the patients classified by stage alone. The KM median survival difference of 34 months among the validation set of patients classified by both the 6p+12p tensor GSVD and stage, is about 62% and more than one year greater than the 21 month difference between the patients classified by stage alone.

Of note, while the discovery set of patients reflects the general OV patient population, with approximately 5%, 7%, 76%, and 12% of the patients diagnosed at stages I, II, III, and IV, respectively, the validation set reflects the high-stage OV patient population, with approximately 20% and 80% of the patients diagnosed at stages III and IV, respectively. The 6p+12p, 7p, and Xq tensor GSVDs, therefore, predict survival both in the general as well as in the high-stage OV patient population. Note also that the discovery and validation sets each include mostly, i.e., >95% high-grade, i.e., grades 2 and higher tumors. Tumor grade does not correlate with survival in either the discovery or the validation set of patients.

It was also found and validated by survival analysis of only the >95% patients with high-grade tumors that these patterns are also independent of the OV tumor's grade. Three groups of significantly different prognoses were observed among the patients classified by a combination of the 6p+12p, 7p, and Xq tensor GSVD classifications, suggesting a possible implementation of the patterns in a pathology laboratory test.

Survival analyses of only the >95% patients with high-grade tumors in the discovery and, separately, validation set give qualitatively the same and quantitatively similar results to those of the analyses of 100% of the patients in each set, respectively. The 6p+12p, 7p, and Xq tensor GSVDs, therefore, predict survival in the high-grade OV patient population, and are independent of the OV tumor's grade as well as the molecular distinctions between high- and low-grade OV tumors.

By using segmentation of the 6p+12p, 7p, and Xq patterns, it was found that the amplifications and deletions identified by these patterns include most known OV-associated CNAs that map to these chromosome arms, as well as several previously unreported, yet frequent focal CNAs. Third, by using gene ontology enrichment analyses of the OV tumor mRNA expression profiles of the patients, it was found that differential mRNA expression between the patients, classified by any one of the three tensor GSVDs, is enriched in ontologies corresponding to one of three hallmarks of cancer: a cell's immortality in 6p+12p, DNA instability in 7p, and cellular immune response suppression in Xq. The differential mRNA expression of genes from these enriched ontologies that are located on any one of the chromosome arms is consistent with the CNAs across that arm. Genes that map to amplifications or deletions on any one pattern, are overexpressed or underexpressed, respectively, in the patients which tumor profiles are classified as highly similar to that pattern. The differential expression of all microRNAs and proteins that map to any one of the chromosome arms is also consistent with the CNAs across that arm.

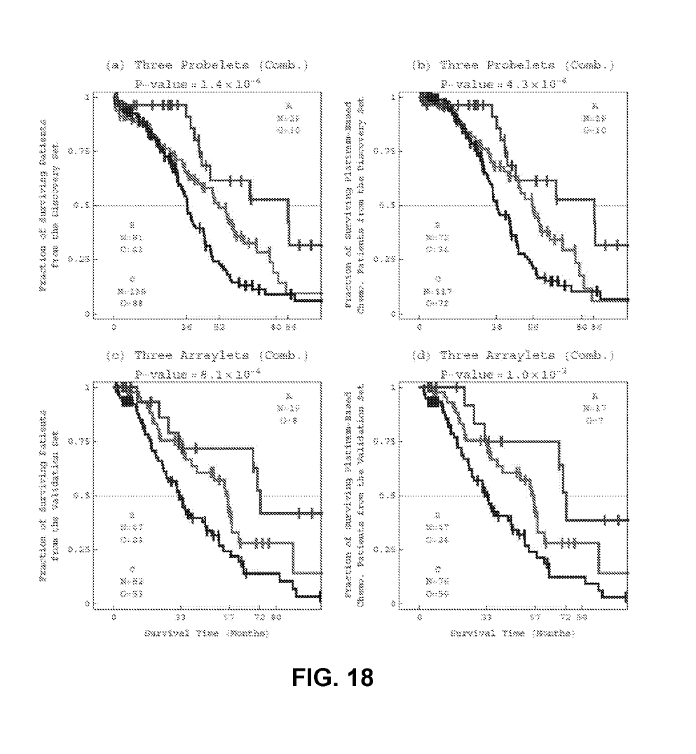

As described in Example 2, three groups of significantly different prognoses among the discovery and, separately, validation set of patients, as well as only the platinum-based chemotherapy patients, were observed and classified by a combination of the three, i.e., 6p+12p, 7p, and Xq, tensor GSVD classifications, each of which is binomial (FIG. 18). In group A, a combination of a low 6p+12p x-probelet coefficient or arraylet correlation, and high 7p and Xq x-probelet coefficients or arraylet correlations is indicative of a patient's significantly longer survival time and better response to platinum-based chemotherapy. In group B, the three combinations where just one of the three binomial classifications differs from that of group A, indicate shorter survival time and worse response to chemotherapy than those of group A. In group C, the four combinations where at least two of the three binomial classifications differ from that of group A, indicate shorter survival time and worse response to chemotherapy than those of group B as well as group A. For example, the KM median survival times of the discovery set of patients classified into groups A, B, and C are 86, 52, and 36 months, such that the median survival time of group A is more than four years greater than, and more than twice that of group C.

This suggests a possible implementation of the 6p+12p, 7p, and Xq patterns in a pathology laboratory test, where a patient's survival and response to platinum-based chemotherapy is predicted based upon the combination of the correlations of the OV tumor's DNA copy-number profile with the 6p+12p, 7p, and Xq patterns.

A. Novel Frequent Focal CNAs Indicating OV Survival

OV tumors exhibit significant CNA variation among them, much more so than, e.g., GBM brain tumors. Very few frequently occurring OV CNAs have been identified to date. In one aspect, CNAs for predicting OV survival are provided.

It was found by using segmentation, that the three tensor GSVD arraylets include most known OV-associated CNAs that map to the corresponding chromosome arms, and several previously unreported yet frequent CNAs in >23% of the patients. For example, the 6p+12p arraylet includes two segments corresponding to the only known OV focal CNAs that map to 6p+12p, 7p, or Xq (see Example 3). One, a deletion (6p11.2), overlaps the 3′ end unique to isoform a of the DNA primase polypeptide 2-encoding Prim2. The other, an amplification (12p12.1-p11.23), contains several genes, including the Kirsten rat sarcoma viral oncogene homolog Kras, one of three human Ras genes, and the 5′ ends of isoforms b and d of the SRY (sex determining region Y)-box 5-encoding Sox5, and is significantly (log-rank test P-value<0.05, and KM median survival time difference≥12 months) correlated with OV survival.

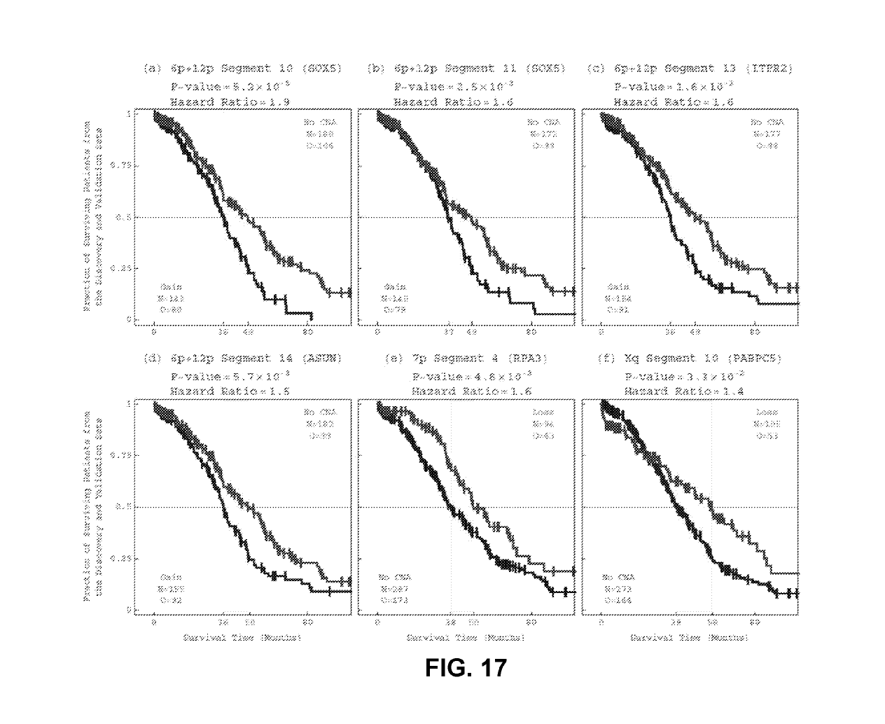

It was also found that the three arraylet patterns include novel frequent focal CNAs (segments<125 probes). Among these, four amplifications and two deletions are significantly correlated with OV survival (FIG. 17). The amplifications flank the segment that contains Kras. Two consecutive segments (12p12.1) contain the 5′ ends of isoforms a and e of Sox5, and exons 5 and 6, the first exons that are common to isoforms a, b, d, and e of Sox5. Two other consecutive segments (12p11.23) contain the inositol 1,4,5-trisphosphate receptor type 2-encoding Itpr2, and the asunder spermatogenesis regulator-encoding Asun. Asun was discovered in a screen of expressed sequence tags on 12p11-p12, which DNA amplification correlated with mRNA overexpression in four human testicular seminomas and one ovarian papillary serous adenocarcinoma cell line, exemplifying human germ cell tumors. Asun and its homologs are essential for nuclear division after DNA replication in the HeLa human cervical cancer cell line, the frog, and the fly. One deletion (7p22.1-p21.3) contains the replication protein A3-encoding Rpa3. The other (Xq21.31) contains the cytoplasmic poly(A)-binding protein 5-encoding Pabpc5, and the sequence tag site DXS241 adjacent to translocation breakpoints observed in premature ovarian failure.

B. Differential Expression Patterns

In embodiments, the present methods provide patterns of differential expression, which may be used to predict or determine an outcome for the patient. In embodiments, the outcome is at least one of a predicted length of survival or a clinical response to therapy. In embodiments, the therapy is administration of an alkylating agent. In embodiments, administration of the alkylating agent comprises a chemotherapy. In embodiments, the chemotherapy is a platinum-based chemotherapy. Differential expression is with reference to genomic features, including, but not limited to genes, proteins encoded by the genes, and mRNA. In embodiments, differential expression is measured by at least one of gene expression, mRNA expression, protein expression, etc. In embodiments, differential expression refers to CNA for a genomic feature.

In embodiments, the differential expression comprises DNA copy-number loss or gain, mRNA overexpression or underexpression, microRNA overexpression or underexpression, or protein overexpression or underexpression for a genomic feature. In embodiments, differential expression refers to a genomic feature of at least one of the 6p+12p, 7p or Xq chromosomes.

In embodiments, differential expression of a genomic feature for 6p+12p, includes, but is not limited to differential expression of at least one of Tnf, Mapk14, Cdkn1A, Rad51AP1, Sox5, Cdkn1B, Kras, Asun, miR-877, miR-200c, and miR-141. In embodiments, differential expression of a genomic feature for 6p+12p includes one or more of:
copy-number loss, or mRNA or protein underexpression of Cdkn1A is correlated with a patient's shorter survival time, and resistance to platinum-based chemotherapy;
copy-number loss, or mRNA or protein underexpression of Mapk14 on 6p is correlated with a patient's shorter survival time, and resistance to platinum-based chemotherapy;
copy-number gain, or mRNA or protein overexpression of Kras on 12p is correlated with a patient's shorter survival time, and resistance to platinum-based chemotherapy;
copy-number gain, or mRNA or protein overexpression of Rad51AP1 on 12p is correlated with a patient's shorter survival time, and resistance to platinum-based chemotherapy;
copy-number loss, or mRNA or protein underexpression of Tnf on 6p is correlated with a patient's shorter survival time, and resistance to platinum-based chemotherapy;
copy-number gain, or mRNA or protein overexpression of Itpr2 on 12p is correlated with a patient's shorter survival time, and resistance to platinum-based chemotherapy;
copy-number loss, or mircoRNA underexpression of miR-877* on 6p is correlated with a patient's shorter survival time, and resistance to platinum-based chemotherapy;
copy-number gain, or microRNA overexpression, of miR-200c, miR-200c*, miR-141, or miR-141* on 12p is correlated with a patient's shorter survival time, and resistance to platinum-based chemotherapy.

In embodiments, differential expression of a genomic feature for 7p, includes, but is not limited to differential expression of at least one of Rpa3 and Pold2. In embodiments, differential expression of a genomic feature for 7p includes one or more of:
copy-number gain, or mRNA overexpression of Pold2 on 7p is correlated with a longer survival time, and sensitivity to platinum-based chemotherapy;
co-occurring copy-number loss, or mRNA underexpression of Rpa3 on 7p is correlated with a longer survival time, and sensitivity to platinum-based chemotherapy.

In embodiments, differential expression of a genomic feature for Xq, includes, but is not limited to differential expression of at least one of Pabpc5, Bcap31, miR-888, miR-224, and miR-452. In embodiments, differential expression of a genomic feature for Xq includes one or more of:
copy-number loss of Pabpc5 is correlated with a longer survival time and/or sensitivity to platinum-based chemotherapy;
gain, or mRNA overexpression of Bcap31 is correlated with a longer survival time and/or sensitivity to platinum-based chemotherapy;
gain, or microRNA overexpression of miR-888 or miR-888*, and miR-452 or miR-452 is correlated with a longer survival time and/or sensitivity to platinum-based chemotherapy.

In embodiments, co-occurring patterns of differential expression are described herein. In embodiments, a co-occurring pattern includes differential expression of one or more genomic features identified above for 6p+12p and 7p. In embodiments, a co-occurring pattern includes differential expression of one or more genomic features identified above for 6p+12p and Xq. In embodiments, a co-occurring pattern includes differential expression of one or more genomic features identified above for 7p and Xq. In embodiments, a co-occurring pattern of differential expression includes one or more of a)-f):
a) co-occurring copy-number loss of Pabpc5, and gain, or mRNA overexpression of Bcap31; or
b) co-occurring copy number loss of Pabpc5, and gain, or mRNA overexpression of Bcap31, and gain, or microRNA overexpression of miR-888, and miR-452; or
c) co-occurring copy number loss of Pabpc5, and gain, or mRNA overexpression of Bcap31, and gain, or microRNA overexpression of miR-888, miR-452, and miR-224; or
d) co-occurring copy-number loss of Pabpc5 and sequence tag site (STS) DXS214, and gain, or mRNA overexpression of Bcap31; or
e) co-occurring copy number loss of Pabpc5, and gain, or mRNA overexpression of Bcap31 and Gabre; or
f) co-occurring copy-number loss from cytogenetic bands 1-14, and gain in cytogenetic bands 16-24;
with at least one of longer survival time and sensitivity to platinum-based chemotherapy.

In embodiments, a co-occurring pattern comprises the differential expression of (c) and further correlating copy-number loss of sequence tag site DXS214 and gain or mRNA overexpression of Bcap31 and Gabre with at least one of longer survival time and sensitivity of platinum-based chemotherapy.

In embodiments, a co-occurring pattern of differential expression includes one or more of a1)-d1):
a1) co-occurring copy-number loss, or mRNA underexpression of Rpa3, and copy-number gain, or mRNA overexpression of Pold2; or
b1) co-occurring copy-number loss, or mRNA underexpression of Rpa3 on 7p and Lig4 on 13q, and copy-number gain, or mRNA overexpression of Pold2; or
c1) co-occurring copy-number loss, or mRNA underexpression of Lig4 on chromosome 13q, and copy-number gain, or mRNA overexpression of Pold2; or
d1) co-occurring copy-number loss from cytogenetic bands 1-7, and gain in cytogenetic bands 11-17;
with at least one of a longer survival time and sensitivity to platinum-based chemotherapy.

In embodiments, a co-occurring pattern of differential expression includes one or more of a2)-g2):
a2) co-occurring copy-number loss on chromosome 6p and gain on chromosome 12p; or
b2) co-occurring copy-number loss, or mRNA or protein under-expression of Cdkn1A and Mapk14 on chromosome 6p, and copy-number gain, or mRNA or protein overexpression of Kras on chromosome 12p; or
c2) co-occurring copy-number loss, or mRNA or protein under-expression of Cdkn1A and Mapk14 on 6p, and copy-number gain, or mRNA or protein overexpression of Kras and Rad51AP1 on 12p; or
d2) co-occurring copy-number loss, or mRNA or protein under-expression of Cdkn1A, Mapk14, and Tnf on chromosome 6p, and copy-number gain, or mRNA or protein overexpression of Kras, Rad51AP1, and Itpr2 on chromosome 12p; or
e2) co-occurring copy-number loss, or microRNA under-expression of miR-877* on chromosome 6p, and copy-number gain, or microRNA overexpression, of miR-200c, miR-200c*, miR-141, or miR-141* on chromosome 12p;
(f2) co-occurring copy-number loss, or mRNA or protein under-expression of Cdkn1A and Mapk14 on chromosome 6p, and copy-number gain, or mRNA or protein overexpression of Rad51AP1 on chromosome 12p;
(g2) co-occurring copy-number loss, or mRNA or protein under-expression of Tnf on chromosome 6p, and copy-number gain, or mRNA or protein overexpression of Itpr2 on chromosome 12p;
with at least one of shorter survival time and resistance to platinum-based chemotherapy.

In embodiments, a co-occurring pattern of differential expression includes one or more of a2)-g2) and additionally at least one of h2)-m2):
(h2) a gain in copy numbers or mRNA or protein overexpression of Sox5; or
(i2) a gain in copy numbers or mRNA or protein overexpression of Asun; or
(j2) a gain in copy numbers or mRNA or protein overexpression of Abcf1; or
(k2) a gain in copy numbers or mRNA or protein overexpression of Cdkn1B; or
(l2) an mRNA or protein under-expression or loss in copy numbers of Bap1; or
(m2) a reduced abundance of Brca1-associated c,
with reduced abundance of the Brca1-associated genome surveillance protein complex (BASC);

In embodiments, a pattern of differential expression includes one or more of:
(1) an increase in copy number of the segment overlapping with the Prim2 gene with at least one of reduced length of patient survival and resistance to platinum-based chemotherapy;
(2) an increase in copy number of the Kras gene with at least one of reduced length of patient survival and resistance to platinum-based chemotherapy;
(3) an increase in copy number of the Sox5 gene with at least one of reduced length of patient survival and resistance to platinum-based chemotherapy;
(4) an increase in copy number of the Itpr2 gene with at least one of reduced length of patient survival and resistance to platinum-based chemotherapy;
(5) an increase in copy number of the Asun gene with at least one of reduced length of patient survival and resistance to platinum-based chemotherapy;
(6) a decrease in copy number of the Rpa3 gene with at least one of increased length of patient survival and sensitivity to platinum-based chemotherapy;
(7) a decrease in copy number of the Pabpc5 gene with at least one of increased length of patient survival and sensitivity to platinum-based chemotherapy;
(8) a decrease in copy number of the DXS214 sequence tag site with at least one of increased length of patient survival and sensitivity to platinum-based chemotherapy;
(9) a decrease in copy number of the Cdkn1A gene with at least one of reduced length of patient survival and resistance to platinum-based chemotherapy;
(10) a decrease in copy number of the Mapk14 gene with at least one of reduced length of patient survival and resistance to platinum-based chemotherapy;
(11) a decrease in copy number of the Tnf gene with at least one of reduced length of patient survival and resistance to platinum-based chemotherapy;
(12) a decrease in copy number of the miR-877 or miR-877* microRNA with at least one of reduced length of patient survival and resistance to platinum-based chemotherapy;
(13) a decrease in copy number of the Abcf1 gene with at least one of reduced length of patient survival and resistance to platinum-based chemotherapy;
(14) an increase in copy number of the Rad51AP1 gene with at least one of reduced length of patient survival and resistance to platinum-based chemotherapy;
(15) an increase in copy number of the miR-200c or miR-200c* with at least one of reduced length of patient survival and resistance to platinum-based chemotherapy;
(16) an increase in copy number of the miR-141c or miR-141c* with at least one of reduced length of patient survival and resistance to platinum-based chemotherapy;
(17) an increase in copy number of the Cdkn1B gene with at least one of reduced length of patient survival and resistance to platinum-based chemotherapy;
(18) an increase in copy number of the Pold2 gene with at least one of increased length of patient survival and sensitivity to platinum-based chemotherapy;
(19) an increase in copy number of the Bcap31 gene with at least one of increased length of patient survival and sensitivity to platinum-based chemotherapy;
(20) an increase in copy number of the miR-888 with at least one of increased length of patient survival and sensitivity to platinum-based chemotherapy;
(21) an increase in copy number of the miR-224 with at least one of increased length of patient survival and sensitivity to platinum-based chemotherapy;
(22) an increase in copy number of the miR-452 with at least one of increased length of patient survival and sensitivity to platinum-based chemotherapy;
(23) an increase in copy number of the Gabre gene with at least one of increased length of patient survival and sensitivity to platinum-based chemotherapy;
(24) a decrease in copy number of the Lig4 gene with at least one of increased length of patient survival and sensitivity to platinum-based chemotherapy;
(25) mRNA underexpression, mRNA or protein underexpression (or loss in copy numbers) of Bap1 with at least one of decreased length of patient survival and resistance to platinum-based chemotherapy;
(26) reduced abundance of BRCA1-associated BAP1, e.g., reduced abundance of the BRCA1-associated genome surveillance protein complex (BASC) with at least one of decreased length of patient survival and resistance to platinum-based chemotherapy.

It will be appreciated that differences in copy number as described above will also apply to differential expression, which includes CNA, mRNA and miRNA expression, and protein expression.

In embodiments, a co-occurring pattern of any one of the genomic features of (1)-(26) is contemplated. As an illustration, and without limitation, the genomic feature of (1) may be combined with any one of the genomic features of (2)-(26). As a further non-limiting illustration, the genomic feature of (1) may be combined with multiple or all of the genomic features of (2)-(26). Any combination or sub-combination of the genomic features of (1)-(24) are contemplated herein. In specific, but not limiting embodiments, a co-occurring pattern is selected from (i) correlating at least two of (2), (4), (6), (9)-(12), (14)-(16), (18), and (24); (ii) correlating at least two of (2), (4), (7), (9)-(12), (14)-(16), (19)-(23); or (iii) correlating at least two of (6)-(7), and (18)-(24). In embodiments, co-occurring patterns of differential expression may include differential expression of genomic features from additional chromosomes such as Lig4 on chromosome 13q.

C. OV Pathogenesis

It was found, by using gene ontology enrichment analyses of the OV tumor mRNA expression profiles of the patients, that differential mRNA expression between the patients, classified by any one of the three tensor GSVDs, is enriched in ontologies corresponding to one of three hallmarks of cancer: cell immortality in 6p+12p, DNA instability in 7p, and cellular immune response suppression in Xq.

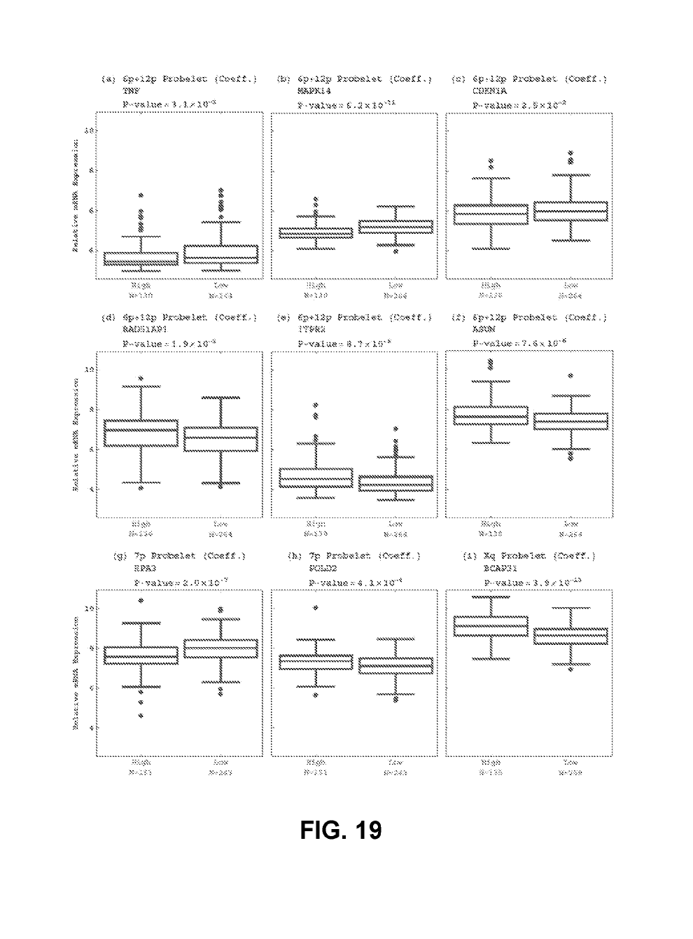

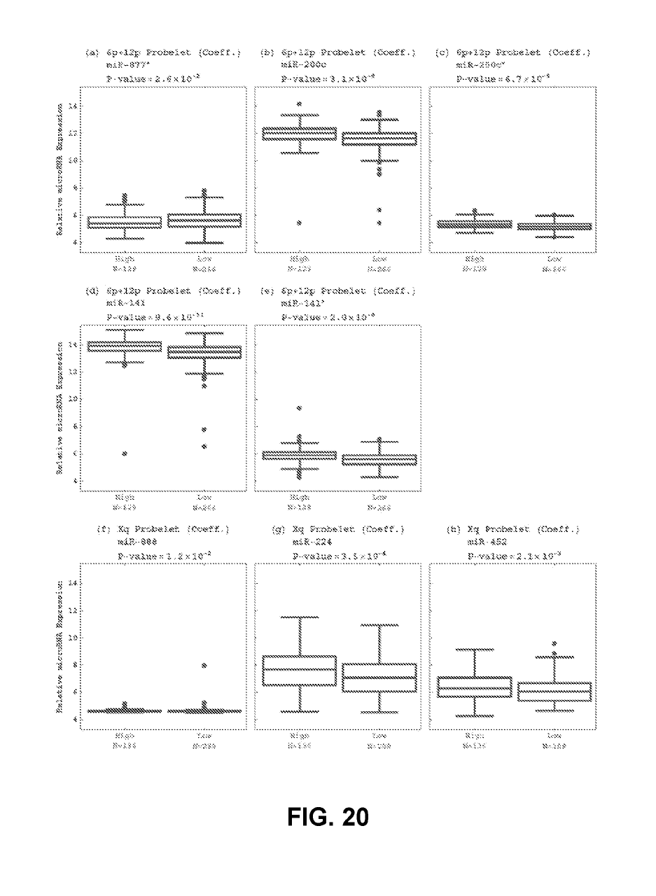

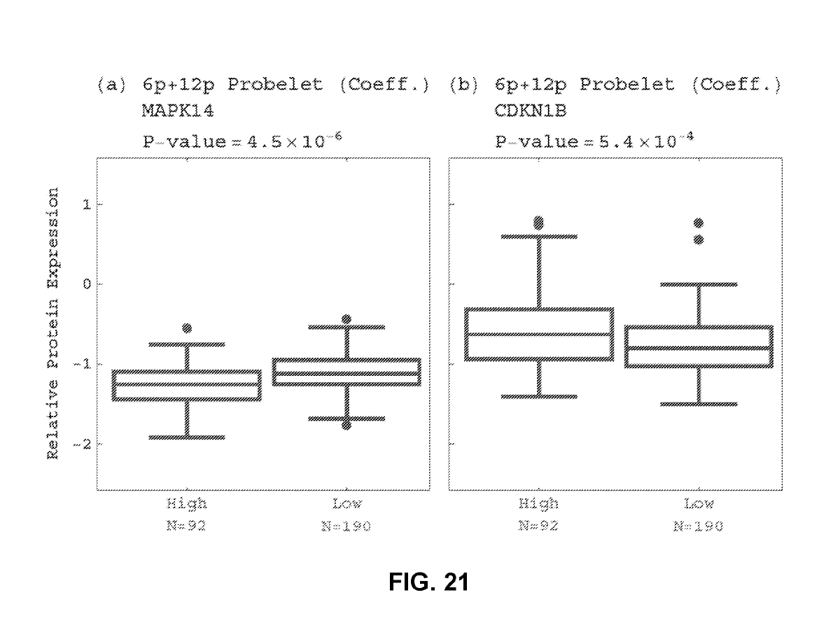

The differential mRNA expression of genes from these enriched ontologies that are located on any one of the chromosome arms is consistent with the CNAs across that arm (FIG. 19). Genes that map to amplifications or deletions on any one arraylet pattern, are overexpressed or underexpressed, respectively, in the patients which tumor profiles are classified, by the corresponding tensor GSVD, as highly similar to that pattern, i.e., patients of high x-probelet coefficients or arraylet correlations. The differential expression of all microRNAs and proteins that map to any one of the chromosome arms is also consistent with the CNAs across that arm (FIGS. 20 and 21). A coherent picture emerges for each pattern, suggesting roles for the CNAs in OV pathogenesis in addition to personalized diagnosis, prognosis, and treatment.

1. 6p+12p

In some embodiments, a cell's transformation and immortality are correlated with a patient's shorter survival. The genes, which are significantly (Mann-Whitney-Wilcoxon P-values<0.05) differentially expressed between the 6p+12p tensor GSVD classes, i.e., in the patient group of high 6p+12p x-probelet coefficient or arraylet correlation, relative to the patient group of low coefficient or correlation, are enriched (hypergeometric P-values<10⁻³) in the ontologies of cellular response to ionizing radiation (GO:0071479), and major histocompatibility (MHC) protein complex (GO:0042611). Most of the GO:0071479 genes are underexpressed, including the p21 cyclin-dependent kinase inhibitor-encoding Cdkn1A, and the p38 mitogen-activated protein kinase-encoding Mapk14, which map to a deletion>45 Mbp on the telomeric part of 6p (6p25.3-p21.1). Also underexpressed is p38, the protein encoded by Mapk14. All GO:0042611 genes, including the tumor necrosis factor-encoding TNF, are underexpressed, and map to the same deletion. The one microRNA that is significantly differentially expressed between the 6p+12p tensor GSVD classes, and maps to the same deletion, is the splicing-dependent microRNA miR-877*, which is encoded by the 13th intron of the ATP-binding cassette subfamily F member 1-encoding gene Abcf1. Both miR-877* and Abcf1 are consistently underexpressed.

One of only two GO:0071479 overexpressed genes is the Rad51-associated protein 1-encoding Rad51AP1, which maps to an amplification>9 Mbp on the telomeric part of 12p (12p13.33-p13.31) that is significantly correlated with OV survival. All four microRNAs that are differentially expressed between the 6p+12p tensor GSVD classes, and map to the same amplification, miR-200c, miR-200c*, miR-141, and miR-141*, are consistently overexpressed. The second protein that is significantly differentially expressed between the 6p+12p tensor GSVD classes is p27. Consistently, the cyclin-dependent kinase inhibitor Cdkn1B, which encodes p27, maps to a 4.5 Mbp amplification (12p13.2-p12.3) that is significantly correlated with OV survival, and its mRNA is overexpressed. The mRNA encoded by Kras is also overexpressed.

Note that while the 6p+12p pattern of CNAs is correlated with survival in the discovery and, separately, validation sets, neither the 6p nor the 12p pattern alone are correlated with survival. Indeed, experiments studying the conditions for the transformation of human normal to tumor cells indicate that cells, where both p21 and p38 are inactive, are susceptible to Ras-mediated transformation. However, the activation of Ras alone induces tumor-suppressing cellular senescence via the activities of either p21 or p38. The 6p+12p pattern, therefore, which includes the loss of the p21-encoding Cdkn1A and the p38-encoding Mapk14 on 6p, and the gain of Kras on 12p, encodes for cellular conditions that combined but not separately can lead to transformation.

In addition, p21 and p38 are necessary for p53-mediated cell cycle arrest and apoptosis, respectively, in response to DNA damage. Overexpression of the p21-encoding Cdkn1A is correlated with a low malignant potential of an ovarian tumor. Rad51AP1 overexpression disrupts cell cycle arrest and apoptosis, can lead to cellular resistance to DNA-damaging cancer therapies, such as platinum-based chemotherapy, and may increase DNA instability. Tnf-induced apoptosis is correlated with downregulation of Itpr2. Overexpression of miR-200c, and miR-141, both of which putatively target the BrcaA1 associated protein-1 oncosuppressor-encoding Bap1, is correlated with OV tumor growth, dedifferentiation, and invasiveness. Overexpression of the Cdkn1B-encoded p27, which can promote cellular migration and even proliferation, is correlated with a poor OV patient's prognosis.

Taken together, previously unrecognized co-occurring deletion of Cdkn1A and Mapk14 on 6p and amplification of Kras on 12p, which encode for human cell transformation, together with deletion of Tnf on 6p, and amplification of Rad51AP1 and ITPR2 on 12p, are correlated with a suppression of cell cycle arrest, senescence, and apoptosis, i.e., a tumor cell's immortality, and a patient's shorter survival time. Note that there already exist drugs that interact with Cdkn1A, Mapk14, and Rad51AP1, even though these genes were not recognized previously as targets for OV drug therapy.

2. 7p

A cell's DNA stability is correlated with a longer survival. The genes that are significantly differentially expressed between the 7p tensor GSVD classes are enriched (hypergeometric P-value)<10⁻¹° in the ontology of DNA strand elongation involved in DNA replication (GO:0006271). Most of these genes are overexpressed, including the DNA polymerase delta subunit 2-encoding Pold2 that is essential for DNA replication and repair, which maps to an amplification>17 Mbp on the centromeric part of 7p (7p14.1-p11.2). Only two genes are underexpressed: Rpa3 on 7p and the DNA ligase IV-encoding Lig4 on 13q. The interaction of p53 with the Rpa3-encoded protein mediates suppression of homologous recombination (HR), the preferred cellular mechanism for DNA double-strand break (DSB) repair during replication. Lig4 is essential for DSB repair via the more error-prone nonhomologous end joining pathway. HR defects are thought to facilitate the significant CNA heterogeneity among OV tumors.

Taken together, previously unrecognized co-occurring deletion and underexpression of Rpa3, and amplification and overexpression of Pold2 on 7p are correlated with DNA DSB repair via HR during replication, i.e., DNA stability, and a longer survival time.

3. Xq

Cellular immune response is correlated with a longer survival. The genes that are differentially expressed between the Xq tensor GSVD classes are enriched (hypergeometric P-value<10⁻⁶) in the ontology of antigen processing and presentation of peptide antigen (GO:0048002). Most of these genes are overexpressed, including the B-cell receptor-associated protein 31-encoding Bcap31, which maps to an amplification>11 Mbp on the telomeric part of Xq (Xq27.3-q28). All three microRNAs that are differentially expressed between the Xq tensor GSVD classes, and map to the same amplification, miR-888, miR-224, and miR-452, together with the gamma-aminobutyric acid (GABA) A receptor epsilon-encoding Gabre, which hosts mir-224 and mir-452 in its introns, are consistently overexpressed. Underexpression of miR-224 was implicated in OV pathogenesis. Pabpc5, which maps to a focal deletion on Xq, is suppressed upon viral infection.

Taken together, previously unrecognized co-occurring deletion of Pabpc5, and amplification and overexpression of Bcap31 on Xq are correlated with a cellular immune response, and a longer survival time.

In embodiments, methods of predicting survival time and/or predicting a clinical response to a treatment regimen such as chemotherapy involve determining at least one indicator of differential expression selected from one or more of: gain in copy numbers of a segment overlapping the Prim2 gene is correlated with poor survival and resistance to platinum-based chemotherapy; gain in copy numbers of Kras is correlated with poor survival and resistance to platinum-based chemotherapy; gain in copy numbers of Sox5 is correlated with poor survival and resistance to platinum-based chemotherapy; gain in copy numbers, or mRNA or protein overexpression of Itpr2 is correlated with poor survival and resistance to platinum-based chemotherapy; gain in copy numbers, or mRNA or protein overexpression of Asun is correlated with poor survival and resistance to platinum-based chemotherapy; loss in copy numbers, or mRNA or protein under-expression of Rpa3 is correlated with a longer survival time, and sensitivity to platinum-based chemotherapy; loss in copy numbers, or mRNA or protein under-expression of Rpa3 is correlated with a longer survival time, and sensitivity to platinum-based chemotherapy; loss in copy numbers, or mRNA or protein under-expression of Pabpc5 is correlated with a longer survival time, and sensitivity to platinum-based chemotherapy; loss in copy numbers of DXS214 is correlated with a longer survival time, and sensitivity to platinum-based chemotherapy; loss in copy numbers, or mRNA or protein under-expression of Cdkn1A is correlated with poor survival and resistance to platinum-based chemotherapy; loss in copy numbers, or mRNA or protein under-expression of Mapk14 is correlated with poor survival and resistance to platinum-based chemotherapy; loss in copy numbers, or mRNA or protein under-expression of Tnf is correlated with poor survival and resistance to platinum-based chemotherapy; loss in copy numbers, or microRNA under-expression of miR-877* or miR-877 is correlated with poor survival and resistance to platinum-based chemotherapy; loss in copy numbers, or mRNA or protein under-expression of Abcf1 is correlated with poor survival and resistance to platinum-based chemotherapy; gain in copy numbers, or mRNA or protein overexpression of Rad51AP1 is correlated with poor survival and resistance to platinum-based chemotherapy; gain in copy numbers, or microRNA overexpression of miR-200c or miR-200c* is correlated with poor survival and resistance to platinum-based chemotherapy; gain in copy numbers, or microRNA overexpression of miR-141 or miR-141* is correlated with poor survival and resistance to platinum-based chemotherapy; gain in copy numbers, or mRNA or protein overexpression of Cdkn1B is correlated with poor survival and resistance to platinum-based chemotherapy; gain in copy numbers, or mRNA or protein overexpression of Pold2 is correlated with a longer survival time, and sensitivity to platinum-based chemotherapy; gain in copy numbers, or mRNA or protein overexpression of Bcap31 is correlated with a longer survival time, and sensitivity to platinum-based chemotherapy; gain in copy numbers, or microRNA overexpression of miR-888 is correlated with a longer survival time, and sensitivity to platinum-based chemotherapy; gain in copy numbers, or microRNA overexpression of miR-224 is correlated with a longer survival time, and sensitivity to platinum-based chemotherapy; gain in copy numbers, or microRNA overexpression of miR-452 is correlated with a longer survival time, and sensitivity to platinum-based chemotherapy; gain in copy numbers, or mRNA or protein overexpression of GABRE is correlated with a longer survival time, and sensitivity to platinum-based chemotherapy; loss in copy numbers, or mRNA or protein under-expression of Lig4 is correlated with a longer survival time, and sensitivity to platinum-based chemotherapy; or any combination of the above.

It will be appreciated that the CNA signatures and expression profiles described above may be used to predict response to platinum-based chemotherapy agents for other cancers where platinum-based chemotherapy is used. For example, the methods described herein may be used to predict response to platinum-based chemotherapy agents for advanced, metastatic forms of colon cancer, small cell and non-small cell lung cancer, breast cancer, adrenocortical cancer, anal cancer, endometrial cancer, non-Hodgkin lymphoma, ovarian cancer, testicular cancer, melanoma and head and neck cancers, among others.

### IV. Reducing the Proliferation or Viability of Cancer Cells

Also described herein are methods for reducing the proliferation or viability of a cancer and methods of treating cancer by modulating the expression level of one or more genes, or modulating the activity of one or more proteins encoded by suitable genes, or modulating the expression level of one or more mRNA encoded by suitable genes. Embodiments of suitable genes include, but are limited to Ckdn1A, Mapk14, Rad51AP1, Kras, Rpa3, Pold2, Pabpc5, Tnf, Prim2, Sox5, Asun, Itpr2, and Bcap31. Embodiments of mRNA include, but are not limited to miR-877, miR-200c, miR-141, miR-888, miR-224, miR-452, or antisense sequences thereof. In some embodiments, it was found that in 6p+12p, deletion of the p21-encoding Cdkn1A and p38-encoding Mapk14 and amplification of Rad51AP1 and Kras encode for human cell transformation and are correlated with a cell's immortality and a patient's shorter survival time. For 7p, Rpa3 deletion and Pold2 amplification are correlated with DNA stability, and a longer survival time. For Xq, Pabpc5 deletion and Bcap31 amplification are correlated with a cellular immune response and a longer survival time. In non-limiting embodiments, the cancer is selected from ovarian serous cystadenocarcinoma, small cell lung cancers, non-small cell lung cancers, testicular cancer, stomach cancers, bladder cancers, colon cancers, breast cancer, adrenocortical cancer, anal cancer, endometrial cancer, non-Hodgkin lymphoma, melanoma, and head and neck cancers.

For example, inhibitors can be used to reduce the expression of one or more genes described herein, or reduce the activity of one or more gene products (e.g., proteins encoded by the genes) described herein. Exemplary inhibitors include, e.g., RNA effector molecules that target a gene, antibodies that bind to a gene product, a dominant negative mutant of the gene product, etc. Inhibition can be achieved at the mRNA level, e.g., by reducing the mRNA level of a target gene using RNA interference. Inhibition can be also achieved at the protein level, e.g., by using an inhibitor or an antagonist that reduces the activity of a protein.

As another example, activators can be used to activate the expression of one or more genes described herein, or increase the activity of one or more gene products (e.g., proteins encoded by the genes) described herein. Exemplary activators include, e.g., RNA effector molecules that target a gene, activators that enhance the interaction between RNA polymerase and a promoter, activators that activate or deactivate receptors, etc. Activation can be achieved at the mRNA level, e.g., by increasing the mRNA level of a target gene. Inhibition can be also achieved at the protein level, e.g., by using an agent that increases the activity of a protein.

In one aspect, the disclosure provides a method for reducing the proliferation or viability of an OV cancer cell comprising: contacting the cell with an inhibitor that (i) downregulates the expression of a gene selected from the group consisting of Rad51AP1, Kras, Rpa3, and/or Pabpc5, and a combination thereof; or (ii) down-regulates the activity of a protein selected from RAD51AP1, KRAS, RPA3, or PABPC5, and a combination thereof, and/or contacting the cell with an activator that up-regulates the expression level of a gene selected from the group consisting of Cdkn1A, Mapk14, Pold2, and Bcap31, or a combination thereof.

In another aspect, the disclosure provides a method of treating OV comprising: administering an inhibitor that (i) downregulates the expression of a gene selected from the group consisting of Rad51AP1, Kras, Rpa3, or Pabpc5, and a combination thereof; or (ii) down-regulates the activity of a protein selected from RAD51AP1, KRAS, RPA3, or PABPC5, and a combination thereof; and/or administering an activator that up-regulates the expression level of a gene selected from the group consisting of Cdkn1A, Mapk14, Pold2, and Bcap31, or a combination thereof.

Exemplary inhibitors that reduce the expression of one or more genes described herein, or reduce the activity of one or more gene products described herein include, e.g., RNA effector molecules that target a gene, antibodies that bind to a gene product, a dominant negative mutant of the gene product, etc.

For the treatment of OV, a therapeutically effective amount of an inhibitor is administered, which is an amount that, upon single or multiple dose administration to a subject (such as a human patient), prevents, cures, delays, reduces the severity of, and/or ameliorating at least one symptom of OV, prolongs the survival of the subject beyond that expected in the absence of treatment, or increases the responsiveness or reduces the resistance of a subject to another therapeutic treatment (e.g., increasing the sensitivity or reducing the resistance to a chemotherapeutic drug). In another embodiment, a therapeutically effective amount of an activator is administered, which is an amount that, upon single or multiple dose administration to a subject (such as a human patient), prevents, cures, delays, reduces the severity of, and/or ameliorating at least one symptom of OV, prolongs the survival of the subject beyond that expected in the absence of treatment, or increases the responsiveness or reduces the resistance of a subject to another therapeutic treatment (e.g., increasing the sensitivity or reducing the resistance to a chemotherapeutic drug).

The term “treatment” or “treating” refers to a therapeutic, preventative or prophylactic measures.

Also described herein are the use of the inhibitors and/or activators described herein for reducing the proliferation or viability of an OV cancer cell, or for treating OV; and the use of the inhibitors described herein in the manufacture of a medicament for reducing the proliferation or viability of an OV cancer cell, or for treating OV.

1. RNA Effector Molecules

In certain embodiments, the inhibitor is an RNA effector molecule, such as an antisense RNA, or a double-stranded RNA that mediates RNA interference. In certain other embodiments, the activator is an RNA effector molecule that mediates RNA regulation. RNA effector molecules that are suitable for the subject technology have been disclosed in detail in WO 2011/005786, and is described briefly below.

RNA effector molecules are ribonucleotide agents that are capable of reducing or preventing the expression of a target gene within a host cell, or ribonucleotide agents capable of forming a molecule that can reduce the expression level of a target gene within a host cell. A portion of a RNA effector molecule, wherein the portion is at least 10, at least 12, at least 15, at least 17, at least 18, at least 19, or at least 20 nucleotide long, is substantially complementary to the target gene. The complementary region may be the coding region, the promoter region, the 3′ untranslated region (3′-UTR), and/or the 5′-UTR of the target gene. Preferably, at least 16 contiguous nucleotides of the RNA effector molecule are complementary to the target sequence (e.g., at least 17, at least 18, at least 19, or more contiguous nucleotides of the RNA effector molecule are complementary to the target sequence). The RNA effector molecules interact with RNA transcripts of target genes and mediate their selective degradation or otherwise prevent their translation.

RNA effector molecules can comprise a single RNA strand or more than one RNA strand. Examples of RNA effector molecules include, e.g., double stranded RNA (dsRNA), microRNA (miRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids and aptamers. The RNA effector molecule can be single-stranded or double-stranded. A single-stranded RNA effector molecule can have double-stranded regions and a double-stranded RNA effector can have single-stranded regions. Preferably, the RNA effector molecules are double-stranded RNA, wherein the antisense strand comprises a sequence that is substantially complementary to the target gene.

Complementary sequences within a RNA effector molecule, e.g., within a dsRNA (a double-stranded ribonucleic acid) may be fully complementary or substantially complementary. Generally, for a duplex up to 30 base pairs, the dsRNA comprises no more than 5, 4, 3 or 2 mismatched base pairs upon hybridization, while retaining the ability to regulate the expression of its target gene.

In some embodiments, the RNA effector molecule comprises a single-stranded oligonucleotide that interacts with and directs the cleavage of RNA transcripts of a target gene. For example, single stranded RNA effector molecules comprise a 5′ modification including one or more phosphate groups or analogs thereof to protect the effector molecule from nuclease degradation. The RNA effector molecule can be a single-stranded antisense nucleic acid having a nucleotide sequence that is complementary to a “sense” nucleic acid of a target gene, e.g., the coding strand of a double-stranded cDNA molecule or a RNA sequence, e.g., a pre-mRNA, mRNA, miRNA, or pre-miRNA. Accordingly, an antisense nucleic acid can form hydrogen bonds with a sense nucleic acid target.

Given a coding strand sequence (e.g., the sequence of a sense strand of a cDNA molecule), antisense nucleic acids can be designed according to the rules of Watson-Crick base pairing. The antisense nucleic acid can be complementary to the coding or noncoding region of a RNA, e.g., the region surrounding the translation start site of a pre-mRNA or mRNA, e.g., the 5′ UTR. An antisense oligonucleotide can be, for example, about 10 to 25 nucleotides in length (e.g., 10, 11, 12, 13, 14, 15, 16, 17, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase its biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

In certain embodiments, the RNA effector comprises a double-stranded ribonucleic acid (dsRNA), wherein said dsRNA (a) comprises a sense strand and an antisense strand that are substantially complementary to each other; and (b) wherein said antisense strand comprises a region of complementarity that is substantially complementary to one of the target genes, and wherein said region of complementarity is from 10 to 30 nucleotides in length.

In some embodiments, RNA effector molecule is a double-stranded oligonucleotide. Typically, the duplex region formed by the two strands is small, about 30 nucleotides or less in length. Such dsRNA is also referred to as siRNA. For example, the siRNA may be from 15 to 30 nucleotides in length, from 10 to 26 nucleotides in length, from 17 to 28 nucleotides in length, from 18 to 25 nucleotides in length, or from 19 to 24 nucleotides in length, etc.

The duplex region can be of any length that permits specific degradation of a desired target RNA through a RISC pathway, but will typically range from 9 to 36 base pairs in length, e.g., 15 to 30 base pairs in length. For example, the duplex region may be 15 to 30 base pairs, 15 to 26 base pairs, 15 to 23 base pairs, 15 to 22 base pairs, 15 to 21 base pairs, 15 to 20 base pairs, 15 to 19 base pairs, 15 to 18 base pairs, 15 to 17 base pairs, 18 to 30 base pairs, 18 to 26 base pairs, 18 to 23 base pairs, 18 to 22 base pairs, 18 to 21 base pairs, 18 to 20 base pairs, 19 to 30 base pairs, 19 to 26 base pairs, 19 to 23 base pairs, 19 to 22 base pairs, 19 to 21 base pairs, 19 to 20 base pairs, 20 to 30 base pairs, 20 to 26 base pairs, 20 to 25 base pairs, 20 to 24 base pairs, 20 to 23 base pairs, 20 to 22 base pairs, 20 to 21 base pairs, 21 to 30 base pairs, 21 to 26 base pairs, 21 to 25 base pairs, 21 to 24 base pairs, 21 to 23 base pairs, or 21 to 22 base pairs.

The two strands forming the duplex structure of a dsRNA can be from a single RNA molecule having at least one self-complementary region, or can be formed from two or more separate RNA molecules. Where the duplex region is formed from two strands of a single molecule, the molecule can have a duplex region separated by a single stranded chain of nucleotides (a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are formed by separate RNA strands, the two strands can be optionally covalently linked. Where the two strands are connected covalently by means other than a hairpin loop, the connecting structure is referred to as a “linker.”

A double-stranded oligonucleotide can include one or more single-stranded nucleotide overhangs, which are one or more unpaired nucleotide that protrudes from the terminus of a duplex structure of a double-stranded oligonucleotide, e.g., a dsRNA. A double-stranded oligonucleotide can comprise an overhang of at least one nucleotide; alternatively the overhang can comprise at least two nucleotides, at least three nucleotides, at least four nucleotides, at least five nucleotides or more. The overhang(s) can be on the sense strand, the antisense strand or any combination thereof. Furthermore, the nucleotide(s) of an overhang can be present on the 5′ end, 3′ end, or both ends of either an antisense or sense strand of a dsRNA.

In one embodiment, at least one end of a dsRNA has a single-stranded nucleotide overhang of 1 to 4, generally 1 or 2 nucleotides.

The overhang can comprise a deoxyribonucleoside or a nucleoside analog. Further, one or more of the internucleoside linkages in the overhang can be replaced with a phosphorothioate. In some embodiments, the overhang comprises one or more deoxyribonucleoside or the overhang comprises one or more dT, e.g., the sequence 5′-dTdT-3′ or 5′-dTdTdT-3′. In some embodiments, overhang comprises the sequence 5′-dT*dT-3, wherein * is a phosphorothioate internucleoside linkage.

An RNA effector molecule as described herein can contain one or more mismatches to the target sequence. Preferably, a RNA effector molecule as described herein contains no more than three mismatches. If the antisense strand of the RNA effector molecule contains one or more mismatches to a target sequence, it is preferable that the mismatch(s) is (are) not located in the center of the region of complementarity, but are restricted to be within the last 5 nucleotides from either the 5′ or 3′ end of the region of complementarity. For example, for a 23-nucleotide RNA effector molecule agent RNA, the antisense strand generally does not contain any mismatch within the central 13 nucleotides.

In some embodiments, the RNA effector molecule is a promoter-directed RNA (pdRNA) which is substantially complementary to a noncoding region of an mRNA transcript of a target gene. In one embodiment, the pdRNA is substantially complementary to the promoter region of a target gene mRNA at a site located upstream from the transcription start site, e.g., more than 100, more than 200, or more than 1,000 bases upstream from the transcription start site. In another embodiment, the pdRNA is substantially complementary to the 3′-UTR of a target gene mRNA transcript. In one embodiment, the pdRNA comprises dsRNA of 18-28 bases optionally having 3′ di- or tri-nucleotide overhangs on each strand. In another embodiment, the pdRNA comprises a gapmer consisting of a single stranded polynucleotide comprising a DNA sequence which is substantially complementary to the promoter or the 3′-UTR of a target gene mRNA transcript, and flanking the polynucleotide sequences (e.g., comprising the 5 terminal bases at each of the 5′ and 3′ ends of the gapmer) comprises one or more modified nucleotides, such as 2′ MOE, 2′OMe, or Locked Nucleic Acid bases (LNA), which protect the gapmer from cellular nucleases.

pdRNA can be used to selectively increase, decrease, or otherwise modulate expression of a target gene. Without being limited to theory, it is believed that pdRNAs modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are known, see, e.g., WO 2009/046397.

In some embodiments, the RNA effector molecule comprises an aptamer which binds to a non-nucleic acid ligand, such as a small organic molecule or protein, e.g., a transcription or translation factor, and subsequently modifies (e.g., inhibits) activity. An aptamer can fold into a specific structure that directs the recognition of a targeted binding site on the non-nucleic acid ligand. Aptamers can contain any of the modifications described herein.

In some embodiments, the RNA effector molecule comprises an antagomir. Antagomirs are single stranded, double stranded, partially double stranded or hairpin structures that target a microRNA. An antagomir consists essentially of or comprises at least 10 or more contiguous nucleotides substantially complementary to an endogenous miRNA and more particularly a target sequence of an miRNA or pre-miRNA nucleotide sequence. Antagomirs preferably have a nucleotide sequence sufficiently complementary to a miRNA target sequence of about 12 to 25 nucleotides, such as about 15 to 23 nucleotides, to allow the antagomir to hybridize to the target sequence. More preferably, the target sequence differs by no more than 1, 2, or 3 nucleotides from the sequence of the antagomir. In some embodiments, the antagomir includes a non-nucleotide moiety, e.g., a cholesterol moiety, which can be attached, e.g., to the 3′ or 5′ end of the oligonucleotide agent.

In some embodiments, antagomirs are stabilized against nucleolytic degradation by the incorporation of a modification, e.g., a nucleotide modification. For example, in some embodiments, antagomirs contain a phosphorothioate comprising at least the first, second, and/or third internucleotide linkages at the 5′ or 3′ end of the nucleotide sequence. In further embodiments, antagomirs include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, antagomirs include at least one 2′-O-methyl-modified nucleotide.

In some embodiments, the RNA effector molecule is a promoter-directed RNA (pdRNA) which is substantially complementary to a noncoding region of an mRNA transcript of a target gene. The pdRNA can be substantially complementary to the promoter region of a target gene mRNA at a site located upstream from the transcription start site, e.g., more than 100, more than 200, or more than 1,000 bases upstream from the transcription start site. Also, the pdRNA can substantially complementary to the 3′-UTR of a target gene mRNA transcript. For example, the pdRNA comprises dsRNA of 18 to 28 bases optionally having 3′ di- or tri-nucleotide overhangs on each strand. The dsRNA is substantially complementary to the promoter region or the 3′-UTR region of a target gene mRNA transcript. In another embodiment, the pdRNA comprises a gapmer consisting of a single stranded polynucleotide comprising a DNA sequence which is substantially complementary to the promoter or the 3′-UTR of a target gene mRNA transcript, and flanking the polynucleotide sequences (e.g., comprising the five terminal bases at each of the 5′ and 3′ ends of the gapmer) comprising one or more modified nucleotides, such as 2′MOE, 2′OMe, or Locked Nucleic Acid bases (LNA), which protect the gapmer from cellular nucleases.

Expressed interfering RNA (eiRNA) can be used to selectively increase, decrease, or otherwise modulate expression of a target gene. Typically, eiRNA, the dsRNA is expressed in the first transfected cell from an expression vector. In such a vector, the sense strand and the antisense strand of the dsRNA can be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Methods for making and using eiRNA effector molecules are known in the art. See, e.g., WO 2006/033756; U.S. Patent Pubs. No. 2005/0239728 and No. 2006/0035344.

In some embodiments, the RNA effector molecule comprises a small single-stranded Piwi-interacting RNA (piRNA effector molecule) which is substantially complementary to a target gene, and which selectively binds to proteins of the Piwi or Aubergine subclasses of Argonaute proteins. A piRNA effector molecule can be about 10 to 50 nucleotides in length, about 25 to 39 nucleotides in length, or about 26 to 31 nucleotides in length. See, e.g., U.S. Patent Application Pub. No. 2009/0062228.

MicroRNAs are a highly conserved class of small RNA molecules that are transcribed from DNA in the genomes of plants and animals, but are not translated into protein. Pre-microRNAs are processed into miRNAs. Processed microRNAs are single stranded ˜17 to 25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. MicroRNAs cause post-transcriptional silencing of specific target genes, e.g., by inhibiting translation or initiating degradation of the targeted mRNA. In some embodiments, the miRNA is completely complementary with the target nucleic acid. In other embodiments, the miRNA has a region of noncomplementarity with the target nucleic acid, resulting in a “bulge” at the region of noncomplementarity. In some embodiments, the region of noncomplementarity (the bulge) is flanked by regions of sufficient complementarity, e.g., complete complementarity, to allow duplex formation. For example, the regions of complementarity are at least 8 to 10 nucleotides long (e.g., 8, 9, or 10 nucleotides long).

miRNA can inhibit gene expression by, e.g., repressing translation, such as when the miRNA is not completely complementary to the target nucleic acid, or by causing target RNA degradation, when the miRNA binds its target with perfect or a high degree of complementarity. In further embodiments, the RNA effector molecule can include an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene.

The miRNA can comprise naturally occurring nucleobases, sugars, and covalent internucleotide (backbone) linkages, or comprise one or more non-naturally-occurring features that confer desirable properties, such as enhanced cellular uptake, enhanced affinity for the endogenous miRNA target, and/or increased stability in the presence of nucleases. In some embodiments, an miRNA designed to bind to a specific endogenous miRNA has substantial complementarity, e.g., at least 70%, 80%, 90%, or 100% complementary, with at least 10, 20, or 25 or more bases of the target miRNA. Exemplary oligonucleotide agents that target miRNAs and pre-miRNAs are described, for example, in U.S. Patent Pubs. No. 20090317907, No. 20090298174, No. 20090291907, No. 20090291906, No. 20090286969, No. 20090236225, No. 20090221685, No. 20090203893, No. 20070049547, No. 20050261218, No. 20090275729, No. 20090043082, No. 20070287179, No. 20060212950, No. 20060166910, No. 20050227934, No. 20050222067, No. 20050221490, No. 20050221293, No. 20050182005, and No. 20050059005.

A miRNA or pre-miRNA can be 10 to 200 nucleotides in length, for example from 16 to 80 nucleotides in length. Mature miRNAs can have a length of 16 to 30 nucleotides, such as 21 to 25 nucleotides, particularly 21, 22, 23, 24, or 25 nucleotides in length. miRNA precursors can have a length of 70 to 100 nucleotides and can have a hairpin conformation. In some embodiments, miRNAs are generated in vivo from pre-miRNAs by the enzymes cDicer and Drosha. miRNAs or pre-miRNAs can be synthesized in vivo by a cell-based system or can be chemically synthesized. miRNAs can comprise modifications which impart one or more desired properties, such as superior stability, hybridization thermodynamics with a target nucleic acid, targeting to a particular tissue or cell-type, and/or cell permeability, e.g., by an endocytosis-dependent or -independent mechanism. Modifications can also increase sequence specificity, and consequently decrease off-site targeting.

Optionally, an RNA effector may biochemically modified to enhance stability or other beneficial characteristics.

Oligonucleotides can be modified to prevent rapid degradation of the oligonucleotides by endo- and exo-nucleases and avoid undesirable off-target effects. The nucleic acids featured in the invention can be synthesized and/or modified by methods well established in the art, such as those described in CURRENT PROTOCOLS IN NUCLEIC ACID CHEMISTRY (Beaucage et al., eds., John Wiley & Sons, Inc., NY). Modifications include, for example, (a) end modifications, e.g., 5′ end modifications (phosphorylation, conjugation, inverted linkages, etc.), or 3′ end modifications (conjugation, DNA nucleotides, inverted linkages, etc.); (b) base modifications, e.g., replacement with stabilizing bases, destabilizing bases, or bases that base pair with an expanded repertoire of partners, removal of bases (abasic nucleotides), or conjugated bases; (c) sugar modifications (e.g., at the 2′ position or 4′ position) or replacement of the sugar; as well as (d) internucleoside linkage modifications, including modification or replacement of the phosphodiester linkages. Specific examples of oligonucleotide compounds useful in this invention include, but are not limited to RNAs containing modified backbones or no natural internucleoside linkages. RNAs having modified backbones include, among others, those that do not have a phosphorus atom in the backbone. Specific examples of oligonucleotide compounds useful in this invention include, but are not limited to oligonucleotides containing modified or non-natural internucleoside linkages. Oligonucleotides having modified internucleoside linkages include, among others, those that do not have a phosphorus atom in the internucleoside linkage.

Modified internucleoside linkages include (e.g., RNA backbones) include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Additionally, both the sugar and the internucleoside linkage may be modified, i.e., the backbone, of the nucleotide units are replaced with novel groups. One such oligomeric compound, an RNA mimetic that has been shown to have excellent hybridization properties, is referred to as a peptide nucleic acid (PNA).

Modified oligonucleotides can also contain one or more substituted sugar moieties. The RNA effector molecules, e.g., dsRNAs, can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Other modifications include 2′-methoxy (2′-OCH₃), 2′-aminopropoxy (2′-OCH₂CH₂CH₂NH₂) and 2′-fluoro (2′-F).

The oligonucleotides can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to oligonucleotide molecules has been shown to increase oligonucleotide molecule stability in serum, and to reduce off-target effects. Elmen et al., 33 Nucl. Acids Res. 439-47 (2005); Mook et al., 6 Mol. Cancer Ther. 833-43 (2007); Grunweller et al., 31 Nucl. Acids Res. 3185-93 (2003); U.S. Pat. No. 6,268,490; U.S. Pat. No. 6,670,461; U.S. Pat. No. 6,794,499; U.S. Pat. No. 6,998,484; U.S. Pat. No. 7,053,207; U.S. Pat. No. 7,084,125; and U.S. Pat. No. 7,399,845.

2. Activator Molecules

In certain embodiments, the activator is an molecule or agent that is effective to increase expression of one or more genes. In general, the activator is an agent that is effective to increase initiation of transcription binding factors and/or decrease transcription inhibitors. In one embodiment, the activator is an activator protein that modulates expression of the selected gene or genes to be upregulated.

3. Delivery Methods of RNA Effector Molecules and/or Activators

The discussion below is with reference to delivery of RNA effector molecules. However, it will be understood that the delivery methods described below are applicable to activators. The delivery of RNA effector molecules to cells can be achieved in a number of different ways. Several suitable delivery methods are well known in the art. For example, the skilled person is directed to WO 2011/005786, which discloses exemplary delivery methods can be used in this invention at pages 187-219, the teachings of which are incorporated herein by reference.

A reagent that facilitates RNA effector molecule uptake may be used. For example, an emulsion, a cationic lipid, a non-cationic lipid, a charged lipid, a liposome, an anionic lipid, a penetration enhancer, a transfection reagent or a modification to the RNA effector molecule for attachment, e.g., a ligand, a targeting moiety, a peptide, a lipophilic group, etc.

For example, RNA effector molecules can be delivered using a drug delivery system such as a nanoparticle, a dendrimer, a polymer, a liposome, or a cationic delivery system. Positively charged cationic delivery systems facilitate binding of a RNA effector molecule (negatively charged) and also enhance interactions at the negatively charged cell membrane to permit efficient cellular uptake. Cationic lipids, dendrimers, or polymers can either be bound to RNA effector molecules, or induced to form a vesicle, liposome, or micelle that encases the RNA effector molecule. See, e.g., Kim et al., 129 J. Contr. Release 107-16 (2008). Methods for making and using cationic-RNA effector molecule complexes are well within the abilities of those skilled in the art. See e.g., Sorensen et al 327 J. Mol. Biol. 761-66 (2003); Verma et al., 9 Clin. Cancer Res. 1291-1300 (2003); Arnold et al., 25 J. Hypertens. 197-205 (2007).

The RNA effector molecules described herein can be encapsulated within liposomes or can form complexes thereto, in particular to cationic liposomes. Alternatively, the RNA effector molecules can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride, or acceptable salts thereof.

The lipid to RNA ratio (mass/mass ratio) (e.g., lipid to dsRNA ratio) can be in ranges of from about 1:1 to about 50:1, from about 1:1 to about 25:1, from about 3:1 to about 15:1, from about 4:1 to about 10:1, from about 5:1 to about 9:1, or about 6:1 to about 9:1, inclusive.

A cationic lipid of the formulation can comprise at least one protonatable group having a pKa of from 4 to 15. The cationic lipid can be, for example, N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), 2,2-Dilinoleyl-4-dimethylaminoethyl-[1,3]-dioxolane, or a mixture thereof. The cationic lipid can comprise from about 20 mol % to about 70 mol %, inclusive, or about 40 mol % to about 60 mol %, inclusive, of the total lipid present in the particle. In one embodiment, cationic lipid can be further conjugated to a ligand.

A non-cationic lipid can be an anionic lipid or a neutral lipid, such as distearoyl-phosphatidylcholine (DSPC), dioleoylphosphatidylcholine (DOPC), dipalmitoyl-phosphatidylcholine (DPPC), dioleoylphosphatidylglycerol (DOPG), dipalmitoyl-phosphatidylglycerol (DPPG), dioleoyl-phosphatidylethanolamine (DOPE), palmitoyloleoyl-phosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, inclusive, of about 10 mol %, to about 58 mol %, inclusive, if cholesterol is included, of the total lipid present in the particle.

4. Antibodies

In certain embodiments, the inhibitor is an antibody that binds to a gene product described herein (e.g., a protein encoded by the gene), such as a neutralizing antibody that reduces the activity of the protein.

The term “antibody” refers to an immunoglobulin or fragment thereof, and encompasses any such polypeptide comprising an antigen-binding fragment of an antibody. The term includes but is not limited to polyclonal, monoclonal, monospecific, polyspecific, humanized, human, single-chain, chimeric, synthetic, recombinant, hybrid, mutated, grafted, and in vitro generated antibodies.

An antibody may also refer to antigen-binding fragments of an antibody. Examples of antigen-binding fragments include, but are not limited to, Fab fragments (consisting of the V_{L}, V_{H}, C_{L} and C_{H}1 domains); Fd fragments (consisting of the V_{H} and C_{H}1 domains); Fv fragments (referring to a dimer of one heavy and one light chain variable domain in tight, non-covalent association); dAb fragments (consisting of a V_{H} domain); isolated CDR regions; (Fab′)₂ fragments, bivalent fragments (comprising two Fab fragments linked by a disulphide bridge at the hinge region), scFv (referring to a fusion of the V_{L} and V_{H} domains, linked together with a short linker), and other antibody fragments that retain antigen-binding function. The part of the antigen that is specifically recognized and bound by the antibody is referred to as the “epitope.”

An antigen-binding fragment of an antibody can be produced by conventional biochemical techniques, such as enzyme cleavage, or recombinant DNA techniques known in the art. These fragments may be produced by proteolytic cleavage of intact antibodies by methods well known in the art, or by inserting stop codons at the desired locations in the vectors using site-directed mutagenesis, such as after C_{H}1 to produce Fab fragments or after the hinge region to produce (Fab′)₂ fragments. For example, Papain digestion of antibodies produces two identical antigen-binding fragments, called “Fab” fragments, each with a single antigen-binding site, and a residual “Fc” fragment. Pepsin treatment of an antibody yields an F(ab′)₂ fragment that has two antigen-combining sites and is still capable of cross-linking antigen. Single chain antibodies may be produced by joining V_{L} and V_{H} coding regions with a DNA that encodes a peptide linker connecting the V_{L} and V_{H} protein fragments

An antigen-binding fragment/domain may comprise an antibody light chain variable region (V_{L}) and an antibody heavy chain variable region (V_{H}); however, it does not have to comprise both. Fd fragments, for example, have two V_{H} regions and often retain some antigen-binding function of the intact antigen-binding domain. Examples of antigen-binding fragments of an antibody include (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region; (3) a Fd fragment having the two V_{H} and C_{H}1 domains; (4) a Fv fragment having the V_{L} and V_{H} domains of a single arm of an antibody, (5) a dAb fragment (Ward et al., (1989) Nature 341:544-546), that has a V_{H} domain; (6) an isolated complementarity determining region (CDR), and (7) a single chain Fv (scFv). Although the two domains of the Fv fragment, V_{L} and V_{H}, are coded for by separate genes, they can be joined, using recombinant DNA methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are evaluated for function in the same manner as are intact antibodies.

Antibodies described herein, or an antigen-binding fragment thereof, can be prepared, for example, by recombinant DNA technologies and/or hybridoma technology. For example, a host cell may be transfected with one or more recombinant expression vectors carrying DNA fragments encoding the immunoglobulin light and heavy chains of the antibody, or an antigen-binding fragment of the antibody, such that the light and heavy chains are expressed in the host cell and, preferably, secreted into the medium in which the host cell is cultured, from which medium the antibody can be recovered. Antibodies derived from murine or other non-human species can be humanized, e.g., by CDR drafting.

Standard recombinant DNA methodologies may be used to obtain antibody heavy and light chain genes or a nucleic acid encoding the heavy or light chains, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N. Y., (1989), Ausubel, F. M. et al. (eds.) Current Protocols in Molecular Biology, Greene Publishing Associates, (1989) and in U.S. Pat. No. 4,816,397 by Boss et al.

5 Combination Therapy

The inhibitors described herein may be used in combination with another therapeutic agent. Further, the methods of treatment described herein may be carried out in combination with another treatment regimen, such as chemotherapy, radiotherapy, surgery, etc.

Suitable chemotherapeutic drugs include, e.g., alkylating agents, anti-metabolites, anti-mitototics, alkaloids (e.g., plant alkaloids and terpenoids, or vinca alkaloids), podophyllotoxin, taxanes, topoisomerase inhibitors, cytotoxic antibiotics, or a combination thereof. Examples of these chemotherapeutic drugs include platinum-based drugs, bevacizumab, paclitaxel, docetaxel, pegylated liposomal doxorubicin, topotecan, letrozole, tamoxifen citrate, topotecan hydrochloride, and trametinib. Examples of platinum-based drugs include, but are not limited to cisplatin and carboplatin.

The inhibitors described herein can also be administered in combination with radiotherapy or surgery. For example, an inhibitor can be administered prior to, during or after surgery or radiotherapy. Administration during surgery can be as a bathing solution for the operation site.

Additionally, the RNA effector molecules described herein may be used in combination with additional RNA effector molecules that target additional genes (such as a growth factor, or an oncogene) to enhance efficacy. For example, certain oncogenes are known to increase the malignancy of a tumor cell. Some oncogenes, usually involved in early stages of cancer development, increase the chance that a normal cell develops into a tumor cell. Accordingly, one or more oncogenes may be targeted in addition to Cdkn1A, Mapk14, Rad51AP1, Kras, Rpa3, Pold2, Pabpc5, and Bcap31. Commonly seen oncogenes include growth factors or mitogens (such as Platelet-derived growth factor), receptor tyrosine kinases (such as HER2/neu, also known as ErbB-2), cytoplasmic tyrosine kinases (such as the Src-family, Syk-ZAP-70 family and BTK family of tyrosine kinases), regulatory GTPases (such as Ras), cytoplasmic serine/threonine kinases (such as cyclin dependent kinases) and their regulatory subunits, and transcription factors (such as myc).

6 Administration

Inhibitors and activators described herein may be formulated into pharmaceutical compositions. The pharmaceutical compositions usually one or more pharmaceutical carrier(s) and/or excipient(s). A thorough discussion of such components is available in Gennaro (2000) Remington: The Science and Practice of Pharmacy (20th edition). Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Formulation of the pharmaceutical composition will vary according to the route of administration selected.

The amounts of an inhibitor and/or activator in a given dosage will vary according to the size of the individual to whom the therapy is being administered as well as the characteristics of the disorder being treated. In exemplary treatments, it may be necessary to administer about 1 mg/day, about 5 mg/day, about 10 mg/day, about 20 mg/day, about 50 mg/day, about 75 mg/day, about 100 mg/day, about 150 mg/day, about 200 mg/day, about 250 mg/day, about 400 mg/day, about 500 mg/day, about 800 mg/day, about 1000 mg/day, about 1600 mg/day or about 2000 mg/day. The doses may also be administered based on weight of the patient, at a dose of 0.01 to 50 mg/kg. The glycoprotein may be administered in a dose range of 0.015 to 30 mg/kg, such as in a dose of about 0.015, about 0.05, about 0.15, about 0.5, about 1.5, about 5, about 15 or about 30 mg/kg.

The compositions described herein may be administered to a subject orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well.

Standard dose-response studies, first in animal models and then in clinical testing, can reveal optimal dosages for particular diseases and patient populations.

To facilitate a better understanding of the subject technology, the following examples of preferred embodiments are given. In no way should the following examples be read to limit, or to define, the scope of the subject technology.

### Example 1

According to some embodiments, a generalized singular value decomposition (GSVD) was used to identify a global pattern of tumor-exclusive co-occurring CNAs that is correlated and possibly coordinated with OV survival. This pattern is revealed by GSVD comparison of array comparative genomic hydridization (aCGH) data from patient-matched OV and normal blood samples from The Cancer Genome Atlas (TCGA).

FIG. 3 is a diagram of a tensor generalized singular value decomposition (GSVD) of the patient- and platform-matched DNA copy-number profiles of the 6p+12p chromosome arms, according to some embodiments. For each chromosome arm or combination of two chromosome arms, the structure of the tumor and normal discovery datasets (D₁ and D₂) is that of two third-order tensors with one-to-one mappings between the column dimensions but different row dimensions. The patients, platforms, probes, and tissue types, each represent a degree of freedom. The tensor GSVD is depicted in a raster display, with relative copy-number gain, no change, and loss, explicitly showing the first through the 5th, and the 245th through the 249th 6p+12p x-probelets, both 6p+12p y-probelets, and the first through the 10th, and the 489th through the 498th 6p+12p tumor and normal arraylets. This display shows that the significance of a subtensor in the tumor dataset relative to that of the corresponding subtensor in the normal dataset, i.e., the tensor GSVD angular distance, equals the row mode GSVD angular distance, i.e., the significance of the corresponding tumor arraylet in the tumor dataset relative to that of the normal arraylet in the normal dataset. The tensor GSVD angular distances for the 498 pairs of 6p+12p arraylets are depicted in a bar chart display, where the angular distance corresponding to the first pair of arraylets is ˜π/4. For the 6p+12p combination of two chromosome arms, it was found that the most significant subtensor in the tumor dataset (which corresponds to the coefficient of largest magnitude in R₁) is a combination of (i) the first y-probelet, which is approximately invariant across the platforms, (ii) the first x-probelet, which classifies the discovery set of patients into two groups of high and low coefficients, of significantly and robustly different prognoses, and (iii) the first, most tumor-exclusive tumor arraylet, which classifies the validation set of patients into two groups of high and low correlations of significantly different prognoses consistent with the x-probelet's classification of the discovery set.

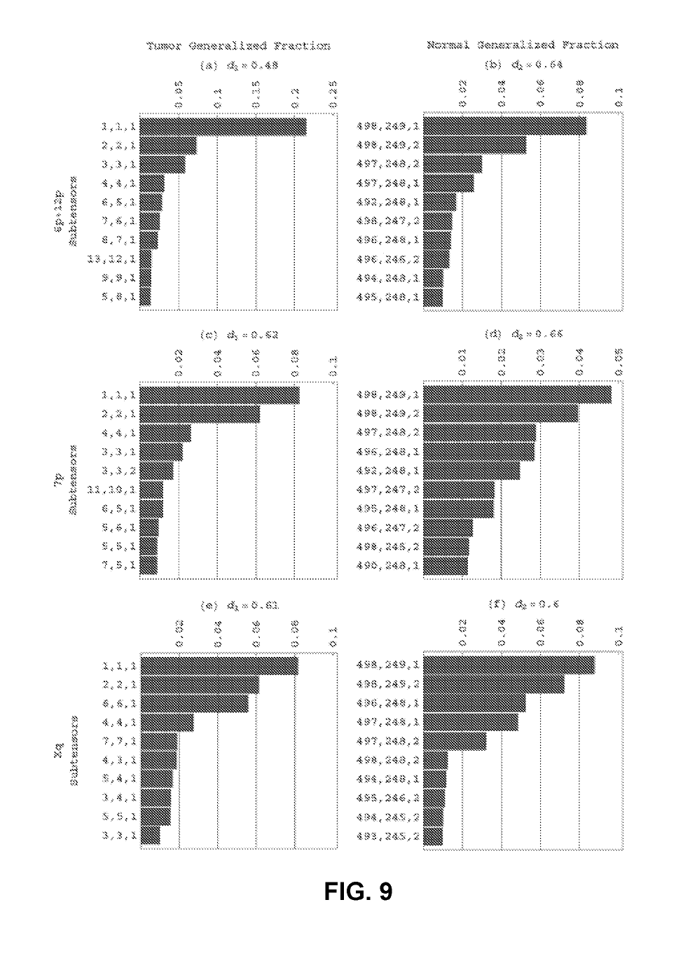

FIG. 4 is a diagram illustrating a GSVD of biological data, according to some embodiments. The tensor GSVD of the patient- and platform-matched DNA copy-number profiles of the 7p chromosome arm is depicted in a raster display. The raster display is depicted with relative copy-number gain, no change, and loss, explicitly showing the first through the 5th, and the 245th through the 249th 7p x-probelets, both 7p y-probelets, and the first through the 10th, and the 489th through the 498th 7p tumor and normal arraylets. The display shows that the significance of a subtensor in the tumor dataset relative to that of the corresponding subtensor in the normal dataset, i.e., the tensor GSVD angular distance, equals the row mode GSVD angular distance, i.e., the significance of the corresponding tumor arraylet in the tumor dataset relative to that of the normal arraylet in the normal dataset. The tensor GSVD angular distances for the 498 pairs of 7p arraylets are depicted in a bar chart display (FIG. 9), where the angular distance corresponding to the first pair of arraylets is ˜π/4. For the 7p chromosome arm, the most significant subtensor in the tumor dataset is a combination of (i) the first y-probelet, which is approximately invariant across the platforms, (ii) the first x-probelet, which classifies the discovery set of patients into two groups of high and low coefficients, of significantly and robustly different prognoses, and (iii) the first, most tumor-exclusive tumor arraylet, which classifies the validation set of patients into two groups of high and low correlations of significantly different prognoses consistent with the x-probelet's classification of the discovery set.

FIG. 5 is a diagram illustrating the tensor GSVD of the patient- and platform-matched DNA copy-number profiles of the Xq chromosome arm, according to some embodiments. The tensor GSVD is depicted in a raster display, with relative copy-number gain, no change, and loss, explicitly showing the first through the 5th, and the 245th through the 249th Xq x-probelets, both Xq y-probelets, and the first through the 10th, and the 489th through the 498th Xq tumor and normal arraylets. The tensor GSVD angular distances for the 498 pairs of Xq arraylets are depicted in a bar chart display (FIG. 9), where the angular distance corresponding to the first pair of arraylets is ˜π/4.

The significance of the probelet in the tumor data set relative to its significance in the normal data set is depicted in a bar chart display (FIG. 9). Bar charts of the ten subtensors Sᵢ(a, b, c) that are most significant in the 6p+12p (a) tumor, and (b) normal, 7p (c) tumor, and (d) normal, and Xq (e) tumor, and (f) normal datasets, in terms of the fractions P_{i,abc}, i.e., the subtensors which correspond to the coefficients of largest magnitudes are shown in FIG. 9. The most significant subtensor in each of the tumor datasets, e.g., is S₁(1, 1, 1), which is a combination or an outer product of the first, most tumor-exclusive tumor arraylet, and the first x- and y-probelets. The most significant subtensor in each of the normal datasets is S₂(498, 249, 1), which is a combination or an outer product of the 498th, most normal-exclusive normal arraylet, the 249th x-probelet and the first y-probelet. The tensor generalized Shannon entropy d, of each dataset is also noted.

### Example 2

According to embodiments described above, a GSVD has been used to identify a global pattern of tumor-exclusive co-occurring CNAs that is correlated and possibly coordinated with OV survival. This pattern is revealed by GSVD comparison of array comparative genomic hydridization (aCGH) data from discovery and validation patient profiles from The Cancer Genome Atlas (TCGA).

The discovery set of patients reflects the general primary, high-grade OV patient population, with approximately 5%, 7%, 76%, and 12% of the patients diagnosed at stages I, II, III, and IV, and 218, i.e., ˜88%, treated with platinum-based chemotherapy, i.e., cisplatin, carboplatin, or oxaliplatin, and 240 of the 249, i.e., >95% of the tumors at grades 2 and higher.

We selected primary OV tumor and normal DNA copy-number profiles of a set of 249 TCGA patients. Each profile was measured in two replicates by the same set of two DNA microarray platforms.

Each profile in the discovery datasets lists log₂ of TCGA level 1 background-subtracted intensity in the sample relative to the male Promega DNA reference, with signal to background≥2.5 for both the sample and reference in ≥90% of the 391,190 autosomal probes and ≥65% of the 10,911 X chromosome probes that match between the two Agilent Human array CGH (aCGH) DNA microarray platforms, G4447A and G4124A. Tumor and normal probes were selected with valid data in ≥99% of the tumor or normal arrays of each platform, respectively. For each chromosome arm or combination of two chromosome arms, and for each platform, the <0.5% missing data entries in the tumor and normal profiles were estimated by using the SVD, as previously described. Each profile was then centered at its copy-number median, and normalized by its copy-number sMAD.

For the validation dataset, we selected 131 and 41 stage III-IV OV aCGH profiles measured by the Agilent Human aCGH G4447A and G4124A microarray platforms, respectively, corresponding to 148 primary OV tumors. Of the 148 patients, 140, i.e., ˜95%, were treated with platinum-based chemotherapy, and 144, i.e., >95% of the tumors are high-grade, i.e., grades 2 and higher tumors. Each profile lists log₂ of TCGA level 1 background-subtracted intensity in the sample relative to the male Promega DNA reference, with signal to background≥2.5 for both the sample and reference in ≥99.5% of the 391,190 autosomal probes and ≥96.5% of the 10,911 X chromosome probes that match between the platforms. Medians of the profiles of samples from the same patient were then taken.

FIGS. 6-8 show tumor-exclusive and platform-consistent DNA copy-number alterations (CNAs) correlated with OV patients' survival, in some embodiments. A plot of the first 6p+12p tumor arraylet describes a pattern of tumor-exclusive and platform-consistent co-occurring CNAs across the combination of the two chromosome arms 6p+12p (see (a)). The probes are ordered, and their copy numbers are colored according to each probe's chromosomal band location. Segments (black lines) amplified and deleted include most known OV-associated CNAs that map to 6p+12p (black), including an amplification of Kras and a deletion of Prim2. CNAs previously unrecognized in OV include a deletion of the p38-encoding Mapk14, and p21-encoding Cdkn1A, and an amplification of Rad51AP1, a deletion of Tnf, and focal amplifications of Asun, Itpr2, and the 5′ ends of isoforms a and e, and exons 5 and 6 of Sox5. A high 6p+12p arraylet correlation is significantly correlated with a patient's shorter survival time. A plot of the first 6p+12p x-probelet describes the classification of the discovery set of patients into two groups of high and low coefficients (see (b)). A high 6p+12p x-probelet coefficient is significantly and robustly correlated with a patient's shorter survival time. A raster display of the 6p+12p tumor profiles, where medians of the profiles of the same patient measured by the two platforms were taken, with relative gain, no change, and loss of DNA copy numbers is shown in (c). A plot of the first 7p tumor arraylet describes a pattern of CNAs across the chromosome arm 7p (see (d)). CNAs previously unrecognized in OV include a focal deletion of Rpa3 and an amplification of Pold2. A high 7p arraylet correlation is significantly correlated with a patient's longer survival time. A plot of the first 7p x-probelet describes the classification of the discovery set of patients into two groups of high and low coefficients is shown in (e). A high 7p x-probelet coefficient is significantly and robustly correlated with a patient's longer survival time. A raster display of the 7p tumor profiles is shown in (f). A plot of the first Xq tumor arraylet is shown in (g). CNAs previously unrecognized in OV include a focal deletion of Pabpc5 and an amplification of Bcap31. A high Xq arraylet correlation is significantly correlated with a patient's longer survival time. A plot of the first Xq x-probelet describes the classification of the discovery set of patients into two groups of high and low coefficients (see (h)). A high Xq x-probelet coefficient is significantly and robustly correlated with a patient's longer survival time. A raster display of the Xq tumor profiles is shown in (i).

### Example 3

Survival analysis was used to identify CNAs that may be related to predictors of OV survival and/or response to therapy (e.g. platinum-based chemotherapy), in some embodiments.

Kaplan-Meier (KM) curves of the discovery set of 249 patients classified by the standard OV indicators are shown in FIG. 10: (a) tumor stage at diagnosis, the best predictor of OV survival to date, (b) residual disease after surgery, i.e., no (No) or some (Yes) macroscopic disease, (c) outcome of subsequent therapy, i.e., complete remission (CR) or not (No). (d) neoplasm status, i.e., with (W) tumor or without (WO).

FIG. 11 shows KM curves of survival analysis for the validation set of 148 stage III-IV patients classified by (a) tumor stage at diagnosis, (b) residual disease after surgery, i.e., no (No) or some (Yes) macroscopic disease, (c) outcome of subsequent therapy, i.e., complete remission (CR) or not (No). (d) neoplasm status, i.e., with (W) tumor or without (WO).

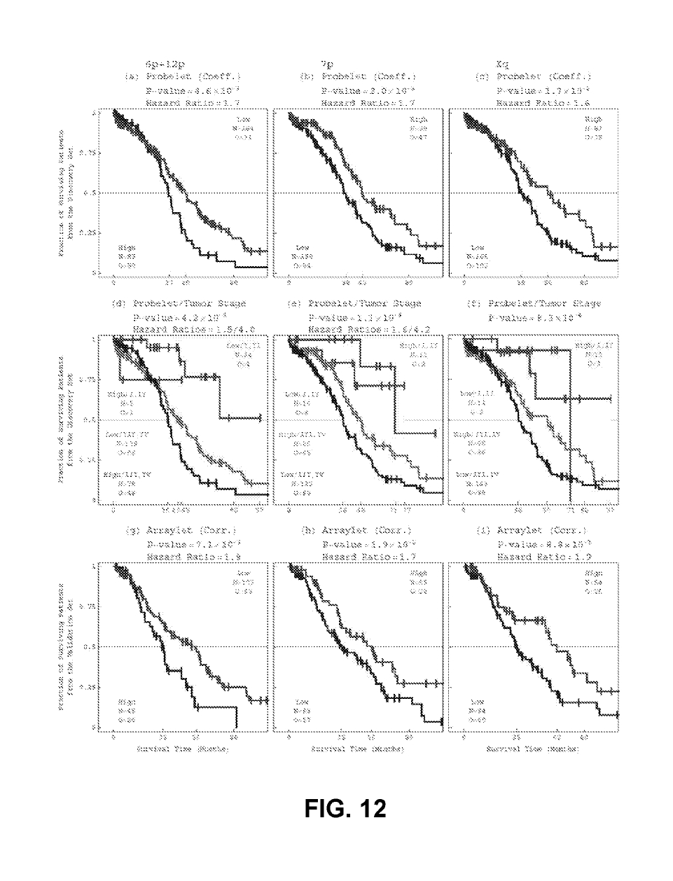

FIG. 12 shows survival analyses of the discovery and validation sets of patients classified by tensor GSVD, or tensor GSVD and tumor stage at diagnosis. KM curves of the discovery set of 249 patients classified by the 6p+12p x-probelet coefficient (see (a), show a median survival time difference of 11 months, with the corresponding log-rank test P-value<10⁻². The univariate Cox proportional hazard ratio is 1.7. KM curve (b) shows survival analyses of the 249 patients classified by the 7p x-probelet coefficient. KM curve (c) shows survival analysis of the 249 patients classified by the Xq x-probelet coefficient. KM curve (d) shows survival analysis of the 249 patients classified by both the 6p+12p tensor GSVD and tumor stage at diagnosis, show the bivariate Cox hazard ratios of 1.5 and 4.0, which do not differ significantly from the corresponding univariate hazard ratios of 1.7 and 4.4, respectively. This means that the 6p+12p tensor GSVD is independent of stage, the best predictor of OV survival to date. The 61 months KM median survival time difference is about 85% and more than two years greater than the 33 month difference between the patients classified by stage alone. This means that the tensor GSVD and stage combined make a better predictor than stage alone. KM curve (e) shows survival analysis for the 249 patients classified by both the 7p tensor GSVD and stage. KM curve (f) shows survival analysis for the 249 patients classified by both the Xq tensor GSVD and stage. KM curves of the validation set of 148 stage III-IV patients classified by the 6p+12p arraylet correlation (see (g)), show a median survival time difference of 22 months, with the corresponding log-rank test P-value<10⁻², and the univariate Cox proportional hazard ratio 1.9. This validates the survival analyses of the discovery set of 249 patients. KM curve (h) shows survival analyses of the 148 patients classified by the 7p arraylet correlation. KM curve (i) shows survival analysis for the 148 patients classified by the Xq arraylet correlation.

FIG. 13 shows survival analyses of the platinum-based chemotherapy patients in the discovery and validation sets classified by tensor GSVD, or tensor GSVD and tumor stage at diagnosis. KM curves of only the 218, i.e., ˜88% platinum-based chemotherapy patients in the discovery set, classified by the 6p+12p x-probelet coefficient, show a median survival time difference of 14 months, with the corresponding log-rank test P-value<10⁻³ (see (a)). The univariate Cox proportional hazard ratio is 2.0. KM curve (b) shows survival analyses of the 218 patients classified by the 7p x-probelet coefficient. KM curve (c) shows survival analysis for the 218 patients classified by the Xq x-probelet coefficient. The 218 patients classified by both the 6p+12p tensor GSVD and tumor stage at diagnosis, show the bivariate Cox hazard ratios of 1.8 and 4.1, which do not differ significantly from the corresponding univariate hazard ratios of 2.0 and 4.4, respectively (see KM curve (d). This means that the 6p+12p tensor GSVD is independent of stage, the best predictor of OV survival to date. KM curve (e) shows survival analysis for the 218 patients classified by both the 7p tensor GSVD and stage. KM curve (f) shows survival analysis for the 218 patients classified by both the Xq tensor GSVD and stage. KM curves of only the 140, i.e., ˜95% platinum-based chemotherapy patients in the validation set, classified by the 6p+12p arraylet correlation, show a median survival time difference of 18 months, with the univariate Cox proportional hazard ratio 1.8 (see (g)). This validates the survival analyses of the 218 chemotherapy patients in the discovery set. KM curve (h) shows survival analyses of the 148 patients classified by the 7p arraylet correlation. KM curve (i) shows survival analysis for the 148 patients classified by the Xq arraylet correlation.

FIG. 14 shows survival analyses of the validation set of patients classified by tensor GSVD and tumor stage at diagnosis. KM curves of the validation set of 148 stage III-IV patients classified by both the 6p+12p tensor GSVD and tumor stage at diagnosis, show the bivariate Cox hazard ratios of 1.9 and 1.8, which are the same as the corresponding univariate ratios (see (a)). This means that the 6p+12p tensor GSVD is independent of stage, the best predictor of OV survival to date. The 34 months KM median survival time difference is about 62% and more than one year greater than the 21 month difference between the patients classified by stage alone. This means that the tensor GSVD and stage combined make a better predictor than stage alone. KM curve (b) shows survival analysis for the 148 patients classified by both the 7p tensor GSVD and stage. KM curve (c) shows survival analysis for the 148 patients classified by both the Xq tensor GSVD and stage.

FIG. 15 shows survival analyses of the discovery set of patients classified by tensor GSVD and standard OV indicators other than stage. KM curves of the discovery set of 249 patients classified by both the (a) 6p+12p, (b) 7p, or (c) Xq tensor GSVD, and residual disease after surgery, the (d) 6p+12p, (e) 7p, or (f) Xq tensor GSVD, and outcome of subsequent therapy, and (g) 6p+12p, (h) 7p, or (i) Xq tensor GSVD, and neoplasm status.

FIG. 16 shows survival analyses of the validation set of patients classified by tensor GSVD and standard OV indicators other than stage. KM curves of the validation set of 148 stage III-IV patients classified by both the (a) 6p+12p, (b) 7p, or (c) Xq tensor GSVD, and residual disease after surgery, the (d) 6p+12p, (e) 7p, or (f) Xq tensor GSVD, and outcome of subsequent therapy, and (g) 6p+12p, (h) 7p, or (i) Xq tensor GSVD, and neoplasm status.

FIG. 17 shows survival analyses of the discovery and validation sets of patients classified by the novel frequent focal CNAs included in the tensor GSVD arraylets. Six novel frequent focal CNAs that are included in the tensor GSVD arraylets are significantly correlated with OV survival. Two amplified consecutive segments (12p12.1) contain (a) the 5′ ends of isoforms a and e of Sox5, and (b) exons 5 and 6, the first exons that are common to isoforms a, b, d, and e of Sox5. Two other amplified consecutive segments (12p11.23) contain (c) Itpr2 and (d) Asun. One deletion (7p22.1-p21.3) contains (e) Rpa3. Another deletion (Xq21.31) contains (f) Pabpc5, and the sequence tag site DXS241 adjacent to translocation breakpoints observed in premature ovarian failure.

FIG. 18 shows survival analyses of the discovery and validation sets of patients, as well as only the platinum-based chemotherapy patients in the discovery and validation sets, classified by the 6p+12p, 7p, and Xq tensor GSVD combined. KM curves of the discovery set of 249 patients classified by combination of the 6p+12p, 7p, and Xq x-probelet coefficients, show median survival times of 86, 52, and 36 months for the groups A, B, and C, respectively, with the corresponding log-rank test P-value<10⁻³ is shown in (a). KM survival analysis of only the 218, i.e., ˜88% platinum-based chemotherapy patients in the discovery set, classified by combination of the three tensor GSVDs, gives qualitatively the same and quantitatively similar results to those of the analyses of 100% of the patients (see (b)). This means that the combination of the three tensor GSVDs predicts survival in the platinum-based chemotherapy patient population. KM curves of the validation set of 148 stage III-IV patients classified by combination of the 6p+12p, 7p, and Xq arraylet correlation coefficients, show median survival times of 72, 57, and 33 months for the groups A, B, and C, respectively, with the corresponding log-rank test P-value<10⁻³ (see (c)). This validates the survival analyses of the discovery set of 249 patients. KM survival analysis of only the 140, i.e., ˜95% platinum-based chemotherapy patients in the validation set, classified by combination of the three tensor GSVDs are shown in (d).

### Example 4

To compare the variation in DNA copy numbers with that in gene expression, we used mRNA expression profiles that were available for 394 of the 397 TCGA patients in the discovery and validation sets. Each profile lists TCGA level 3 mRNA expression for 11,457 autosomal and X chromosome genes on the Affymetrix Human Genome U133A Array platform with UCSC coordinates and GO annotations. Medians of the profiles of samples from the same patient were taken. To examine the possible relations between a tensor GSVD class and the OV pathogenesis, we assessed the enrichment of the subsets of genes that are differentially expressed between the tensor GSVD classes in any one of the multiple GO annotations. The P-value of a given enrichment was calculated assuming hypergeometric probability distribution of the annotations among the genes in the global set, and of the subset of annotations among the subset of genes, as previously described (Alter et al., PNAS USA, 2003, 100:3351-3356].

FIG. 19 shows differential mRNA expression between the tensor GSVD classes is consistent with the CNAs. Differential mRNA expression is shown for: (a) Tnf, (b) Mapk14, and (c) Cdkn1A, which are deleted in the 6p+12p arraylet, are significantly (Mann-Whitney-Wilcoxon P-value<0.05) underexpressed in the tensor GSVD class of a high 6p+12p x-probelet coefficient, or arraylet correlation relative to the tensor GSVD class of a low 6p+12p x-probelet coefficient, or arraylet correlation. (d) Rad51AP1, (e) Itpr2, and (f) Asun, which are amplified in the 6p+12p arraylet, are significantly overexpressed in the tensor GSVD class of a high 6p+12p x-probelet coefficient, or arraylet correlation. (g) Rpa3, which is deleted, and (h) Pold2, which is amplified, in the 7p arraylet, are significantly underexpressed and overexpressed, respectively, in the tensor GSVD class of a high 7p x-probelet coefficient, or arraylet correlation. (i) Bcap31, which is amplified in the Xq arraylet, is significantly overexpressed in the tensor GSVD class of a high Xq x-probelet coefficient, or arraylet correlation.

To compare with the variation in microRNA expression, we used microRNA expression profiles that were available for 395 of the 397 patients. Each profile lists TCGA level 3 microRNA expression for 639 autosomal and X chromosome microRNAs on the Agilent Human microRNA Array 8×15K platform with UCSC coordinates. Medians of the profiles of samples from the same patient were taken.

FIG. 20 shows differential microRNA expression between the tensor GSVD classes is consistent with the CNAs. Differential microRNA expression is shown for: (a) mir-877*, which is deleted, and (b) mir-200c, (c) mir-200c*, (d) mir-141, and (e) mir-141*, which are amplified in the 6p+12p arraylet, are significantly (Mann-Whitney-Wilcoxon P-value<0.05) underexpressed and overexpressed, respectively, in the tensor GSVD class of a high 6p+12p x-probelet coefficient, or arraylet correlation relative to the tensor GSVD class of a low 6p+12p x-probelet coefficient, or arraylet correlation. (f) mir-888, (g) mir-224, and (h) mir-452, which are amplified in the Xq arraylet, are significantly overexpressed in the tensor GSVD class of a high Xq x-probelet coefficient, or arraylet correlation.

To compare with the variation in protein expression, we used protein expression profiles that were available for 282 of the 397 patients. Each profile lists TCGA level 3 protein expression for the 175 antibodies on the MD Anderson Reverse Phase Protein Array (RPPA), which probe for the abundance levels of 136 proteins encoded by autosomal and X chromosome genes.

FIG. 21 shows differential protein expression between the tensor GSVD classes is consistent with the CNAs. Relative protein expression is shown for: (a) MAPK14, which is deleted, and (b) CDKN1B, which is amplified in the 6p+12p arraylet, are significantly (Mann-Whitney-Wilcoxon P-value<0.05) underexpressed and overexpressed, respectively, in the tensor GSVD class of a high 6p+12p x-probelet coefficient, or arraylet correlation relative to the tensor GSVD class of a low 6p+12p x-probelet coefficient, or arraylet correlation.

As seen in FIGS. 19-21, the CNAs are consistent with differential mRNA, microRNA, and protein expression between the tensor GSVD classes. The mRNA and protein encoded by, e.g., Mapk14, which is deleted in the 6p+12p arraylet, are both significantly (Mann-Whitney-Wilcoxon P-values<10⁻⁵) underexpressed in the tensor GSVD class of a high 6p+12p x-probelet coefficient, or arraylet correlation relative to the tensor GSVD class of a low 6p+12p x-probelet coefficient, or arraylet correlation. The microRNA mir-877* that maps to the same deletion as Mapk14 is also significantly (Mann-Whitney-Wilcoxon P-value<0.05) underexpressed.

### Example 5

### Discovery Datasets: Pairs of Column-Matched but Row-Independent Tensors

The discovery set of patients reflects the general primary, high-grade OV patient population, with approximately 5%, 7%, 76%, and 12% of the patients diagnosed at stages I, II, III, and IV, and 218, i.e., ˜88%, treated with platinum-based chemotherapy, i.e., cisplatin, carboplatin, or oxaliplatin, and 240 of the 249, i.e., >95% of the tumors at grades 2 and higher.

Each profile in the discovery datasets lists log₂ of TCGA level 1 background-subtracted intensity in the sample relative to the male Promega DNA reference, with signal to background≥2.5 for both the sample and reference in ≥90% of the 391,190 autosomal probes and ≥65% of the 10,911 X chromosome probes that match between the two Agilent Human array CGH (aCGH) DNA microarray platforms, G4447A and G4124A. Tumor and normal probes were selected with valid data in ≥99% of the tumor or normal arrays of each platform, respectively. For each chromosome arm or combination of two chromosome arms, and for each platform, the <0.5% missing data entries in the tumor and normal profiles were estimated by using the SVD, as previously described. Each profile was then centered at its copy-number median, and normalized by its copy-number sMAD.

### Tensor GSVD

Lemma A. The tensor GSVD exists for any two, e.g., third-order tensors Dᵢ ϵ [symbol] ^{K}^{ᵢ}^{×L×M} of the same column dimensions L and M but different row dimensions K₁, where Kᵢ≥LM for i=1, 2, if the tensors unfold into full column-rank matrices, Dᵢ ϵ [symbol] ^{K}^{ᵢ}^{×LM}, Dᵢₓ ϵ [symbol] ^{K}^{ᵢ}^{M×L}, and D_{iy} ϵ [symbol] ^{K}^{ᵢ}^{L×M}, each preserving the Kᵢ-row dimension, L-x-, or M-y-column dimension, respectively.

Proof.

The tensor GSVD of Eq. (1), of the pair of third-order tensors Dᵢ, is constructed from the GSVDs of Eqs. (2) and (3), of the pairs of full column-rank matrices Dᵢ, Dᵢₓ, and D_{iy}, where i=1, 2. From the existence of the GSVDs of Eqs. (2) and (3) [5, 6], the orthonormal column bases vectors of Uᵢ, as well as the normalized x- and y-row bases vectors of the invertible Vₓ^{T} or V_{y}^{T}, exist, and, therefore, the tensor GSVD of Eq. (1) also exists. Note that the proof holds for tensors of higher-than-third order.

Lemma B. The tensor GSVD has the same uniqueness properties as the GSVD.

Proof.

From the uniqueness properties of the GSVDs of Eqs. (2) and (3), the orthonormal column bases vectors u_{i,a}, and the normalized row bases vectors V_{x,b}^{T} and V_{y,c}^{T} of the tensor GSVD of Eq. (1) are unique, except in degenerate subspaces, defined by subsets of equal generalized singular values σᵢ, σᵢₓ, and σ_{iy}, respectively, and up to phase factors of ±1. The tensor GSVD, therefore, has the same uniqueness properties as the GSVD. Note that the proof holds for tensors of higher-than-third order.

For two second-order tensors, the tensor GSVD reduces to the GSVD of the corresponding matrices. Proof. For two second-order tensors, e.g., the matrices Dᵢ ϵ [symbol] ^{K}^{ᵢ}^{×L}, the tensor GSVD of Eq. (1) is

$${D}_{i} = {R}_{i} {\times }_{a} {U}_{i} {\times }_{b} {V}_{x} = {U}_{i} {R}_{i} {V}_{x}^{T} \qquad ( A1 )$$

The row- and x-column mode GSVDs of Eqs. (2) and (3) are identical, because unfolding each matrix Dᵢ while preserving either its Kᵢ-row dimension, or L-x-column dimension results in Dᵢ, up to permutations of either its columns or rows, respectively,

Dᵢ=UᵢΣᵢVₓ^{T}=Dᵢₓ,i−1,2. (A2)

From the uniqueness properties of the tensor GSVD of Eq. (A1), and the GSVDs of Eq. (A2) it follows that Rᵢ=Σᵢ, and that for two second-order tensors, i.e., matrices, the tensor GSVD is equivalent to the GSVD.

Theorem A. The tensor GSVD of the tensor D₁ϵ [symbol] ^{LM×L×M}, which row mode unfolding gives the identity matrix D₁=I ϵ [symbol] ^{LM×L×M}, and a tensor D₂ of the same column dimensions reduces to the HOSVD of D₂.

Proof.

Consider the GSVD of Eq. (2), of the matrices D₁=I and D₂, as computed by using the QR decomposition of the appended D₁ and D₂, and the SVD of the block of the resulting column-wise orthonormal Q that corresponds to D₂, i.e., Q₂=U_{Q}_{₂} Σ_{Q}_{₂} V_{Q}_{₂}^{T},

$$[ \begin{matrix}{D}_{1} \\ {D}_{2}\end{matrix} ] = [ \begin{matrix}I \\ {D}_{2}\end{matrix} ] = QR = {[ \begin{matrix}{Q}_{1} \\ {Q}_{2}\end{matrix} ]}^{R} = {[ \begin{matrix}{R}^{- 1} \\ {\sum }_{{Q}_{2}} {\sum }_{{Q}_{2}} {V}_{{Q}_{2}}^{T}\end{matrix} ]}^{R} , \qquad ( A3 )$$

where R is upper triangular and, therefore, invertible. Since Q is column-wise orthonormal, V_{Q}_{₂}^{T}, is orthonormal, and Σ_{Q}_{₂} is positive diagonal, it follows that

$$\begin{matrix}I = {Q}_{1}^{T} Q 1 + {Q}_{2}^{T} Q 2 \\ = {R}^{- T} {R}^{- 1} + ( {V}_{1} {\sum }_{Q 2}^{2} {V}_{Q 2}^{T} \\ = {( {V}_{Q 2}^{T} R )}^{- 1} + {( {V}_{Q 2}^{T} R )}^{- 1} + {\sum }_{Q 2}^{2} ,\end{matrix} \\ {( I - {\sum }_{Q 2}^{2} )}^{- 1} = ( {V}_{Q 2}^{T} R ) {( {V}_{Q 2}^{T} R )}^{T} , \qquad ( A4 )$$

and that

$${( I - {\sum }_{Q 2}^{2} )}^{\frac{1}{2}} {V}_{{Q}_{2}}^{T}$$

R is orthonormal. The GSVD of Eq. (2) factors the matrix D₂ into a column-wise or-thonormal U_{Q}_{₂}, a positive diagonal

$${\sum }_{{Q}_{2}} {( I - {\sum }_{Q 2}^{2} )}^{- \frac{1}{2}}$$

and an orthonormal

$${( I - {\sum }_{{Q }_{2}}^{2} )}^{\frac{1}{2}} {V}_{{Q}_{2}}^{T} R ,$$

and is, therefore, reduced to the SVD of D₂.

This proof holds for the GSVDs of Eq. (3). This is because the x- and y-column unfoldings of the tensor D₁ϵ [symbol] ^{LM×L×M}, which row mode unfolding gives the identity matrix D₁=ϵ [symbol] ^{LM×LM}, gives

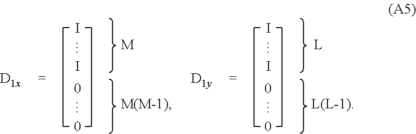

The GSVDs of Eqs. (2) and (3), of any one of the matrices D₁, D₁ₓ, or D_{1y} with the corresponding full column-rank matrices D₂, D₂ₓ, or D_{2y}, are, therefore, reduced to the SVDs of D₂, D₂ₓ, or D_{2y}, respectively.

The tensor GSVD of Eq. (1), where the orthonormal column bases vectors u_{2,a}, and the normalized row bases vectors v_{x,b}^{T}, and v_{y,c}^{T} in the factorization of the tensor D₂ are computed via the SVDs of the unfolded tensor is, therefore, reduced to the HOSVD of D₂. Note that the proof holds for tensors of higher-than-third order.

The “tensor generalized Shannon entropy” of each dataset,

0≤dᵢ=−(2 log LM)⁻¹Σₐ₌₁^{LM}Σ_{b=1}^{L}Σ_{c=1}^{M}P_{i,abc} log P_{i,abc}≤1,i=1,2, (Â6)

measures the complexity of each dataset from the distribution of the overall information among the different subtensors. An entropy of zero corresponds to an ordered and redundant dataset in which all the information is captured by a single subtensor. An entropy of one corresponds to a disordered and random dataset in which all subtensors are of equal significance.

### V. Sequence Listing

Table 3 below describes exemplary sequences for use herein. All sequences are human.

TABLE 3
Sequences

| SEQ ID NO | SEQ ID NO | |
|---|---|---|
| nucleic acid | amino acid | Description (gene, chromosome and cytogenetic band location) |
| 1 | 2 | Prim2 segment overlapping Prim2 gene, NC_000006.12; |
| | | chromosome 6p, segment 4, band 6p11.2; coordinates in hg19 |
| | | are chr6: 57,360,339-58,614,002 |
| 7 | 8 | Kras NC_000012.12; chromosome 12p, segment 12, band |
| | | 12p12.1-p11.23 |
| 11 | 12 | Sox5 NC_000012.12; chromosome 12p, segment 8-11, band |
| | | 12p12.1-p11.23 |
| 21 | 22 | Itpr2 NC_000012.12; chromosome 12p, segment 12, 13, band |
| | | 12p11.23 |
| 24 | 25 | Asun NC_000012.12; chromosome 12p, segment 13, 14, band |
| | | 12p11.23 |
| 25 | 26 | Rpa3 NC_000007.14; chromosome 7p, segment 4, band |
| | | 7p22.1-p21.3 |
| 27 | 28 | Pabpc5 NC_000023.11; chromosome Xq, segment 10, band |
| | | Xq21.31 |
| 29 | 30 | Dxs214 probe; sequence tag site chromosome Xq, segment 10, |
| | | band Xq21.31 |
| 31 | 32 | Cdkn1A NC_000006.12; chromosome 6p, segment 2, band |
| | | 6p25.3-p21.1 |
| 41 | 42 | Mapk14 NC_000006.12; chromosome 6p, segment 2, band |
| | | 6p25.3-p21.1 |
| 49 | 50 | Tnf NC_000006.12; chromosome 6p, segment 2, band 6p25.3- |
| | | p21.1 |
| 12 | 13 | miR-877 NC_000006.12; chromosome 6p, segment 2, band |
| | | 6p25.3-p21.1 |
| 52 | 53 | Abcf1 NC_000006.12; chromosome 6p, segment 2, band |
| | | 6p25.3-p21.1 |
| 56 | 57 | Rad51AP1 NC_000012.12; chromosome 12p, segment 5, 4, |
| | | band 12p13.33-p13.31 |
| 60 | 61 | miR-200c NC_000012.12; chromosome 12p, segment 5, band |
| | | 12p13.33-p13.31 |
| 61 | 62 | miR-141 NC_000012.12; chromosome 12p, segment 5, band |
| | | 12p13.33-p13.31 |
| 62 | 63 | Cdkn1B NC_000012.12; chromosome 12p, segment 7, band |
| | | 12p13.2-p12.3 |
| 64 | 65 | Pold2 NC_000007.14; chromosome 7p, segment 15, band |
| | | 7p14.1-p11.2 |
| 70 | 71 | Bcap31 NC_000023.11; chromosome Xq, segment 25, band |
| | | Xq27.3-q28 |
| 78 | 79 | miR-888 NC_000023.11; chromosome Xq, segment 25, band |
| | | Xq27.3-q28 |
| 79 | 80 | miR-224 NC_000023.11; chromosome Xq, segment 25, band |
| | | Xq27.3-q28 |
| 80 | 81 | miR-452 NC_000023.11; chromosome Xq, segment 25, band |
| | | Xq27.3-q28 |
| 81 | 82 | Gabre NC_000023.11; chromosome Xq, segment 25, band |
| | | Xq27.3-q28 |
| 83 | 84 | Bap1 NC_000003.12; chromosome 3p |
| 85 | 86 | Brca1 NC_000017.11; chromosome 17 |
| 96 | 97 | Lig4 NC_000013.11; chromosome 13 |
| | | chromosome 12p; segment 10; band 12p12.1-p11.23 |
| | | chromosome 12p; segment 11; band 12p12.1-p11.23 |
| | | chromosome 12p; segment 13; band 12p11.23 |
| | | chromosome 12p; segment 14; band 12p11.23 |
| | | chromosome 7p; segment 4; band 7p22.1-p21.3 |
| | | chromosome Xq; segment 10; band Xq21.31 |

The sequences provided in the table above are exemplary and variants which may exist are known to those of skill in the art. For example, some variants of the genes listed above are disclosed in the NCBI Reference Sequence Database (ncbi.nlm.nih.gov).

Affymetrix microarray probes, which are mapped to a known genomic coordinate, were used to determine differential expression. The UCSC genome browser was used to identify genes and genomic features for the regions identified as having differential expression. Exemplary sequences were obtained from the UCSC genome browser for the relevant genes and genomic features. It will be appreciated that the relevant genes and genomic features may include variations and alternative specific sequences as known in the art.

It will be also appreciated by persons skilled in the art that numerous variations and/or modifications may be made to the specific embodiments disclosed herein, without departing from the scope or spirit of the disclosure as broadly described. The present embodiments are, therefore, to be considered in all respects illustrative and not restrictive of the subject technology.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “about”, as used here, refers to +/−5% of a value.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

All publications and patents, and NCBI gene ID sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

| Number and Element | | Chromosome | | GenBank Accession | | |
|---|---|---|---|---|---|---|
| Type | | Segment | Name | Numbers | SEQ ID NO: | Organism |
| 1 | Gene | 6p11.2 | PRIM2 | NM_000947.4 | 1 | Homo sapiens |
| | | | | NP_000938.2 | 2 | Homo sapiens |
| | | | | NM_001282487.1 | 3 | Homo sapiens |
| | | | | NP_001269416.1 | 4 | Homo sapiens |
| | | | | NM_001282488.1 | 5 | Homo sapiens |
| | | | | NP_001269417.1 | 6 | Homo sapiens |
| 2 | Gene | 12p12.1-p11.23 | KRAS | NM_004985.4 | 7 | Homo sapiens |
| | | | | NP_004976.2 | 8 | Homo sapiens |
| | | | | NM_033360.3 | 9 | Homo sapiens |
| | | | | NP_203524.1 | 10 | Homo sapiens |
| 3 | Gene | 12p12.1-p11.23 | SOX5 | NM_001261414.1 | 11 | Homo sapiens |
| | | | | NP_001248343.1 | 12 | Homo sapiens |
| | | | | NM_001261415.1 | 13 | Homo sapiens |
| | | | | NP_001248344.1 | 14 | Homo sapiens |
| | | | | NM_006940.4 | 15 | Homo sapiens |
| | | | | NP_008871.3 | 16 | Homo sapiens |
| | | | | NM_152989.3 | 17 | Homo sapiens |
| | | | | NP_694534.1 | 18 | Homo sapiens |
| | | | | NM_178010.2 | 19 | Homo sapiens |
| | | | | NP_821078.1 | 20 | Homo sapiens |
| 4 | Gene | 12p11.23 | ITPR2 | NM_002223.3 | 21 | Homo sapiens |
| | | | | NP_002214.2 | 22 | Homo sapiens |
| 5 | Gene | 12p11.23 | ASUN | NM_018164.2 | 23 | Homo sapiens |
| | | | | NP_060634.2 | 24 | Homo sapiens |
| 6 | Gene | 7p22.1-p21.3 | RPA3 | NM_002947.4 | 25 | Homo sapiens |
| | | | | NP_002938.1 | 26 | Homo sapiens |
| 7 | Gene | Xq21.31 | PABPC5 | NM_080832.2 | 27 | Homo sapiens |
| | | | | NP_543022.1 | 28 | Homo sapiens |
| 8 | Sequence Tag Site | Xq21.31 | DXS214 | | 29 | probe |
| | | | | | 30 | probe |
| 9 | Gene | 6p25.3-p21.1 | CDKN1A | NM_000389.4 | 31 | Homo sapiens |
| | | | | NP_000380.1 | 32 | Homo sapiens |
| | | | | NM_001220777.1 | 33 | Homo sapiens |
| | | | | NP_001207706.1 | 34 | Homo sapiens |
| | | | | NM_001220778.1 | 35 | Homo sapiens |
| | | | | NP_001207707.1 | 36 | Homo sapiens |
| | | | | NM_001291549.1 | 37 | Homo sapiens |
| | | | | NP_001278478.1 | 38 | Homo sapiens |
| | | | | NM_078467.2 | 39 | Homo sapiens |
| | | | | NP_510867.1 | 40 | Homo sapiens |
| 10 | Gene | 6p25.3-p21.1 | MAPK14 | NM_001315.2 | 41 | Homo sapiens |
| | | | | NP_001306.1 | 42 | Homo sapiens |
| | | | | NM_139012.2 | 43 | Homo sapiens |
| | | | | NP_620581.1 | 44 | Homo sapiens |
| | | | | NM_139013.2 | 45 | Homo sapiens |
| | | | | NP_620582.1 | 46 | Homo sapiens |
| | | | | NM_139014.2 | 47 | Homo sapiens |
| | | | | NP_620583.1 | 48 | Homo sapiens |
| 11 | Gene | 6p25.3-p21.1 | TNF | NM_000594.3 | 49 | Homo sapiens |
| | | | | NP_000585.2 | 50 | Homo sapiens |
| 12 | microRNA | 6p25.3-p21.1 | miR-877* | NR_030615.1 | 51 | Homo sapiens |
| 13 | Gene | 6p25.3-p21.1 | ABCF1 | NM_001025091.1 | 52 | Homo sapiens |
| | | | | NP_001020262.1 | 53 | Homo sapiens |
| | | | | NM_001090.2 | 54 | Homo sapiens |
| | | | | NP_001081.1 | 55 | Homo sapiens |
| 14 | Gene | 12p13.33-p13.31 | RAD51AP1 | NM_001130862.1 | 56 | Homo sapiens |
| | | | | NP_001124334.1 | 57 | Homo sapiens |
| | | | | NM_006479.4 | 58 | Homo sapiens |
| | | | | NP_006470.1 | 59 | Homo sapiens |
| 15 | microRNA | 12p13.33-p13.31 | miR-200c, miR- | NR_029779.1 | 60 | Homo sapiens |
| 16 | microRNA | 12p13.33-p13.31 | miR-141, miR- | NR_029682.1 | 61 | Homo sapiens |
| 17 | Gene | 12p13.2-p12.3 | CDKN1B | NM_004064.4 | 62 | Homo sapiens |
| | | | | NP_004055.1 | 63 | Homo sapiens |
| 18 | Gene | 7p14.1-p11.2 | POLD2 | NM_001127218.2 | 64 | Homo sapiens |
| | | | | NP_001120690.1 | 65 | Homo sapiens |
| | | | | NM_001256879.1 | 66 | Homo sapiens |
| | | | | NP_001243808.1 | 67 | Homo sapiens |
| | | | | NM_006230.3 | 68 | Homo sapiens |
| | | | | NP_006221.2 | 69 | Homo sapiens |
| 19 | Gene | Xq27.3-q28 | BCAP31 | NM_001139441.1 | 70 | Homo sapiens |
| | | | | NP_001132913.1 | 71 | Homo sapiens |
| | | | | NM_001139457.2 | 72 | Homo sapiens |
| | | | | NP_001132929.1 | 73 | Homo sapiens |
| | | | | NM_001256447.1 | 74 | Homo sapiens |
| | | | | NP_001243376.1 | 75 | Homo sapiens |
| | | | | NM_005745.7 | 76 | Homo sapiens |
| | | | | NP_005736.3 | 77 | Homo sapiens |
| 20 | microRNA | Xq27.3-q28 | miR-888 | NR_030592.1 | 78 | Homo sapiens |
| 21 | microRNA | Xq27.3-q28 | miR-224 | NR_029638.1 | 79 | Homo sapiens |
| 22 | microRNA | Xq27.3-q28 | miR-452 | NR_029973.1 | 80 | Homo sapiens |
| 23 | Gene | Xq27.3-q28 | GABRE | NM_004961.3 | 81 | Homo sapiens |
| | | | | NP_004952.2 | 82 | Homo sapiens |
| 24 | Gene | | BAP1 | NM_004656.3 | 83 | Homo sapiens |
| | | | | NP_004647.1 | 84 | Homo sapiens |
| 25 | Gene | | BRCA1 | NM_007294.3 | 85 | Homo sapiens |
| | | | | NP_009225.1 | 86 | Homo sapiens |
| | | | | NM_007297.3 | 87 | Homo sapiens |
| | | | | NP_009228.2 | 88 | Homo sapiens |
| | | | | NM_007298.3 | 89 | Homo sapiens |
| | | | | NP_009229.2 | 90 | Homo sapiens |
| | | | | NM_007299.3 | 91 | Homo sapiens |
| | | | | NP_009230.2 | 92 | Homo sapiens |
| | | | | NM_007300.3 | 93 | Homo sapiens |
| | | | | NP_009231.2 | 94 | Homo sapiens |
| | | | | NR_027676.1 | 95 | Homo sapiens |
| 26 | Gene | | LIG4 | NM_001098268.1 | 96 | Homo sapiens |
| | | | | NP_001091738.1 | 97 | Homo sapiens |
| | | | | NM_002312.3 | 98 | Homo sapiens |
| | | | | NP_002303.2 | 99 | Homo sapiens |
| | | | | NM_206937.1 | 100 | Homo sapiens |
| | | | | NP_996820.1 | 101 | Homo sapiens |

What is claimed is:

1. A method of determining an estimated outcome of, or predicting a clinical response to, chemotherapy for a patient having ovarian serous cystadenocarcinoma (OV), comprising:
detecting, in a biological sample from a patient having OV, indicators of differential expression, between cancer cells and normal cells, of at least one of (a) at least two nucleic acid sequences selected from the group consisting of SEQ ID NO: 1, SEQ ID NO: 7, SEQ ID NO: 21, SEQ ID NO: 25, SEQ ID NO: 27, SEQ ID NO: 29, SEQ ID NO: 31, SEQ ID NO: 41, SEQ ID NO: 47, SEQ ID NO: 56, SEQ ID NO: 64, SEQ ID NO: 70, SEQ ID NO: 81, SEQ ID NO: 96; (b) amino acid sequences encoded by the nucleic acid sequences of (a); or (c) sequences of at least two microRNAs selected from the group consisting of SEQ ID NO: 51, SEQ ID NO: 60, SEQ ID NO: 61, SEQ ID NO: 78, SEQ ID NO: 79, and SEQ ID NO: 80;
calculating, by a processor, a weighted sum based on the value of the indicators of differential expression; and
estimating, by the processor and based on the weighted sum, a predicted length of survival of the patient or a predicted clinical response to chemotherapy for the patient.

2. The method of claim 1, further comprising recommending administering a treatment based on the predicted length of survival or the predicted clinical response.

3. The method of claim 1, further comprising recommending a treatment regimen based on the predicted length of survival or the predicted clinical response.

4. The method of claim 1, wherein differential expression for the nucleic acid sequences is differential copy numbers of nucleic acid sequences in cancer cells relative to normal cells.

5. The method of claim 4, wherein the differential copy number is an increase in copy number in cancer cells relative to normal cells.

6. The method of claim 1, wherein the amino acid sequences are selected from the group consisting of SEQ ID NO: 8, SEQ ID NO: 22, SEQ ID NO: 26, SEQ ID NO: 28, SEQ ID NO: 32, SEQ ID NO: 42, SEQ ID NO: 50, SEQ ID NO: 57, SEQ ID NO: 65, SEQ ID NO: 71, SEQ ID NO: 82, and SEQ ID NO: 97;
wherein the indicator of differential expression is of the amino acid sequences.

7. The method of claim 1, wherein the predicted length of survival or the predicted clinical response is based on members of each of at least one set of indicators of differential expression selected from sets (a)-(e) below:
a. co-occurring copy-number loss of SEQ ID NO: 27 and gain, or mRNA overexpression of SEQ ID NO: 70; or
b. co-occurring (i) copy number loss of SEQ ID NO: 27 and (ii) gain, or mRNA overexpression of SEQ ID NO: 70, and (iii) gain, or microRNA overexpression of SEQ ID NO: 78, and SEQ ID NO: 80; or
c. co-occurring copy number loss of SEQ ID NO: 27, and gain, or mRNA overexpression of SEQ ID NO: 70, and gain, or microRNA overexpression of SEQ ID NO: 78, SEQ ID NO: 80, SEQ ID NO: 79; or
d. co-occurring copy-number loss of SEQ ID NO: 27 SEQ ID NO: 29, and gain, or mRNA overexpression of SEQ ID NO: 70; or
e. co-occurring copy number loss of SEQ ID NO: 27, and gain, or mRNA overexpression of SEQ ID NO: 70 and SEQ ID NO: 81.

8. The method of claim 7, wherein the predicted length of survival or the predicted clinical response is based on (c) and further based on (i) copy-number gain or loss of SEQ ID NO: 29, or (ii) mRNA overexpression of SEQ ID NO: 70 and SEQ ID NO: 81.

9. The method of claim 1, wherein the predicted length of survival or the predicted clinical response is based on members of each of at least one set of indicators of differential expression selected from sets (a1)-(d1) below:
a1) co-occurring copy-number loss, or mRNA underexpression of SEQ ID NO: 25, and copy-number gain, or mRNA overexpression of SEQ ID NO: 64 or
b1) co-occurring copy-number loss, or mRNA underexpression of SEQ ID NO: 25 and SEQ ID NO: 96, and copy-number gain, or mRNA overexpression of SEQ ID NO: 64; or
c1) co-occurring copy-number loss, or mRNA underexpression of SEQ ID NO: 96 on chromosome 13q, and copy-number gain, or mRNA overexpression of SEQ ID NO: 64;
d1) co-occurring copy-number loss from SEQ ID NO: 1, SEQ ID NO: 7, SEQ ID NO: 10, SEQ ID NO: 21, SEQ ID NO: 23, SEQ ID NO: 25, SEQ ID NO: 27, and copy number gain in SEQ ID NO: 39, SEQ ID NO: 51, SEQ ID NO: 52, SEQ ID NO: 56, SEQ ID NO: 60, SEQ ID NO: 61, and SEQ ID NO: 62.

10. The method of claim 1, wherein the predicted length of survival or the predicted clinical response is based on members of each of at least one set of indicators of differential expression selected from sets (a2)-(g2) below:
a2) co-occurring copy-number loss on chromosome 6p and gain on chromosome 12p; or
b2) co-occurring copy-number loss, or mRNA or protein under-expression of SEQ ID NO: 31 and SEQ ID NO: 4, and copy-number gain, or mRNA or protein overexpression of SEQ ID NO: 7; or
c2) co-occurring copy-number loss, or mRNA or protein under-expression of SEQ ID NO: 31 and SEQ ID NO: 41, and copy-number gain, or mRNA or protein overexpression SEQ ID NO: 7 and SEQ ID NO: 56; or
d2) co-occurring copy-number loss, or mRNA or protein under-expression of SEQ ID NO: 31, SEQ ID NO: 41 and SEQ ID NO: 39, and copy-number gain, or mRNA or protein overexpression of SEQ ID NO: 7, SEQ ID NO: 56, and SEQ ID NO: 21; or
e2) co-occurring copy-number loss, or microRNA under-expression of SEQ ID NO: 51, and copy-number gain, or microRNA overexpression, of SEQ ID NO: 60, or SEQ ID NO: 61;
(f2) co-occurring copy-number loss, or mRNA or protein under-expression of SEQ ID NO: 31 and SEQ ID NO: 41, and copy-number gain, or mRNA or protein overexpression of SEQ ID NO: 56;
(g2) co-occurring copy-number loss, or mRNA or protein under-expression of SEQ ID NO: 39, and copy-number gain, or mRNA or protein overexpression of SEQ ID NO: 21.

11. The method of claim 10, wherein the predicted length of survival or the predicted clinical response is based on members of each of at the least one set of indicators selected from sets (a2)-(g2) and is further based on members of each of at least one set of indicators of differential expression selected from
(h2) a gain in copy numbers or mRNA or protein overexpression of SEQ ID NO: 10; or
(i2) a gain in copy numbers or mRNA or protein overexpression of SEQ ID NO: 23; or
(j2) a gain in copy numbers or mRNA or protein overexpression of SEQ ID NO: 52; or
(k2) a gain in copy numbers or mRNA or protein overexpression of SEQ ID NO: 62; or
(l2) a mRNA or protein under-expression or loss in copy numbers of SEQ ID NO: 83; or
(m2) a reduced abundance of Brca1 (SEQ ID NO: 85)-associated genome surveillance protein complex (BASC).

12. A method of determining an estimated outcome or predicting a clinical response to chemotherapy for a patient having ovarian serous cystadenocarcinoma (OV), comprising;
detecting, in a biological sample from a patient having OV, indicators of differential copy numbers, between cancer cells and normal cells, of at least one of (a) at least two nucleic acid sequences selected from the group consisting of SEQ ID NO: 1, SEQ ID NO: 7, SEQ ID NO: 21, SEQ ID NO: 25, SEQ ID NO: 27, SEQ ID NO: 29, SEQ ID NO: 31, SEQ ID NO: 41, SEQ ID NO: 47, SEQ ID NO: 56, SEQ ID NO: 64, SEQ ID NO: 70, SEQ ID NO: 81, SEQ ID NO: 96; (b) amino acid sequences encoded by the nucleic acid sequences of (a); or (c) sequences of at least two microRNAs selected from the group consisting of SEQ ID NO: 51, SEQ ID NO: 60, SEQ ID NO: 61, SEQ ID NO: 78, SEQ ID NO: 79, and SEQ ID NO: 80; and
calculating, by a processor, a weighted sum based on the value of the indicators of differential copy numbers; and
estimating, by the processor and based on the weighted sum, a predicted length of survival of the patient or a predicted clinical response to chemotherapy for the patient.

13. The method of claim 12, wherein the nucleic acid sequences are selected from SEQ ID NO: 31, SEQ ID NO: 41, SEQ ID NO: 39, SEQ ID NO: 64, SEQ ID NO: 70.

14. The method of claim 12, wherein the nucleic acid sequences are selected from SEQ ID NO: 56, SEQ ID NO: 62, SEQ ID NO: 7, SEQ ID NO: 21, SEQ ID NO: 25, SEQ ID NO: 27.

15. The method of claim 12, wherein copy numbers of the nucleic acid sequences are selected from SEQ ID NO: 56, SEQ ID NO: 62, SEQ ID NO: 7, SEQ ID NO: 21, SEQ ID NO: 25, SEQ ID NO: 27.

16. The method of claim 12, wherein copy numbers of the nucleic acid sequences are selected from SEQ ID NO 31, SEQ ID NO: 41, SEQ ID NO: 39, SEQ ID NO: 64, SEQ ID NO: 70.

17. The method of claim 12, wherein the predicted length of survival or the predicted clinical response is based a combination of (a) a decrease in copy numbers of SEQ ID NO: 31 and SEQ ID NO: 41 in cancer cells relative to the copy numbers of SEQ ID NO: 31 and SEQ ID NO: 41 in normal cells; and (b) an increase in copy numbers of SEQ ID NO: 7 and SEQ ID NO: 56 in cancer cells relative to the copy number of SEQ ID NO 7 and SEQ ID NO 56, reflecting a decreased length of survival relative to a length of survival of patients without this pattern of increased and decreased copy number.

18. The method of claim 12, wherein the predicted length of survival or the predicted clinical response is based a combination of (a) a decrease in SEQ ID NO: 25 copy number relative to the SEQ ID NO: 25 copy number in normal cells; and (b) an increase in SEQ ID NO: 64 copy number relative to the SEQ ID NO: 64 copy number in normal cells; reflecting an increased length of survival relative to the length of survival of patients without this pattern of increased and decreased copy number.

19. The method of claim 12, wherein the combination of (a) a decrease in SEQ ID NO: 27 copy number relative to the SEQ ID NO: 27 copy number in normal cells; and (b) an increase in SEQ ID NO: 70 copy number relative to the SEQ ID NO: 70 copy number in normal cells; reflects an increased length of survival relative to length of survival of patients without this pattern of increased and decreased copy number.

20. The method of claim 12, wherein the estimating further comprises evaluating at least one of tumor stage at diagnosis, residual disease after surgery, therapy outcome, or neoplasm status.

21. A method for treating a patient having ovarian serous cystadenocarcinoma (OV), comprising:
administering, in a patient having OV, a treatment based on a predicted length of survival or a predicted clinical response to chemotherapy, wherein the predicted length of survival or the predicted response to chemotherapy is determined by:
(1) detecting, in a biological sample from a patient having OV, indicators of differential expression, between cancer cells and normal cells, of at least one of (a) at least two nucleic acid sequences selected from the group consisting of SEQ ID NO: 1, SEQ ID NO: 7, SEQ ID NO: 21, SEQ ID NO: 25, SEQ ID NO: 27, SEQ ID NO: 29, SEQ ID NO: 31, SEQ ID NO: 41, SEQ ID NO: 47, SEQ ID NO: 56, SEQ ID NO: 64, SEQ ID NO: 70, SEQ ID NO: 81, SEQ ID NO: 96; (b) amino acid sequences encoded by the nucleic acid sequences of (a); or (c) at least two microRNA sequences selected from the group consisting of SEQ ID NO: 51, SEQ ID NO: 60, SEQ ID NO: 61, SEQ ID NO: 78, SEQ ID NO: 79, and SEQ ID NO: 80;
(2) calculating, by a processor, a weighted sum based on the value of the indicators of differential expression; and
(3) estimating, by the processor and based on the weighted sum, a predicted length of survival of the patient or a predicted clinical response to chemotherapy for the patient.

22. The method of claim 21, wherein the indicator of differential expression for the nucleic acid sequences is differential copy numbers in cancer cells relative to in normal cells.

23. The method of claim 21, wherein the amino acid sequences are of proteins selected from SEQ ID NO: 8, SEQ ID NO: 22, SEQ ID NO: 26, SEQ ID NO: 28, SEQ ID NO: 32, SEQ ID NO: 42, SEQ ID NO: 50, SEQ ID NO: 57, SEQ ID NO: 65, SEQ ID NO: 71, and SEQ ID NO: 82, SEQ ID NO: 97; and
wherein the indicator of differential expression indicates differential protein expression in cancer cells relative to normal cells.

24. The method of claim 21, wherein the microRNA sequences are selected from the group consisting of SEQ ID NO: 51, SEQ ID NO: 60, SEQ ID NO: 61, SEQ ID NO: 78, SEQ ID NO: 79, and SEQ ID NO: 80.

25. The method of claim 21, wherein the microRNA sequences are selected from the group consisting of SEQ ID NO: 51, SEQ ID NO: 60, SEQ ID NO: 61, SEQ ID NO: 78, SEQ ID NO: 79, and SEQ ID NO: 80; and
wherein the indicator of differential expression is differential microRNA expression in cancer cells relative to normal cells.
